(19) United States
(12) Patent Application Publication
(10) Pub. No.: US 2009/0036474 A1
(43) Pub. Date: Feb. 5, 2009

(54) Quinazoline derivatives for use against cancer

(76) Inventors: Patrick Ple, Cedex (FR); Frederic Henri Jung, Cedex (FR)

Correspondence Address: MORGAN LEWIS & BOCKIUS LLP, 1111 PENNSYLVANIA AVENUE NW, WASHINGTON, DC, 20004, US

(21) Appl. No.: 11/665,115

(22) PCT Filed: Oct. 7, 2005
(86) PCT No.: PCT/GB05/03881

§ 371 Date: Apr. 11, 2007

(30) Foreign Application Priority Data
Oct. 12, 2004 (EP) 04292418.3

(51) Int. Cl. A61K 31/517; C07D 403/12; A61P 9/00

(52) U.S. Cl. 514/266.23; 544/284

(57) ABSTRACT

The invention concerns quinazoline derivatives of Formula (I) or a pharmaceutically-acceptable salt, solvate or pro-drug thereof, wherein each of p, R¹, q, R², R³, R⁴, R⁵, Ring A, X¹, R⁶, r and R⁷ has any of the meanings defined in the description; processes for their preparation, pharmaceutical compositions containing them and their use in the manufacture of a medicament for use in the treatment of cell proliferative disorders or in the treatment of disease states associated with angiogenesis and/or vascular permeability.

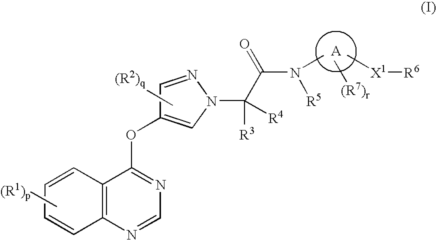

The invention concerns certain novel quinazoline derivatives, or pharmaceutically-acceptable salts thereof, which possess anti-cancer activity and are accordingly useful in methods of treatment of the human or animal body. The invention also concerns processes for the manufacture of said quinazoline derivatives, to pharmaceutical compositions containing them and to their use in therapeutic methods, for example in the manufacture of medicaments for use in the prevention or treatment of cancers in a warm-blooded animal such as man, including use in the prevention or treatment of solid tumour disease.

Many of the current treatment regimes for the abnormal cell growth found in cell proliferation diseases such as psoriasis and cancer utilise compounds which inhibit DNA synthesis. Such compounds are toxic to cells generally but their toxic effect on rapidly dividing cells such as tumour cells can be beneficial. Alternative approaches to anti-cancer agents which act by mechanisms other than the inhibition of DNA synthesis have the potential to display enhanced selectivity of action.

Eukaryotic cells are continually responding to many diverse extracellular signals that enable communication between cells within an organism. These signals regulate a wide variety of physical responses in the cell including proliferation, differentiation, apoptosis and motility. The extracellular signals take the form of a diverse variety of soluble factors including growth factors as well as paracrine, autocrine and endocrine factors. By binding to specific transmembrane receptors, growth factor ligands communicate extracellular signals to the intracellular signalling pathways, thereby causing the individual cell to respond to extracellular signals. Many of these signal transduction processes utilise the reversible process of the phosphorylation of proteins involving specific kinases and phosphatases.

As phosphorylation is such an important regulatory mechanism in the signal transduction process, it is not surprising that aberrations in the process result in abnormal cell differentiation, transformation and growth. For example, it has been discovered that a cell may become cancerous by virtue of the transformation of a portion of its DNA into an oncogene. Several such oncogenes encode proteins which are receptors for growth factors, for example tyrosine kinase enzymes. Tyrosine kinases may also be mutated to constitutively active forms that result in the transformation of a variety of human cells. Alternatively, the over-expression of normal tyrosine kinase enzymes may also result in abnormal cell proliferation.

Tyrosine kinase enzymes may be divided into two groups: —the receptor tyrosine kinases and the non-receptor tyrosine kinases. About 90 tyrosine kinase have been identified in the human genome, of which about 60 are of the receptor type and about 30 are of the non-receptor type. These can be categorised into 20 receptor tyrosine kinase sub-families according to the families of growth factors that they bind and into 10 non-receptor tyrosine kinase sub-families (Robinson et al, Oncogene, 2000, 19, 5548-5557). The classification includes the EGF family of receptor tyrosine kinases such as the EGF, TGFα, Neu and erbB receptors, the insulin family of receptor tyrosine kinases such as the insulin and IGF1 receptors and insulin-related receptor (IRR) and the Class III family of receptor tyrosine kinases such as the platelet-derived growth factor (PDGF) receptor tyrosine kinases, for example the PDGFα and PDGFβ receptors, the stem cell factor receptor tyrosine kinase (SCF RTK (commonly known as c-Kit), the fms-related tyrosine kinase 3 (Flt3) receptor tyrosine kinase and the colony-stimulating factor 1 receptor (CSF-1R) tyrosine kinase.

It has been discovered that such mutated and over-expressed forms of tyrosine kinases are present in a large proportion of common human cancers such as the leukaemias, breast cancer, prostate cancer, non-small cell lung cancer (NSCLC) including adenocarcinomas and squamous cell cancer of the lung, gastrointestinal cancer including colon, rectal and stomach cancer, bladder cancer, oesophageal cancer, ovarian cancer and pancreatic cancer. As further human tumour tissues are tested, it is expected that the widespread prevalence and relevance of tyrosine kinases will be further established. For example, it has been shown that EGFR tyrosine kinase is mutated and/or over-expressed in several human cancers including in tumours of the lung, head and neck, gastrointestinal tract, breast, oesophagus, ovary, uterus, bladder and thyroid.

Platelet-derived growth factor (PDGF) is a major mitogen for connective tissue cells and other cell types. The PDGF receptors comprising PDGFα and PDGFβ receptor isozymes display enhanced activity in blood vessel disease (for example atherosclerosis and restenosis, for example in the process of restenosis subsequent to balloon angioplasty and heart arterial by-pass surgery). Such enhanced PDGF receptor kinase activity is also observed in other cell proliferative disorders such as fibrotic diseases (for example kidney fibrosis, hepatic cirrhosis, lung fibrosis and multicystic renal dysplasia), glomerulonephritis, inflammatory diseases (for example rheumatoid arthritis and inflammatory bowel disease), multiple sclerosis, psoriasis, hypersensitivity reactions of the skin, allergic asthma, insulin-dependent diabetes, diabetic retinopathy and diabetic nephropathy.

The PDGF receptors can also contribute to cell transformation in cancers and leukaemias by autocrine stimulation of cell growth. It has been shown that PDGF receptor kinases are mutated and/or over-expressed in several human cancers including in tumours of the lung (non-small cell lung cancer and small cell lung cancer), gastrointestine (such as colon, rectal and stomach tumours), prostate, breast, kidney, liver, brain (such as glioblastoma), oesophagus, ovary, pancreas and skin (such as dermatofibrosarcoma protruberans) and in leukaemias and lymphomas such as chronic myelogenous leukaemia (CML), chronic myelomonocytic leukaemia (CMML), acute lymphocyte leukaemia (ALL) and multiple myeloma. Enhanced cell signalling by way of the PDGF receptor tyrosine kinases can contribute to a variety of cellular effects including cell proliferation, cellular mobility and invasiveness, cell permeability and cellular apoptosis.

Accordingly, antagonism of the activity of PDGF receptor kinases is expected to be beneficial in the treatment of a number of cell proliferative disorders such as cancer, especially in inhibiting tumour growth and metastasis and in inhibiting the progression of leukaemia.

In addition, PDGF is involved in angiogenesis, the process of forming new blood vessels, that is critical for continuing tumour growth. Normally, angiogenesis plays an important role in processes such as embryonic development, wound healing and several components of female reproductive function. However, undesirable or pathological angiogenesis has been associated with a number of disease states including diabetic retinopathy, psoriasis, cancer, rheumatoid arthritis, atheroma, Kaposi's sarcoma and haemangioma. Angiogenesis is stimulated via the promotion of the growth of endothelial cells. Several polypeptides with in vitro endothelial cell growth promoting activity have been identified including acidic and basic fibroblast growth factors (aFGF and bFGF) and vascular endothelial growth factor (VEGF). By virtue of the restricted expression of its receptors, the growth factor activity of VEGF, in contrast to that of aFGF and bFGF, is relatively specific towards endothelial cells. Recent evidence indicates that VEGF is an important stimulator of both normal and pathological angiogenesis and vascular permeability. This cytokine induces a vascular sprouting phenotype by inducing endothelial cell proliferation, protease expression and migration which subsequently leads to the formation of capillary tubes that promote the formation of the hyperpermeable, immature vascular network which is characteristic of pathological angiogenesis. The receptor tyrosine kinase (RTK) sub-family that binds VEGF comprises the kinase insert domain-containing receptor KDR (also referred to as Flk-1), the fms-like tyrosine kinase receptor Flt-1 and the fms-like tyrosine kinase receptor Flt-4. Two of these related RTKs, namely Flt-1 and KDR, have been shown to bind VEGF with high affinity.

Accordingly, antagonism of the activity of VEGF is expected to be beneficial in the treatment of a number of disease states that are associated with angiogenesis and/or increased vascular permeability such as cancer, especially in inhibiting the development of tumours.

It is disclosed in International Patent Application WO 00/21955 that certain 4-(3-pyrazolyloxy)- and 4-(3-pyrazolylamino)-quinazoline derivatives possess antiangiogenic and/or vascular permeability reducing activity based on antagonism of the activity of VEGF. There is no mention therein of 4-(4-pyrazolyloxy)quinazoline derivatives.

It is known that several compounds with PDGF receptor kinase inhibitory activity are progressing toward clinical development. The 2-anilinopyrimidine derivative known as imatinib (STI571; Nature Reviews, 2002, 1, 493-502; Cancer Research, 1996, 56, 100-104) has been shown to inhibit PDGF receptor kinase activity although its current clinical use is for the treatment of CML based on its additional activity as an inhibitor of BCR-ABL kinase. STI571 inhibits the growth of glioblastoma tumours arising from injection into the brains of nude mice of the human glioblastoma lines U343 and U87 (Cancer Research, 2000, 60, 5143-5150). The compound also inhibits the in vivo growth of dermatofibrosarcoma protruberans cell cultures (Cancer Research, 2001, 61, 5778-5783). Based on the PDGF receptor kinase inhibitory activity of the compound, clinical trials are being carried out in glioblastoma and in prostate cancer. Several other PDGF receptor kinase inhibitors are being investigated including quinoline, quinazoline and quinoxaline derivatives (Cytokine & Growth Factor Reviews, 2004, 15, 229-235).

It is further known from International Patent Application WO 92/20642 that certain aryl and heteroaryl compounds inhibit EGF and/or PDGF receptor tyrosine kinase. There is the disclosure of certain quinazoline derivatives therein but no mention is made of 4-(4-pyrazolyloxy)quinazoline derivatives.

It is stated in U.S. Pat. No. 5,476,851 that certain pyrazolo[3,4-g]quinoxaline derivatives possess PDGF receptor kinase inhibitory activity.

It is stated in International Patent Application WO 01/40217 that certain N-(2-quinolyl)benzimidazole derivatives are selective inhibitors of PDGF receptor kinase that are useful in the treatment of cell proliferation disorders.

It is stated in International Patent Application WO 02/12242 that certain bicyclic pyrazole derivatives are useful for treating diseases linked to disregulated protein kinases and in International Patent Application WO 03/097609 that certain tricyclic 3-aminopyrazole derivatives possess PDGF receptor kinase inhibitory activity.

As stated above, although STI571 is the only compound with PDGF receptor kinase inhibitory activity that appears to have yet reached the market, that compound possesses approximately equipotent activity against various other kinase enzymes. There is still a need for further compounds with PDGF receptor kinase inhibitory activity that may be useful for the treatment of cell proliferation disorders such as cancer.

We have now found that surprisingly certain novel 4-(4-pyrazolyloxy)quinazoline derivatives possess potent activity against cell proliferative disorders. It is believed that the compounds provide a useful treatment of cell proliferative disorders, for example to provide an anti-tumour effect, by way of a contribution from inhibition of PDGF receptor tyrosine kinases.

A further characteristic of hyperproliferative diseases such as cancer is damage to the cellular pathways that control progress through the cell cycle which, in normal eukaryotic cells, involves an ordered cascade of protein phosphorylation. As for signal transduction mechanisms, several families of protein kinases appear to play critical roles in the cell cycle cascade. The most widely studied of these cell cycle regulators is the cyclin dependent kinase family (the CDKs). Activity of specific CDKs at specific times is essential both to initiate and coordinate progress through the cell cycle. For example, the CDK4 protein appears to control entry into the cell cycle (the G0-G1-S transition) by phosphorylating the retinoblastoma gene product pRb which stimulates the release of the transcription factor E2F from pRb which, in turn, acts to increase the transcription of genes necessary for entry into S phase. The catalytic activity of CDK4 is stimulated by binding to a partner protein, Cyclin D. One of the first demonstrations of a direct link between cancer and the cell cycle was made with the observation that the Cyclin D1 gene was amplified and Cyclin D protein levels increased in many human tumours.

More recently, protein kinases that are structurally distinct from the CDK family have been identified which play critical roles in regulating the cell cycle and which also appear to be important in oncogenesis. They include the human homologues of the Drosophila aurora and S. cerevisiae Ipll proteins. The three human homologues of these genes Aurora-A, Aurora-B and Aurora-C encode cell cycle regulated serine-threonine protein kinases that show a peak of expression and kinase activity through G2 and mitosis. Several observations implicate the involvement of human aurora proteins in cancer, especially Aurora-A and Aurora-B. Abrogation of Aurora-A expression and function by antisense oligonucleotide treatment of human tumour cell lines leads to cell cycle arrest and exerts an anti-proliferative effect. Additionally, small molecule inhibitors of Aurora-A and Aurora-B have been demonstrated to have an anti-proliferative effect in human tumour cells.

It is disclosed in International Patent Application WO 02/00649 that certain quinazoline derivatives that carry a 5-membered heteroaryl group linked to the 4-position of the quinazoline ring by a NH group possess Aurora kinase inhibitory activity. There is no mention therein of 4-(4-pyrazolyloxy)quinazoline derivatives. It is disclosed in International Patent Applications WO 03/055491 and WO 04/058781 that certain 4-(3-pyrazolylamino)quinazoline derivatives possess Aurora kinase inhibitory activity. There is no mention therein of 4-(4-pyrazolyloxy)quinazoline derivatives.

It is stated in International Patent Application PCT/GB2004/001614 (published subsequently as WO 2004/094410) that certain 4-(4-pyrazolylamino)quinazoline derivatives possess Aurora kinase inhibitory activity. There is no disclosure therein of 4-(4-pyrazolyloxy)quinazoline derivatives.

As stated above, we have now found that surprisingly certain novel 4-(4-pyrazolyloxy)quinazoline derivatives possess potent activity against cell proliferative disorders. Without wishing to imply that the compounds disclosed in the present invention possess pharmacological activity only by virtue of an effect on one or two biological processes, it is believed that the compounds provide a useful treatment of cell proliferative disorders, for example to provide an anti-tumour effect, by way of a contribution from inhibition of PDGF receptor tyrosine kinases. In particular, it is believed that the compounds of the present invention provide a useful treatment of cell proliferative disorders by way of a contribution from inhibition of the PDGFα and/or PDGFβ receptor tyrosine kinases.

Generally the compounds of the present invention possess potent inhibitory activity against the PDGF receptor family of tyrosine kinases, for example the PDGFα and/or PDGFβ receptor tyrosine kinases, and against VEGF receptor tyrosine kinases, for example KDR and Flt-1, whilst possessing less potent inhibitory activity against other tyrosine kinase enzymes such as the EGF receptor tyrosine kinase. Furthermore, certain compounds of the present invention possess substantially better potency against the PDGF receptor family of tyrosine kinases, particularly against the PDGFβ receptor tyrosine kinase, and against VEGF receptor tyrosine kinases, particularly against KDR, than against EGF receptor tyrosine kinase. Such compounds possess sufficient potency that they may be used in an amount sufficient to inhibit the PDGF receptor family of tyrosine kinases, particularly PDGFβ receptor tyrosine kinase, and to inhibit VEGF receptor tyrosine kinases, particularly KDR, whilst demonstrating little activity against EGF receptor tyrosine kinase.

According to one aspect of the invention there is provided a quinazoline derivative of the Formula I

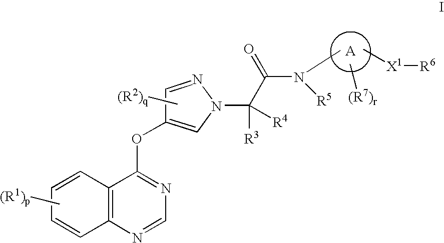

wherein p is 0, 1, 2 or 3;

each R¹ group, which may be the same or different, is selected from halogeno, trifluoromethyl, cyano, hydroxy, mercapto, amino, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino and di-[(1-6C)alkyl]amino,

or from a group of the formula:

Q¹-X²—

wherein X² is a direct bond or is selected from O, S, SO, SO₂, N(R⁸), CO, CON(R⁸), N(R⁸)CO, OC(R⁸)₂ and N(R⁸)C(R⁸)₂, wherein each R⁸ is hydrogen or (1-8C)alkyl, and Q¹ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, (3-8C)cycloalkenyl, heteroaryl or heterocyclyl group within a R¹ substituent optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

—X³—R⁹

wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl or N,N′,N′-tri-[(1-6C)alkyl]ureido-(1-6C)alkyl, or from a group of the formula:

—X⁴-Q²

wherein X⁴ is a direct bond or is selected from O, CO and N(R¹¹), wherein. R¹¹ is hydrogen or (1-8C)alkyl, and Q² is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,

and wherein any aryl, heteroaryl or heterocyclyl group within a substituent on R¹ optionally bears a (1-3C)alkylenedioxy group,

and wherein any heterocyclyl group within a R¹ substituent optionally bears 1 or 2 oxo or thioxo substituents,

and wherein any CH, CH₂ or CH₃ group within a R¹ substituent optionally bears on each said CH, CH₂ or CH₃ group one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′1′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within a R¹ substituent are optionally separated by the insertion into the chain of a group selected from O, S, SO, SO₂, N(R¹²), CO, CH(OR¹²), CON(R¹²), N(R¹²)CO, N(R¹²)CON(R¹²), SO₂N(R¹²), N(R¹²)SO₂, CH═CH and C≡C wherein R¹² is hydrogen or (1-8C)alkyl, or, when the inserted group is N(R¹²), R¹² may also be (2-6C)alkanoyl;

q is 0, 1 or 2;

each R² group, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino and di-[(1-6C)alkyl]amino;

R³ is hydrogen, (1-8C)alkyl, (2-8C)alkenyl or (2-8C)alkynyl;

R⁴ is hydrogen, (1-8C)alkyl, (2-8C)alkenyl, (2-8C)alkynyl, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl, di-[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkoxycarbonyl-(1-6C)alkyl, (2-6C)alkanoylamino-(1-6C)alkyl or N-(1-6C)alkyl-(2-6C)alkanoylamino-(1-6C)alkyl;

or R³ and R⁴ together with the carbon atom to which they are attached form a (3-8C)cycloalkyl group;

R⁵ is hydrogen, (1-8C)alkyl, (2-8C)alkenyl or (2-8C)alkynyl or a group of the formula:

—X⁵—R¹³

wherein X⁵ is a direct bond or is selected from O and N(R¹⁴), wherein R¹⁴ is hydrogen or (1-8C)alkyl, and R¹³ is halogeno-(1-6C)alkyl, hydroxy-(1-6C)alkyl, (1-6C)alkoxy-(1-6C)alkyl or cyano-(1-6C)alkyl;

Ring A is a 6-membered monocyclic or a 10-membered bicyclic aryl ring or a 5- or 6-membered monocyclic or a 9- or 10-membered bicyclic heteroaryl ring with up to three ring heteroatoms selected from oxygen, nitrogen and sulphur;

X¹ is a direct bond or is selected from O, S, SO, SO₂, N(R¹⁵), CO, CH(OR¹⁵), CON(R¹⁵), N(R¹⁵)CO, N(R¹⁵)CON(R¹⁵), SO₂N(R¹⁵), N(R¹⁵)SO₂, C(R¹⁵)₂O, C(R¹⁵)₂S, C(R¹⁵)₂N(R¹⁵) and C(R¹⁵)₂C(R¹⁵)₂N(R¹⁵), wherein each R¹⁵ is hydrogen or (1-8C)alkyl;

R⁶ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, carboxy-(1-6C)alkyl, (1-6C)alkoxycarbonyl-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulphamoyl-(1-6C)alkyl, N-(1-6C)alkylsulphamoyl-(1-6C)alkyl, N,N-di-[(1-6C)alkyl]sulphamoyl-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl, N,N′,N′-tri-[(1-6C)alkyl]ureido-(1-6C)alkyl, (1-6C)alkanesulphonylamino-(1-6C)alkyl or N-(1-6C)alkyl-(1-6C)alkanesulphonylamino-(1-6C)alkyl,

or R⁶ is al, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,

or, when X¹ is a direct bond, R⁶ may be carboxy, (1-6C)alkoxycarbonyl, sulphamoyl, N-(1-6C)alkylsulphamoyl or N,N-di-[(1-6C)alkyl]sulphamoyl,

or the —X¹—R⁶ group and one R⁷ group together form a bivalent group that spans adjacent ring positions on Ring A selected from OC(R²⁰)₂O, OC(R²⁰)₂C(R²⁰)₂O, OC(R²⁰)₂C(R²⁰)₂, C(R²⁰)₂OC(R²⁰)₂, C(R²⁰)₂C(R²⁰)₂C(R²⁰)₂C(R²⁰)C(R²⁰)₂C(R²⁰)₂, OC(R²⁰)₂N(R²¹), OC(R²⁰)₂C(R²⁰)₂N(R²¹), N(R²¹)C(R²⁰)₂N(R²¹), N(R²¹)C(R²⁰)₂C(R²⁰)₂, N(R²¹)C(R²⁰)₂C(R²⁰)₂C(R²⁰)₂, C(R²⁰)₂N(R²⁰)C(R²⁰)₂, CO.N(R²¹)C(R²⁰)₂, N(R²¹)CO.C(R²⁰)₂, N(R²¹)C(R²⁰)₂CO, CO.N(R²¹)CO, N(R²¹)N(R²¹)CO, N(R²¹)CO.N(R²¹), O.CO.N(R²¹), O.CO.C(R²⁰)₂ and CO.OC(R²⁰)₂ wherein each R²⁰ is hydrogen, (1-8C)alkyl, (2-8C)alkenyl or (2-8C)alkynyl, and wherein R²¹ is hydrogen, (1-8C)alkyl, (2-8C)alkenyl, (2-8C)alkynyl or (2-6C)alkanoyl,

and wherein any aryl, (3-8C)cycloalkyl, (3-8C)cycloalkenyl, heteroaryl or heterocyclyl group within the R⁶ group optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

—X⁶—R¹⁶

wherein X⁶ is a direct bond or is selected from O and N(R¹⁷), wherein R¹⁷ is hydrogen or (1-8C)alkyl, and R¹⁶ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:

—X⁷-Q³

wherein X⁷ is a direct bond or is selected from O, CO and N(R¹⁸), wherein R¹⁸ is hydrogen or (1-8C)alkyl, and Q³ is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,

and wherein any heterocyclyl group within the R⁶ group optionally bears 1 or 2 oxo or thioxo substituents,

and wherein any CH, CH₂ or CH₃ group within the R⁶ group optionally bears on each said CH, CH₂ or CH₃ group one or more halogeno or (1-8C)alkyl substituents and/or a substituent selected from hydroxy, mercapto, amino, cyano, carboxy, carbamoyl, ureido, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within the R⁶ group are optionally separated by the insertion into the chain of a group selected from O, S, SO, SO₂, N(R¹⁹), N(R¹⁹)CO, CON(R¹⁹), N(R¹⁹)CON(R¹⁹), CO, CH(OR¹⁹), N(R¹⁹)SO₂, SO₂N(R¹⁹), CH═CH and C≡C wherein R¹⁹ is hydrogen or (1-8C)alkyl;

r is 0, 1 or 2; and

each R⁷ group, which may be the same or different, is selected from halogeno, trifluoromethyl, cyano, hydroxy, mercapto, amino, carboxy, carbamoyl, sulphamoyl, ureido, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, hydroxy-(1-6C)alkyl and (1-6C)alkoxy-(1-6C)alkyl;

or a pharmaceutically-acceptable salt, solvate or pro-drug thereof.

In this specification the generic term “(1-8C)alkyl” includes both straight-chain and branched-chain alkyl groups such as propyl, isopropyl and tert-butyl, and also (3-8C)cycloalkyl groups such as cyclopropyl, cyclobutyl, cyclopentyl, cyclohexyl and cycloheptyl, and also (3-6C)cycloalkyl-(1-2C)alkyl groups such as cyclopropylmethyl, 2-cyclopropylethyl, cyclobutylmethyl, 2-cyclobutylethyl, cyclopentylmethyl, 2-cyclopentylethyl, cyclohexylmethyl and 2-cyclohexylethyl. However references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only. An analogous convention applies to other generic terms, for example (1-6C)alkoxy includes (3-6C)cycloalkyloxy groups and (3-5C)cycloalkyl-(1-2C)alkoxy groups, for example methoxy, ethoxy, propoxy, isopropoxy, cyclopropyloxy, cyclobutyloxy, cyclopentyloxy, cyclohexyloxy, cyclopropylmethoxy, 2-cyclopropylethoxy, cyclobutylmethoxy, 2-cyclobutylethoxy and cyclopentylmethoxy; (1-6C)alkylamino includes (3-6C)cycloalkylamino groups and (3-5C)cycloalkyl-(1-2C)alkylamino groups, for example methylamino, ethylamino, propylamino, cyclopropylamino, cyclobutylamino, cyclohexylamino, cyclopropylmethylamino, 2-cyclopropylethylamino, cyclobutylmethylamino, 2-cyclobutylethylamino and cyclopentylmethylamino; and di-[(1-6Calkyl]amino includes di-[(3-6C)cycloalkyl]amino groups and di-[(3-5C)cycloalkyl-(1-2C)alkyl]amino groups, for example dimethylamino, diethylamino, dipropylamino, N-cyclopropyl-N-methylamino, N-cyclobutyl-N-methylamino, N-cyclohexyl-N-ethylamino, N-cyclopropylmethyl-N-methylamino, N-(2-cyclopropylethyl)-N-methylamino and N-cyclopentylmethyl-N-methylamino.

It is to be understood that, insofar as certain of the compounds of Formula I defined above may exist in optically active or racemic forms by virtue of one or more asymmetric carbon atoms, the invention includes in its definition any such optically active or racemic form to which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect heteroaryl rings within the definition of Ring A or heterocyclic groups within the R¹ and R⁶ groups that bear 1 or 2 oxo or thioxo substituents. It is to be understood that the present invention includes in its definition any such tautomeric form, or a mixture thereof, which possesses the above-mentioned activity and is not to be limited merely to any one tautomeric form utilised within the formulae drawings or named in the Examples.

In structural Formula I, it is to be understood that there is a hydrogen atom at the 2-position on the quinazoline ring. It is to be understood thereby that the R¹ substituents may only be located at the 5-, 6-, 7- or 8-positions on the quinazoline ring i.e. that the 2-position remains unsubstituted. Conveniently, the R¹ substituents may only be located at the 5-, 6- or 7-positions on the quinazoline ring.

In structural Formula I, it is further to be understood that any R² group that may be present on the pyrazolyl group may be located at any available position. Conveniently, there is a single R² group. More conveniently, no R² group is present (q=0).

In structural Formula I, it is to be understood that the —X¹—R⁶ group may be located at any available position on Ring A. For example, the —X¹—R⁶ group may be located at the 3- or 4-position (relative to the CON(R⁵) group) when Ring A is a 6-membered ring or, for example, it may be located at the 3-position (relative to the CON(R⁵) group) when Ring A is a 5-membered ring.

In structural Formula I, it is to be understood that when, for example, X¹ is a C(R¹⁵)₂O linking group, it is the carbon atom, not the oxygen atom, of the C(R¹⁵)₂O linking group which is attached to Ring A and the oxygen atom is attached to the R⁶ group. It is further to be understood that when a heteroatom in an X¹ group is attached to the R⁶ group, there are at least two carbon atoms between the heteroatom in the X¹ group and any heteroatom in the R⁶ group. For example, if X¹ is an NH group and R⁶ is a hydroxy-(1-6C)alkyl group, the —X¹—R⁶ group so formed may be a 2-hydroxyethylamino group but not a hydroxymethylamino group.

In structural Formula I, it is further to be understood that any R⁷ group that may be present on Ring A may be located at any available position. Conveniently, there is a single R⁷ group. More conveniently, no R⁷ group is present (r=0).

Suitable values for the generic radicals referred to above include those set out below.

A suitable value for R⁶ when it is aryl or for the aryl group within a R⁶ group, or for any one of the ‘Q’ groups (Q¹ to Q³) when it is aryl or for the aryl group within a ‘Q’ group is, for example, phenyl or naphthyl, preferably phenyl.

A suitable value for R⁶ or Q¹ when it is (3-8C)cycloalkyl or for the (3-8C)cycloalkyl group within a R⁶ or Q¹ group is, for example, cyclopropyl, cyclobutyl, cyclopentyl, cyclohexyl, cycloheptyl, bicyclo[2.2.1]heptyl or cyclooctyl.

A suitable value for the (3-8C)cycloalkyl group formed when R³ and R⁴ together with the carbon atom to which they are attached form a (3-8C)cycloalkyl group is, for example, cyclopropyl, cyclobutyl, cyclopentyl, cyclohexyl or cycloheptyl.

A suitable value for R⁶ or Q¹ when it is (3-8C)cycloalkenyl or for the (3-8C)cycloalkenyl group within a R⁶ or Q¹ group is, for example, cyclobutenyl, cyclopentenyl, cyclohexenyl, cycloheptenyl or cyclooctenyl.

A suitable value for R⁶ when it is heteroaryl or for the heteroaryl group within a R⁶ group, or for any one of the ‘Q’ groups (Q¹ to Q³) when it is heteroaryl or for the heteroaryl group within a ‘Q’ group is, for example, an aromatic 5- or 6-membered monocyclic ring or a 9- or 10-membered bicyclic ring with up to five ring heteroatoms selected from oxygen, nitrogen and sulphur, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl.

A suitable value for R⁶ when it is heterocyclyl or for the heterocyclyl group within a R⁶ group, or for any one of the ‘Q’ groups (Q¹ to Q³) when it is heterocyclyl or for the heterocyclyl group within a ‘Q’ group is, for example, a non-aromatic saturated or partially saturated 3 to 10-membered monocyclic or bicyclic ring with up to five heteroatoms selected from oxygen, nitrogen and sulphur, for example oxiranyl, oxetanyl, tetrahydrofuranyl, tetrahydropyranyl, oxepanyl, tetrahydrothienyl, 1,1-dioxotetrahydrothienyl, tetrahydrothiopyranyl, 1,1-dioxotetrahydrothiopyranyl, aziridinyl, azetidinyl, pyrrolinyl, pyrrolidinyl, imidazolinyl, imidazolidinyl, pyrazolinyl, pyrazolidinyl, morpholinyl, tetrahydro-1,4-thiazinyl, 1,1-dioxotetrahydro-1,4-thiazinyl, piperidinyl, homopiperidinyl, piperazinyl, homopiperazinyl, 2-azabicyclo[2.2.1]heptyl, quinuclidinyl, chromanyl, isochromanyl, indolinyl, isoindolinyl, dihydropyridinyl, tetrahydropyridinyl, dihydropyrimidinyl or tetrahydropyrimidinyl, preferably tetrahydrofuranyl, tetrahydropyranyl, tetrahydrothiopyranyl, pyrrolinyl, pyrrolidinyl, morpholinyl, piperidinyl, piperazinyl, indolinyl or isoindolinyl. A suitable value for such a group which bears 1 or 2 oxo or thioxo substituents is, for example, 2-oxopyrrolidinyl, 2-thioxopyrrolidinyl, 2-oxoimidazolidinyl, 2-thioxoimidazolidinyl, 2-oxopiperidinyl, 4-oxo-1,4-dihydropyridinyl, 2,5-dioxopyrrolidinyl, 2,5-dioxoimidazolidinyl or 2,6-dioxopiperidinyl.

A suitable value for a R⁶ or ‘Q’ group when it is heteroaryl-(1-6C)alkyl is, for example, heteroarylmethyl, 2-heteroarylethyl and 3-heteroarylpropyl. The invention comprises corresponding suitable values for R⁶ or ‘Q’ groups when, for example, rather than a heteroaryl-(1-6C)alkyl group, an aryl-(1-6C)alkyl, (3-8C)cycloalkyl-(1-6C)alkyl, (3-8C)cycloalkenyl-(1-6C)alkyl or heterocyclyl-(1-6C)alkyl group is present.

A suitable value for Ring A when it is a 6-membered monocyclic or a 10-membered bicyclic aryl ring or a 5- or 6-membered monocyclic or a 9- or 10-membered bicyclic heteroaryl ring with up to three ring heteroatoms selected from oxygen, nitrogen and sulphur is, for example, phenyl, naphthyl, furyl, pyrrolyl, thienyl, oxazolyl, isoxazolyl, imidazolyl, pyrazolyl, thiazolyl, isothiazolyl, oxadiazolyl, thiadiazolyl, triazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, benzotriazolyl, quinolyl, isoquinolyl, quinazolinyl, quinoxalinyl, cinnolinyl or naphthyridinyl. Conveniently, Ring A is a phenyl, furyl, pyrrolyl, thienyl, oxazolyl, isoxazolyl, imidazolyl, pyrazolyl, thiazolyl, pyridyl, pyrimidinyl, pyrazinyl or pyridazinyl ring. More conveniently, Ring A is a phenyl, pyridyl, pyrimidinyl, pyrazinyl or pyridazinyl ring.

Suitable values for any of the ‘R’ groups (R¹ to R²¹), or for various groups within an R¹, R², R⁷ or —X¹—R⁶ substituent include: —
for halogeno fluoro, chloro, bromo and iodo;
for (1-8C)alkyl: methyl, ethyl, propyl, isopropyl, tert-butyl, cyclobutyl, cyclohexyl, cyclohexylmethyl and 2-cyclopropylethyl;
for (2-8C)alkenyl: vinyl, isopropenyl, allyl and but-2-enyl;
for (2-8C)alkynyl: ethynyl, 2-propynyl and but-2-ynyl;
for (1-6C)alkoxy: methoxy, ethoxy, propoxy, isopropoxy and butoxy;
for (2-6C)alkenyloxy: vinyloxy and allyloxy;
for (2-6C)alkynyloxy: ethynyloxy and 2-propynyloxy;
for (1-6C)alkylthio: methylthio, ethylthio and propylthio;
for (1-6C)alkylsulphinyl: methylsulphinyl and ethylsulphinyl;
for (1-6C)alkylsulphonyl: methylsulphonyl and ethylsulphonyl;
for (1-6C)alkylamino: methylamino, ethylamino, propylamino, isopropylamino and butylamino;
for di-[(1-6C)alkyl]amino: dimethylamino, diethylamino, N-ethyl-N-methylamino and diisopropylamino;
for (1-6C)alkoxycarbonyl: methoxycarbonyl, ethoxycarbonyl, propoxycarbonyl and tert-butoxycarbonyl;
for N-(1-6C)alkylcarbamoyl: N-methylcarbamoyl, N-ethylcarbamoyl and N-propylcarbamoyl;
for N,N-di-[(1-6C)alkyl]carbamoyl: N,N-dimethylcarbamoyl, N-ethyl-N-methylcarbamoyl and N-diethylcarbamoyl;
for (2-6C)alkanoyl: acetyl, propionyl and isobutyryl;
for (2-6C)alkanoyloxy: acetoxy and propionyloxy;
for (2-6C)alkanoylamino: acetamido and propionamido;
for N-(1-6C)alkyl-(2-6C)alkanoylamino: N-methylacetamido and N-methylpropionamido;
for N′-(1-6C)alkylureido: N′-methylureido and N′-ethylureido;
for N′,N′-di-[(1-6C)alkyl]ureido: N′,N′-dimethylureido and N′-methyl-N′-ethylureido;
for N-(1-6C)alkylureido: N-methylureido and N-ethylureido;
for N,N′-di-[(1-6C)alkyl]ureido: N,N′-dimethylureido, N-methyl-N′-ethylureido and N-ethyl-N′-methylureido;
for N,N′,N′-di-[(1-6C)alkyl]ureido: N,N′,N′-trimethylureido; N-ethyl-N′,N′-dimethylureido and N-methyl-N′,N′-diethylureido;
for N-(1-6C)alkylsulphamoyl: N-methylsulphamoyl and N-ethylsulphamoyl;
for N,N-di-[(1-6C)alkyl]sulphamoyl: N,N-dimethylsulphamoyl;
for (1-6C)alkanesulphonylamino: methanesulphonylamino and ethanesulphonylamino;
for N-(1-6C)alkyl-(1-6C)alkanesulphonylamino: N-methylmethanesulphonylamino and N-methylethanesulphonylamino;
for halogeno-(1-6C)alkyl: chloromethyl, 2-fluoroethyl, 2-chloroethyl, 1-chloroethyl, 2,2-difluoroethyl, 2,2,2-trifluoroethyl, 3-fluoropropyl, 3-chloropropyl, 3,3-difluoropropyl and 3,3,3-trifluoropropyl;
for hydroxy-(1-6C)alkyl: hydroxymethyl, 2-hydroxyethyl, 1-hydroxyethyl and 3-hydroxypropyl;
for mercapto-(1-6C)alkyl: mercaptomethyl, 2-mercaptoethyl, 1-mercaptoethyl and 3-mercaptopropyl;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for (1-6C)alkylthio-(1-6C)alkyl: methylthiomethyl, ethylthiomethyl, 2-methylthioethyl, 1-methylthioethyl and 3-methylthiopropyl;
for (1-6C)alkylsulphinyl-(1-6C)alkyl: methylsulphinylmethyl, ethylsulphinylmethyl, 2-methylsulphinylethyl, 1-methylsulphinylethyl and 3-methylsulphinylpropyl;
for (1-6C)alkylsulphonyl-(1-6C)alkyl: methylsulphonylmethyl, ethylsulphonylmethyl, 2-methylsulphonylethyl, 1-methylsulphonylethyl and 3-methylsulphonylpropyl;
for cyano-(1-6C)alkyl: cyanomethyl, 2-cyanoethyl, 1-cyanoethyl and 3-cyanopropyl;
for amino-(1-6C)alkyl: aminomethyl, 2-aminoethyl, 1-aminoethyl, 3-aminopropyl, 1-aminopropyl and 5-aminopropyl;
for (1-6C)alkylamino-(1-6C)alkyl: methylaminomethyl, ethylaminomethyl, 1-methylaminoethyl, 2-methylaminoethyl, 2-ethylaminoethyl and 3-methylaminopropyl;
for di-[(1-6C)alkyl]amino-(1-6C)alkyl: dimethylaminomethyl, diethylaminomethyl, 1-dimethylaminoethyl, 2-dimethylaminoethyl and 3-dimethylaminopropyl;
for (2-6C)alkanoylamino-(1-6C)alkyl: acetamidomethyl, propionamidomethyl, 2-acetamidoethyl and 1-acetamidoethyl;
for N-(1-6C)alkyl-(2-6C)alkanoylamino-(1-6C)alkyl: N-methylacetamidomethyl, N-methylpropionamidomethyl, 2-(N-methylacetamido)ethyl and 1-(N-methylacetamido)ethyl;
for (1-6C)alkoxycarbonylamino-(1-6C)alkyl: methoxycarbonylaminomethyl, ethoxycarbonylaminomethyl, tert-butoxycarbonylaminomethyl and 2-methoxycarbonylaminoethyl.
for ureido-(1-6C)alkyl: ureidomethyl, 2-ureidoethyl and 1-ureidoethyl;
for N′-(1-6C)alkylureido-(1-6C)alkyl: N′-methylureidomethyl, 2-(N′-methylureido)ethyl and 1-(N′-methylureido)ethyl;
for N′,N′-di-[(1-6C)alkyl]ureido-(1-6C)alkyl: N′,N′-dimethylureidomethyl, 2-(N′,N′-dimethylureido)ethyl and 1-(N′,N′-dimethylureido)ethyl;
for N-(1-6C)alkylureido-(1-6C)alkyl: N-methylureidomethyl, 2-(N-methylureido)ethyl and 1-(N-methylureido)ethyl;
for N,N′-di-[(1-6C)alkyl]ureido-(1-6C)alkyl: N,N′-dimethylureidomethyl, 2-(N,N′-dimethylureido)ethyl and 1-(N,N′-dimethylureido)ethyl;
for N,N′,N′-di-[(1-6C)alkyl]ureido-(1-6C)alkyl: N,N′,N′-trimethylureidomethyl, 2-(N,N′,N′-trimethylureido)ethyl and 1-(N,N′,N′-trimethylureido)ethyl;
for carboxy-(1-6C)alkyl: carboxymethyl, 1-carboxyethyl, 2-carboxyethyl, 3-carboxypropyl and 4-carboxybutyl;
for (1-6C)alkoxycarbonyl-(1-6C)alkyl: methoxycarbonylmethyl, ethoxycarbonylmethyl, tert-butoxycarbonylmethyl, 1-methoxycarbonylethyl, 1-ethoxycarbonylethyl, 2-methoxycarbonylethyl, 2-ethoxycarbonylethyl, 3-methoxycarbonylpropyl and 3-ethoxycarbonylpropyl;
for carbamoyl-(1-6C)alkyl: carbamoylmethyl, 1-carbamoylethyl, 2-carbamoylethyl and 3-carbamoylpropyl;
for N-(1-6C)alkylcarbamoyl-(1-6C)alkyl: N-methylcarbamoylmethyl, N-ethylcarbamoylmethyl, N-propylcarbamoylmethyl, 1-(N-methylcarbamoyl)ethyl, 1-(N-ethylcarbamoyl)ethyl, 2-(N-methylcarbamoyl)ethyl, 2-(N-ethylcarbamoyl)ethyl and 3-(N-methylcarbamoyl)propyl;
for N,N-di-[(1-6C)alkyl]carbamoyl-(1-6C)alkyl: N,N-dimethylcarbamoylmethyl, N-ethyl-N-methylcarbamoylmethyl, N,N-diethylcarbamoylmethyl, 1-(N,N-dimethylcarbamoyl)ethyl, 1-(N,N-diethylcarbamoyl)ethyl, 2-(N,N-dimethylcarbamoyl)ethyl, 2-(N,N-diethylcarbamoyl)ethyl, 3-(N,N-dimethylcarbamoyl)propyl and 4-(N,N-dimethylcarbamoyl)butyl;
for sulphamoyl-(1-6C)alkyl: sulphamoylmethyl, 1-sulphamoylethyl, 2-sulphamoylethyl and 3-sulphamoylpropyl;
for N-(1-6C)alkylsulphamoyl-(1-6C)alkyl: N-methylsulphamoylmethyl, 1-(N-methylsulphamoyl)ethyl, 2-(N-methylsulphamoyl)ethyl, and 3-(N-methylsulphamoyl)propyl;
for N,N-di-[(1-6C)alkyl]sulphamoyl-(1-6C)alkyl: N,N-dimethylsulphamoylmethyl, 1-(N,N-dimethylsulphamoyl)ethyl, 2-(N,N-dimethylsulphamoyl)ethyl and 3-(N,N-dimethylsulphamoyl)propyl;
for (1-6C)alkanesulphonylamino-(1-6C)alkyl: methanesulphonylaminomethyl, 2-(methanesulphonylamino)ethyl and 1-(methanesulphonylamino)ethyl; and
for N-(1-6C)alkyl-(1-6C)alkanesulphonylamino-(1-6C)alkyl: N-methylmethanesulphonylaminomethyl, 2-(N-methylmethanesulphonylamino)ethyl and 1-(N-methylmethanesulphonylamino)ethyl.

A suitable value for a (1-3C)alkylenedioxy group that may be present within a R¹ group is, for example, methylenedioxy, ethylidenedioxy, isopropylidenedioxy or ethylenedioxy and the oxygen atoms thereof occupy adjacent ring positions.

When, as defined hereinbefore, an R¹ group forms a group of the formula Q¹-X²— and, for example, X² is a OC(R⁸)₂ linking group, it is the carbon atom, not the oxygen atom, of the OC(R⁸)₂ linking group which is attached to the quinazoline ring and the oxygen atom is attached to the Q¹ group.

A suitable (2-6C)alkylene chain within a R¹ substituent or within a R⁶ group is, for example, an ethylene, trimethylene, tetramethylene or pentamethylene chain.

As defined hereinbefore, adjacent carbon atoms in any (2-6C)alkylene chain within a R¹ substituent or within a R⁶ group may be optionally separated by the insertion into the chain of a group such as O, CON(R¹²) or CON(R¹⁹) respectively, and C≡C. For example, insertion of an O atom into the alkylene chain within a 4-methoxybutoxy group gives rise to, for example, a 2-(2-methoxyethoxy)ethoxy group, for example, insertion of a C≡C group into the ethylene chain within a 2-hydroxyethoxy group gives rise to a 4-hydroxybut-2-ynyloxy group and, for example, insertion of a CONH group into the ethylene chain within a 3-methoxypropoxy group gives rise to, for example, a 2-(2-methoxyacetamido)ethoxy group.

When, as defined hereinbefore, any CH, CH₂ or CH₃ group within a R¹ substituent or within a R⁶ group optionally bears on each said CH, CH₂ or CH₃ group one or more halogeno or (1-8C)alkyl substituents, there is suitably 1 halogeno or (1-8C)alkyl substituent present on each said CH group, there are suitably 1 or 2 such substituents present on each said CH₂ group and there are suitably 1, 2 or 3 such substituents present on each said CH₃ group.

When, as defined hereinbefore, any CH, CH₂ or CH₃ group within a R¹ substituent optionally bears on each said CH, CH₂ or CH₃ group a substituent as defined hereinbefore, suitable R¹ substituents so formed include, for example, hydroxy-substituted (1-8C)alkyl groups such as hydroxymethyl, 1-hydroxyethyl and 2-hydroxyethyl, hydroxy-substituted (1-6C)alkoxy groups such as 2-hydroxypropoxy and 3-hydroxypropoxy, (1-6C)alkoxy-substituted (1-6C)alkoxy groups such as 2-methoxyethoxy and 3-ethoxypropoxy, hydroxy-substituted amino-(2-6C)alkoxy groups such as 3-amino-2-hydroxypropoxy, hydroxy-substituted (1-6C)alkylamino-(2-6C)alkoxy groups such as 2-hydroxy-3-methylaminopropoxy, hydroxy-substituted di-[(1-6C)alkyl]amino-(2-6C)alkoxy groups such as 3-dimethylamino-2-hydroxypropoxy, hydroxy-substituted amino-(2-6C)alkylamino groups such as 3-amino-2-hydroxypropylamino, hydroxy-substituted (1-6C)alkylamino-(2-6C)alkylamino groups such as 2-hydroxy-3-methylaminopropylamino and hydroxy-substituted di-[(1-6C)alkyl]amino-(2-6C)alkylamino groups such as 3-dimethylamino-2-hydroxypropylamino.

When, as defined hereinbefore, any CH, CH₂ or CH₃ group within a R⁶ group optionally bears on each said CH, CH₂ or CH₃ group a substituent as defined hereinbefore, suitable R⁶ groups so formed include, for example, hydroxy-substituted (1-6C)alkylamino-(1-6C)alkyl groups such as 2-hydroxy-3-methylaminopropyl and 2-hydroxyethylaminomethyl and hydroxy-substituted di-[(1-6C)alkyl]amino-(1-6C)alkyl groups such as 3-dimethylamino-2-hydroxypropyl and di-(2-hydroxyethyl)aminomethyl.

It is further to be understood that when, as defined hereinbefore, any CH, CH₂ or CH₃ group within a R¹ substituent or within a R⁶ group optionally bears on each said CH, CH₂ or CH₃ group a substituent as defined hereinbefore, such an optional substituent may be present on a CH, CH₂ or CH₃ group within the hereinbefore defined substituents that may be present on an aryl, heteroaryl or heterocyclyl group within a R¹ substituent or within the R⁶ group. For example, if R¹ or the R⁶ group includes an aryl or heteroaryl group that is substituted by a (1-8C)alkyl group, the (1-8C)alkyl group may be optionally substituted on a CH, CH₂ or CH₃ group therein by one of the hereinbefore defined substituents therefor. For example, if R¹ or the R⁶ group includes a heteroaryl group that is substituted by, for example, a (1-6C)alkylamino-(1-6C)alkyl group, the terminal CH₃ group of the (1-6C)alkylamino group may be further substituted by, for example, a (1-6C)alkylsulphonyl group or a (2-6C)alkanoyl group. For example, the R¹ group or the R⁶ group may be a heteroaryl group such as a thienyl group that is substituted by a N-(2-methylsulphonylethyl)aminomethyl group such that R¹ or R⁶ is, for example, a 5-[N-(2-methylsulphonylethyl)aminomethyl]thien-2-yl group. Further, for example, if R¹ or the R⁶ group includes a heterocyclyl group such as a piperidinyl or piperazinyl group that is substituted on a nitrogen atom thereof by, for example, a (2-6C)alkanoyl group, the terminal CH₃ group of the (2-6C)alkanoyl group may be further substituted by, for example, a di-[(1-6C)alkyl]amino group. For example, the R¹ or R⁶ group may be a N-(2-dimethylaminoacetyl)piperidin-4-yl group or a 4-(2-dimethylaminoacetyl)piperazin-1-yl group. Further, for example, if R¹ or the R⁶ group includes a heterocyclyl group such as a azetidinyl, piperidinyl or piperazinyl group that is substituted on a nitrogen atom thereof by, for example, a (2-6C)alkanoyl group, a CH₂ group of the (2-6C)alkanoyl group may be further substituted by, for example, a hydroxy group. For example, the R¹ or R⁶ group may be a N-(2-hydroxypropionyl)piperidin-4-yl group.

As defined hereinbefore, the —X¹—R⁶ group and one R⁷ group together may form a bivalent group, for example OC(R²⁰)₂O, that spans adjacent ring positions on Ring A. When Ring A is, for example, a phenyl group, a suitable group so formed is a 2,3-methylenedioxyphenyl or a 3,4-methylenedioxyphenyl group. When a further optional R⁷ group is present, for example a halogeno group, a suitable group so, formed is, for example, a 6-fluoro-2,3-methylenedioxyphenyl group. Further, when Ring A is, for example, a phenyl group and the —X¹—R⁶ group and one R⁷ group together form, for example, a OC(R²⁰)₂C(R²⁰)₂ group, a suitable group so formed is, for example, a 2,3-dihydrobenzofuran-5-yl group or a 2,3-dihydrobenzofuran-6-yl group. Further, when Ring A is, for example, a phenyl group and the —X¹—R⁶ group and one R⁷ group together form, for example, a N(R²¹)C(R²⁰)₂C(R²⁰)₂ group, a suitable group so formed is, for example, an indolin-5-yl group or a indolin-6-yl group. Further, when Ring A is, for example, a phenyl group and the —X¹—R⁶ group and one R⁷ group together form, for example, a N(R²¹)CO.C(R²⁰)₂ group, a suitable group so formed is, for example, a 2-oxoindolin-5-yl group or a 2-oxoindolin-6-yl group.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulphuric, trifluoroacetic, citric or maleic acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine. A further suitable pharmaceutically-acceptable salt of a compound of the Formula I is, for example, a salt formed within the human or animal body after administration of a compound of the Formula I.

A suitable pharmaceutically-acceptable solvate of a compound of the Formula I is, for example, a hydrate such as a hemi-hydrate, a mono-hydrate, a di-hydrate or a tri-hydrate or an alternative quantity thereof.

The compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in vivo cleavable amide derivatives that may be formed at a carboxy group or an amino group in a compound of the Formula I.

Accordingly, the present invention includes those compounds of the Formula I as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I may be a synthetically-produced compound or a metabolically-produced compound.

A suitable pharmaceutically-acceptable pro-drug of a compound of the Formula I is one that is based on reasonable medical judgement as being suitable for administration to the human or animal body without undesirable pharmacological activities and without undue toxicity.

Various forms of pro-drug have been described, for example in the following documents: —
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula I that possesses a carboxy group is, for example, an in vivo cleavable ester thereof. An in vivo cleavable ester of a compound of the Formula I containing a carboxy group is, for example, a pharmaceutically-acceptable ester which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I that possesses a hydroxy group is, for example, an in vivo cleavable ester or ether thereof. An in vivo cleavable ester or ether of a compound of the Formula I containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include (X-acyloxyalkyl groups such as acetoxymethyl and pivaloyloxymethyl groups.

A suitable pharmaceutically-acceptable pro-drug of a compound of the Formula I that possesses a carboxy group is, for example, an in vivo cleavable amide thereof, for example an amide formed with an amine such as ammonia, a (14C)alkylamine such as methylamine, a di-(1-4C)alkylamine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically-acceptable pro-drug of a compound of the Formula I that possesses an amino group is, for example, an in vivo cleavable amide derivative thereof. Suitable pharmaceutically-acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I may be exerted in part by one or more metabolites that are formed within the human or animal body after administration of a compound of the Formula I. As stated hereinbefore, the in vivo effects of a compound of the Formula I may also be exerted by way of metabolism of a precursor compound (a pro-drug).

According to an aspect of the invention there is provided a quinazoline derivative of the Formula I

wherein p is 0, 1, 2 or 3;

each R¹ group, which may be the same or different, is selected from halogeno, trifluoromethyl, cyano, hydroxy, mercapto, amino, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino and di-[(1-6C)alkyl]amino, or from a group of the formula:

Q¹-X²—

wherein X² is a direct bond or is selected from O, S, SO, SO₂, N(R⁸), CO, CON(R⁸), N(R⁸)CO, OC(R⁸)₂ and N(R⁸)C(R⁸)₂, wherein each R⁸ is hydrogen or (1-8C)alkyl, and Q¹ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, (3-8C)cycloalkenyl, heteroaryl or heterocyclyl group within a R¹ substituent optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

—X³—R⁹

wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C) alkyl, N,N′-di-[(1-6C)alkyl]ureido-(1-6C)alkyl or N,N′,N′-tri-[(1-6C)alkyl]ureido-(1-6C)alkyl, or from a group of the formula:

—X⁴-Q²

wherein X⁴ is a direct bond or is selected from O, CO and N(R¹¹), wherein R¹¹ is hydrogen or (1-8C)alkyl, and Q² is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,

and wherein any aryl, heteroaryl or heterocyclyl group within a substituent on R¹ optionally bears a (1-3C)alkylenedioxy group,

and wherein any heterocyclyl group within a R¹ substituent optionally bears 1 or 2 oxo or thioxo substituents,

and wherein any CH, CH₂ or CH₃ group within a R¹ substituent optionally bears on each said CH, CH₂ or CH₃ group one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within a R¹ substituent are optionally separated by the insertion into the chain of a group selected from O, S, SO, SO₂, N(R¹²), CO, CH(OR¹²), CON(R¹²), N(R¹²)CO, N(R¹²)CON(R¹²), SO₂N(R¹²), N(R¹²)SO₂, CH═CH and C≡C wherein R¹² is hydrogen or (1-8C)alkyl, or, when the inserted group is N(R¹²), R¹² may also be (2-6C)alkanoyl;

q is 0, 1 or 2;

each R² group, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino and di-[(1-6C)alkyl]amino;

R³ is hydrogen, (1-8C)alkyl, (2-8C)alkenyl or (2-8C)alkynyl;

R⁴ is hydrogen, (1-8C)alkyl, (2-8C)alkenyl, (2-8C)alkynyl, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl, di-[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkoxycarbonyl-(1-6C)alkyl, (2-6C)alkanoylamino-(1-6C)alkyl or N-(1-6C)alkyl-(2-6C)alkanoylamino-(1-6C)alkyl;

or R³ and R⁴ together with the carbon atom to which they are attached form a (3-8C)cycloalkyl group;

R⁵ is hydrogen, (1-8C)alkyl, (2-8C)alkenyl or (2-8C)alkynyl or a group of the formula:

—X⁵—R¹³

wherein X⁵ is a direct bond or is selected from O and N(R¹⁴), wherein R¹⁴ is hydrogen or (1-8C)alkyl, and R¹³ is halogeno-(1-6C)alkyl, hydroxy-(1-6C)alkyl, (1-6C)alkoxy-(1-6C)alkyl or cyano-(1-6C)alkyl;

Ring A is a 6-membered monocyclic or a 10-membered bicyclic aryl ring or a 5- or 6-membered monocyclic or a 9- or 10-membered bicyclic heteroaryl ring with up to three ring heteroatoms selected from oxygen, nitrogen and sulphur;

X¹ is a direct bond or is selected from O, S, SO, SO₂, N(R¹⁵), CO, CH(OR¹⁵), CON(R¹⁵), N(R¹⁵)CO, N(R⁵)CON(R¹⁵), SO₂N(R¹⁵), N(R¹⁵)SO₂, C(R¹⁵)₂O, C(R¹⁵)₂S and C(R¹⁵)₂N(R¹⁵), wherein R¹⁵ is hydrogen or (1-8C)alkyl;

R⁶ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, carboxy-(1-6C)alkyl, (1-6C)alkoxycarbonyl-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulphamoyl-(1-6C)alkyl, N-(1-6C)alkylsulphamoyl-(1-6C)alkyl, N,N-di-[(1-6C)alkyl]sulphamoyl-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl, N,N′,N′-tri-[(1-6C)alkyl]ureido-(1-6C)alkyl, (1-6C)alkanesulphonylamino-(1-6C) alkyl or N-(1-6C)alkyl-(1-6C)alkanesulphonylamino-(1-6C)alkyl,

or R⁶ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,

or, when X¹ is a direct bond, R⁶ may be carboxy, (1-6C)alkoxycarbonyl, sulphamoyl, N-(1-6C)alkylsulphamoyl or N,N-di-[(1-6C)alkyl]sulphamoyl,

or the —X¹—R⁶ group and one R⁷ group together form a bivalent group that spans adjacent ring positions on Ring A selected from OC(R²⁰)₂O, OC(R²⁰)₂C(R²⁰)₂O, OC(R²⁰)₂C(R²⁰)₂, C(R²⁰)₂OC(R²⁰)₂, OC(R²⁰)₂N(R²¹), N(R²¹)C(R²⁰)₂N(R²¹), N(R²¹)C(R²⁰)₂C(R²⁰)₂, C(R²⁰)₂N(R²¹)C(R²⁰)₂, CO.N(R²⁰)C(R²⁰)₂, N(R²⁰)CO.C(R²⁰)₂, N(R²¹)C(R²⁰)₂CO, CO.N(R²⁰)CO, N(R²¹)N(R²⁰)CO, N(R²⁰)CO.N(R²⁰), O.CO.N(R²⁰), O.CO.C(R²⁰)₂ and CO.OC(R²⁰)₂ wherein each R²⁰ is hydrogen, (1-8C)alkyl, (2-8C)alkenyl or (2-8C)alkynyl, and wherein R²¹ is hydrogen, (1-8C)alkyl, (2-8C)alkenyl, (2-8C)alkynyl or (2-6C)alkanoyl,

and wherein any aryl, (3-8C)cycloalkyl, (3-8C)cycloalkenyl, heteroaryl or heterocyclyl group within the R⁶ group optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

—X⁶—R¹⁶

wherein X⁶ is a direct bond or is selected from O and N(R¹⁷), wherein R¹⁷ is hydrogen or (1-8C)alkyl, and R¹⁶ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:

—X⁷-Q³

wherein X⁷ is a direct bond or is selected from O, CO and N(R¹⁸), wherein R¹⁸ is hydrogen or (1-8C)alkyl, and Q³ is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,

and wherein any heterocyclyl group within the R⁶ group optionally bears 1 or 2 oxo or thioxo substituents,

and wherein any CH, CH₂ or CH₃ group within the R⁶ group optionally bears on each said CH, CH₂ or CH₃ group one or more halogeno or (1-8C)alkyl substituents and/or a substituent selected from hydroxy, mercapto, amino, cyano, carboxy, carbamoyl, ureido, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within the R⁶ group are optionally separated by the insertion into the chain of a group selected from O, S, SO, SO₂, N(R¹⁹), N(R¹⁹)CO, CON(R¹⁹), N(R¹⁹)CON(R¹⁹), CO, CH(OR¹⁹), N(R¹⁹)SO₂, SO₂N(R¹⁹), CH═CH and C≡C wherein R¹⁹ is hydrogen or (1-8C)alkyl;

r is 0, 1 or 2; and

each R⁷ groups which may be the same or different, is selected from halogeno, trifluoromethyl, cyano, hydroxy, mercapto, amino, carboxy, carbamoyl, sulphamoyl, ureido, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino;

or a pharmaceutically-acceptable salt, solvate or pro-drug thereof.

Particular novel compounds of the invention include, for example, quinazoline derivatives of the Formula I, or pharmaceutically-acceptable salts, solvates or pro-drugs thereof, wherein, unless otherwise stated, each of p, R¹, q, R², R³, R⁴, R⁵, Ring A, X¹, R⁶, r and R⁷ has any of the meanings defined hereinbefore or in paragraphs (a) to (xx) hereinafter: —

(a) p is 1, 2 or 3, and each R¹ group, which may be the same or different, is selected from halogeno, trifluoromethyl, cyano, hydroxy, amino, (1-8C)alkyl, (2-8C)alkenyl, (2-8C)alkynyl, (1-6C)alkoxy, (2-6C)alkenyloxy, (2-6C)alkynyloxy, (1-6C)alkylamino and di-[(1-6C)alkyl]amino, or from a group of the formula:

Q¹-X²—

wherein X² is a direct bond or is selected from O, N(R⁸), CON(R⁸), N(R⁸)CO and OC(R⁸)₂ wherein R⁸ is hydrogen or (1-8C)alkyl, and Q¹ is aryl, aryl-(1-6C)alkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a substituent on R¹ optionally bears 1, 2 or 3 substituents, which may be the same or different, selected from halogeno, trifluoromethyl, hydroxy, amino, carbamoyl, (1-8C)alkyl, (2-8C)alkenyl, (2-8C)alkynyl, (1-6C)alkoxy, (1-6C)alkylsulphonyl, (1-6C)alkylamino, di-[(1-6C)alkyl]amino, (2-6C)alkanoyl, N-(1-6C)alkylcarbamoyl, N,N-di-[(1-6C)alkyl]carbamoyl, (2-6C)alkanoylamino and N-(1-6C)alkyl-(2-6C)alkanoylamino, or from a group of the formula:

—X³—R⁹

wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, (1-6C)alkoxy-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:

—X⁴-Q²

wherein X⁴ is a direct bond or is selected from O, CO and N(R¹¹), wherein R¹¹ is hydrogen or (1-8C)alkyl, and Q² is heterocyclyl or heterocyclyl-(1-6C)alkyl which optionally bears 1 or 2 substituents, which may be the same or different, selected from halogeno, (1-8C)alkyl and (1-6C)alkoxy,

and wherein any heterocyclyl group within a substituent on R¹ optionally bears a (1-3C)alkylenedioxy group,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more halogeno or (1-8C)alkyl groups and/or a substituent selected from hydroxy, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within a R¹ substituent are optionally separated by the insertion into the chain of a group selected from O, N(R¹²), CON(R¹²), N(R¹²)CO, CH═CH and C≡C wherein R¹² is hydrogen or (1-8C)alkyl, or, when the inserted group is N(R¹²), R¹² may also be (2-6C)alkanoyl;

(b) p is 1 and the R¹ group is located at the 5-, 6- or 7-position or p is 2 and the R¹ groups, which may be the same or different, are located at the 5- and 7-positions or at the 6- and 7-positions and each R¹ group is selected from fluoro, chloro, trifluoromethyl, hydroxy, amino, methyl, ethyl, propyl, butyl, vinyl, allyl, but-3-enyl, ethynyl, 2-propynyl, but-3-ynyl, methoxy, ethoxy, propoxy, isopropoxy, butoxy, allyloxy, but-3-enyloxy, ethynyloxy, 2-propynyloxy, but-3-ynyloxy, methylamino, ethylamino, propylamino, dimethylamino, diethylamino and dipropylamino, or from a group of the formula:

Q¹-X²—

wherein X² is a direct bond or is selected from O, NH, CONH, NHCO and OCH₂ and Q¹ is phenyl, benzyl, cyclopropylmethyl, 2-thienyl, 1-imidazolyl, 1,2,3-triazol-1-yl, 1,2,4-triazol-1-yl, 2-, 3- or 4-pyridyl, 2-imidazol-1-ylethyl, 3-imidazol-1-ylpropyl, 2-(1,2,3-triazolyl)ethyl, 3-(1,2,3-triazolyl)propyl, 2-(1,2,4-triazolyl)ethyl, 3-(1,2,4-triazolyl)propyl, 2-, 3- or 4-pyridylmethyl, 2-(2-, 3- or 4-pyridyl)ethyl, 3-(2-, 3- or 4-pyridyl)propyl, tetrahydrofuran-3-yl, 3- or 4-tetrahydropyranyl, 1-, 2- or 3-pyrrolidinyl, morpholino, 1,1-dioxotetrahydro-4H-1,4-thiazin-4-yl, piperidino, piperidin-3-yl, piperidin-4-yl, 1-, 3- or 4-homopiperidinyl, piperazin-1-yl, homopiperazin-1-yl, 1-, 2- or 3-pyrrolidinylmethyl, morpholinomethyl, piperidinomethyl, 3- or 4-piperidinylmethyl, 1-, 3- or 4-homopiperidinylmethyl, 2-pyrrolidin-1-ylethyl, 3-pyrrolidin-2-ylpropyl, pyrrolidin-2-ylmethyl, 2-pyrrolidin-2-ylethyl, 3-pyrrolidin-1-ylpropyl, 4-pyrrolidin-1-ylbutyl, 2-morpholinoethyl, 3-morpholinopropyl, 4-morpholinobutyl, 2-(1,1-dioxotetrahydro-4H-1,4-thiazin-4-yl)ethyl, 3-(1,1-dioxotetrahydro-4H-1,4-thiazin-4-yl)propyl, 2-piperidinoethyl, 3-piperidinopropyl, 4-piperidinobutyl, 2-piperidin-3-ylethyl, 3-piperidin-3-ylpropyl, 2-piperidin-4-ylethyl, 3-piperidin-4-ylpropyl, 2-homopiperidin-1-ylethyl, 3-homopiperidin-1-ylpropyl, 2-(1,2,3,6-tetrahydropyridin-1-yl)ethyl, 3-(1,2,3,6-tetrahydropyridin-1-yl)propyl, 4-(1,2,3,6-tetrahydropyridin-1-yl)butyl, 2-piperazin-1-ylethyl, 3-piperazin-1-ylpropyl, 4-piperazin-1-ylbutyl, 2-homopiperazin-1-ylethyl or 3-homopiperazin-1-ylpropyl,

and wherein any aryl, (3-8C)cycloalkyl, heteroaryl or heterocyclyl group within a substituent on R¹ optionally bears 1, 2 or 3 substituents, which may be the same or different, selected from fluoro, chloro, trifluoromethyl, hydroxy, amino, carbamoyl, methyl, ethyl, allyl, 2-propynyl, methoxy, methylsulphonyl, methylamino, dimethylamino, acetyl, propionyl, isobutyryl, N-methylcarbamoyl, N,N-dimethylcarbamoyl, methylenedioxy, ethylidendioxy and isopropylidenedioxy, or optionally bears 1 substituent selected from a group of the formula:

—X³—R⁹

wherein X³ is a direct bond or is selected from O and NH and R⁹ is 2-fluoroethyl, 2,2-difluoroethyl, 2,2,2-trifluoroethyl, 3-fluoropropyl, 3,3-difluoropropyl, 3,3,3-trifluoropropyl, 2-hydroxyethyl, 3-hydroxypropyl, 2-methoxyethyl, 3-methoxypropyl, cyanomethyl, aminomethyl, 2-aminoethyl, 3-aminopropyl, methylaminomethyl, 2-methylaminoethyl, 3-methylaminopropyl, 2-ethylaminoethyl, 3-ethylaminopropyl, dimethylaminomethyl, 2-dimethylaminoethyl, 3-dimethylaminopropyl, acetamidomethyl or N-methylacetamidomethyl, and from a group of the formula:

—X⁴-Q²

wherein X⁴ is a direct bond or is selected from O, CO and NH and Q² is pyrrolidin-1-ylmethyl, 2-pyrrolidin-1-ylethyl, 3-pyrrolidin-1-ylpropyl, morpholinomethyl, 2-morpholinoethyl, 3-morpholinopropyl, piperidinomethyl, 2-piperidinoethyl, 3-piperidinopropyl, piperazin-1-ylmethyl, 2-piperazin-1-ylethyl or 3-piperazin-1-ylpropyl, each of which optionally bears 1 or 2 substituents, which may be the same or different, selected from fluoro, chloro, methyl and methoxy,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more fluoro, chloro or methyl groups or a substituent selected from hydroxy, amino, cyano, methoxy, methylsulphonyl, methylamino, dimethylamino, diisopropylamino, N-ethyl-N-methylamino, N-isopropyl-N-methylamino, acetyl, acetamido and N-methylacetamido,

and wherein adjacent carbon atoms in any (2-6C)alkylene chain within a R¹ substituent are optionally separated by the insertion into the chain of a group selected from O, NH, N(Me), N(COMe), CONH, NHCO, CH═CH and C≡C;

(c) each of p and R¹ has any of the meanings defined in paragraphs (a) and (b) hereinbefore except that when R¹ is a group of the formula:

Q¹-X²—

X² may not be a direct bond;
(d) p is 1 and the R¹ group is located at the 5-, 6- or 7-position or p is 2 and the R¹ groups, which may be the same or different, are located at the 5- and 7-positions or at the 6- and 7-positions and each R¹ is selected from hydroxy, amino, methyl, ethyl, propyl, butyl, vinyl, ethynyl, methoxy, ethoxy, propoxy, isopropoxy, butoxy, but-3-enyloxy, methylamino, ethylamino, dimethylamino, diethylamino, cyclopentyloxy, cyclohexyloxy, phenoxy, benzyloxy, tetrahydrofuran-3-yloxy, tetrahydropyran-3-yloxy, tetrahydropyran-4-yloxy, cyclopropylmethoxy, 2-imidazol-1-ylethoxy, 3-imidazol-1-ylpropoxy, 2-(1,2,3-triazol-1-yl)ethoxy, 3-(1,2,3-triazol-1-yl)propoxy, 2-(1,2,4-triazol-1-yl)ethoxy, 3-(1,2,4-triazol-1-yl)propoxy, pyrid-2-ylmethoxy, pyrid-3-ylmethoxy, pyrid-4-ylmethoxy, 2-pyrid-2-ylethoxy, 2-pyrid-3-ylethoxy, 2-pyrid-4-ylethoxy, 3-pyrid-2-ylpropoxy, 3-pyrid-3-ylpropoxy, 3-pyrid-4-ylpropoxy, pyrrolidin-1-yl, morpholino, piperidino, piperazin-1-yl, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, piperidin-4-ylmethoxy, 2-piperidin-3-ylethoxy, 3-piperidin-3-ylpropoxy, 2-piperidin-4-ylethoxy, 3-piperidin-4-ylpropoxy, 2-homopiperidin-1-ylethoxy, 3-homopiperidin-1-ylpropoxy, 2-(1,2,3,6-tetrahydropyridin-1-yl)ethoxy 3-(1,2,3,6-tetrahydropyridin-1-yl)propoxy, 4-(1,2,3,6-tetrahydropyridin-1-yl)butoxy, 2-piperazin-1-ylethoxy, 3-piperazin-1-ylpropoxy, 4-piperazin-1-ylbutoxy, 2-homopiperazin-1-ylethoxy, 3-homopiperazin-1-ylpropoxy, 2-pyrrolidin-1-ylethylamino, 3-pyrrolidin-1-ylpropylamino, 4-pyrrolidin-1-ylbutylamino, pyrrolidin-3-ylamino, pyrrolidin-2-ylmethylamino, 2-pyrrolidin-2-ylethylamino, 3-pyrrolidin-2-ylpropylamino, 2-morpholinoethylamino, 3-morpholinopropylamino, 4-morpholinobutylamino, 2-(1,1-dioxotetrahydro-4H-1,4-thiazin-4-yl)ethylamino, 3-(1,1-dioxotetrahydro-4H-1,4-thiazin-4-yl)propylamino, 2-piperidinoethylamino, 3-piperidinopropylamino, 4-piperidinobutylamino, piperidin-3-ylamino, piperidin-4-ylamino, piperidin-3-ylmethylamino, 2-piperidin-3-ylethylamino, piperidin-4-ylmethylamino, 2-piperidin-4-ylethylamino, 2-homopiperidin-1-ylethylamino, 3-homopiperidin-1-ylpropylamino, 2-piperazin-1-ylethylamino, 3-piperazin-1-ylpropylamino, 4-piperazin-1-ylbutylamino, 2-homopiperazin-1-ylethylamino or 3-homopiperazin-1-ylpropylamino,

and wherein any phenyl, imidazolyl, triazolyl, pyridyl or heterocyclyl group within a substituent on R¹ optionally bears 1 or 2 substituents, which may be the same or different, selected from fluoro, chloro, trifluoromethyl, hydroxy, amino, carbamoyl, methyl, ethyl, methoxy, ethoxy, N-methylcarbamoyl, N,N-dimethylcarbamoyl, methylenedioxy, ethylidendioxy and isopropylidenedioxy, and a pyrrolidin-2-yl, piperidin-3-yl, piperidin-4-yl, piperazin-1-yl or homopiperazin-1-yl group within a R¹ substituent is optionally N-substituted with allyl, 2-propynyl, methylsulphonyl, ethylsulphonyl, acetyl, propionyl, isobutyryl, 2-fluoroethyl, 2,2-difluoroethyl, 2,2,2-trifluoroethyl, 3-fluoropropyl, 3,3-difluoropropyl, 3,3,3-trifluoropropyl, 2-methoxyethyl, 3-methoxypropyl, cyanomethyl, 2-aminoethyl, 3-aminopropyl, 2-methylaminoethyl, 3-methylaminopropyl, 2-dimethylaminoethyl, 3-dimethylaminopropyl, 2-pyrrolidin-1-ylethyl, 3-pyrrolidin-1-ylpropyl, 2-morpholinoethyl, 3-morpholinopropyl, 2-piperidinoethyl, 3-piperidinopropyl, 2-piperazin-1-ylethyl or 3-piperazin-1-ylpropyl, the last 8 of which substituents each optionally bears 1 or 2 substituents, which may be the same or different, selected from fluoro, chloro, methyl and methoxy,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more fluoro, chloro or methyl groups or a substituent selected from hydroxy, amino, methoxy, methylsulphonyl, methylamino, dimethylamino, diisopropylamino, N-ethyl-N-methylamino, N-isopropyl-N-methylamino, N-methyl-N-propylamino, acetamido and N-methylacetamido,

and wherein adjacent carbon atoms in any (2-6C)alkylene chain within a R¹ substituent are optionally separated by the insertion into the chain of a group selected from O, NI, N(Me), CH═CH and C≡C;

(e) p is 1 and the R¹ group is located at the 7-position or p is 2 and the R¹ groups, which may be the same or different, are located at the 6- and 7-positions and each R¹ is selected from hydroxy, amino, methyl, ethyl, methoxy, ethoxy, propoxy, isopropoxy, butoxy, methylamino, ethylamino, dimethylamino, diethylamino, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino,

and wherein adjacent carbon atoms in any (2-6C)alkylene chain within a R¹ substituent are optionally separated by the insertion into the chain of a group selected from O, NH, CH═CH and C≡C;

(f) p is 1 and the R¹ group is located at the 5-position or p is 2 and the R¹ groups, which may be the same or different, are located at the 5- and 7-positions and each R¹ is selected from hydroxy, amino, methyl, ethyl, methoxy, ethoxy, propoxy, isopropoxy, butoxy, methylamino, ethylamino, dimethylamino, diethylamino, tetrahydrofuran-3-yloxy, tetrahydropyran-4-yloxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, 3-piperidinyloxy, 4-piperidinyloxy, piperidin-3-ylmethoxy, piperidin-4-ylmethoxy, 2-piperidin-3-ylethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy, 3-homopiperazin-1-ylpropoxy, cyclobutyloxy, cyclopentyloxy and cyclohexyloxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino,

and wherein adjacent carbon atoms in any (2-6C)alkylene chain within a R¹ substituent are optionally separated by the insertion into the chain of a group selected from O, NH, CH═CH and C≡C;

(g) p is 2 and the R¹ groups, which may be the same or different, are located at the 6- and 7-positions and the R¹ group at the 6-position is selected from hydroxy, methoxy, ethoxy and propoxy, and the R¹ group at the 7-position is selected from methoxy, ethoxy, propoxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino; methoxy, methylsulphonyl, methylamino, dimethylamino, diisopropylamino, N-ethyl-N-methylamino and N-isopropyl-N-methylamino;

(h) p is 2 and the R¹ groups, which may be the same or different, are located at the 5- and 7-positions and the R¹ group at the 5-position is selected from methoxy, ethoxy, propoxy, isopropoxy, butoxy, tetrahydrofuran-3-yloxy, tetrahydropyran-4-yloxy, pyrrolidin-3-yloxy, pyrrolidin-2-ylmethoxy, 3-piperidinyloxy, 4-piperidinyloxy, piperidin-3-ylmethoxy, piperidin-4-ylmethoxy, cyclobutyloxy, cyclopentyloxy and cyclohexyloxy, and the R¹ group at the 7-position is selected from hydroxy, methoxy, ethoxy, propoxy, isopropoxy, butoxy, 2-pyrrolidin-1-ylethoxy, 3-pyrrolidin-1-ylpropoxy, 4-pyrrolidin-1-ylbutoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, 2-piperidin-3-ylethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;

(i) q is 0;
(j) q is 1 and the R² group is selected from halogeno, trifluoromethyl, cyano, hydroxy, amino, (1-8C)alkyl, (2-8C)alkenyl, (2-8C)alkynyl, (1-6C)alkoxy, (1-6C)alkylamino and di-[(1-6C)alkyl]amino;
(k) q is 1 or 2 and each R² group, which may be the same or different, is selected from fluoro, chloro, trifluoromethyl, cyano, hydroxy, amino, methyl, methoxy, methylamino and dimethylamino;
(l) q is 1 and the R² group is selected from fluoro, chloro, trifluoromethyl, hydroxy, amino, methyl, methoxy, methylamino and dimethylamino;
(m) R³ is hydrogen, methyl or ethyl;
(n) R³ is hydrogen;
(o) R⁴ is hydrogen, methyl, ethyl, propyl, 2-fluoroethyl, 2,2-difluoroethyl, 2,2,2-trifluoroethyl, 3-fluoropropyl, 3,3-difluoropropyl, 3,3,3-trifluoropropyl, 2-hydroxyethyl, 3-hydroxypropyl, 2-methoxyethyl, 3-methoxypropyl, cyanomethyl, 2-cyanoethyl, aminomethyl, 2-aminoethyl, 3-aminopropyl, methylaminomethyl, 2-methylaminoethyl, 3-methylaminopropyl, 2-ethylaminoethyl, 3-ethylaminopropyl, dimethylaminomethyl, 2-dimethylaminoethyl, 3-dimethylaminopropyl, acetamidomethyl or N-methylacetamidomethyl;
(p) R⁴ is hydrogen, methyl or ethyl;
(q) R⁴ is hydrogen;
(r) R³ and R⁴ together with the carbon atom to which they are attached form a cyclopropyl, cyclobutyl, cyclopentyl or cyclohexyl group;
(s) R⁵ is hydrogen, methyl, ethyl, propyl, allyl, 2-propynyl, 2-fluoroethyl, 2,2-difluoroethyl, 2,2,2-trifluoroethyl, 3-fluoropropyl, 3,3-difluoropropyl, 3,3,3-trifluoropropyl, 2-hydroxyethyl, 3-hydroxypropyl, 2-methoxyethyl, 3-methoxypropyl, cyanomethyl, 2-cyanoethyl or 3-cyanopropyl;
(t) R⁵ is hydrogen, methyl or ethyl;
(u) R⁵ is hydrogen;
(v) Ring A is a 6-membered monocyclic aryl ring or a 5- or 6-membered monocyclic heteroaryl ring with up to three ring heteroatoms selected from oxygen, nitrogen and sulphur;
(w) Ring A is a phenyl ring,
(x) Ring A is a 6-membered monocyclic heteroaryl ring with up to three nitrogen heteroatoms;
(y) Ring A is a 5-membered monocyclic heteroaryl ring with up to three ring heteroatoms selected from oxygen, nitrogen and sulphur;
(z) Ring A is a phenyl, furyl, pyrrolyl, thienyl, oxazolyl, isoxazolyl, imidazolyl, pyrazolyl, thiazolyl, pyridyl, pyrimidinyl, pyrazinyl or pyridazinyl ring;
(aa) Ring A is a phenyl, pyridyl, pyrimidinyl, pyrazinyl or pyridazinyl ring;
(bb) when Ring A is a 6-membered ring, the —X¹—R⁶ group is located at the 3- or 4-position (relative to the CON(R⁵) group) or, when Ring A is a 5-membered ring, the —X¹—R⁶ group is located at the 3-position (relative to the CON(R⁵) group);
(cc) Ring A is a phenyl, pyridyl, pyrimidinyl, pyrazinyl or pyridazinyl ring and the —X¹—R⁶ group is located at the 3- or 4-position (relative to the CON(R⁵) group);
(dd) Ring A is a 9- or 10-membered bicyclic heteroaryl ring with up to three ring heteroatoms selected from oxygen, nitrogen and sulphur;
(ee) Ring A is a benzofuranyl, indolyl, benzothienyl, benzoxazolyl, benzimidazolyl, benzothiazolyl, indazolyl, benzotriazolyl, 1H-pyrrolo[3,2-b]pyridinyl, quinolyl, isoquinolyl, quinazolinyl, quinoxalinyl or naphthyridinyl ring;
(ff) Ring A is a indolyl, benzoxazolyl, benzimidazolyl, benzothiazolyl, indazoyl, benzotriazolyl, quinolyl, isoquinolyl, quinoxalinyl or naphthyridinyl ring;
(gg) X¹ is a direct bond or is selected from O, S, SO, SO₂, N(R¹⁵) and CO, wherein R¹⁵ is hydrogen, methyl or ethyl, provided that, when a heteroatom in an X¹ group is attached to the R⁶ group, there are at least two carbon atoms between the heteroatom in the X¹ group and any heteroatom in the R⁶ group;
(hh) X¹ is selected from CON(R¹⁵), N(R¹⁵)CO, N(R¹⁵)CON(R¹⁵), SO₂N(R¹⁵), N(R¹⁵)SO₂, C(R¹⁵)₂O, C(R¹⁵)₂S and C(R¹⁵)₂N(R¹⁵), wherein R¹⁵ is hydrogen, methyl or ethyl, provided that, when a heteroatom in an X¹ group is attached to the R⁶ group, there are at least two carbon atoms between the heteroatom in the X¹ group and any heteroatom in the R⁶ group;
(ii) X¹ is a direct bond;
(jj) X¹ is selected from O, S, SO, SO₂, NH and CO, provided that, when a heteroatom in an X¹ group is attached to the R⁶ group, there are at least two carbon atoms between the heteroatom in the X¹ group and any heteroatom in the R⁶ group;
(kk) R⁶ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, carboxy-(1-6C)alkyl, (1-6C)alkoxycarbonyl-(1-6C)alkyl, carbamoyl-(1-6C)alkyl, N-(1-6C)alkylcarbamoyl-(1-6C)alkyl or N,N-di-[(1-6C)alkyl]carbamoyl-(1-6C)alkyl, or R⁶ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within the R⁶ group optionally bears 1, 2 or 3 substituents, which may be the same or different, selected from halogeno, trifluoromethyl, cyano, hydroxy, amino, carboxy, carbamoyl, ureido, (1-8C)alkyl, (2-8C)alkenyl, (2-8C)alkynyl, (1-6C)alkoxy, (1-6C)alkylamino and di-[(1-6C)alkyl]amino, or from a group of the formula:

—X⁶—R¹⁶

wherein X⁶ is a direct bond or is selected from O and N(R¹⁷), wherein R¹⁷ is hydrogen or (1-8C)alkyl, and R¹⁶ is halogeno-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl or di-[(1-6C)alkyl]amino-(1-6C)alkyl,

and wherein any heterocyclyl group within the R⁶ group optionally bears 1 or 2 oxo or thioxo substituents,

and wherein any CH, CH₂ or CH₃ group within the R⁶ group optionally bears on each said CH, CH₂ or CH₃ group one or more halogeno or (1-8C)alkyl substituents and/or a substituent selected from hydroxy, amino, cyano, carboxy, carbamoyl, ureido, (3-8C)alkenyl, (3-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoylamino and N-(1-6C)alkyl-(2-6C)alkanoylamino;

(ll) R⁶ is hydroxy-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or R⁶ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within the R⁶ group optionally bears 1 or 2 substituents, which may be the same or different, selected from halogeno, trifluoromethyl, cyano, hydroxy, amino, (1-8C)alkyl, (1-6C)alkoxy, (1-6C)alkylamino and di-[(1-6C)alkyl]amino, or from a group of the formula:

—X⁶—R¹⁶

wherein X⁶ is a direct bond and R¹⁶ is halogeno-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl or di-[(1-6C)alkyl]amino-(1-6C)alkyl,

and wherein any CH, CH₂ or CH₃ group within the R⁶ group optionally bears on each said CH, CH₂ or CH₃ group 1, 2 or 3 halogeno or (1-8C)alkyl substituents and/or a substituent selected from hydroxy, amino, cyano, (3-8C)alkenyl, (3-8C)alkynyl, (1-6C)alkoxy, (1-6C)alkylsulphonyl, (1-6C)alkylamino, di-[(1-6C)alkyl]amino, (2-6C)alkanoylamino and N-(1-6C)alkyl-(2-6C)alkanoylamino;

(mm) R⁶ is hydroxy-(1-6C)alkyl, (1-6C)alkoxy-(1-6C)alkyl, amino-(1-6C)alkyl, (1-6C)alkylamino-(1-6C)alkyl or di-[(1-6C)alkyl]amino-(1-6C)alkyl, or R⁶ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within the R⁶ group optionally bears 1 or 2 substituents, which may be the same or different, selected from halogeno, trifluoromethyl, hydroxy, amino, (1-8C)alkyl, (1-6C)alkoxy, (1-6C)alkylamino, di-[(1-6C)alkyl]amino, hydroxy-(1-6C)alkyl, amino-(1-6C)alkyl, (1-6C)alkylamino-(1-6C)alkyl and di-[(1-6C)alkyl]amino-(1-6C)alkyl;

(nn) R⁶ is hydroxymethyl, 1-hydroxyethyl, 2-hydroxyethyl, 1-hydroxy-1-methylethyl, 3-hydroxypropyl, cyanomethyl, 1-cyanoethyl, 2-cyanoethyl, 1-cyano-1-methylethyl, 3-cyanopropyl, aminomethyl, 1-aminoethyl, 2-aminoethyl, 1-amino-1-methylethyl, 3-aminopropyl, methylaminomethyl, 1-methyl aminoethyl, 2-methylaminoethyl, 1-methylamino-1-methylethyl, 3-methylaminopropyl, ethylaminomethyl, 1-ethylaminoethyl, 2-ethylaminoethyl, 1-ethylamino-1-methylethyl, 3-ethylaminopropyl, dimethylaminomethyl, 1-dimethylaminoethyl, 2-dimethylaminoethyl, 1-dimethylamino-1-methylethyl or 3-dimethylaminopropyl, or R⁶ is phenyl, benzyl, cyclopropyl, cyclobutyl, cyclopentyl, cyclohexyl, furyl, thienyl, oxazolyl, imidazolyl, thiazolyl, pyridyl, pyrimidinyl, tetrahydrofuranyl, tetrahydropyranyl, tetrahydrothiopyranyl, pyrrolinyl, pyrrolidinyl, imidazolidinyl, pyrazolidinyl, morpholinyl, tetrahydro-1,4-thiazinyl, piperidinyl, homopiperidinyl, piperazinyl, homopiperazinyl, indolinyl, isoindolinyl, pyrrolinylmethyl, pyrrolidinylmethyl, imidazolidinylmethyl, pyrazolidinylmethyl, morpholinylmethyl, 2-(morpholinyl)ethyl, tetrahydro-1,4-thiazinylmethyl, 2-(tetrahydro-1,4-thiazinyl)ethyl, piperidinylmethyl, 2-(piperidinyl)ethyl, homopiperidinylmethyl, piperazinylmethyl, 2-(piperazinyl)ethyl or homopiperazinylmethyl,

and wherein any aryl, (3-8C)cycloalkyl, heteroaryl or heterocyclyl group within the R⁶ group optionally bears 1 or 2 substituents, which may be the same or different, selected from fluoro, chloro, trifluoromethyl, hydroxy, amino, methyl, ethyl, methoxy, ethoxy, methylamino, dimethylamine, hydroxymethyl, 2-hydroxyethyl, aminomethyl, 2-aminoethyl, methylaminomethyl, 2-methylaminoethyl, dimethylaminomethyl and 2-dimethylaminoethyl;

(oo) R⁶ is hydroxymethyl, 1-hydroxyethyl, 2-hydroxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, dimethylaminomethyl, 1-dimethylaminoethyl or 2-dimethylaminoethyl, or R⁶ is phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, morpholinylmethyl, 2-(morpholinyl)ethyl, piperidinylmethyl, 2-(piperidinyl)ethyl, homopiperidinylmethyl, piperazinylmethyl, 2-(piperazinyl)ethyl or homopiperazinylmethyl,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl;

(pp) X¹ is a direct bond and R⁶ is sulphamoyl, N-(1-6C)alkylsulphamoyl or N,N-di-[(1-6C)alkyl]sulphamoyl;
(qq) the —X¹—R⁶ group and one R⁷ group together form a bivalent group that spans adjacent ring positions on Ring A selected from OC(R²⁰)₂O, OC(R²⁰)₂C(R²⁰)₂, C(R²⁰)₂OC(R²⁰)₂, C(R²⁰)₂C(R²⁰)₂C(R²⁰)₂, C(R²⁰)₂C(R²⁰)₂C(R²⁰)₂C(R²⁰)₂, OC(R²⁰)₂N(R²¹), OC(R²⁰)₂C(R²⁰)₂N(R²¹), N(R²¹)C(R²⁰)₂C(R²⁰)₂, N(R²¹)C(R²⁰)₂C(R²⁰)₂C(R²⁰)₂ and C(R²⁰)₂N(R²¹)C(R²⁰)₂, wherein each of R²⁰ and R²¹ is hydrogen, (1-8C)alkyl, (2-8C)alkenyl or (2-8C)alkynyl;
(rr) the —X¹—R⁶ group and one R⁷ group together form a bivalent group that spans adjacent ring positions on Ring A selected from OC(R²⁰)₂O, OC(R²⁰)₂C(R²⁰)₂, C(R²⁰)₂OC(R²⁰)₂, OC(R²⁰)₂N(R²¹), N(R²¹)C(R²⁰)₂N(R²¹), N(R²¹)C(R²⁰)₂C(R²⁰)₂ and C(R²⁰)₂N(R²⁰)C(R²⁰)₂, wherein each of R²⁰ and R²¹ is hydrogen, (1-8C)alkyl, (2-8C)alkenyl or (2-8C)alkynyl;
(ss) the —X¹—R⁶ group and one R⁷ group together form a bivalent group that spans adjacent ring positions on Ring A selected from OC(R²⁰)₂O, OC(R²⁰)₂C(R²⁰)₂O, C(R²⁰)₂, OC(R²⁰)₂N(R²¹), N(R²¹)C(R²⁰)₂N(R²¹) and C(R²⁰)₂N(R²¹)C(R²⁰)₂, wherein each of R²⁰ and R²¹ is hydrogen, methyl, ethyl or propyl;
(tt) the —X¹—R⁶ group and one R⁷ group together form a bivalent group that spans adjacent ring positions on Ring A selected from OCH₂O, OCH₂CH₂O, CH₂OCH₂, OCH₂NH, NHCH₂NH and CH₂NHCH₂;
(uu) r is 0;
(vv) r is 1 or 2 and each R⁷ group, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino, di-[(1-6C)alkyl]amino, (2-6C)alkanoylamino and N-(1-6C)alkyl-(2-6C)alkanoylamino;
(ww) r is 0 or r is 1 or 2 and each R⁷ group, which may be the same or different, is selected from fluoro, chloro, trifluoromethyl, cyano, hydroxy, amino, methyl, methoxy, methylamino and dimethylamino; and
(xx) r is 0 or r is 1 and the R⁷ group is selected from fluoro, chloro, trifluoromethyl, hydroxy, amino, methyl, methoxy, methylamino and dimethylamino.

A particular compound of the invention is a quinazoline derivative of the Formula I wherein: —

p is 2 and the R¹ groups, which may be the same or different, are located at the 6- and 7-positions and the R¹ group at the 6-position is selected from hydroxy, methoxy, ethoxy and propoxy, and the R¹ group at the 7-position is selected from methoxy, ethoxy, propoxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;

q is 0;

each of R³, R⁴ and R⁵ is hydrogen;

Ring A is a phenyl, pyridyl, pyrimidinyl, pyrazinyl or pyridazinyl ring and the —X¹—R⁶ group is located at the 3- or 4-position (relative to the CON(R⁵) group);

X¹ is a direct bond or is selected from O, S, SO, SO₂, N(R¹⁵) and CO, wherein R¹⁵ is hydrogen, methyl or ethyl, provided that, when a heteroatom in an X¹ group is attached to the R⁶ group, there are at least two carbon atoms between the heteroatom in the X¹ group and any heteroatom in the R⁶ group;

R⁶ is hydroxymethyl, 1-hydroxyethyl, 2-hydroxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, dimethylaminomethyl, 1-dimethylaminoethyl or 2-dimethylaminoethyl, or R⁶ is phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, morpholinylmethyl, 2-(morpholinyl)ethyl, piperidinylmethyl, 2-(piperidinyl)ethyl, homopiperidinylmethyl, piperazinylmethyl, 2-(piperazinyl)ethyl or homopiperazinylmethyl,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl; and

r is 0 or r is 1 and the R⁷ group is selected from fluoro, chloro, trifluoromethyl, hydroxy, amino, methyl, methoxy; methylamino and dimethylamino;

or a pharmaceutically-acceptable salt, solvate or pro-drug thereof.

A further particular compound of the invention is a quinazoline derivative of the Formula I wherein: —

p is 2 and the first R¹ group is a 6-methoxy group and the second R¹ group is located at the 7-position and is selected from methoxy, ethoxy, 2-methoxyethoxy, 3-methoxypropoxy, 2-methylsulphonylethoxy, 3-methylsulphonylpropoxy, 2-(2-methoxyethoxy)ethoxy, 2-pyrrolidin-1-ylethoxy, 3-pyrrolidin-1-ylpropoxy, 2-[(3 RS,4SR)-3,4-methylenedioxypyrrolidin-1-yl]ethoxy, 3-[(3RS,4SR)-3,4-methylenedioxypyrrolidin-1-yl]propoxy, 2-morpholinoethoxy, 3-morpholinopropoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 2-piperidin-3-ylethoxy, 2-(N-methylpiperidin-3-yl)ethoxy, 3-piperidin-3-ylpropoxy, 3-(N-methylpiperidin-3-yl)propoxy, 2-piperidin-4-ylethoxy, 2-(N-methylpiperidin-4-yl)ethoxy, 3-piperidin-4-ylpropoxy, 3-(N-methylpiperidin-4-yl)propoxy, 2-(1,2,3,6-tetrahydropyridin-1-yl)ethoxy, 3-(1,2,3,6-tetrahydropyridin-1-yl)propoxy, 2-(4-hydroxypiperidin-1-yl)ethoxy, 3-(4-hydroxypiperidin-1-yl)propoxy, 2-piperazin-1-ylethoxy, 3-piperazin-1-ylpropoxy, 4-piperazin-1-ylbutoxy, 2-(4-methylpiperazin-1-yl)ethoxy, 3-(4-methylpiperazin-1-yl)propoxy, 4-(4-methylpiperazin-1-yl)butoxy, 2-(4-allylpiperazin-1-yl)ethoxy, 3-(4-allylpiperazin-1-yl)propoxy, 2-(4-prop-2-ynylpiperazin-1-yl)ethoxy, 3-(4-prop-2-ynylpiperazin-1-yl)propoxy, 2-(4-methylsulphonylpiperazin-1-yl)ethoxy, 3-(4-methylsulphonylpiperazin-1-yl)propoxy, 2-(4-acetylpiperazin-1-yl)ethoxy, 3-(4-acetylpiperazin-1-yl)propoxy, 4-(4-acetylpiperazin-1-yl)butoxy, 2-(4-isobutyrylpiperazin-1-yl)ethoxy, 3-(4-isobutyrylpiperazin-1-yl)propoxy, 4-(4-isobutyrylpiperazin-1-yl)butoxy, 2-[4-(2-fluoroethyl)piperazin-1-yl]ethoxy, 3-[4-(2-fluoroethyl)piperazin-1-yl]propoxy, 2-[4-(2,2,2-trifluoroethyl)piperazin-1-yl]ethoxy, 3-[4-(2,2,2-trifluoroethyl)piperazin-1-yl]propoxy, 2-(4-cyanomethylpiperazin-1-yl)ethoxy, 3-(4-cyanomethylpiperazin-1-yl)propoxy, 2-[2-(4-methylpiperazin-1-yl)ethoxy]ethoxy, 2-(4-pyridyloxy)ethoxy, 3-pyridylmethoxy and 2-cyanopyrid-4-ylmethoxy;

q is 0;

each of R³, R⁴ and R⁵ is hydrogen;

Ring A is phenyl or pyridyl and the —X¹—R⁶ group is located at the 3- or 4-position (relative to the CON(R⁵) group);

X¹ is a direct bond or O, provided that, when X¹ is O, there are at least two carbon atoms between that O heteroatom and any heteroatom in the R⁶ group;

R⁶ is hydroxymethyl, 1-hydroxyethyl, 2-hydroxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, dimethylaminomethyl, 1-dimethylaminoethyl or 2-dimethylaminoethyl, or R⁶ is phenyl, benzyl, cyclopropyl, cyclopentyl, cyclohexyl, pyrrolidinyl, morpholinyl, tetrahydro-1,4-thiazinyl, piperidinyl, piperazinyl, pyrrolidinylmethyl, morpholinylmethyl, piperidinylmethyl or piperazinylmethyl,

and wherein any aryl, (3-8C)cycloalkyl or heterocyclyl group within the R⁶ group optionally bears a substituent selected from fluoro, chloro, trifluoromethyl, hydroxy, amino, methyl, methoxy, methylamino and dimethylamino; and

r is 0 or r is 1 and the R⁷ group is located at an available 3- or 4-position (relative to the CON(R⁵) group) and is selected from fluoro, chloro, trifluoromethyl, hydroxy, amino, methyl, methoxy, methylamino and dimethylamino;

or a pharmaceutically-acceptable salt, solvate or pro-drug thereof.

A further particular compound of the invention is a quinazoline derivative of the Formula I wherein: —

p is 2 and the first R¹ group is a 6-methoxy group and the second R¹ group is located at the 7-position and is selected from methoxy, ethoxy and 2-methoxyethoxy;

q is 0;

each of R³, R⁴ and R⁵ is hydrogen;

Ring A is phenyl and the —X¹—R⁶ group is located at the 3- or 4-position (relative to the CON(R⁵) group) and is selected from hydroxymethyl, 1-hydroxyethyl, 2-hydroxyethyl, methylaminomethyl, 1-methylaminoethyl, 2-methylaminoethyl, dimethylaminomethyl, 1-dimethylaminoethyl and 2-dimethylaminoethyl, or Ring A is 3-pyridyl and the —X¹—R⁶ group is located at the 4-position (each relative to the CON(R⁵) group) and is a 2-dimethylaminoethoxy group; and

r is 0 or r is 1 and the R⁷ group is located at an available 3- or 4-position-(relative to the CON(R⁵) group) and is selected from fluoro, chloro, methoxy, methylamino and dimethylamino;

or a pharmaceutically-acceptable salt, solvate or pro-drug thereof.

A further particular compound of the invention is a quinazoline derivative of the Formula I wherein: —

p is 2 and the R¹ groups, which may be the same or different, are located at the 5- and 7-positions and the R¹ group at the 5-position is selected from methoxy, ethoxy, propoxy, isopropoxy, butoxy, tetrahydrofuran-3-yloxy, tetrahydropyran-4-yloxy, pyrrolidin-3-yloxy, pyrrolidin-2-ylmethoxy, 3-piperidinyloxy, 4-piperidinyloxy, piperidin-3-ylmethoxy, piperidin-4-ylmethoxy, cyclobutyloxy, cyclopentyloxy and cyclohexyloxy, and the R¹ group at the 7-position is selected from hydroxy, methoxy, ethoxy, propoxy, isopropoxy, butoxy, 2-pyrrolidin-1-ylethoxy, 3-pyrrolidin-1-ylpropoxy, 4-pyrrolidin-1-ylbutoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, 2-piperidin-3-ylethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;

q is 0;

each of R³, R⁴ and R⁵ is hydrogen;

Ring A is a phenyl, pyridyl, pyrimidinyl, pyrazinyl or pyridazinyl ring and the —X¹—R⁶ group is located at the 3- or 4-position (relative to the CON(R⁵) group);

X¹ is a direct bond or is selected from O, S, SO, SO₂, N(R¹⁵) and CO, wherein R¹⁵ is hydrogen, methyl or ethyl, provided that, when a heteroatom in an X¹ group is attached to the R⁶ group, there are at least two carbon atoms between the heteroatom in the X¹ group and any heteroatom in the R⁶ group;

R⁶ is hydroxymethyl, 1-hydroxyethyl, 2-hydroxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, dimethylaminomethyl, 1-dimethylaminoethyl or 2-dimethylaminoethyl, or R⁶ is phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, morpholinylmethyl, 2-(morpholinyl)ethyl, piperidinylmethyl, 2-(piperidinyl)ethyl, homopiperidinylmethyl, piperazinylmethyl, 2-(piperazinyl)ethyl or homopiperazinylmethyl,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl; and

r is 0 or r is 1 and the R⁷ group is selected from fluoro, chloro, trifluoromethyl, hydroxy, amino, methyl, methoxy, methylamino and dimethylamino;

or a pharmaceutically-acceptable salt, solvate or pro-drug thereof.

A further particular compound of the invention is a quinazoline derivative of the Formula I wherein: —

p is 1 and the R¹ group is located at the 5-position and is selected from methoxy, ethoxy, propoxy, isopropoxy, tetrahydropyran-4-yloxy, 4-piperidinyloxy and N-methylpiperidin-4-yloxy, or p is 2 and the first R¹ group is located at the 5-position and is selected from the group of substituents listed immediately above, and the second R¹ group is located at the 7-position and is selected from methoxy, ethoxy, 2-methoxyethoxy, 3-methoxypropoxy, 2-methylsulphonylethoxy, 3-methylsulphonylpropoxy, 2-(2-methoxyethoxy)ethoxy, 2-pyrrolidin-1-ylethoxy, 3-pyrrolidin-1-ylpropoxy, 2-[(3RS,4SR)-3,4-methylenedioxypyrrolidin-1-yl]ethoxy, 3-[(3RS,4SR)-3,4-methylenedioxypyrrolidin-1-yl]propoxy, 2-morpholinoethoxy, 3-morpholinopropoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 2-piperazin-1-ylethoxy, 3-piperazin-1-ylpropoxy, 4-piperazin-1-ylbutoxy, 2-(4-methylpiperazin-1-yl)ethoxy, 3-(4-methylpiperazin-1-yl)propoxy, 2-(4-allylpiperazin-1-yl)ethoxy, 3-(4-allylpiperazin-1-yl)propoxy, 2-(4-prop-2-ynylpiperazin-1-yl)ethoxy, 3-(4-prop-2-ynylpiperazin-1-yl)propoxy; 2-(4-acetylpiperazin-1-yl)ethoxy, 3-(4-acetylpiperazin-1-yl)propoxy, 2-(4-isobutyrylpiperazin-1-yl)ethoxy, 3-(4-isobutyrylpiperazin-1-yl)propoxy, 2-[4-(2,2,2-trifluoroethyl)piperazin-1-yl]ethoxy and 3-[4-(2,2,2-trifluoroethyl)piperazin-1-yl]propoxy;

q is 0;

each of R³, R⁴ and R⁵ is hydrogen;

Ring A is a phenyl or pyridyl and the —X¹—R⁶ group is located at the 3- or 4-position (relative to the CON(R⁵) group);

X¹ is a direct bond or O, provided that, when X¹ is O, there are at least two carbon atoms between that O heteroatom and any heteroatom in the R⁶ group;

R⁶ is hydroxymethyl, 1-hydroxyethyl, 2-hydroxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, dimethylaminomethyl, 1-dimethylaminoethyl or 2-dimethylaminoethyl, or R⁶ is phenyl, benzyl, cyclopropyl, cyclopentyl, cyclohexyl, pyrrolidinyl, morpholinyl, tetrahydro-1,4-thiazinyl, piperidinyl, piperazinyl, pyrrolidinylmethyl, morpholinylmethyl, piperidinylmethyl, piperazinylmethyl

and wherein any aryl, (3-8C)cycloalkyl or heterocyclyl group within the R⁶ group optionally bears a substituent selected from fluoro, chloro, trifluoromethyl, hydroxy, amino, methyl, methoxy, methylamino and dimethylamino; and

r is 0 or r is 1 and the R⁷ group is located at an available 3- or 4-position (relative to the CON(R⁵) group) and is selected from fluoro, chloro, trifluoromethyl, hydroxy, amino, methyl, methoxy, methylamino and dimethylamino;

or a pharmaceutically-acceptable salt, solvate or pro-drug thereof.

A further particular compound of the invention is a quinazoline derivative of the Formula I wherein: —

p is 2 and the first R¹ group is a 5-methoxy group and the second R¹ group is located at the 7-position and is selected from methoxy, ethoxy and 2-methoxyethoxy;

q is 0;

each of R³, R⁴ and R⁵ is hydrogen;

Ring A is phenyl and the —X¹—R⁶ group is located at the 3- or 4-position (relative to the CON(R⁵) group) and is selected from hydroxymethyl, 1-hydroxyethyl, 2-hydroxyethyl, methylaminomethyl, 1-methylaminoethyl, 2-methylaminoethyl, dimethylaminomethyl, 1-dimethylaminoethyl and 2-dimethylaminoethyl, or Ring A is 3-pyridyl and the —X¹—R⁶ group is located at the 4-position (each relative to the CON(R⁵) group) and is a 2-dimethylaminoethoxy group; and

r is 0 or r is 1 and the R⁷ group is located at an available 3- or 4-position (relative to the CON(R⁵) group) and is selected from fluoro, chloro, methoxy, methylamino and dimethylamino;

or a pharmaceutically-acceptable salt, solvate or pro-drug thereof.

Further particular compounds of the invention are quinazoline derivatives of the Formula I wherein each of p, R¹, q, R², R³, R⁴, R⁵, Ring A, r and R⁷ has any of the meanings defined hereinbefore in the various definitions of particular compounds of the invention provided that X¹ is a direct bond and R⁶ is sulphamoyl;

or pharmaceutically-acceptable salts, solvates or pro-drugs thereof.

Further particular compounds of the invention are quinazoline derivatives of the Formula I wherein each of p, R¹, q, R², R³, R⁴, R⁵, Ring A, r and R⁷ has any of the meanings defined hereinbefore in the various definitions of particular compounds of the invention provided that the —X¹—R⁶ group and one R⁷ group together form a bivalent group that spans adjacent ring positions on Ring A selected from OCH₂O, OCH₂CH₂O, CH₂OCH₂, OCH₂NH, NHCH₂NH and CH₂NHCH₂;

or pharmaceutically-acceptable salts, solvates or pro-drugs thereof.

Further particular compounds of the invention are quinazoline derivatives of the Formula I wherein each of p, R¹, q, R², R³, R⁴, R⁵, r and R⁷ has any of the meanings defined hereinbefore in the various definitions of particular compounds of the invention provided that Ring A is phenyl and the —X¹—R⁶ group and one R⁷ group together form a OCH₂O bivalent group that spans the 2,3- or 3,4-positions on said phenyl ring;

or pharmaceutically-acceptable salts, solvates or pro-drugs thereof.

A further particular compound of the invention is a quinazoline derivative of the Formula I wherein: —

p is 2 and the R¹ groups, which may be the same or different, are located at the 6- and 7-positions and are selected from methoxy, ethoxy, propoxy, 2-hydroxyethoxy, 3-hydroxypropoxy, 2-methoxyethoxy, 3-methoxypropoxy, 2-methylsulphonylethoxy, 3-methylsulphonylpropoxy and 2-(2-methoxyethoxy)ethoxy;

q is 0 or q is 1 and the R² group is fluoro, chloro, methyl or methoxy;

each of R³, R⁴ and R⁵ is hydrogen;

Ring A is phenyl and the —X¹—R⁶ group is located at the 3- or 4-position (relative to the CON(R⁵) group) and is selected from hydroxymethyl, aminomethyl, methylaminomethyl, ethylaminomethyl, propylaminomethyl, isopropylaminomethyl, cyclopropylaminomethyl, cyclopropylmethylaminomethyl, dirmethylaminomethyl, diethylaminomethyl, N-ethyl-N-methylaminomethyl, N-cyclopropyl-N-methylaminomethyl, N-cyclopropylmethyl-N-methylaminomethyl, azetidin-1-ylmethyl, pyrrolidin-1-ylmethyl, morpholinylmethyl, piperidinylmethyl, homopiperidinylmethyl, piperazinylmethyl, homopiperazinylmethyl and 2-methoxyethoxy; and

r is 0 or r is 1 and any R⁷ group that is present is located at an available 3-, 4- or 5-position (relative to the CON(R⁵) group) and is selected from fluoro, chloro, methyl, ethyl, methoxy, methylamino and dimethylamino;

or a pharmaceutically-acceptable salt, solvate or pro-drug thereof.

A further particular compound of the invention is a quinazoline derivative of the Formula I wherein: —

p is 2 and the first R¹ group is a 6-methoxy group and the second R¹ group is located at the 7-position and is selected from methoxy, ethoxy, 2-hydroxyethoxy and 2-methoxyethoxy;

q is 0 or q is 1 and the R² group is fluoro;

each of R³, R⁴ and R⁵ is hydrogen;

Ring A is phenyl and the —X¹—R⁶ group is located at the 3-position (relative to the CON(R⁵) group) and is selected from hydroxymethyl, methylaminomethyl, ethylaminomethyl, propylaminomethyl, isopropylaminomethyl, cyclopropylaminomethyl, dimethylaminomethyl, diethylaminomethyl, N-ethyl-N-methylaminomethyl and N-cyclopropyl-N-methylaminomethyl;

and r is 0 or r is 1 and any R⁷ group that is present is located at the 4-position (relative to the CON(R⁵) group) and is selected from fluoro, chloro, methyl, methoxy and dimethylamino;

or a pharmaceutically-acceptable salt, solvate or pro-drug thereof.

A further particular compound of the invention is a quinazoline derivative of the Formula I wherein: —

p is 2 and the first R¹ group is a 6-methoxy group and the second R¹ group is located at the 7-position and is selected from methoxy, ethoxy, 2-hydroxyethoxy and 2-methoxyethoxy;

q is 0 or q is 1 and the R² group is fluoro;

each of R³, R⁴ and R⁵ is hydrogen;

Ring A is 2-pyridyl and the —X¹—R⁶ group is located at the 4-, 5- or 6-position (relative to the pyridyl nitrogen heteroatom) and is selected from cyclopropylamino, 2-hydroxyethylamino, 2-methoxyethylamino, N-cyclopropyl-N-methylamino, pyrrolidin-1-yl, piperidino, morpholino, piperazin-1-yl, methylaminomethyl, ethylaminomethyl, propylaminomethyl, isopropylaminomethyl, cyclopropylaminomethyl, dimethylaminomethyl, N-ethyl-N-methylaminomethyl and N-cyclopropyl-N-methylaminomethyl;

and r is 0;

or a pharmaceutically-acceptable salt, solvate or pro-drug thereof.

Particular compounds of the invention are, for example, the quinazoline derivatives of the Formula I that are disclosed within Examples 1, 6(1), 6(2), 9(2), 9(3), 9(5), 9(7) and 9(11) that are set out hereinafter.

Further particular compounds of the invention are, for example, the quinazoline derivatives of the Formula I that are disclosed within Examples 9(8), 9(18), 17(13), 17(17), 17(19), 20(3), 21, 33(6), 35(1), 35(5), 35(17), 37(13), 37(17), 37(24), 37(25), 37(26) and 40 that are set out hereinafter.

A quinazoline derivative of the Formula I, or a pharmaceutically-acceptable salt, solvate or pro-drug thereof, may be prepared by any process known to be applicalble to the preparation of chemically-related compounds. Such processes, when used to prepare a quinazoline derivative of the Formula I are provided as a further feature of the invention and are illustrated by the following representative process variants in which, unless otherwise stated, each of p, R¹, q, R², R³, R⁴, R⁵, Ring A, X¹, R⁶, r and R⁷ has any of the meanings defined hereinbefore. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

(a) The reaction of a quinazoline of the Formula II

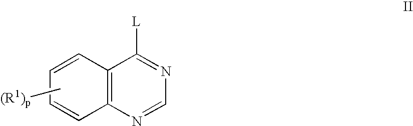

wherein L is a displaceable group and p and R¹ have any of the meanings defined hereinbefore except that any functional group is protected if necessary, with a pyrazole of the Formula III

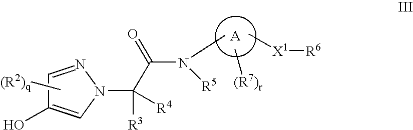

wherein q, R², R³, R⁴, R⁵, Ring A, X¹, R⁶, r and R⁷ have any of the meanings defined hereinbefore except that any functional group is protected if necessary, whereafter any protecting group that is present is removed.

The reaction may conveniently be carried out in the presence of a suitable acid or in the presence of a suitable base. A suitable acid is, for example, an inorganic acid such as, for example, hydrogen chloride or hydrogen bromide. A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal amide, for example sodium hexamethyldisilazane, or, for example, an alkali metal hydride, for example sodium hydride.

A suitable displaceable group L is, for example, a halogeno, alkoxy, aryloxy or sulphonyloxy group, for example a chloro, bromo, methoxy, phenoxy, pentafluorophenoxy, methanesulphonyloxy or toluene-4-sulphonyloxy group. The reaction is conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxane, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 0 to 250° C., preferably in the range 0 to 150° C.

Typically, the quinazoline of the Formula II may be reacted with a compound of the Formula III in the presence of an aprotic solvent such as N,N-dimethylformamide, conveniently in the presence of a base, for example potassium carbonate or sodium hexamethyldisilazane, and at a temperature in the range, for example, 0 to 150° C., preferably in the range, for example, 0 to 70° C.

The quinazoline derivative of the Formula I may be obtained from this process in the form of the free base or alternatively it may be obtained in the form of a salt with the acid of the formula H-L wherein L has the meaning defined hereinbefore. When it is desired to obtain the free base from the salt, the salt may be treated with a suitable base, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide.

Protecting groups may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

A carboxy protecting group may be the residue of an ester-forming aliphatic or arylaliphatic alcohol or of an ester-forming silanol (the said alcohol or silanol preferably containing 1-20 carbon atoms). Examples of carboxy protecting groups include straight or branched chain (1-12C)alkyl groups (for example isopropyl, and tert-butyl); lower alkoxy-lower alkyl groups (for example methoxymethyl, ethoxymethyl and isobutoxymethyl); lower acyloxy-lower alkyl groups, (for example acetoxymethyl, propionyloxymethyl, butyryloxymethyl and pivaloyloxymethyl); lower alkoxycarbonyloxy-lower alkyl groups (for example 1-methoxycarbonyloxyethyl and 1-ethoxycarbonyloxyethyl); aryl-lower alkyl groups (for example benzyl, 4-methoxybenzyl, 2-nitrobenzyl, 4-nitrobenzyl, benzhydryl and phthalidyl); tri(lower alkyl)silyl groups (for example trimethylsilyl and tert-butyldimethylsilyl); tri(lower alkyl)silyl-lower alkyl groups (for example trimethylsilylethyl); and (2-6C)alkenyl groups (for example allyl). Methods particularly appropriate for the removal of carboxyl protecting groups include for example acid-, base-, metal- or enzymically-catalysed cleavage.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl, hydrogenation for groups such as benzyl and photolytically for groups such as 2-nitrobenzyloxycarbonyl.

The reader is referred to Advanced Organic Chemistry, 4th Edition, by J. March, published by John Wiley & Sons 1992, for general guidance on reaction conditions and reagents and to Protective Groups in Organic Synthesis, 2^{nd} Edition, by T. Green et al., also published by John Wiley & Son, for general guidance on protecting groups.

Quinazoline starting materials of the Formula II may be obtained by conventional procedures such as those disclosed in International Patent Applications WO 01/94341, WO 02/00649, WO 02/16352 and WO 03/055491. For example, a 1,4-dihydroquinolin-4-one of the Formula IV

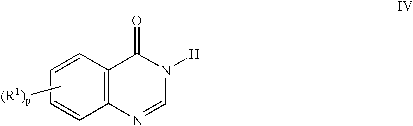

wherein p and R¹ have any of the meanings defined hereinbefore except that any functional group is protected if necessary, may be reacted with a halogenating agent such as thionyl chloride, phosphoryl chloride or a mixture of carbon tetrachloride and triphenylphosphine whereafter any protecting group that is present is removed.

The 4-chloroquinazoline so obtained may be converted, if required, into a 4-pentafluorophenoxyquinazoline by reaction with pentafluorophenol in the presence of a suitable base such as potassium carbonate and in the presence of a suitable solvent such as N,N-dimethylformamide.

Pyrazole starting materials of the Formula III may be obtained by conventional procedures, for example using procedures analogous to those described in International Patent Applications WO 02/00649, WO 03/055491 and PCT/GB2004/001614 (published subsequently as WO 2004/094410). For example, an acetic acid of the Formula V

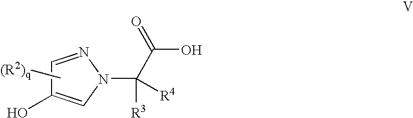

or a reactive derivative thereof, wherein q, R², R³ and R⁴ have any of the meanings defined hereinbefore except that any functional group is protected if necessary, may be reacted with an amine of the Formula VI

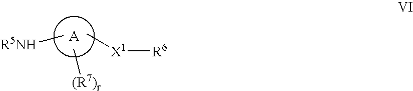

wherein R⁵, Ring A, X¹, R⁶, r and R⁷ have any of the meanings defined hereinbefore except that any functional group is protected if necessary, whereafter any protecting group that is present is removed.

A suitable reactive derivative of an acetic acid of the Formula V is, for example, an acyl halide, for example an acyl chloride formed by the reaction of the acid with an inorganic acid chloride, for example thionyl chloride; a mixed anhydride, for example an anhydride formed by the reaction of the acid with a chloroformate such as isobutyl chloroformate; an active ester, for example an ester formed by the reaction of the acid with a phenol such as pentafluorophenol, with an ester such as pentafluorophenyl trifluoroacetate or with an alcohol such as methanol, ethanol, isopropanol, butanol or N-hydroxybenzotriazole; an acyl azide, for example an azide formed by the reaction of the acid with an azide such as diphenylphosphoryl azide; an acyl cyanide, for example a cyanide formed by the reaction of an acid with a cyanide such as diethylphosphoryl cyanide; or the product of the reaction of the acid with a carbodiimide such as dicyclohexylcarbodiimide or 1-(3-dimethylaminopropyl)-3-ethylcarbodiimide or with a uronium compound such as 2-(7-azabenzotriazol-1-yl)-1,1,3,3-tetramethyluronium hexafluorophosphate(V) or 2-(benzotriazol-1-yl)-1,1,3,3-tetramethyluronium tetrafluoroborate.

The reaction is conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxane, an aromatic solvent such as toluene. Conveniently, the reaction is conveniently carried out in the presence of a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 0 to 120° C., preferably at or near ambient temperature.

Acetic acid derivatives of the Formula V and amines of the Formula VI may be obtained by conventional procedures such as those disclosed in the Examples that are set out hereinafter.

(b) The coupling, conveniently in the presence of a suitable base, of a quinazoline of the Formula VII

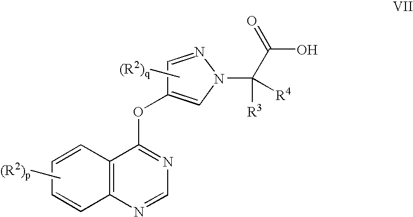

or a reactive derivative thereof as defined hereinbefore, wherein p, R¹, q, R², R³ and R⁴ have any of the meanings defined hereinbefore except that any functional group is protected if necessary, with an amine of the Formula VI

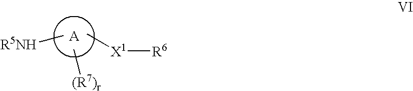

wherein R⁵, Ring A, X¹, R⁶, r and R⁷ have any of the meanings defined hereinbefore except that any functional group is protected if necessary, whereafter any protecting group that is present is removed.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal amide, for example sodium hexamethyldisilazane, or, for example, an alkali metal hydride, for example sodium hydride.

The reaction is conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxane, an aromatic solvent such as toluene. Conveniently, the reaction is conveniently carried out in the presence of a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 0 to 120° C., preferably at or near ambient temperature. Quinazoline derivatives of the Formula VII and amines of the Formula VI may be obtained by conventional procedures such as those disclosed in the Examples that are set out hereinafter.

(c) For the production of those compounds of the Formula I wherein at least one R¹ group is a group of the formula

Q¹-X²—

wherein Q¹ is an aryl-(1-6C)alkyl, (3-7C)cycloalkyl-(1-6C)alkyl, (3-7C)cycloalkenyl-(1-6C)alkyl, heteroaryl-(1-6C)alkyl or heterocyclyl-(1-6C)alkyl group or an optionally substituted alkyl group and X² is an oxygen atom, the coupling, conveniently in the presence of a suitable dehydrating agent, of a quinazoline of the Formula VIII

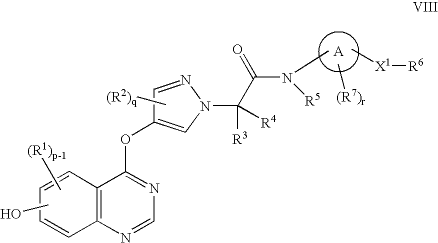

wherein each of p, R¹, q, R², R³, R⁴, R⁵, Ring A, X¹, R⁶, r and R⁷ has any of the meanings defined hereinbefore except that any functional group is protected if necessary, with an appropriate alcohol wherein any functional group is protected if necessary, whereafter any protecting group that is present is removed.

A suitable dehydrating agent is, for example, a carbodiimide reagent such as dicyclohexylcarbodiimide or 1-(3-dimethylaminopropyl)-3-ethylcarbodiimide or a mixture of an azo compound such as diethyl or di-tert-butyl azodicarboxylate and a phosphine such as triphenylphosphine. The reaction is conveniently carried out in the presence of a suitable inert solvent or diluent, for example a halogenated solvent such as methylene chloride, chloroform or carbon tetrachloride and at a temperature in the range, for example, 10 to 150° C., preferably at or near ambient temperature.

The reaction is conveniently carried out in the presence of a suitable inert solvent or diluent, for example a halogenated solvent such as methylene chloride, chloroform or carbon tetrachloride and at a temperature in the range, for example, 10 to 150° C., preferably at or near ambient temperature.

Quinazoline derivatives of the Formula III may be obtained by conventional procedures.

(d) For the production of those compounds of the Formula I wherein the —X¹—R⁶ group is an amino-substituted (1-6C)alkyl group (such as a dimethylaminomethyl, 2-dimethylaminoethyl or 4-methylpiperazin-1-ylmethyl group), the reaction, conveniently in the presence of a suitable base as defined hereinbefore, of a compound of the Formula I wherein the —X¹—R⁶ group is a halogeno-substituted (1-6C)alkyl group with an appropriate amine or with a nitrogen-containing heterocyclyl compound.

The reaction is conveniently carried out in the presence of a suitable inert solvent or diluent as defined hereinbefore and at a temperature in the range, for example, 10 to 180° C., conveniently in the range 20 to 120° C., more conveniently at or near ambient temperature.

Compounds of the Formula I wherein the —X¹—R⁶ group is a halogeno-substituted (1-6C)alkyl group may be obtained by any of the representative process variants (a), (b) or (c) that are described hereinbefore or by methods described within the accompanying Examples that are set out hereinafter.

(e) For the production of those compounds of the Formula I wherein the —X¹—R⁶ group is an amino-substituted (1-6C)alkyl group (such as a methylaminomethyl, 2-methylaminoethyl or 2-hydroxyethylaminomethyl group), the reductive amination of a compound of the Formula I wherein the —X¹—R⁶ group is a formyl or (2-6C)alkanoyl group.

A suitable reducing agent for the reductive amination reaction is, for example, a hydride reducing agent, for example an alkali metal aluminium hydride such as lithium aluminium hydride or, preferably, an alkali metal borohydride such as sodium borohydride, sodium cyanoborohydride, sodium triethylborohydride, sodium trimethoxyborohydride and sodium triacetoxyborohydride. The reaction is conveniently performed in a suitable inert solvent or diluent, for example tetrahydrofuran and diethyl ether for the more powerful reducing agents such as lithium aluminium hydride, and, for example, methylene chloride or a protic solvent such as methanol and ethanol for the less powerful reducing agents such as sodium triacetoxyborohydride and sodium cyanoborohydride. The reaction is performed at a temperature in the range, for example, 10 to 80° C., conveniently at or near ambient temperature.

Compounds of the Formula I wherein the —X¹—R⁶ group is a formyl or (2-6C)alkanoyl group may be obtained by a conventional adaptation of any of the representative process variants (a), (b) or (c) that are described hereinbefore or by methods described within the accompanying Examples that are set out hereinafter.

(f) For the production of those compounds of the Formula I wherein an R¹ group is an amino-substituted or heterocyclyl-substituted (1-6C)alkoxy group (such as a 2-dimethylaminoethoxy or 2-pyrrolidin-1-ylethoxy group), the reaction, conveniently in the presence of a suitable base as defined hereinbefore, of a compound of the Formula I wherein the R¹ group is a halogeno-substituted (1-6C)alkoxy group with an appropriate amine or with a nitrogen-containing heterocyclyl compound.

The reaction is conveniently carried out in the presence of a suitable inert solvent or diluent as defined hereinbefore and at a temperature in the range, for example, 10 to 180° C., conveniently in the range 20 to 120° C., more conveniently at or near ambient temperature.

Compounds of the Formula I wherein the R¹ group is a halogeno-substituted (1-6C)alkoxy group may be obtained by any of the representative process variants (a), (b) or (c) that are described hereinbefore or by methods described within the accompanying Examples that are set out hereinafter.

When a pharmaceutically-acceptable salt of a quinazoline derivative of the Formula I is required, for example an acid-addition salt, it may be obtained by, for example, reaction of said quinazoline derivative with a suitable acid.

When a pharmaceutically-acceptable pro-drug of a quinazoline derivative of the Formula I is required, it may be obtained using a conventional procedure. For example, an in vivo cleavable ester of a quinazoline derivative of the Formula I may be obtained by, for example, reaction of a compound of the Formula I containing a carboxy group with a pharmaceutically-acceptable alcohol or by reaction of a compound of the Formula I containing a hydroxy group with a pharmaceutically-acceptable carboxylic acid. For example, an in vivo cleavable amide of a quinazoline derivative of the Formula I may be obtained by, for example, reaction of a compound of the Formula I containing a carboxy group with a pharmaceutically-acceptable amine or by reaction of a compound of the Formula I containing an amino group with a pharmaceutically-acceptable carboxylic acid.

Many of the intermediates defined herein are novel and these are provided as a further feature of the invention. For example, many compounds of the Formulae III, VI and VII are novel compounds.

### Biological Assays

The following assays can be used to measure the effects of the compounds of the present invention as inhibitors of PDGFRα, PDGFRβ and KDR tyrosine kinase enzymes, as inhibitors in vitro of the phosphorylation of PDGFR expressed on MG63 osteosarcoma cells, as inhibitors in vitro of the proliferation of MG63 osteosarcoma cells, as inhibitors in vitro of the proliferation of human umbilical vein endothelial cells (HUVECs), and as inhibitors in vivo of the growth in nude mice of xenografts of human tumour tissue such as CaLu-6 and Colo205.

### (a) In Vitro Enzyme Assays

The ability of test compounds to inhibit the phosphorylation of a tyrosine containing polypeptide substrate by the tyrosine kinase enzymes PDGFRα, PDGFRβ and KDR was assessed using conventional ELISA assays. DNA encoding the PDGFRα, PDGFRβ or KDR receptor cytoplasmic domains may be obtained by total gene synthesis (International Biotechnology Lab., 1987, 5(3), 19-25) or by cloning. The DNA fragments may be expressed in a suitable expression system to obtain polypeptide with tyrosine kinase activity. For example, PDGFRα, PDGFRβ and KDR receptor cytoplasmic domains, obtained by expression of recombinant protein in insect cells, can be shown to display intrinsic tyrosine kinase activity. In the case of the VEGF receptor KDR (Genbank Accession No. L04947), a DNA fragment encoding most of the cytoplasmic domain, commencing with methionine 806 and including the termination codon may be cloned into a baculovirus transplacement vector [for example pAcYM1 (see The Baculovirus Expression System: A Laboratory Guide, L. A. King and R. D. Possee, Chapman and Hall, 1992) or pAc360 or pBlueBacHis (available from Invitrogen Corporation)]. This recombinant construct may be co-transfected into insect cells [for example Spodoptera frugiperda 21(Sf21) or Spodoptera frugiperda 9(Sf9)] with viral DNA (for example Pharmingen BaculoGold) to prepare recombinant baculovirus. Details of the methods for the assembly of recombinant DNA molecules and the preparation and use of recombinant baculovirus can be found in standard texts, for example Sambrook et al., 1989, Molecular cloning—A Laboratory Manual, 2nd edition, Cold Spring Harbour Laboratory Press and O'Reilly et al., 1992, Baculovirus Expression Vectors—A Laboratory Manual, W. H. Freeman and Co, New York).

For expression, Sf9 cells were infected with plaque-pure KDR recombinant virus and harvested 48 hours later. Harvested cells were washed with ice cold phosphate buffered saline solution (PBS) containing 10 mM sodium phosphate pH7.4 buffer, 138 mM sodium chloride and 2.7 mM potassium chloride) and resuspended in ice cold cell diluent comprising 20 mM Hepes pH7.5 buffer, 150 mM sodium chloride, 10% v/v glycerol, 1% v/v Triton X100, 1.5 mM magnesium chloride, 1 mM ethylene glycol-bis(βaminoethyl ether) N,N,N′,N′-tetraacetic acid (EGTA) and 1 mM PMSF (phenylmethylsulphonyl fluoride) [the PMSF is added just before use from a freshly-prepared 100 mM solution in methanol] using 1 ml cell diluent per 10 million cells. The suspension was centrifuged for 10 minutes at 13,000 rpm at 4° C. The supernatant (stock enzyme solution) was removed and stored in aliquots at −70° C.

A substrate solution [100 μl of a 2 μg/ml solution of the poly-amino acid Poly(Glu, Ala, Tyr) 6:3:1 (Sigma-Aldrich Company Ltd., Poole, Dorset; Catalogue No. P3899) in phosphate buffered saline (PBS)] was added to each well of a number of Nunc 96-well MaxiSorp immunoplates (Nunc, Roskilde, Denmark; Catalogue No. 439454) and the plates were sealed and stored at 4° C. for 16 hours. The excess of substrate solution was discarded and the wells were washed in turn with PBS containing 0.05% v/v Tween 20 (PBST; 300 μl/well) and twice with Hepes pH7.4 buffer (50 mM, 300 μl/well) before being blotted dry.

Each test compound was dissolved in DMSO and diluted with a 10% solution of DMSO in distilled water to give a series of dilutions (from 40 μM to 0.0012 μM). Aliquots (25 μl) of each dilution of test compound were transferred to wells in the washed assay plates. “Maximum” control wells contained diluted DMSO instead of compound. Aliquots (25 μl) of an aqueous manganese chloride solution (40 mM) containing adenosine-5′-triphosphate (ATP) was added to all test wells except the “blank” control wells which contained magnesium chloride without ATP. For PDGFRα enzyme, an ATP concentration of 14 μM was used; for PDGFRβ enzyme, an ATP concentration of 2.8 μM was used and for KDR enzyme, an ATP concentration of 8 μM was used.

Active human PDGFRα and PDGFRβ recombinant enzyme that had been expressed in Sf9 insect cells was obtained from Upstate Biotechnology Inc., Milton Keynes, UK (product 14-467 for PDGFRα, product 14-463 for PDGFRβ). Active human KDR recombinant enzyme was expressed in Sf9 insect cells as described above.

Each kinase enzyme was diluted immediately prior to use with an enzyme diluent comprising 100 mM Hepes pH7.4 buffer, 0.1 mM sodium orthovanadate, 0.1% Triton X-100 and 0.2 mM dithiothreitol. Aliquots (50 μl) of freshly diluted enzyme were added to each well and the plates were agitated at ambient temperature for 20 minutes. The solution in each well was discarded and the wells were washed twice with PBST. Mouse IgG anti-phosphotyrosine antibody (Upstate Biotechnology Inc.; product 05-321; 100 μl) was diluted by a factor of 1:3667 with PBST containing 0.5% w/v bovine serum albumin (BSA) and aliquots were added to each well. The plates were agitated at ambient temperature for 1.5 hours. The supernatant liquid was discarded and each well was washed with PBST (×2). Horse radish peroxidase (HRP)-linked sheep anti-mouse Ig antibody (Amersham Pharmacia Biotech, Chalfont St Giles, Buckinghamshire, UK; Catalogue No. NXA 931; 100 μl) was diluted by a factor of 1:550 with PBST containing 0.5% w/v BSA and added to each well. The plates were agitated at ambient temperature for 1.5 hours. The supernatant liquid was discarded and the wells were washed with PBST (×2). A sodium perborate (PCSB) capsule (Sigma-Aldrich Company Ltd., Poole, Dorset, UK; Catalogue No. P4922) was dissolved in distilled water (100 ml) to provide phosphate-citrate pH5 buffer (50 mM) containing 0.03% sodium perborate. An aliquot (50 ml) of this buffer was mixed with a 50 mg tablet of 2,2′-azinobis(3-ethylbenzothiazoline-6-sulphonic acid) (ABTS; Roche Diagnostics Ltd., Lewes, East Sussex, UK; Catalogue No. 1204 521). An aliquot (100 μl) of the resultant solution was added to each well. The plates were agitated at ambient temperature for about 20 minutes until the optical density value of the “maximum” control wells, as measured at 405 nm using a plate reading spectrophotometer, was approximately 1.0. “Blank” (no ATP) and “maximum” (no compound) control values were used to determine the dilution range of test compound that gave 50% inhibition of enzyme activity.

(b) In Vitro phospho-Tyr751 PDGFRβELISA Assay

This assay uses a conventional ELISA method to determine the ability of test compounds to inhibit phosphorylation of tyrosine in PDGFRβ.

An MG63 osteosarcoma cell line [American Type Culture Collection (ATCC) CCL 1427] was routinely maintained at 37° C. with 7.5% CO₂ in Dulbecco's modified Eagle's growth medium (DMEM; Sigma-Aldrich; Catalogue No. D6546) containing 10% foetal calf serum (FCS; Sigma-Aldrich; Catalogue No. F7524) and 2 mM L-glutamine (Invitrogen Ltd., Paisley, UK; Catalogue No. 25030-024).

For the assay, the cells were detached from the culture flask using a trypsin/ethylenediaminetetraacetic acid (EDTA) mixture (Invitrogen Ltd.; Catalogue No. 15400-054) and resuspended in test media comprising DMEM without phenol red (Sigma-1-Aldrich; Catalogue No. D5921) containing 1% charcoal-stripped-foetal calf serum (FCS) (Sigma-Aldrich; Catalogue No. F7524, stripped by incubation with dextran-coated activated charcoal at 55° C. for 30 minutes with continuous stirring followed by removal of the charcoal by centrifugation and filter sterilisation) and 2 mM L-glutamine (Invitrogen Ltd., Catalogue No. 25030-024) to give 6×10⁴ cells per ml. Aliquots (100 μl) were seeded into each of the wells of columns 2-12 (excluding column 1) and rows B-G (excluding rows A and H) of a clear 96 well tissue culture plate (Corning Life Sciences, Koolhovenlaan, The Netherlands; Catalogue No. 3595) to give a density of about 6000 cells per well. Aliquots (100 μl) of culture media were placed in the outer wells to minimise edge effects. The cells were incubated overnight at 37° C. with 7.5% CO₂ to allow the cells to adhere to the wells.

Test compounds were prepared as 10 mM stock solutions in DMSO and serially diluted as required with test media to give a range of concentrations. Aliquots (50 μl) of each compound concentration were added to the cells in each well. Control cells received a dilution of DMSO only. The cells were incubated for 90 minutes at 37° C. with 7.5% CO₂.

The resultant cells were stimulated with PDGF_{BB} using the following procedure. A lyophilised powder of PDGF_{BB}, (Sigma-Aldrich; Catalogue No. P4306) was mixed with 4 mM aqueous hydrochloric acid containing 0.1% filter-sterilised BSA to provide a stock solution of 10 μg/ml of PDGF_{BB}. A dilution of this stock solution into test medium provided a 200 ng/ml PDGF_{BB} solution. Aliquots thereof (50 μl) were added to compound treated cells and to one set of control wells to give the “maximum” control. The “minimum” controls received media only. The cells were incubated at 37° C. with 7.5% CO₂ for 5 minutes. The solution from the wells was removed and the cells were lysed by the addition of 120 μl/well of RIPA buffer comprising 60 mM tris(hydroxymethyl)aminomethane hydrochloride (Tris-HCl), 150 mM sodium chloride, 1 mM EDTA, 1% v/v Igepal CA-630, 0.25% sodium deoxycholate, 1% v/v phosphatase inhibitor cocktail 1 P2850, 1% phosphatase inhibitor cocktail 2 P5726 and 0.5% v/v protease inhibitor cocktail P8340 (all chemicals and inhibitor cocktails were obtainable from the Sigma-Aldrich Company Ltd.). The resultant tissue culture plates were shaken for 5 minutes at ambient temperature to ensure full lysis and then frozen at −20° C. until required.

MaxiSorp ELISA plates (Nunc; Catalogue No. 439454) were coated with PDGFβ antibody (R&D Systems, Abingdon, Oxfordshire, UK; Catalogue No. AF385 comprising lyophilised antibody made up with 100 μl PBS to a final concentration of 100 μl/ml). The antibody was diluted at 1:40 into carbonate-bicarbonate buffer (Sigma-Aldrich; Catalogue No. C3041; one capsule dissolved in 100 ml of distilled water) to give a 2.5 μg/ml solution. Aliquots (100 μl) were added to each well and the plates were placed at 4° C. for 16 hours. The wells were washed 5 times (1 minute soak each time) with 300 μl per well of PBST. The wells were treated with 50 μl of 3% BSA in PBST at ambient temperature for 1 hour and subsequently washed twice with 300 μl per well of PBST.

The tissue culture plates with frozen cell lysate were allowed to warm to 0° C. Aliquots (50 μl) of the MG63 cell lysate were added to the ELISA plates. Each sample was duplicated on separate plates. The ELISA plates were agitated at ambient temperature for 2 hours. The wells were washed twice with 300 μl per well of PBST. A 1:1000 dilution of both total PDGFRβ antibody (Upstate Biotechnology Inc.; Catalogue No. 06-498) and phospho PDGFRβ antibody (Cell Signaling Technology, Beverley, Mass., USA; Catalogue No. 3161) was made into 1% BSA in PBST. Aliquots (50 μl) of the antibody solutions were added to each of the wells. Plates receiving the total antibody were labelled as ‘total antibody control’ plates, and plates receiving the phosphospecific antibody were labelled as ‘phospho antibody’ plates. The plates were agitated at ambient temperature for 1 hour. The plates were washed twice with 300 μl per well of PBST. A 1:2000 dilution of anti-rabbit horseradish peroxidase conjugated secondary antibody (Cell Signaling Technology; Catalogue No. 7074) was made into 1% BSA in PBST. Aliquots (50 μl) of the resultant dilution were added to each well and the plates were agitated at ambient temperature for 1 hour. The plates were washed 5 times with 300 μl per well of PBST. Chemiluminescent substrate was made up according to manufacturers instructions (Pierce Biotechnology Inc., Rockford Ill., USA; Catalogue No. 34080). Aliquots (50 μl) of chemiluminescent substrate solution were added to each of the wells, the plates were agitated for 2 minutes and luminescence was read on a SpectraFluor Plus plate reader (Tecan UK Ltd., Reading, Berkshire, UK). Analysis for each of the compounds was completed by determining a ratio of the ‘phospho antibody’ plate reading to the ‘total antibody’ plate reading for each test sample and these ratios were plotted to determine the IC₅₀ value of each test compound.

### (c) In Vitro MG63 Osteosarcoma Proliferation Assay

This assay determined the ability of a test compound to inhibit the proliferation of MG63 osteosarcoma cells (ATCC CCL 1427):

MG63 cells were seeded at 1.5×10³ cells per well into 96-well clear tissue culture-treated assay plates (Corning Life Sciences; Catalogue No. 3595) to which had been added 60 μl per well of test medium comprising DMEM without phenol red, 1% charcoal-stripped FCS and 2 mM glutamine and the cells were incubated overnight at 37° C. with 7.5% CO₂.

Test compounds were solubilised in DMSO to provide a 10 mM stock solution. Aliquots of the stock solution were diluted with the test medium described above and 20 μl aliquots of each dilution were added to appropriate wells. Serial dilutions were made to give a range of test concentrations. Control wells to which DMSO solution only was added were included on each plate. Each plate was duplicated. A lyophilised powder of PDGF_{BB} was mixed with 4 mM aqueous hydrochloric acid containing 0.1% filter-sterilised BSA to provide a stock solution of 10 μg/ml of PDGF_{BB}. A dilution of this stock solution into test medium provided a 250 ng/ml PDGF_{BB} solution. Aliquots (20 μl) thereof were added to one set of control wells to give the “maximum” control. Aliquots (20 μl) thereof were added to one set of the duplicate compound-treated plates and these were denoted as the “PDGF_{BB} stimulated” plates. The second set of duplicate compound-treated plates received media only and these were denoted as the “basal” plates. The “minimum” controls received media only. The plates were incubated at 37° C. with 7.5% CO₂ for 72 hours.

BrdU labelling reagent (Roche Diagnostics Ltd., Lewes, East Sussex, UK; Catalogue No. 647 229) was diluted by a factor of 1:100 in DMEM medium containing 1% charcoal stripped FCS and aliquots (10 μl) were added to each well to give a final concentration of 10 μM. The plates were incubated at 37° C. for 2 hours. The medium was decanted. A denaturating solution (FixDenat solution, Roche Diagnostics Ltd.; Catalogue No. 647 229; 200 μl) was added to each well and the plates were agitated at ambient temperature for 30 minutes. The supernatant was decanted and the wells were washed with PBS (200 μl per well). Anti-BrdU-Peroxidase solution (Roche Diagnostics Ltd.; Catalogue No. 647 229) was diluted by a factor of 1:100 in antibody diluent (Roche Diagnostics Ltd., Catalogue No. 647 229) and 100 μl of the resultant solution was added to each well. The plates were agitated at ambient temperature for 90 minutes. The wells were washed with PBS (×3; 300 μl) to ensure removal of non-bound antibody conjugate. The plates were blotted dry and tetramethylbenzidine substrate solution (Roche Diagnostics Ltd.; Catalogue No. 647 229; 100 μl) was added to each well. The plates were gently agitated on a plate shaker while the colour developed during a 10 to 20 minute period. Aqueous sulphuric acid (1M; 50 μl) was added to the appropriate wells to stop any further reaction and the absorbance of the wells was measured at 450 nm. The extent of inhibition of cellular proliferation at a range of concentrations of each test compound was determined and an anti-proliferative IC₅₀ value was derived.

### (d) In Vitro HUVEC Proliferation Assay

This assay determines the ability of a test compound to inhibit the growth factor-stimulated proliferation of human umbilical vein endothelial cells (HUVECs).

HUVECs were isolated in MCDB 131 (Gibco BRL) and 7.5% v/v foetal calf serum (FCS) and were plated out (at passage 2 to 8) in a mixture of MCDB 131, 2% v/v FCS, 3 μg/ml heparin and 1 μg/ml hydrocortisone, at a concentration of 1000 cells/well in 96 well plates. After a minimum of 4 hours, the cells were dosed with the appropriate growth factor (for example VEGF) and with the test compound. The cultures were incubated for 4 days at 37° C. under 7.5% CO₂. On day 4, the cell cultures were pulsed with 1 μCi/well of tritiated-thymidine (Amersham product TRA 61) and incubated for 4 hours. The cells were harvested using a 96-well plate harvester (Tomtek) and assayed for incorporation of tritium with a Beta plate counter. Incorporation of radioactivity into cells, expressed as counts per minute (cpm), was used to measure inhibition of growth factor-stimulated cell proliferation by each test compound.

### (e) In Vivo Solid Tumour Disease Model

This test measures the capacity of compounds to inhibit solid tumour growth.

CaLu-6 tumour xenografts were established in the flank of female athymic Swiss nu/nu mice, by subcutaneous injection of 1×10⁶ CaLu-6 cells/mouse in 100 μl of a 50% (v/v) solution of Matrigel in serum free culture medium. Ten days after cellular implant, mice were allocated to groups of 8-10 animals having comparable group mean tumour volumes. Tumours were measured using vernier calipers and volumes were calculated using the formula

(l×w)×√(l×w)×(π/6)

where l is the longest diameter and w the diameter perpendicular to the longest. Test compounds were administered orally once daily for a minimum of 21 days, and control animals received compound diluent only. Tumours were measured twice weekly. The level of growth inhibition was calculated by comparison of the mean tumour volume of the control group versus the treatment group using a Student's T test and/or a Mann-Whitney Rank Sum Test.

Although the pharmacological properties of the compounds of the Formula I vary with structural change as expected, in general activity possessed by compounds of the Formula I, may be demonstrated at the following concentrations or doses in one or more of the above tests (a), (b), (c), (d) and (e): —

Test (a): —
IC₅₀ versus PDGFRα tyrosine kinase in the range, for example,
0.1 nM-5 μM;
IC₅₀ versus PDGFRβ tyrosine kinase in the range, for example,
0.1 nM-5 M;
IC₅₀ versus KDR tyrosine kinase in the range, for example,
0.1 nM-5 μM;

Test (b): —
IC₅₀ versus phospho-Tyr751 PDGFRβ in the range, for example,
0.1 nM-1 μM;

Test (c): —
IC₅₀ versus MG63 osteosarcoma proliferation in the range, for example,
1 nM-5 μM;

Test (d): —
IC₅₀ versus HUVEC proliferation in the range, for example,
1 nM-5 μM;

Test (e): —
xenograft activity in the range, for example, 1-200 mg/kg/day.

For example, the quinazoline compound disclosed within Example 1 possesses activity in Test (a) with an IC₅₀ versus PDGFRα tyrosine kinase of approximately 7 nM, with an IC₅₀ versus PDGFRβ tyrosine kinase of approximately 0.5 nM, and with an IC₅₀ versus KDR tyrosine kinase of approximately 30 nM.

For example, the quinazoline compound disclosed within Example 2 possesses activity in Test (a) with an IC₅₀ versus PDGFRα tyrosine kinase of approximately 100 nM, with an IC₅₀ versus PDGFRβ tyrosine kinase of approximately 10 nM, and with an IC₅₀ versus KDR tyrosine kinase of approximately 1 nM; activity in Test (b) with an IC₅₀ versus phospho-Tyr751 PDGFR, of approximately 3 nM; and activity in Test (c) with an IC₅₀ versus MG63 osteosarcoma proliferation of approximately 40 nM.

For example, the quinazoline compound disclosed as Compound No. 8 within Example 9 possesses activity in Test (a) with an IC₅₀ versus PDGFRα tyrosine kinase of approximately 170 nM, with an IC₅₀ versus PDGFR, tyrosine kinase of approximately 100 nM, and with an IC₅₀ versus KDR-tyrosine kinase of approximately 10 nM; and activity in Test (b) with an IC₅₀ versus phospho-Tyr751 PDGFRβ of approximately 0.5 nM.

For example, the quinazoline compound disclosed as Compound No. 3 within Example 20 possesses activity in Test (a) with an IC₅₀ versus PDGFRα tyrosine kinase of approximately 240 nM, with an IC₅₀ versus PDGFR, tyrosine kinase of approximately 50 nM, and with an IC₅₀ versus KDR tyrosine kinase of approximately 10 nM; and activity in Test (b) with an IC₅₀ versus phospho-Tyr751 PDGFR, of less than 1 mM.

For example, the quinazoline compound disclosed as Compound No. 1 within within Example 35 possesses activity in Test (a) with an IC₅₀ versus KDR tyrosine kinase of approximately 35 nM; and activity in Test (b) with an IC₅₀ versus phospho-Tyr751 PDGFR, of approximately 1 nM.

For example, the quinazoline compound disclosed as Compound No. 13 within within Example 37 possesses activity in Test (a) with an IC₅₀ versus KDR tyrosine kinase of approximately 25 nM; and activity in Test (b) with an IC₅₀ versus phospho-Tyr751 PDGFRβ of approximately 3 nM.

For example, the quinazoline compound disclosed as Compound No. 26 within within Example 37 possesses activity in Test (a) with an IC₅₀ versus KDR tyrosine kinase of approximately 1 nM; and activity in Test (b) with an IC₅₀ versus phospho-Tyr751 PDGFRβ of approximately 1 nM.

For example, the quinazoline compound disclosed within Example 40 possesses activity in Test (a) with an IC₅₀ versus KDR tyrosine kinase of approximately 2 nM; and activity in Test (b) with an IC₅₀ versus phospho-Tyr751 PDGFRβ of approximately 1 nM. No untoward toxicological effects are expected when a compound of Formula I, or a pharmaceutically-acceptable salt thereof, as defined hereinbefore is administered at the dosage ranges defined hereinafter.

According to a further aspect of the invention there is provided a pharmaceutical composition which comprises a quinazoline derivative of the Formula I, or a pharmaceutically-acceptable salt, solvate or pro-drug thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 1 mg to 1 g of active agent (more suitably from 1 to 250 mg, for example from 1 to 100 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the disease state, the age and sex of the animal or patient and the route of administration, according to well known principles of medicine.

In using a compound of the Formula I for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Similarly, for administration by inhalation, a dose in the range, for example, 1 mg/kg to 25 mg/kg body weight will be used. Oral administration is however preferred, particularly in tablet form. Typically, unit dosage forms will contain about 10 mg to 0.5 g of a compound of this invention.

As stated above, antagonism of the activity of PDGF receptor kinases, particularly inhibition of the PDGFα (and/or PDGFβ receptor tyrosine kinases, is expected to be beneficial in the treatment of a number of cell proliferative disorders such as cancer, especially in inhibiting tumour growth and metastasis and in inhibiting the progression of leukaemia. IT addition, antagonism of the activity of VEGF is expected to be beneficial in the treatment of a number of disease states that are associated with angiogenesis and/or increased vascular permeability such as cancer, especially in inhibiting the development of tumours.

We have now found that the novel 4-(4-pyrazolyloxy)quinazoline derivatives described herein possess potent activity against cell proliferative disorders. It is believed that the compounds provide a useful treatment of cell proliferative disorders, for example to provide an anti-tumour effect, by way of a contribution from inhibition of PDGF receptor tyrosine kinases and/or by way of a contribution from inhibition of VEGF receptor tyrosine kinases.

According to this further aspect of the invention there is provided a quinazoline derivative of the Formula I, or a pharmaceutically-acceptable salt, solvate or pro-drug thereof, as defined hereinbefore for use as a medicament in a warm-blooded animal such as man.

According to a further aspect of the invention, there is provided a quinazoline derivative of the Formula I, or a pharmaceutically-acceptable salt, solvate or pro-drug thereof, as defined hereinbefore for use in the treatment (or prophylaxis) of cell proliferative disorders or in the treatment (or prophylaxis) of disease states associated with angiogenesis and/or vascular permeability.

According to a further aspect of the invention, there is provided the use of a quinazoline derivative of the Formula I, or a pharmaceutically-acceptable salt, solvate or pro-drug thereof, as defined hereinbefore in the manufacture of a medicament for use in the treatment (or prophylaxis) of cell proliferative disorders or in, the treatment (or prophylaxis) of disease states associated with angiogenesis and/or vascular permeability.

According to this aspect of the invention there is also provided a method for the treatment (or prophylaxis) of cell proliferative disorders in a warm-blooded animal in need of such treatment (or prophylaxis) or for the treatment (or prophylaxis) of disease states associated with angiogenesis and/or vascular permeability in a warm-blooded animal in need of such treatment (or prophylaxis) which comprises administering to said animal an effective amount of a quinazoline derivative of the Formula I, or a pharmaceutically-acceptable salt, solvate or pro-drug thereof, as defined hereinbefore.

Suitable cell proliferative disorders include neoplastic disorders, for example, cancers of the lung (non-small cell lung cancer, small cell lung cancer and bronchioalveolar cancer), gastrointestine (such as colon, rectal and stomach tumours), prostate, breast, kidney, liver, brain (such as glioblastoma), bile duct, bone, bladder, head and neck, oesophagus, ovary, pancreas, testes, thyroid, cervix and vulva and skin (such as dermatofibrosarcoma protruberans) and in leukaemias and lymphomas such as chronic myelogenous leukaemia (CML), chronic myelomonocytic leukaemia (CMML), acute lymphocytic leukaemia (ALL), chronic neutrophilic leukaemia (CNL), acute myelogenous leukaemia (AML) and multiple myeloma.

According to this aspect of the invention there is also provided a method for treating cell proliferative disorders (such as solid tumour disease) in a warm-blooded animal in need of such treatment which comprises administering to said animal an effective amount of a quinazoline derivative of the Formula I, or a pharmaceutically-acceptable salt, solvate or pro-drug thereof, as defined hereinbefore.

Other suitable cell proliferative disorders include non-malignant disorders such as blood vessel disease (for example atherosclerosis and restenosis, for example in the process of restenosis subsequent to balloon angioplasty and heart arterial by-pass surgery), fibrotic diseases (for example kidney fibrosis, hepatic cirrhosis, lung fibrosis and multicystic renal dysplasia), glomerulonephritis, benign prostatic hypertrophy, inflammatory diseases (for example rheumatoid arthritis and inflammatory bowel disease), multiple sclerosis, psoriasis, hypersensitivity reactions of the skin, allergic asthma, insulin-dependent diabetes, diabetic retinopathy and diabetic nephropathy.

Suitable disease states associated with angiogenesis and/or vascular permeability include, for example, the undesirable or pathological angiogenesis seen in diabetic retinopathy, psoriasis, cancer, rheumatoid arthritis atheroma, Kaposi's sarcoma and haemangioma.

According to a further aspect of the invention there is provided a quinazoline derivative of the Formula I, or a pharmaceutically-acceptable salt, solvate or pro-drug thereof, as defined hereinbefore for use in the treatment (or prevention) of those tumours which are sensitive to inhibition of PDGF receptor enzymes (such as PDGFα and/or PDGFβ receptor tyrosine kinase) and/or which are sensitive to inhibition of VEGF receptor tyrosine kinases (such as KDR and/or Flt-1 receptor tyrosine kinase) that are involved in the signal transduction steps which lead to the proliferation, survival, invasiveness and migratory ability of tumour cells.

According to a further feature of this aspect of the invention there is provided the use of a quinazoline derivative of the Formula I, or a pharmaceutically-acceptable salt, solvate or pro-drug thereof, as defined hereinbefore in the manufacture of a medicament for use in the treatment (or prevention) of those tumours which are sensitive to inhibition of PDGF receptor enzymes (such as PDGFα and/or PDGFβ receptor tyrosine kinase) and/or which are sensitive to inhibition of VEGF receptor tyrosine kinases (such as KDR and/or Flt-1 receptor tyrosine kinase) that are involved in the signal transduction steps which lead to the proliferation, survival, invasiveness and migratory ability of tumour cells.

According to a further feature of this aspect of the invention there is provided a method for the treatment (or prevention) of a warm-blooded animal having tumours which are sensitive to inhibition of PDGF receptor enzymes (such as PDGFα and/or PDGFβ receptor tyrosine kinase) and/or which are sensitive to inhibition of VEGF receptor tyrosine kinases (such as KDR and/or Flt-1 receptor tyrosine kinase) that are involved in the signal transduction steps which lead to the proliferation, survival, invasiveness and migratory ability of tumour cells which comprises administering to said animal an effective amount of a quinazoline derivative of the Formula I, or a pharmaceutically-acceptable salt, solvate or pro-drug thereof, as defined hereinbefore.

According to a further aspect of the invention there is provided a quinazoline derivative of the Formula I, or a pharmaceutically-acceptable salt, solvate or pro-drug thereof, as defined hereinbefore for use in providing a PDGF receptor enzyme inhibitory effect (such as a PDGFα and/or PDGFβ receptor tyrosine kinase inhibitory effect) and/or a VEGF receptor tyrosine kinase inhibitory effect (such as a KDR and/or Flt-1 receptor tyrosine kinase inhibitory effect).

According to a further feature of this aspect of the invention there is provided the use of a quinazoline derivative of the Formula I, or a pharmaceutically-acceptable salt, solvate or pro-drug thereof, as defined hereinbefore in the manufacture of a medicament for use in providing a PDGF receptor enzyme inhibitory effect (such as a PDGFα and/or PDGFβ receptor tyrosine kinase inhibitory effect) and/or a VEGF receptor tyrosine kinase inhibitory effect (such as a KDR and/or Flt-1 receptor tyrosine kinase inhibitory effect).

According to a further aspect of the invention there is also provided a method for inhibiting a PDGF receptor enzyme (such as the PDGFα and/or PDGFβ receptor tyrosine kinase) and/or inhibiting a VEGF receptor tyrosine kinase (such as the KDR and/or Flt-1 receptor tyrosine kinase) which comprises administering an effective amount of a quinazoline derivative of the Formula I, or a pharmaceutically-acceptable salt, solvate or pro-drug thereof, as defined hereinbefore.

The anti-cancer treatment defined hereinbefore may be applied as a sole therapy or may involve, in addition to the quinazoline derivative of the invention, conventional surgery or radiotherapy or chemotherapy. Such chemotherapy may include one or more of the following categories of anti-tumour agents: —

(i) other antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyqyuinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)) and inhibitors of cell signalling through MEK, AKT and/or PI3K kinases;
(v) other antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a quinazoline derivative of the formula I as defined hereinbefore and an additional anti-tumour agent as defined hereinbefore for the conjoint treatment of cancer.

Basis for another aspect of the invention arises from the disclosure in International Patent Application WO 01/74360 that VEGF receptor tyrosine kinase inhibitors, provided that they possess suitable pharmacokinetic properties which provide reasonable bioavailability, lead to a sustained increase in blood pressure when administered to warm-blooded animals, particularly when administered chronically. In general, the compounds of the present invention possess inhibitory activity against VEGF receptor tyrosine kinases such as KDR tyrosine kinase. Accordingly, it is expected that, in general, compounds of the present invention will cause a sustained increase in blood pressure when administered to a warm-blooded animal such as man. In order to attenuate such a hypertensive effect, the anti-cancer treatment defined hereinbefore may involve the administration, in addition to the quinazoline derivative of the invention, of a conventional anti-hypertensive agent. Such an anti-hypertensive agent may include one or more of the following categories of anti-hypertensive agents

(i) calcium channel blockers such as amlodipine, diltiazem and felodipine;
(ii) angiotensin converting enzyme inhibitors (ACE-Inhibitors) such as captopril, enalapril, lisinopril and quinapril;
(iii) angiotensin-II receptor antagonists (A-II antagonists) such as candesartan, losartan and valsartan;
(iv) β-blockers such as atenolol, metoprolol and timolol;
(v) α-blockers such as doxazosin, prazosin and tamsulosin;
(vi) “vasodilators”, which include cerebral vasodilators, coronary vasodilators and peripheral vasodilators, such as cinnarizine, fenoxedil, pentifylline and dipyridamole; and
(vii) “diuretics”, which include benzothiadiazine derivatives, diuretic organomercurials, diuretic purines, diuretic steroids, diuretic sulfonamide derivatives and diuretic uracils, for example amiloride, bendroflumethiazide, hydrochlorothiazide, clopamide and furosemide.

According to this aspect of the invention there is provided a pharmaceutical product for use in the treatment of cancer which comprises a quinazoline derivative of the formula I as defined hereinbefore and an anti-hypertensive agent as defined hereinbefore.

Although the compounds of the Formula I are primarily of value as therapeutic agents for use in warm-blooded animals (including man), they are also useful whenever it is required to inhibit the effects of PDGF receptor tyrosine kinase enzymes. Thus, they are useful as pharmacological standards for use in the development of new biological tests and in the search for new pharmacological agents.

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulphate, work-up procedures were carried out after removal of residual solids by filtration, evaporations were carried out by rotary evaporation in vacuo;

(iv) yields, where present, are not necessarily the maximum attainable, and, when necessary, reactions were repeated if a larger amount of the reaction product was required;

(v) in general, the structures of the end-products of the Formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using a Bruker Spectrospin DPX300 spectrometer operating at a field strength of 300 MHz; the following abbreviations have been used: s, singlet; d, doublet; t, triplet; q, quartet; m, multiplet; br, broad;

(vi) unless stated otherwise compounds containing an asymmetric carbon and/or sulphur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of a 1% aqueous acetic acid or a 1% aqueous ammonium hydroxide (d=0.88) solution and acetonitrile;

(x) where certain compounds were obtained as an acid-addition salt, for example a mono-hydrochloride salt or a di-hydrochloride salt, the stoichiometry of the salt was based on the number and nature of the basic groups in the compound; generally, elemental analysis data were not obtained to determine the exact stoichiometry of the salt;

(xi) the following abbreviations have been used: —
DMSO dimethylsulphoxide
DMF N,N-dimethylformamide
DMA N,N-dimethylacetamide
THF tetrahydrofuran

### EXAMPLE 1

### N-(3,4-methylenedioxyphenyl)-2-[4-(6,7-dimethoxyquinazolin-4-yloxy)pyrazol-1-yl]acetamide

Diisopropylethylamine (0.058 g) and 2-(7-azabenzotriazol-1-yl)-1,1,3,3-tetramethyluronium hexafluorophosphate(V) (0.167 g) were added in turn to a stirred mixture of 2-[4-(6,7-dimethoxyquinazolin-4-yloxy)pyrazol-1-yl]acetic acid (0.132 g), 3,4-methylenedioxyaniline (European Patent Application No. 0549263, Example 3 thereof; 0.066 g) and DMF (1.3 ml) and the resultant mixture was stirred at ambient temperature for 16 hours. Water was added and the precipitate was recovered by filtration and purified by preparative HPLC using a Waters ‘Symmetry’ C18 reversed-phase column (5 microns silica, 19 mm diameter, 100 mm length) and decreasingly polar mixtures of water (containing 2% acetic acid) and acetonitrile as eluent. There was thus obtained the title compound as a solid (0.065 g); ¹H NMR: (DMSOd₆) 3.98 (s, 3H), 3.99 (s, 3H), 5.0 (s, 2H), 6.0 (s, 2H), 6.88 (d, 1H), 6.98 (m, 1H), 7.3 (d, 1H), 7.39 (s, 1H), 7.54 (s, 1H), 7.67 (s, 1H), 8.11 (s, 1H), 8.65 (s, 1H), 10.26 (s, 1H); Mass Spectrum: M+H⁺ 450.

The 2-[4-(6,7-dimethoxyquinazolin-4-yloxy)pyrazol-1-yl]acetic acid used as a starting material was prepared as follows: —

A mixture of 4-(tert-butyldimethylsilyloxy)-1H-pyrazole (European Patent Application No. 0921120, pages 34 and 35 thereof; 2.77 g), tert-butyl bromoacetate (2.87 g), potassium carbonate (3.86 g) and DMF (40 ml) was stirred at ambient temperature for 21 hours. The resultant mixture was evaporated and the residue was partitioned between ethyl acetate and water. The organic phase was washed with a saturated aqueous sodium chloride solution, dried over magnesium sulphate and evaporated. There was thus obtained tert-butyl 2-[4-(tert-butyldimethylsilyloxy)pyrazol-1-yl]acetate as a pale yellow liquid (4.37 g);

¹H NMR: (DMSOd₆) 0.16 (s, 6H), 0.94 (s, 9H), 1.42 (s, 9H), 4.77 (s, 2H), 7.14 (s, 1H), 7.4 (s, 1H); Mass Spectrum: M+H+313.

Tetra-n-butylammonium fluoride (1.1 M in THF; 1.1 ml) and acetic acid (0.144 g) were added in turn to a solution of tert-butyl 2-[4-(tert-butyldimethylsilyloxy)pyrazol-1-yl]acetate (0.312 g) in THF (2 ml) that had been cooled to 5° C. The resultant mixture was stirred at ambient temperature for 1 hour. The reaction mixture was diluted with a saturated aqueous sodium bicarbonate solution and extracted with ethyl acetate. The organic phase was recovered, washed with water, dried over magnesium sulphate and evaporated. The residual oil was triturated under diethyl ether. There was thus obtained tert-butyl 2-(4-hydroxypyrazol-1-yl)acetate (0.165 g); ¹H NMR: (DMSOd₆) 1.4 (s, 9H), 4.72 (s, 2H), 7.01 (s, 1H), 7.18 (s, 1H), 8.42 (s, 1H).

Sodium hydride (60% dispersion in mineral oil, 0.024 g) was added portionwise to a stirred mixture of tert-butyl 2-(4-hydroxypyrazol-1-yl)acetate (0.029 g) and DMF (3 ml) and the resultant mixture was stirred at ambient temperature for 15 minutes and heated at 40° C. for a further 15 minutes. 4-Chloro-6,7-dimethoxyquinazoline (European Patent Application No. 0566226, Example 1 thereof; 0.112 g) was added and the resultant mixture was heated to 60° C. for 2 hours. The mixture was cooled to ambient temperature, diluted with an aqueous ammonium chloride solution and extracted with a 1:1 mixture of diethyl ether and ethyl acetate. The organic phase was washed with a saturated aqueous sodium chloride solution, dried over magnesium sulphate and evaporated. There was thus obtained tert-butyl 2-[4-(6,7-dimethoxyquinazolin-4-yloxy)pyrazol-1-yl]acetate (0.17 g); ¹H NMR: (DMSOd₆) 1.45 (s, 9H), 3.98 (s, 3H), 3.99 (s, 3H), 4.96 (s, 2H), 7.39 (s, 1H), 7.53 (s, 1H), 7.67 (s, 1H), 8.1 (s, 1H), 8.64 (s, 1H); Mass Spectrum: M+H⁺ 387.

A mixture of a solution of the material so obtained in methylene chloride (5 ml) and an excess of a 20:1 mixture of trifluoroacetic acid and water was stirred at ambient temperature for 3 hours. The resultant mixture was evaporated. The residue was dissolved in ethyl acetate and diethyl ether was added. The resultant precipitate was recovered by filtration, suspended in methylene chloride and treated with an excess of diisopropylethylamine. The resultant solution was evaporated and the residual oil was triturated under a mixture of methylene chloride, diethyl ether and ethyl acetate. The solid so obtained was dried under vacuum at 50° C. for 1 hour. There was thus obtained 2-[4-(6,7-dimethoxyquinazolin-4-yloxy)pyrazol-1-yl]acetic acid (0.115 g); ¹H NMR: (DMSOd₆) 3.98 (s, 3H), 3.99 (s, 3H), 4.94 (s, 2H), 7.38 (s, 1H), 7.53 (s, 1H), 7.65 (s, 1H), 8.06 (s, 1H), 8.65 (s, 1H); Mass Spectrum: M+H⁺ 331.

### EXAMPLE 2

### N-(2,3-methylenedioxyphenyl)-2-[4-(6,7-dimethoxyquinazolin-4-yloxy)pyrazol-1-yl]acetamide

Using an analogous procedure to that described in Example 1, 2-[4-(6,7-dimethoxyquinaolin-4-yloxy)pyrazol-1-yl]acetic acid was reacted with 2,3-methylenedioxyaniline (J. Med. Chem., 1979, 22, 1354) to give the title compound in 61% yield; ¹H NMR: (DMSOd₆) 3.98 (s, 3H), 3.99 (s, 3H), 5.09 (d, 2H), 6.06 (s, 2H), 6.75 (d, 1H), 6.82 (m, 1H), 7.31 (d, 1H), 7.39 (s, 1H), 7.54 (s, 1H), 7.68 (s, 1H), 8.11 (s, 1H), 8.65 (s, 1H), 10.08 (s, 1H); Mass Spectrum: M+H⁺ 450.

### EXAMPLE 3

### N-(6-fluoro-2,3-methylenedioxyphenyl)-2-[4-(6,7-dimethoxyquinazolin-4-yloxy)pyrazol-1-yl]acetamide

Using an analogous procedure to that described in Example 1, 2-[4-(6,7-dimethoxyquinazolin-4-yloxy)pyrazol-1-yl]acetic acid was reacted with 6-fluoro-2,3-methylenedioxyaniline (International Patent Application WO 03/008409, Example 10, Note [4] thereof) to give the title compound in 24% yield; ¹H NMR: (DMSOd₆) 3.98 (s, 3H), 3.99 (s, 3H), 5.09 (s, 2H), 6.07 (d, 2H), 6.73 (m, 1H), 6.82 (m, 1H), 7.39 (s, 1H), 7.54 (s, 1H), 7.67 (s, 1H), 8.11 (s, 1H), 8.65 (s, 1H), 10.22 (br s, 1H); Mass Spectrum: M+H⁺ 468.

### EXAMPLE 4

### N-(2,3-methylenedioxyphenyl)-2-[4-(5,7-dimethoxyquinazolin-4-yloxy)pyrazol-1-yl]acetamide

Using an analogous procedure to that described in Example 1, 2-[4-(5,7-dimethoxyquinazolin-4-yloxy)pyrazol-1-yl]acetic acid was reacted with 2,3-methylenedioxyaniline to give the title compound in 82% yield; ¹H NMR: (DMSOd₆) 3.94 (s, 3H), 3.96 (s, 3H), 4.03 (s, 2H), 5.06 (s, 2H), 6.06 (s, 2H), 6.75 (d, 1H), 6.77 (d, 1H), 6.81 (m, 1H), 6.96 (d, 1H), 7.3 (d, 1H), 7.58 (s, 1H), 8.0 (s, 1H), 8.6 (s, 1H), 10.07 (s, 1H); Mass Spectrum: M+H⁺ 450.

The 2-[4-(5,7-dimethoxyquinazolin-4-yloxy)pyrazol-1-yl]acetic acid used as a starting material was prepared as follows: —

Phosphoryl chloride (0.551 g) was added to a mixture of 5,7-dimethoxy-3,4-dihydroquinazolin-4-one (International Patent Application WO 01/94341, Example 1 thereof; 0.618 g), diisopropylethylamine (1 ml) and 1,2-dichloroethane (20 ml) and the resultant mixture was stirred and heated to 80° C. for 3.5 hours. The mixture was evaporated and the residue was purified by column chromatography on silica using a 4:1 mixture of methylene chloride and ethyl acetate as eluent. There was thus obtained 4-chloro-5,7-dimethoxyquinazoline as a solid (0.52 g); ¹H NMR: (DMSOd₆) 3.96 (s, 3H), 3.97 (s, 3H), 6.86 (d, 1H), 7.04 (d, 1H), 8.84 (s, 1H); Mass Spectrum: M+H⁺ 225.

A mixture of 4-chloro-5,7-dimethoxyquinazoline (0.258 g), tert-butyl 2-(4-hydroxypyrazol-1-yl)acetate (0.228 g), potassium carbonate (0.317 g) and DMF (2.6 ml) was stirred and heated to 90° C. for 1 hour. The resultant mixture was cooled to ambient temperature, diluted with ethyl acetate and filtered. The organic phase was evaporated and the residual oil was triturated under diethyl ether to give tert-butyl 2-[4-(5,7-dimethoxyquinazolin-4-yloxy)pyrazol-1-yl]acetate as a solid (0.378 g); ¹H NMR: (DMSOd₆) 1.45 (s, 9H), 3.94 (s, 3H), 3.96 (s, 3H), 4.93 (s, 2H), 6.76 (s, 1H), 6.95 (s, 1H), 7.57 (s, 1H), 7.97 (s, 1H); 8.59 (s, 1H); Mass Spectrum: M+H⁺ 387.

A mixture of the material so obtained, a 5% aqueous solution of trifluoroacetic acid (10 ml) and methylene chloride (5 ml) was stirred at ambient temperature for 4 hours. The solvent was evaporated and the residue was triturated under diethyl ether. The resultant solid was isolated and suspended in methylene chloride (15 ml). An excess diisopropylethylamine was added to obtain a clear solution. The solution was evaporated and the residual oil was triturated under ethyl acetate (15 ml). The solid so obtained was isolated and dried under vacuum. There was thus obtained 2-[4-(5,7-dimethoxyquinazolin-4-yloxy)pyrazol-1-yl]acetic acid (0.305 g); ¹H NMR: (DMSOd₆) 3.94 (s, 3H), 3.96 (s, 3H), 6.77 (s, 1H), 6.95 (s, 1H), 7.55 (s, 1H), 7.96 (s, 1H), 8.59 (s, 1H); Mass Spectrum: M+H⁺ 331.

### EXAMPLE 5

### N-(3,4-ethylenedioxyphenyl)-2-[4-(6,7-dimethoxyquinazolin-4-yloxy)pyrazol-1-yl]acetamide

Using an analogous procedure to that described in Example 1, 2-[4-(6,7-dimethoxyquinazolin-4-yloxy)pyrazol-1-yl]acetic acid was reacted with 3,4-ethylenedioxyaniline to give the title compound in 71% yield; ¹H NMR: (DMSOd₆) 3.98 (s, 3H), 3.99 (s, 3H), 4.17-4.26 (m, 4H), 4.98 (d, 2H), 6.81 (d, 1H), 6.98 (m, 1H), 7.23 (d, 1H), 7.39 (s, 1H), 7.54 (s, 1H), 7.67 (s, 1H), 8.1 (s, 1H), 8.65 (s, 1H), 10 18 (s, 1H); Mass Spectrum: M+H⁺ 464.

### EXAMPLE 6

Using an analogous procedure to that described in Example 1, the appropriate 2-(pyrazol-1-yl)acetic acid was reacted with the appropriate aniline to give the compounds described in Table I. Unless otherwise stated, each aniline was commercially available.

TABLE I

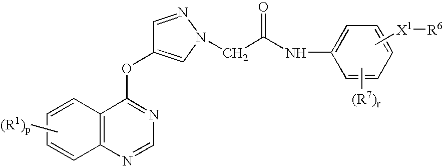

| No. & | | | |
|---|---|---|---|
| Note | (R¹)ₚ | (R⁷)ᵣ | X¹—R⁶ |
| [1] | 6,7-dimethoxy | H | 3-hydroxymethyl |
| [2] | 6,7-dimethoxy | H | 4-hydroxymethyl |
| [3] | 6,7-dimethoxy | H | 3-(1-hydroxyethyl) |
| [4] | 6,7-dimethoxy | H | 2-cyanomethyl |
| [5] | 6,7-dimethoxy | H | 3-sulphamoyl |
| [6] | 6,7-dimethoxy | H | 4-sulphamoyl |
| [7] | 6,7-dimethoxy | H | 4-piperidino |
| [8] | 6,7-dimethoxy | H | 4-morpholino |
| [9] | 6,7-dimethoxy | 6-fluoro | 3-hydroxymethyl |

Notes The products gave the characterising data shown below.

[1] ¹H NMR: (DMSOd₆) 3.98 (s, 3H), 3.99 (s, 3H), 4.48 (d, 2H), 5.04 (s, 2H), 5.21 (t, 1H), 7.02 (d, 1H), 7.27 (m. 1H), 7.39 (s, 1H), 7.5 (d, 1H), 7.54 (s, 1H), 7.57 (s, 1H), 7.68 (s, 1H), 8.12 (s, 1H), 8.66 (s, 1H), 10.32 (s, 1H); Mass Spectrum: M+H⁺ 436.

[2] ¹H NMR: (DMSOd₆) 3.98 (s, 3H), 3.99 (s, 3H), 4.44 (d, 2H), 5.03 (s, 2H), 5.12 (t, 1H), 7.27 (d, 2H), 7.4 (s, 1H), 7.54 (s, 1H), 7.55 (d, 2H), 7.68 (s, 1H), 8.12 (s, 1H), 8.66 (s, 1H), 10.31 (s, 1H); Mass Spectrum: M+H⁺ 436.

[3] ¹H NMR: (DMSOd₆) 1.3 (d, 3H), 3.98 (s, 3H), 3.99 (s, 3H), 4.68 (q, 1H), 5.02 (s, 2H), 5.18 (br s, 1H), 7.04 (d, 1H), 7.26 (m, 1H), 7.4 (s, 1H), 7.49 (m, 1H), 7.55 (s, 1H), 7.59 (br s, 1H), 7.67 (s, 1H), 8.12 (s, 1H), 8.66 (s, 1H), 10.32 (s, 1H); Mass Spectrum: M+H⁺ 450.

[4] ¹H NMR: (DMSOd₆) 3.96 (s, 2H), 3.98 (s, 3H), 3.99 (s, 3H), 5.1 (s, 2H), 7.3 (m, 1H), 7.38 (d, 1H), 7.39 (s, 1H), 7.42 (d, 1H), 7.46 (d, 1H), 7.54 (s, 1H), 7.69 (s, 1H), 8.17 (s, 1H), 8.66 (s, 1H), 10.03 (br s, 1H); Mass Spectrum: M+H⁺ 445.

[5] ¹H NMR: (DMSOd₆) 3.98 (s, 2H), 3.99 (s, 3H), 5.08 (s, 2H), 7.39 (s, 1H), 7.52-7.57 (m, 3H), 7.69 (s, 1H), 7.76 (m, 1H), 8.14 (s, 1H), 8.17 (br s. 1H), 8.66 (s, 1H), 10.67 (br s, 1H); Mass Spectrum: M+H⁺ 485.

[6] ¹H NMR: (DMSOd₆) 3.98 (s, 3H), 3.99 (s, 3H), 5.09 (s, 2H), 7.28 (s, 2H), 7.39 (s, 1H), 7.55 (s, 1H), 7.69 (s, 1H), 7.76 (d, 2H), 7.79 (d, 2H), 8.14 (s, 1H), 8.66 (s, 1H), 10.7 (s, 1H); Mass Spectrum: M+H⁺ 485.

[7] ¹H NMR: (DMSOd₆) 1.42-1.69 (m, 6H), 3.04-3.09 (m, 4H), 3.98 (s, 3H), 3.99 (s, 3H), 4.98 (s, 2H), 6.89 (d, 2H), 7.39 (s, 1H), 7.43 (d, 2H), 7.54 (s, 1H), 7.67 (s, 1H), 8.11 (s, 1H), 8.66 (s, 1H), 10.1 (s, 1H); Mass Spectrum: M+H⁺ 489.

[8] ¹H NMR: (DMSOd₆) 3.01-3.09 (m, 4H), 3.68-3.77 (m, 4H), 3.98 (s, 3H), 3.99 (s, 3H), 4.99 (s, 2H), 6.91 (d, 2H), 7.39 (s, 1H), 7.47 (d, 2H), 7.54 (s, 1H), 7.67 (s, 1H), 8.11 (s, 1H), 8.66 (s, 1H), 10.14 (s, 1H); Mass Spectrum: M+H⁺ 491.

[9] At the end of the reaction period, the reaction mixture was poured into a 2N aqueous sodium bicarbonate solution. The resultant precipitate was recovered and dried under vacuum at 50° C. for 2 hours. The material so obtained gave the following characterising data: —

¹H NMR: (DMSOd₆) 3.98 (s, 6H), 4.53 (d, 2H), 5.05 (s, 2H), 5.46 (t, 1H), 7.36 (m, 1H), 7.39 (s, 1H), 7.54 (s, 1H), 7.61 (m, 1H), 7.69 (s, 1H), 7.77 (m, 1H), 8.12 (s, 1H), 8.65 (s, 1H); Mass Spectrum: M+H⁺ 470.

The 6-fluoro-3-hydroxymethylaniline used as a starting material was prepared as follows: —

A mixture of 4-fluoro-3-nitrobenzyl alcohol (0.5 g), 10% platinum-on-carbon catalyst (0.1 g) and ethyl acetate (25 ml) was stirred under 1.8 atmospheres pressure of hydrogen for 1 hour. The reaction mixture was filtered and the filtrate was evaporated. The residue was purified by column chromatography on silica using a 2:1 mixture of methylene chloride and ethyl acetate as eluent. There was thus obtained 6-fluoro-3-hydroxymethylaniline ¹H NMR: (DMSOd₆) 4.41 (d, 2H), 5.19 (m, 3H) 6.42 (m, 1H), 6.77 (s, 1H), 6.95 (m, 1H).

### EXAMPLE 7

### N-(3-oxo-1,3-dihydroisobenzofuran-5-yl)-2-[4-(6,7-dimethoxyquinazolin-4-yloxy)pyrazol-1-yl]acetamide

Using an analogous procedure to that described in Example 1, 2-[4-(6,7-dimethoxyquinazolin-4-yloxy)pyrazol-1-yl]acetic acid was reacted with 5-amino-3-oxo-1,3-dihydroisobenzofuran to give the title compound in 97% yield; ¹H NMR: (DMSOd₆) 3.98 (s, 3H), 3.99 (s, 3H), 4.03 (s, 2H), 5.1 (d, 2H), 5.38 (s, 2H), 7.4 (s, 1H), 7.55 (s, 1H), 7.66 (d, 1H), 7.69 (s, 1H), 7.85 (m, 1H), 8.15 (s, 1H), 8.2 (d, 1H), 8.66 (s, 1H), 10.8 (br s, 1H); Mass Spectrum: M+H⁺ 462.

### EXAMPLE 8

### N-[6-(2-dimethylaminoethoxy)pyridin-3-yl]-2-[4-(6,7-dimethoxyquinazolin-4-yloxy)pyrazol-1-yl]acetamide

A solution of 2-(7-azabenzotriazol-1-yl)-1,1,3,3-tetramethyluronium hexafluorophosphate(V) (0.205 g) in DMF (0.8 ml) was added to a stirred mixture of 2-[4-(6,7-dimethoxyquinazolin-4-yloxy)pyrazol-1-yl]acetic acid (0.149 g), 3-amino-6-(2-dimethylaminoethoxy)pyridine (0.098 g), diisopropylethylamine (0.07 g) and DMF (0.8 ml) and the resultant mixture was stirred at ambient temperature for 1 hour. The mixture was diluted with a saturated aqueous sodium bicarbonate solution (10 mly and stirred at 5° C. for 15 minutes. The resultant mixture was extracted with ethyl acetate and the organic extract was dried over magnesium sulphate and evaporated. The residue was purified by chromatography on silica using a solvent gradient from a 19:1 to a 9:1 mixture of methylene chloride and methanol followed by a 9:1 mixture of methylene chloride and a 7M methanolic ammonia solution as eluent. There was thus obtained the title compound as a solid (0.12 g); ¹H NMR: (DMSOd₆) 2.19 (s, 6H), 2.59 (s, 2H), 3.98 (s, 3H), 3.99 (s, 3H), 4.29 (t, 2H), 5.04 (s, 2H), 6.81 (d, 1H), 7.39 (s, 1H), 7.54 (s, 1H), 7.68 (s, 1H), 7.89 (m, 1H), 8.12 (s, 1H), 8.36 (d, 1H), 8.65 (s, 1H), 10.37 (s, 1H); Mass Spectrum: M+H⁺ 494.

The 3-amino-6-(2-dimethylaminoethoxy)pyridine used as a starting material was prepared as follows: —

Sodium hydride (60% dispersion in mineral oil, 1.32 g) was added portionwise during 20 minutes to a mixture of 2-chloro-5-nitropyridine (4.77 g), 2-dimethylaminoethanol (2.67 g) and THF (50 ml) that had been cooled to 5° C. The resultant mixture was stirred at 5° C. for 1 hour. The mixture was diluted with water and extracted with diethyl ether. The organic solution was extracted with a 2N aqueous hydrochloric acid solution. The resultant aqueous solution was basified to pH9 with potassium carbonate and extracted with diethyl ether. The organic phase so obtained was dried over magnesium sulphate and evaporated. There was thus obtained 2-(2-dimethylaminoethoxy)-5-nitropyridine as an oil (4.5 g); ¹H NMR: (DMSOd₆) 2.2 (s, 6H), 2.64 (t, 2H), 4.84 (t, 2H), 7.03 (m, 1H), 8.46 (m, 1H), 9.07 (m, 1H); Mass Spectrum: M+H⁺ 212.

A mixture of the material so obtained, platinum oxide (0.4 g), ethyl acetate (45 ml) and ethanol (45 ml) was stirred under 6 atmospheres pressure of hydrogen for 2 hours. The catalyst was removed by filtration and the filtrate was evaporated. There was thus obtained 3-amino-6-(2-dimethylaminoethoxy)pyridine (3.8 g), ¹H NMR: (DMSOd₆) 2.17 (s, 6H), 2.53 (t, 2H), 4.15 (t, 2H), 6.51 (m, 1H), 6.98 (m, 1H), 7.47 (m, 1H); Mass Spectrum: M+t+182.

### EXAMPLE 9

Using an analogous procedure to that described in Example 8, the appropriate 2-(pyrazol-1-yl)acetic acid was reacted with the appropriate aniline to give the compounds described in Table II. Unless otherwise stated, each aniline starting material was commercially available. Further, unless otherwise stated, each of the Compounds [16] to [28] below was purified by preparative HPLC using a Waters ‘Symmetry’ C18 reversed-phase column (5 microns silica, 19 mm diameter, 100 mm length) and decreasingly polar mixtures of water (containing 2% acetic acid) and acetonitrile as eluent.

TABLE II

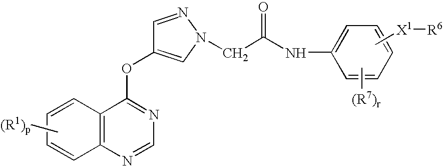

| No. & | | | |
|---|---|---|---|
| Note | (R¹)ₚ | (R⁷)ᵣ | X¹—R⁶ |
| [1] | 6,7-dimethoxy | H | 4-(4-methylpiperazin-1-yl) |
| [2] | 6,7-dimethoxy | H | 3-dimethylaminomethyl |
| [3] | 6,7-dimethoxy | H | 4-dimethylaminomethyl |
| [4] | 6-methoxy- | H | 4-dimethylaminomethyl |
| | 7-ethoxy | | |
| [5] | 6,7-dimethoxy | 4-methoxy | 3-dimethylaminomethyl |
| [6] | 6,7-dimethoxy | 6-methoxy | 3-dimethylaminomethyl |
| [7] | 6,7-dimethoxy | 4-fluoro | 3-dimethylaminomethyl |
| [8] | 6,7-dimethoxy | 4-chloro | 3-dimethylaminomethyl |
| [9] | 6,7-dimethoxy | 4-dimethylamino | 3-dimethylaminomethyl |
| [10] | 6,7-dimethoxy | 6-dimethylamino | 3-dimethylaminomethyl |
| [11] | 6,7-dimethoxy | H | 3-(2-dimethylaminoethyl) |
| [12] | 6,7-dimethoxy | H | 4-(2-dimethylaminoethyl) |
| [13] | 6,7-dimethoxy | H | 4-pyrrolidin-1-ylmethyl |
| [14] | 6-methoxy-7- | H | 4-dimethylaminomethyl |
| | (2-methoxy- | | |
| | ethoxy) | | |
| [15] | 6,7-dimethoxy | 4-methoxy | 3-hydroxymethyl |
| [16] | 6,7-dimethoxy | 6-hydroxy | 3-dimethylaminomethyl |
| [17] | 6,7-dimethoxy | 2-hydroxy | 4-dimethylaminomethyl |
| [18] | 6,7-di-(2- | 4-chloro | 3-dimethylaminomethyl |
| | methoxy- | | |
| | ethoxy) | | |
| [19] | 6,7-dimethoxy | 4-methyl | 3-dimethylaminomethyl |
| [20] | 6,7-dimethoxy | 5-methyl | 3-dimethylaminomethyl |
| [21] | 6,7-dimethoxy | 5-methyl | 3-(N-ethyl- |
| | | | N-methylaminomethyl) |
| [22] | 6,7-dimethoxy | 5-methyl | 3-(N-cyclopropyl- |
| | | | N-methylaminomethyl) |
| [23] | 6,7-dimethoxy | 5-methyl | 3-pyrrolidin-1-ylmethyl |
| [24] | 6,7-dimethoxy | 4-chloro | 3-(2-dimethylaminoethyl) |
| [25] | 6,7-dimethoxy | 4-methoxy | 3-(2-dimethylaminoethyl) |
| [26] | 6,7-dimethoxy | 4-fluoro | 3-hydroxymethyl |
| [27] | 6,7-dimethoxy | 5-chloro | 3-hydroxymethyl |
| [28] | 6,7-dimethoxy | 4-chloro | 3-hydroxymethyl |

Notes The products gave the characterising data shown below.

[1] ¹H NMR: (DMSOd₆) 2.21 (s, 3H), 2.41-2.46 (m, 4H), 3.04-3.1 (m, 4H), 3.98 (s, 3H), 3.99 (s, 3H), 4.99 (d, 2H), 6.9 (d, 2H), 7.39 (s, 1H), 7.45 (d, 2H), 7.54 (s, 1H), 7.67 (s, 1H), 8.11 (s, 1H), 8.66 (s, 1H), 10.12 (s, 1H); Mass Spectrum: M+H⁺ 504.

[2] ¹H NMR: (DMSOd₆) 2.14 (s, 6M), 3.35 (s, 2M), 3.98 (s, 3H), 3.99 (s, 3H), 5.03 (d, 2H), 6.99 (d, 1H), 7.27 (m, 1H), 7.39 (s, 1H), 7.49 (d, 1H), 7.54 (s, 1H), 7.58 (s, 1H), 7.68 (s, 1H), 8.12 (s, 1H), 8.66 (s, 1H), 10.32 (s, 1H); Mass Spectrum: M+H⁺ 463.

The 3-dimethylaminomethylaniline used as a starting material was prepared as follows: —

Triethylamine (3.64 g) was added dropwise to a mixture of 3-nitrobenzyl bromide (2.6 g), dimethylamine hydrochloride (1.96 g) and methylene chloride (26 ml) and the resultant mixture was stirred at ambient temperature for 2 hours. The solvent was evaporated and the residue was diluted with water and extracted with ethyl acetate. The organic phase was washed with water, dried over magnesium sulphate and concentrated. There was thus obtained N,N-dimethyl-N-(3-nitrobenzyl)amine (1.6 g); ¹H NMR: (DMSOd₆) 2.18 (s, 6H), 3.34 (s, 2H), 7.63 (t, 1H), 7.75 (d, 1H), 8.12 (m, 2H); Mass Spectrum: M+H⁺ 181.

Raney nickel (0.8 g) was washed twice with ethanol and added to a solution of N,N-dimethyl-N-(3-nitrobenzyl)amine (1.6 g) in a mixture of methanol (10 ml) and ethanol (50 ml). The mixture was stirred under 1.8 atmospheres pressure of hydrogen at ambient temperature for 1 hour. The reaction mixture was filtered and the filtrate was evaporated. The residue was purified by column chromatography on silica using a solvent gradient from a 19:1 to a 9:1 mixture of methylene chloride and methanol followed by a 9:1 to a 18:3 mixture of methylene chloride and a 7M methanolic ammonia solution as eluent. There was thus obtained 3-dimethylaminomethylaniline (0.85 g); ¹H NMR: (DMSOd₆) 2.11 (s, 6H), 3.2 (s, 2H), 4.96 (br s, 2H), 6.41 (m, 2H), 6.51 (s, 1H), 6.92 (t, 1H); Mass Spectrum: M+11151.

[3] ¹H NMR: (DMSOd₆) 2.11 (s, 6H), 3.3 (s, 2H), 3.98 (s, 3H), 3.99 (s, 3H), 5.03 (d, 2H), 7.23 (d, 2H), 7.39 (s, 1H), 7.54 (s, 1H), 7.55 (d, 2H), 7.68 (s, 1H), 8.12 (s, 1H), 8.66 (s, 1H), 10.32 (s, 1H); Mass Spectrum: M+H⁺ 463.

The 4-dimethylaminomethylaniline used as a starting material was prepared from 4-nitrobenzyl bromide using analogous procedures to those described in Note [2] above for the preparation of 3-dimethylaminomethylaniline. The desired aniline material gave the following characterising data: ¹H NMR: (DMSOd₆) 2.07 (s, 6H), 3.17 (s, 2H), 4.92 (br s, 2H), 6.49 (m, 2H), 6.89 (m, 2H); Mass Spectrum: M+H⁺ 151.

[4] ¹H NMR: (DMSOd₆) 1.43 (t, 3H), 2.14 (s, 6H), 3.36 (s, 2H), 3.98 (s, 3H), 4.25 (q, 2H), 5.03 (s, 2H), 7.24 (d, 2H), 7.37 (s, 1H), 7.54 (s, 1H), 7.55 (d, 2H), 7.67 (s, 1H), 8.12 (s, 1H), 8.64 (s, 1H), 10.33 (s, 1H); Mass Spectrum: M+H⁺ 477.

The 2-[4-(7-ethoxy-6-methoxyquinazolin-4-yloxy)pyrazol-1-yl]acetic acid used as a starting material was prepared as follows: —

Diethyl azodicarboxylate (0.673 ml) was added to a stirred mixture of 4-chloro-7-hydroxy-6-methoxyquinazoline (International Patent Application WO 03/064413, Example 4 thereof; 0.6 g), ethanol (0.182 ml), triphenylphosphine (1.12 g) and methylene chloride (12 ml) and the resultant mixture was stirred at ambient temperature for 10 minutes. The mixture was evaporated and the residue was purified by column chromatography on silica using a solvent gradient from a 17:3 to a 4:1 mixture of methylene chloride and ethyl acetate as eluent. There was thus obtained 4-chloro-7-ethoxy-6-methoxyquinazoline as a solid (0.391 g); ¹H NMR: (CDCl₃) 1.58 (t, 3H), 4.07 (s, 3H), 4.29 (q, 2H), 7.32 (s, 1H), 7.39 (s, 1H), 8.86 (s, 1H).

A mixture of the material so obtained, tert-butyl 2-(4-hydroxypyrazol-1-yl)acetate (0.356 g), potassium carbonate (0.338 g) and DMA (5 ml) was stirred and heated to 100° C. for 1.5 hours. The resultant mixture was cooled to ambient temperature, poured into water and extracted with ethyl acetate. The organic phase was washed with water, dried over magnesium, sulphate and evaporated. The residue was purified by column chromatography on silica using a 1:1 mixture of methylene chloride and ethyl acetate as eluent. There was thus obtained tert-butyl 2-[4-(7-ethoxy-6-methoxyquinazolin-4-yloxy)pyrazol-1-yl]acetate (0.573 g); ¹H NMR: 1.43 (t, 3H), 1.45 (s, 9H), 3.98 (s, 3H), 4.24 (q, 2H), 4.96 (s, 2H), 7.36 (s, 1H), 7.53 (s, 1H), 7.66 (s, 1H), 8.07 (s, 1H), 8.63 (s, 1H); Mass Spectrum: M+H⁺ 401.

A mixture of a solution of the material so obtained, trifluoroacetic acid (3 ml), water (0.1 ml) and methylene chloride (2 ml) was stirred at ambient temperature for 3 hours. The resultant mixture was evaporated. The residue was triturated under ethyl acetate. The resultant solid was recovered by filtration, suspended in methylene chloride and treated with an excess of diisopropylethylamine. The resultant solution was evaporated and the residual oil was triturated under a mixture of methylene chloride and ethyl acetate. The solid so obtained was dried under vacuum. There was thus obtained 2-[4-(7-ethoxy-6-methoxyquinazolin-4-yloxy)pyrazol-1-yl]acetic acid (0.412 g); ¹H NMR: (DMSOd₆) 1.43 (t, 3H), 3.98 (s, 3H), 4.25 (q, 2H), 7.36 (s, 1H), 7.53 (s, 1H), 7.65 (s, 1H), 8.07 (s, 1H), 8.64 (s, 1H); Mass Spectrum: M+H⁺ 345.

[5] ¹H NMR: (DMSOd₆) 2.18 (s, 6H), 3.4 (s; 2H), 3.74 (s, 3H), 3.74-3.98 (s, 3H), 3.99 (s, 3H), 4.99 (s, 2H), 6.94 (d, 1H), 7.39 (s, 1H), 7.5 (m, 1H), 7.54 (s, 1H), 7.55 (d, 1H), 7.67 (s, 1H), 8.12 (s, 1H), 8.66 (s, 1H), 10.19 (s, 1H); Mass Spectrum: M+H⁺ 493.

The 3-dimethylaminomethyl-4-methoxyaniline used as a starting material was prepared as follows: —

Using analogous procedures to those described in the portion of Note [2] above that is concerned with the preparation of starting materials, 2-methoxy-5-nitrobenzyl bromide was converted into N,N-dimethyl-N-(2-methoxy-5-nitrobenzyl)amine; ¹H NMR: (DMSOd₆) 2.2 (s, 6H), 3.44 (s, 2H), 3.93 (s, 3H), 7.21 (m, 1H), 8.17 (m, 2H); Mass Spectrum: M+H⁺ 211; which in turn was converted into 3-dimethylaminomethyl-4-methoxyaniline; ¹H NMR: (DMSOd₆) 2.12 (s, 6H), 3.25 (s, 2H), 3.62 (s, 3H), 4.55 (br s, 2H), 6.41 (m, 1H), 6.59 (m, 1H), 6.66 (m, 1H); Mass Spectrum: M+H⁺ 181.

[6] ¹H NMR: (DMSOd₆) 2.16 (s, 6M), 3.32 (s, 2H), 3.85 (s, 3H), 3.98 (s, 3H), 3.99 (s, 3H), 5.13 (s, 2H), 7.01 (s, 2H), 6.39 (s, 1H), 6.54 (s, 1H), 7.72 (s, 1H), 8.0 (br s, 1H), 8.14 (s, 1H), 8.66 (s, 1H), 9.45 (s, 1H); Mass Spectrum: M+H⁺ 493.

The 3-dimethylaminomethyl-6-methoxyaniline used as a starting material was prepared as follows: —

Using an analogous procedure to that described in the first paragraph of the portion of Example 10 that is concerned with the preparation of starting materials, 4-methoxy-3-nitrobenzaldehyde was reacted with dimethylamine to give N,N-dimethyl-N-(4-methoxy-3-nitrobenzyl)amine; ¹H NMR: (DMSOd₆) 2.14 (s, 6H), 3.38 (s, 2H), 3.91 (s, 3H), 7.31 (d, 1H), 7.56 (m, 1H), 7.75 (s, 1H); Mass Spectrum: M+H⁺ 211; which in turn was hydrogenated using an analogous procedure to that described in the third paragraph of the portion of Example 10 that is concerned with the preparation of starting materials to give 3-dimethylaminomethyl-6-methoxyaniline; ¹H NMR: (DMSOd₆) 2.08 (s, 6H), 3.17 (s, 2H), 3.72 (s, 3H), 4.62 (br s, 2H), 6.41 (d, 1H), 6.59 (s, 1H), 6.68 (d, 1H); Mass Spectrum: M+H⁺ 181.

[7] ¹H NMR: (DMSOd₆) 2.17 (s, 6H), 3.41 (s, 2H), 3.98 (s, 3H), 3.99 (s, 3H), 5.02 (s, 2H), 7.14 (m, 1H), 7.39 (s, 1H), 7.52 (m, 1H), 7.54 (s, 1H), 7.66 (m, 1H), 7.68 (s, 1H), 8.12 (s, 1H), 8.66 (s, 1H), 10.38 (s, 1H); Mass Spectrum: M+H⁺ 481.

The 3-dimethylaminomethyl-4-fluoroaniline used as a starting material was prepared as follows: —

Using an analogous procedure to that described in the first paragraph of the portion of Example 10 that is concerned with the preparation of starting materials, 2-fluoro-5-nitrobenzaldehyde was reacted with dimethylamine. A mixture of two products was obtained. Following chromatographic separation on silica, there were obtained N,N-dimethyl-N-(2-fluoro-5-nitrobenzyl)amine; ¹H NMR: (DMSOd₆) 2.19 (s, 6H), 3.54 (s, 2H), 7.49 (t, 1H), 8.24 (m, 1H), 8.29 (m, 1H); Mass Spectrum: M+H⁺ 199; and N-(2-dimethylamino-5-nitrobenzyl)-N,N-dimethylamine; ¹H NMR: (DMSOd₆) 2.18 (s, 6H), 2.88 (s, 6H), 3.44 (s, 211), 7.05 (d, 1H), 8.03 (m, 1H), 8.28 (m, 1H); Mass Spectrum: M+H⁺ 224. The N,N-dimethyl-N-(2-fluoro-5-nitrobenzyl)amine was hydrogenated using an analogous procedure to that described in the third paragraph of the portion of Example 10 that is concerned with the preparation of starting materials to give 3-dimethylaminomethyl-4-fluoroaniline; ¹H NMR: (DMSOd₆) 2.19 (s, 6H), 3.28 (s, 2H), 4.87 (br s, 2H), 6.42 (m, 1H), 6.56 (m, 1H), 6.78 (t, 1H); Mass Spectrum: M+H⁺ 169.

[8] ¹H NMR: (DMSOd₆) 2.21 (s, 6H), 3.48 (s, 2H), 3.98 (s, 3H), 3.99 (s, 3H), 5.04 (s, 2H), 7.38 (d, 1H), 7.39 (s, 1H), 7.54 (s, 1H), 7.56 (m, 1H), 7.68 (s, 1H), 7.76 (br s, 1H), 8.12 (s, 1H), 8.66 (s, 1H), 10.48 (s, 1H); Mass Spectrum: M+H⁺ 497 and 499.

The 4-chloro-3-dimethylaminomethylaniline used as a starting material was prepared as follows: —

Using an analogous procedure to that described in the first paragraph of the portion of Example 10 that is concerned with the preparation of starting materials, 2-chloro-5-nitrobenzaldehyde was reacted with dimethylamine to give N-(2-chloro-5-nitrobenzyl)-N,N-dimethylamine; ¹H NMR: (DMSOd₆) 2.24 (s, 6H), 3.58 (s, 2H), 7.75 (d, 1H), 8.14 (m, 1H), 8.3 (d, 1H); Mass Spectrum: M+H⁺ 215 and 217; which in turn was hydrogenated using an analogous procedure to that described in the third paragraph of the portion of Example 10 that is concerned with the preparation of starting materials to give 4-chloro-3-dimethylaminomethylaniline; ¹H NMR: (DMSOd₆) 2.16 (s, 6H), 3.31 (s, 2H), 5.17 (br s, 2H), 6.44 (m, 1H), 6.68 (m, 1H), 6.98 (m, 1H); Mass Spectrum: M+H⁺ 185 and 187.

[9] ¹H NMR: (DMSOd₆) 2.2 (bras, 6H), 2.6 (s, 6H), 3.44 (br s, 2H), 3.98 (s, 3H), 3.99 (s, 3H), 5.0 (s, 2H), 7.07 (d, 1H), 7.4 (s, 1H), 7.49 (m, 1H), 7.54 (s, 1H), 7.63 (br s, 1H), 7.67 (s, 1H), 8.11 (s, 1H), 8.66 (s, 1H), 10.23 (br s, 1H); Mass Spectrum: M+H⁺ 506.

The 4-dimethylamino-3-dimethylaminomethylaniline used as a starting material was prepared as follows: —

Using an analogous procedure to that described in the third paragraph of the portion of Example 10 that is concerned with the preparation of starting materials, N-(2-dimethylamino-5-nitrobenzyl)-N,N-dimethylamine was hydrogenated to give 4-dimethylamino-3-dimethylaminomethylaniline; ¹H NMR: (DMSOd₆) 2.14 (s, 6H), 2.49 (s, 6H), 3.48 (s, 2H), 4.69 (br s, 2H), 6.41 (m, 1H), 6.65 (m, 1H), 6.84 (m, 1H); Mass Spectrum: M+H⁺ 194.

[10] ¹H NMR: (DMSOd₆) 2.11 (s, 6H), 2.54 (s, 6H), 3.29 (s, 2H), 3.98 (s, 3H), 3.99 (s, 3H), 5.14 (s, 2H), 6.98 (d, 1H), 7.12 (d, 1H), 7.4 (s, 1H), 7.54 (s, 1H), 7.8 (s, 1H), 8.01 (s, 1H), 8.22 (s, 1H), 8.66 (s, 1H), 9.25 (s, 1H); Mass Spectrum: M+H⁺ 506.

The 6-dimethylamino-3-dimethylaminomethylaniline used as a starting material was prepared as follows: —

Using an analogous procedure to that described in the first paragraph of the portion of Example 10 that is concerned with the preparation of starting materials, 4-fluoro-3-nitrobenzaldehyde was reacted with dimethylamine to give N-(4-dimethylamino-3-nitrobenzyl)-N,N-dimethylamine; ¹H NMR: (DMSOd₆) 2.12 (s, 6H), 2.79 (s, 6H), 3.34 (s, 2H), 7.4 (d, 1H), 7.41 (m, 1H), 7.64 (s, 1H); Mass Spectrum: M+H⁺ 224; which in turn was hydrogenated using an analogous procedure to that described in the third paragraph of the portion of Example 10 that is concerned with the preparation of starting materials to give 6-dimethylamino-3-dimethylaminomethylaniline; ¹H NMR: (DMSOd₆) 2.09 (s, 6H), 2.54 (s, 6H), 3.17 (s, 2H), 4.67 (br s, 2H), 6.44 (m, 1H), 6.6 (s, 1H), 6.81 (d, 1H).

[11] ¹H NMR: (DMSOd₆) 2.27 (s, 6H), 2.51-2.61 (m, 2H), 2.71 (t, 2H), 3.98 (s, 3H), 3.99 (s, 3H), 5.03 (d, 2H), 6.95 (d, 1H), 7.24 (m, 1H), 7.39 (s, 1H), 7.4 (d, 1H), 7.51 (s, 1H), 7.54 (s, 1H), 7.68 (s, 1H), 8.12 (s, 1H), 8.66 (s, 1H), 10.29 (s, 1H); Mass Spectrum: M+H⁺ 477.

The 3-(2-dimethylaminoethyl)aniline used as a starting material was prepared as follows: —

Using analogous procedures to those described in the portion of Note [2] above that is concerned with the preparation of starting materials, 2-(3-nitrophenyl)ethyl bromide was converted into N,N-dimethyl-N-[2-(3-nitrophenyl)ethyl]amine; ¹H NMR: (DMSOd₆) 2.17 (s, 6H), 2.5 (m, 2H), 2.86 (m, 2H), 7.57 (t, 1H), 7.72 (d, 1H), 8.04 (m, 1H), 8.11 (m, 1H); which in turn was converted into 3-(2-dimethylaminoethyl)aniline; ¹H NMR: (DMSOd₆) 2.15 (s, 6H), 2.38 (m, 2H), 2.51 (m, 2H), 4.91 (br s, 2H), 6.35 (m, 3H), 6.89 (t, 1H); Mass Spectrum: M+H⁺ 165.

[12] ¹H NMR: (DMSOd₆) 2.17 (s, 6H), 2.42 (t, 2H), 2.65 (t, 2H), 3.98 (s, 3H), 3.99 (s, 3H), 5.02 (d, 2H), 7.17 (d, 2H), 7.39 (s, 1H), 7.5 (d, 2H), 7.54 (s, 1H), 7.67 (s, 1H), 8.12 (s, 1H), 8.66 (s, 1H), 10.26 (s, 1H); Mass Spectrum: M+H⁺ 477.

The 4-(2-dimethylaminoethyl)aniline used as a starting material was prepared as follows: —

Using analogous procedures to those described in the portion of Note [2] above that is concerned with the preparation of starting materials, 2-(4-nitrophenyl)ethyl bromide was converted into N,N-dimethyl-N-[2-(4-nitrophenyl)ethyl]amine; ¹H NMR: (DMSOd₆) 2.17 (s, 6H), 2.5 (m, 2H), 2.85 (m, 2H), 7.52 (m, 2H), 8.13 (m, 2H); Mass Spectrum: M+H⁺ 195; which in turn was converted into 4-(2-dimethylaminoethyl)aniline; ¹H NMR: (DMSOd₆) 2.14 (s, 6H), 2.33 (m, 2H), 2.5 (m, 2H), 4.8 (br s, 2H), 6.46 (m, 2H), 6.85 (m, 2H); Mass Spectrum: M+H⁺ 165.

[13] ¹H NMR: (DMSOd₆) 1.64-1.76 (m, 4H), 2.27-2.49 (m, 4H), 3.51-3.62 (m, 2H), 3.98 (s, 3H), 3.99 (s, 3H), 5.03 (s, 2H), 7.26 (d, 2H), 7.39 (s, 1H), 7.51-7.58 (m, 3H), 7.68 (s, 1H), 8.12 (s, 1H), 8.66 (s, 1H), 10.32 (s, 1H); Mass Spectrum: M+H⁺ 489.

The 4-pyrrolidin-1-ylmethylaniline used as a starting material was prepared as follows: —

Using analogous procedures to those described in the portion of Note [2] above that is concerned with the preparation of starting materials, 4-nitrobenzyl bromide was converted into 1-(4-nitrobenzyl)pyrrolidine; ¹H NMR: (DMSOd₆) 1.71 (m, 4H), 2.45 (m, 4H), 3.71 (s, 2H), 7.59 (d, 2H), 8.18 (d, 2H); which in turn was converted into 4-(pyrrolidin-1-ylmethyl)aniline; ¹H NMR: (DMSOd₆) 1.64 (m, 4H), 2.35 (m, 4H), 4.89 (s, 2H), 6.48 (d, 2H), 6.9 (d, 2H); Mass Spectrum: M+H⁺ 177.

[14] ¹H NMR: (DMSOd₆) 2.12 (s, 6H), 3.31 (s, 2H), 3.35 (s, 3H), 3.74-3.78 (m, 2H), 3.99 (s, 3H), 3.3-3.36 (m, 2H), 5.03 (s, 2H), 7.24 (d, 2H), 7.41 (s, 1H), 7.54 (s, 1H), 7.5.5 (d, 2H), 7.68 (s, 1H), 8.12 (s, 1H), 8.65 (s, 1H), 10.32 (s, 1H); Mass Spectrum: M+H⁺ 507.

The 2-{4-[6-methoxy-7-(2-methoxyethoxy)quinazolin-4-yloxy]pyrazol-1-yl}acetic acid used as a starting material was prepared as follows: —

Using analogous procedures to those described in the portion of Note [4] above that is concerned with the preparation of starting materials, 4-chloro-7-hydroxy-6-methoxyquinazoline was reacted with 2-methoxyethanol to give 4-chloro-6-methoxy-7-(2-methoxyethoxy)quinazoline in 100% yield; ¹H NMR: (CDCl₃) 3.49 (s, 3H), 3.89 (t, 2H), 4.05 (s, 3H), 4.35 (t, 2H), 7.26 (s, 1H), 7.35 (s, 1H), 8.86 (s, 1H); Mass Spectrum: M+H⁺ 269; which in turn was reacted with tert-butyl 2-(4-hydroxypyrazol-1-yl)acetate to give tert-butyl 2-{4-[6-methoxy-7-(2-methoxyethoxy)quinazolin-4-yloxy]pyrazol-1-yl}acetate in 70% yield; ¹H NMR: (CDCl₃) 1.5 (s, 9H), 3.49 (s, 3H), 3.9 (t, 2H), 4.04 (s, 3H), 4.34 (t, 2H), 4.84 (s, 2H), 7.33 (s, 1H), 7.49 (s, 1H), 7.7 (s, 1H), 7.92 (s, 1H), 8.69 (s, 1H); Mass Spectrum: M+H⁺ 431; which in turn was converted into the required starting material in 82% yield;

¹H NMR: (DMSOd₆) 3.34 (s, 3H), 3.76 (t, 2H), 3.98 (s, 3H), 4.32 (t, 2H), 4.96 (s, 2H), 7.4 (s, 1H), 7.54 (s, 1H), 7.65 (s, 1H), 8.07 (s, 1H), 8.64 (s, 1H); Mass Spectrum: M+H⁺ 375.

[15] ¹H NMR: (DMSOd₆) 3.75 (s, 3H), 3.98 (s, 3H), 3.99 (s, 3H), 4.47 (d, 2H), 5.0 (s, 2H), 5.08 (t, 1H), 6.89 (d, 1H), 7.39 (s, 1H), 7.53 (m, 2H), 7.67 (s, 1H), 8.12 (s, 1H), 8.66 (s, 1H), 10.19 (s, 1H); Mass Spectrum: M+H⁺ 466.

The 3-hydroxymethyl-4-methoxyaniline used as a starting material was prepared as follows: —

A mixture of 2-methoxy-5-nitrobenzaldehyde (3 g), platinum oxide (0.3 g) and methanol (150 ml) was stirred under 2 atmospheres pressure of hydrogen for 3 hours. The catalyst was removed by filtration and the filtrate was evaporated. There was thus obtained 3-hydroxymethyl-4-methoxyaniline as a solid (2.08 g); ¹H NMR: (DMSOd₆) 3.63 (s, 3H), 4.38 to (d, 2H), 4.56 (br s, 2H), 4.82 (t, 1H), 6.38 (m, 1H), 6.62 (d, 1H), 6.67 (m, 1H).

[16] ¹H NMR: (DMSOd₆) 2.33 (br s, 6H), 3.59 (br s, 2H), 3.98 (s, 3H), 3.99 (s, 3H), 5.13 (s, 2H), 6.86 (d, 1H), 6.92 (d, 1H), 7.4 (s, 1H), 7.54 (s, 1H), 7.71 (s, 1H), 7.97 (br s, 1H), 8.15 (s, 1H), 8.66 (s, 1H), 9.43 (s, 1H), 10.07 (br s, 1H); Mass Spectrum: M+H⁺ 479.

The 3-dimethylaminomethyl-6-hydroxyaniline used as a starting material was prepared as follows: —

Under an atmosphere of argon, titanium (IV) isopropoxide (2.84 g) was added portionwise to a stirred 2M solution of dimethylamine in methanol (10 ml). 4-Hydroxy-3-nitrobenzaldehyde (0.865 g) was added portionwise and the resultant mixture was stirred at ambient temperature for 4 hours. Sodium borohydride (0.19 g) was added portionwise and the resultant mixture was stirred at ambient temperature for 1 hour. Water (1 ml) was added and the mixture was evaporated. The residue was purified by column chromatography on silica using a 19:1 mixture of methylene chloride and methanol as eluent. There was thus obtained 4-dimethylaminomethyl-2-nitrophenol as a solid (0.72 g); ¹H NMR: (DMSOd₆+CF₃CO₂D) 2.75 (s, 6H), 4.29 (s, 2H), 7.24 (d, 1H), 7.65 (m, 1H), 8.13 (d, 1H); Mass Spectrum: M+H⁺ 197.

A mixture of the material so obtained, platinum oxide (0.071 g), ethyl acetate (15 ml) and methanol (5 ml) was stirred under 1.8 atmospheres pressure of hydrogen for 40 minutes. The catalyst was removed by filtration and the filtrate was evaporated. The oil so obtained was triturated under diethyl ether to give a solid which was dried under vacuum at 50° C. for 3 hours. There was thus obtained 3-dimethylaminomethyl-6-hydroxyaniline (0.52 g); ¹H NMR: (DMSOd₆) 2.08 (s, 6H); 3.13 (s, 2H), 4.24 (br s, 2H), 6.27 (d, 1H), 6.54 (m, 2H), 8.21 (br s, 1H); Mass Spectrum: M+H⁺ 167.

[17] ¹H NMR: (DMSOd₆) 2.08 (br s, 6H), 3.31 (s, 2H), 3.98 (s, 3H), 3.99 (s, 3H), 5.12 (s, 2H), 6.7 (d, 1H), 6.85 (s, 1H), 7.39 (s, 1H), 7.54 (s, 1H), 7.71 (s, 1H), 7.86 (d, 1H), 8.15 (s, 1H), 8.66 (s, 1H), 9.36 (s, 1H), 9.92 (br s, 1H); Mass Spectrum: M+H⁺ 479.

The 4-dimethylaminomethyl-2-hydroxyaniline used as a starting material was prepared as follows: —

Using analogous procedures to those described in the portion of Note [16] above that is concerned with the preparation of starting materials, 3-hydroxy-4-nitrobenzaldehyde was reacted with dimethylamine to give 5-dimethylaminomethyl-2-nitrophenol in 43% yield;

¹H NMR: (DMSOd₆+CF₃CO₂D) 2.53 (s, 6H), 4.34 (s, 2H), 7.12 (d, 1H), 7.27 (m, 1H), 8.0 (d, 1H); Mass Spectrum: M+H⁺ 197; which in turn was hydrogenated to give 4-dimethylaminomethyl-2-hydroxyaniline in 100% yield; ¹H NMR: (DMSOd₆) 2.07 (s, 6H), 3.13 (s, 2H), 4:37 (br s, 2H), 6.42 (m, 1H), 6.48 (m, 1H), 6.58 (m, 1H), 8.87 (br s, 1H); Mass Spectrum: M+H⁺ 167.

[18] The product was purified by column chromatography on silica using a 19:1 mixture of methylene chloride and a 3M methanolic ammonia solution as eluent and gave the following characterising data: —¹H NMR: (DMSOd₆) 2.26 (br s, 6H), 3.5 (br s, 2H), 3.36 (s, 3H), 3.37 (s, 3H), 3.74-3.79 (m, 4H), 4.3-4.37 (m, 4H), 5.04 (s, 2H), 7.4 (d, 1H), 7.42 (s, 1H), 7.55 (m, 1H), 7.58 (s, 1H), 7.68 (s, 1H), 7.77 (br s, 1H), 8.12 (s, 1H), 8.65 (s, 1H), 10.5 (br s, 1H); Mass Spectrum: M+H⁺ 585 and 587.

The 2-{4-[6,7-di-(2-methoxyethoxy)quinazolin-4-yloxy]pyrazol-1-yl}acetic acid used as a starting material was prepared as follows: —

Using analogous procedures to those described in the second and third paragraphs of the portion of Note [4] above that is concerned with the preparation of starting materials, 4-chloro-6,7-di-(2-methoxyethoxy)quinazoline (U.S. Pat. No. 5,747,498) was reacted with tert-butyl 2-(4-hydroxypyrazol-1-yl)acetate to give tert-butyl 2-{4-[6,7-di-(2-methoxyethoxy)quinazolin-4-yloxy]pyrazol-1-yl}acetate in 79% yield;

¹H NMR: (DMSOd₆+CF₃CO₂D) 1.46 (s, 9H), 3.79 (m, 4H), 4.4 (m, 4H), 5.0 (s, 2H), 7.51 (s, 1H), 7.72 (s, 1H), 7.74 (s, 1H), 8.13 (s, 1H), 9.06 (s, 1H); Mass Spectrum: M+H⁺ 475; which in turn was reacted with trifluoroacetic acid to give the required starting material in 89% yield;

¹H NMR: (DMSOd₆+CF₃CO₂D) 3.8 (m, 4H), 4.2 (m, 4H), 5.04 (s, 2H), 7.53 (s, 1H), 7.73 (s, 1H), 7.77 (s, 1H), 8.15 (s, 1H), 9.13 (s, 1H); Mass Spectrum: M+H⁺ 419.

[19] The product was purified by column chromatography on silica using a 19:1 mixture of methylene chloride and a 3M methanolic ammonia solution as eluent and gave the following characterising data: —¹H NMR: (DMSOd₆+CF₃CO₂D) 2.37 (s, 3H), 2.8 (5, 6H), 4.05 (s, 3H), 4.07 (s, 3H), 4.32 (s, 2H), 5.12 (s, 2H), 7.29 (d, 1H), 7.46 (m, 1H), 7.52 (s, 1H), 7.7 (s, 1H), 7.75 (s, 1H), 7.86 (d, 1H), 8.2 (s, 1H), 9.1 (br s, 1H); Mass Spectrum: M+H⁺ 477.

The 3-dimethylaminomethyl-4-methylaniline used as a starting material was prepared as follows: —

Diborane (2M solution in THF, 24.5 ml) was added dropwise to a stirred solution of N,N-dimethyl-2-methyl-5-nitrobenzamide (3 g) in THF (10 ml). The resultant mixture was stirred and heated to 58° C. for 6 hours. A 6N aqueous hydrochloric acid solution (50 ml) was added and the mixture was stirred at ambient temperature for 16 hours. The mixture was basified by the addition of potassium carbonate and extracted with ethyl acetate. The organic solution was dried over magnesium sulphate and evaporated to leave an oil which was triturated under diethyl ether. There was thus obtained N,N-dimethyl-N-(2-methyl-5-nitrobenzyl)amine as a solid (1.8 g); ¹H NMR: (CDCl₃) 2.27 (s, 6H), 2.45 (s, 3H), 3.43 (s, 2H), 7.29 (m, 1H), 8.02 (m, 1H), 8.16 (m, 1H); Mass Spectrum: M+H⁺ 195.

A mixture of N,N-dimethyl-N-(2-methyl-5-nitrobenzyl)amine (2.4 g), platinum oxide (0.12 g) and ethyl acetate (40 ml) was stirred under 1.8 atmospheres pressure of hydrogen for 30 minutes. The catalyst was removed by filtration and the filtrate was evaporated. The material so obtained was dried under vacuum at ambient temperature for 2 hours. There was thus obtained 3-dimethylaminomethyl-4-methylaniline as a solid (1.85 g); ¹H NMR: (DMSOd₆+CF₃CO₂D) 2.42 (s, 3H), 2.81 (s, 6H), 4.37 (s, 2H), 7.36 (m, 1H), 7.43 (m, 2H); Mass Spectrum: M+H⁺ 165.

[20] The product was purified by column chromatography on silica using a 19:1 mixture of methylene chloride and a 3M methanolic ammonia solution as eluent and gave the following characterising data: —¹H NMR: (DMSOd₆) 2.14 (s, 6H), 2.27 (s, 3H), 3.32 (s, 2H), 3.98 (s, 3H), 3.99 (s, 3H), 5.01 (s, 2H), 6.83 (s, 1H), 7.32 (s, 1H), 7.37 (s, 1H), 7.39 (s, 1H), 7.54 (s, 1H), 7.67 (s, 1H), 8.12 (s, 1H), 8.66 (s, 1H), 10.24 (s, 1H); Mass Spectrum: M+H⁺ 477.

The 3-dimethylaminomethyl-5-methylaniline used as a starting material was prepared as follows: —

A mixture of 1,3-dimethyl-5-nitrobenzene (15.15 g), N-bromosuccinimide (2 g), benzoyl peroxide (0.484 g) and carbon tetrachloride (250 ml) was stirred and heated to reflux. Further portions of N-bromosuccinimide (totalling 21 g) were added portionwise during 4 hours to the heated reaction mixture. The mixture was cooled to ambient temperature. Petroleum ether (b.p. 60-80° C.) was added. The mixture was filtered and the filtrate was evaporated to give an oil (25 g) which was shown by NMR analysis to be a mixture of 3-methyl-5-nitrobenzyl bromide (76%), unreacted starting material (˜19%) and 3-bromomethyl-5-nitrobenzyl bromide (˜15%). This mixture was used in the next step.

A portion (2.3 g) of the oil so obtained was dissolved in ethanol (5 ml) and dimethylamine (6 equivalents) was added portionwise in order to prevent a significant exotherm. The resultant reaction mixture was stirred at ambient temperature for 12 hours. The mixture was evaporated and the residue was purified by column chromatography on silica using increasingly polar mixtures of methylene chloride and diethyl ether as eluent. There was thus obtained N,N-dimethyl-N-(3-methyl-5-nitrobenzyl)amine (0.98 g); ¹H NMR: (DMSOd₆) 2.17 (s, 6H), 2.43 (s, 3M), 3.48 (s, 2H), 7.58 (s, 1H), 7.94 (m, 2H); Mass Spectrum: M+H⁺ 195.

Using an analogous procedure to that described in the last paragraph of the portion of Note [19] above that is concerned with the preparation of starting materials, N,N-dimethyl-N-(3-methyl-5-nitrobenzyl)amine was hydrogenated to give 3-dimethylaminomethyl-5-methylaniline in 94% yield; ¹H N: (DMSOd₆) 2.09 (s, 6H), 2.12 (s, 3H), 3.16 (s, 2H), 4.87 (s, 2H), 6.24 (s, 2H), 6.31 (s, 1H).

[21] The product was purified by column chromatography on silica using a 19:1 mixture of methylene chloride and a 3M methanolic ammonia solution as eluent and gave the following characterising data: —¹H NMR: (DMSOd₆) 1.02 (s, 3H), 2.09 (br s, 3H), 2.27 (s, 3H), 2.32-2.44 (m, 2H), 3.37 (br s, 2H), 3.98 (s, 3H), 3.99 (s, 3H), 5.01 (s, 2H), 6.83 (s, 1H), 7.34 (s, 1H), 7.37 (s, 1H), 7.39 (s, 1H), 7.54 (s, 1H), 7.67 (s, 1H), 8.12 (s, 1H), 8.66 (s, 1H), 10.24 (s, 1H); Mass Spectrum: M+H⁺ 491.

The 3-(N-ethyl-N-methylaminomethyl)-5-methylaniline used as a starting material was prepared as follows: —

Using analogous procedures to those described in the portion of Note [20] above that is concerned with the preparation of starting materials, 3-methyl-5-nitrobenzyl bromide was converted into N-ethyl-N-methyl-N-(3-methyl-5-nitrobenzyl)amine; ¹H NMR: (DMSOd₆) 1.03 (t, 3H), 2.11 (s, 3H), 2.41 (q, 2H), 2.42 (s, 3H), 3.54 (s, 2H), 7.58 (s, 1H), 7.94 (s, 2H); which in turn was converted into 3-(N-ethyl-N-methylaminomethyl)-5-methylaniline; ¹H NMR: (DMSOd₆) 1.01 (t, 3H), 2.08 (s, 3H), 2.12 (s, 3H), 2.36 (q, 2H), 3.26 (br s, 2H), 4.88 (br s, 2H), 6.25 (s, 2H), 6.33 (s, 1H).

[22] ¹H NMR: (DMSOd₆) 0.31-0.37 (m, 2H), 0.43-0.49 (m, 2H), 1.7-1.77 (m, 1H), 2.13 (s, 3H), 2.26 (s, 3H), 3.53 (s, 2H), 3.98 (s, 3H), 3.99 (s, 3H), 5.0 (s, 2H), 6.78 (s, 1H), 7.29 (s, 1H), 7.34 (s, 1H), 7.4 (s, 1H), 7.54 (s, 1H), 7.67 (s, 1H), 8.11 (s, 1H), 8.66 (s, 1H), 10.23 (s, 1H); Mass Spectrum: M+H⁺ 503.

The 3-(N-cyclopropyl-N-methylaminomethyl)-5-methylaniline used as a starting material was prepared as follows: —

Using an analogous procedure to that described in the second paragraph of the portion of Note [20] above that is concerned with the preparation of starting materials, 3-methyl-5-nitrobenzyl bromide was converted into N-cyclopropyl-N-(3-methyl-5-nitrobenzyl)amine; ¹H NMR: (DMSOd₆) 0.25 (m, 2H), 0.35 (m, 2H), 2.03 (m, 1H), 2.88 (br s, 1H), 3.8 (s, 3H), 7.6 (s, 1H), 7.92 (s, 1H), 7.99 (s, 1H).

Sodium triacetoxyborohydride (1.78 g) was added portionwise to a stirred mixture of N-cyclopropyl-N-(3-methyl-5-nitrobenzyl)amine (1.44 g), formaldehyde (37% aqueous solution, 0.81 ml), acetic acid (0.48 ml), methylene chloride (20 ml) and methanol (101) and the reaction mixture was stirred at ambient temperature for 2 hours. The mixture was evaporated and the residue was purified by column chromatography on silica using increasingly polar mixtures of methylene chloride and diethyl ether as eluent. There was thus obtained N-cyclopropyl-N-methyl-N-(3-methyl-5-nitrobenzyl)amine (1.32 g); ¹H NMR: (DMSOd₆) 0.35 (m, 2H), 0.46 (m, 2H), 1.77 (m, 1H), 2.17 (s, 3H), 2.42 (s, 3H), 3.7 (s, 2H), 7.54 (s, 1H), 7.88 (s, 1H), 7.95 (s, 1H); Mass Spectrum: M+H⁺ 221.

Using an analogous procedure to that described in the last paragraph of the portion of Note [19] above that is concerned with the preparation of starting materials, N-cyclopropyl-N-methyl-N-(3-methyl-5-nitrobenzyl)amine was hydrogenated to give 3-(N-cyclopropyl-N-methylaminomethyl)-5-methylaniline in 91% yield; ¹H NMR: (DMSOd₆) 0.32 (m, 2H), 0.44 (m, 2H), 1.69 (m, 1H), 2.1 (s, 3H), 2.11 (s, 3H), 3.95 (s, 2H), 4.86 (s, 2H), 6.2 (s, 1H), 6.23 (s, 1H), 6.26 (s, 1H).

[23] The product was purified by column chromatography on silica using a 19:1 mixture of methylene chloride and a 3M methanolic ammonia solution as eluent and gave the following characterising data: —¹H NMR: (DMSOd₆) 1.7 (br s, 4H), 2.27 (s, 3H), 2.36-2.52 (br s, 4H), 3.52 (br s, 2H), 3.98 (s, 3H), 3.99 (s, 3H), 5.01 (s, 2H), 6.84 (s, 1H), 7.33 (s, 1H), 7.36-7.43 (m, 2H), 7.54 (s, 1H), 7.67 (s, 1H), 8.12 (s, 1H), 8.66 (s, 1H), 10.24 (s, 1H); Mass Spectrum: M+H⁺ 503.

The 5-methyl-3-pyrrolidin-1-ylmethylaniline used as a starting material was prepared as follows: —

Using analogous procedures to those described in the portion of Note [20] above that is concerned with the preparation of starting materials, 3-methyl-5-nitrobenzyl bromide was converted into N-(3-methyl-5-nitrobenzyl)pyrrolidine; ¹H NMR: (DMSOd₆) 1.72 (m, 4H), 2.43 (s, 3H), 2.44 (s, 4H), 3.66 (s, 2H), 7.59 (s, 1H), 7.94 (s, 2H); which in turn was converted into 5-methyl-3-pyrrolidin-1-ylmethylaniline; ¹H NMR: (DMSOd₆) 1.66 (m, 4H), 2.12 (s, 3H), 2.36 (m, 4H) 3.33 (br s, 2H), 4.86 (br s, 2H), 6.23 (s, 1H), 6.24 (s, 1H), 6.33 (s, 1H).

[24] ¹H NMR: (DMSOd₆) 2.26 (s, 6H), 2.5-2.55 (m, 2H), 2.78-2.85 (m, 2H), 3.98 (s, 3H), 3.99 (s, 3H), 5.04 (s, 2H), 7.37 (d, 1H), 7.39 (s, 1H), 7.46 (m, 1H), 7.54 (s, 1H), 7.64 (d, 1H), 7.68 (s, 1H), 8.12 (s, 1H), 8.66 (s, 1H), 10.24 (s, 1H); Mass Spectrum: M+H⁺ 511 and 513.

The 4-chloro-3-(2-dimethylaminoethyl)aniline used as a starting material was prepared as follows: —

A solution of 2-(2-chlorophenyl)acetic acid (10 g) in concentrated sulphuric acid (40 ml) was stirred and cooled to −10° C. A mixture of fuming nitric acid (2.8 ml) and concentrated sulphuric acid (7.2 ml) was added at such a rate that the temperature of the reaction mixture did not exceed 0° C. The resultant mixture was stirred at 0° C. for 3 hours. The mixture was slowly poured into a stirred mixture of ice and water. The resultant solid was isolated, dissolved in diethyl ether and dried over magnesium sulphate. The solution was filtered and the filtrate was evaporated. The solid so obtained as dried under vacuum at 50° C. for 2 hours. There was thus obtained 2-(2-chloro-5-nitrophenyl)acetic acid (11.5 g); ¹H NMR (DMSOd₆) 3.91 (s, 2H), 7.77 (d, 1H), 8.16 (m, 1H), 8.37 (d, 1H).

Diborane (1M solution in THF; 80 ml) was added portionwise to a stirred solution of 2-(2-chloro-5-nitrophenyl)acetic acid (8.64 g) in THF (20 ml) and the resultant mixture was stirred at ambient temperature for 48 hours. Water was added carefully to destroy any excess reducing agent. A 2N aqueous hydrochloric acid solution was added and the mixture was extracted with diethyl ether. The organic solvent was evaporated and the residue was purified by column chromatography on silica using a 19:1 mixture of methylene chloride and methanol as eluent. There was thus obtained 2-(2-chloro-5-nitrophenyl)ethanol (6.4 g); ¹H NMR: (DMSOd₆) 2.97 (t, 2H), 3.69 (q, 2H), 4.82 (t, 1H), 7.73 (d, 1H), 8.1 (m, 1H), 8.24 (d, 1H).

A solution of 2-(2-chloro-5-nitrophenyl)ethanol (0.81 g) in methylene chloride (5 ml) was stirred and cooled to 5° C. 1,1,1-Tris(acetoxy)-1,1-dihydro-1,2-benziodoxol-3-(1H)-one (0.5M solution in methylene chloride; 8 ml) was added and the reaction mixture was stirred at ambient temperature for 1 hour. A 1M aqueous sodium bicarbonate solution (5 ml) and a 1M aqueous sodium bisulphite solution (5 ml) were added in turn and the mixture was stirred at ambient temperature for 10 minutes. The mixture was extracted with methylene chloride and the organic solution was evaporated. The residue was purified by column chromatography on silica using increasingly polar mixtures of petroleum ether (b.p. 40-60° C.) and methylene chloride as eluent. There was thus obtained 2-(2-chloro-5-nitrophenyl)acetaldehyde (0.38 g); ¹H NMR: (DMSOd₆) 4.16 (s, 2H), 7.78 (d, 1H), 8.18 (m, 1H), 8.33 (d, 1H), 9.74 (s, 1H).

Using an analogous procedure to that described in the second paragraph of the portion of Note [22] above that is concerned with the preparation of starting materials, 2-(2-chloro-5-nitrophenyl)acetaldehyde was reacted with dimethylamine in the presence of sodium triacetoxyborohydride. There was thus obtained N-[2-(2-chloro-5-nitrophenyl)ethyl]-N,N-dimethylamine in 91% yield; ¹H NMR: (DMSO₆) 2.2 (s, 6H), 2.5 (t, 2H), 2.95 (t, 2H), 7.72 (d, 1H), 8.08 (m, 1H), 8.28 (d, 1H).

A mixture of the material so obtained, 10% platinum-on-carbon catalyst (0.15 g) and ethyl acetate (25 ml) was stirred under 1.8 atmospheres pressure of hydrogen for 1 hour. The reaction mixture was filtered and the filtrate was evaporated. There was thus obtained 4-chloro-3-(2-dimethylaminoethyl)aniline in 100% yield; ¹H NMR: (DMSOd₆) 2.18 (s, 6H), 2.37 (t, 2H), 2.64 (t, 2H), 5.13 (s, 2H), 6.4 (m, 1H), 6.5 (m, 1H), 6.97 (m, 1H); Mass Spectrum: M+H⁺ 199 and 201.

[25] ¹H NMR: (DMSOd₆) 2.22 (s, 6H), 2.4-2.5 (m, 2H), 2.63-2.7 (m, 2H), 3.76 (s, 2H), 3.98 (s, 3H), 3.99 (s, 3H), 4.99 (s, 2H), 6.92 (d, 1H), 7.38-7.44 (m, 3H), 7.54 (s, 1H), 7.67 (s, 1H), 8.11 (s, 1H), 8.66 (s, 1H), 10.14 (s, 1H); Mass Spectrum: M+H⁺ 507.

The 3-(2-dimethylaminoethyl)-4-methoxyaniline used as a starting material was prepared as follows: —

A 2M solution of dimethylamine in methanol (3 ml) and dimethylamine hydrochloride (0.162 g) were added in turn to a solution of 2-(5-amino-2-methoxyphenyl)acetonitrile (Bull. Soc. Chim. Fr., 1963, 2022-5; 0.162 g) in ethanol (4 ml). The resultant mixture was hydrogenated under 5 atmospheres pressure of hydrogen over platinum oxide catalyst (0.04 g) at ambient temperature for 12 hours. The mixture was filtered and the residue was concentrated to give an oil (0.18 g) which was used without further purification; ¹H NMR: (DMSOd₆+CF₃CO₂D) 2.85 (s, 6H), 2.99 (m, 2H), 3.21 (m, 2H), 7.13 (m, 1H), 7.24 (m, 1H), 7.31 (m, 1H); Mass Spectrum: M+H⁺ 195.

[26] ¹H NMR: (DMSOd₆) 3.98 (s, 3H), 3.99 (s, 3H), 4.53 (s, 2H), 5.03 (s, 2H), 5.35 (br s, 1H), 7.11 (t, 1H), 7.39 (s, 1H), 7.56 (m, 2H), 7.68 (m, 2H), 8.12 (s, 1H), 8.66 (s, 1H); Mass Spectrum: M+H⁺ 454.

4-Fluoro-3-hydroxymethylaniline is described in J. Med. Chem., 1990, 33, 327.

[27] The product was purified by column chromatography on silica using a 19:1 mixture of methylene chloride and methanol as eluent and gave the following characterising data ¹H NMR: (DMSOd₆) 3.98 (2s, 6H), 4.48 (d, 2H), 5.05 (s, 2H), 5.38 (t, 1H), 7.07 (s, 1H), 7.38 (s, 1H), 7.43 (s, 1H), 7.54 (s, 1H), 7.69 (s, 2H), 8.13 (s, 1H), 8.66 (s, 1H); Mass Spectrum: M+H⁺ 470 and 472.

5-Chloro-3-hydroxymethylaniline is described in J. Med. Chem., 1984, 27, 1111.

[28] The product was purified by column chromatography on silica using a 19:1 mixture of methylene chloride and methanol as eluent and gave the following characterising data: —

¹H NMR: (DMSOd₆) 3.98 (2s, 6H), 4.53 (d, 2H), 5.04 (s, 2H), 5.46 (t, 1H), 7.37 (m, 1H), 7.39 (s, 1H), 7.54 (s, 1H), 7.61 (m, 1H), 7.67 (s, 1H), 7.77 (m, 1H), 8.13 (s, 1H), 8.63 (s, 1H); Mass Spectrum: M+H⁺ 470 and 472.

4-Chloro-3-hydroxymethylaniline is described in Liebig's Annalen der Chemie, 1986, 438.

### EXAMPLE 10

### N-(3-methylaminomethylphenyl)-2-[4-(6,7-dimethoxyquinazolin-4-yloxy)pyrazol-1-yl]acetamide

A mixture of N-[3-(N-tert-butoxycarbonyl-N-methylaminomethyl)phenyl]-2-[4-(6,7-dimethoxyquinazolin-4-yloxy)pyrazol-1-yl]acetamide (0.19 g), trifluoroacetic acid (4 ml) and methylene chloride (2 ml) was stirred at ambient temperature for 1 hour. The resultant mixture was evaporated and the residue was triturated under a mixture of methylene chloride and diethyl ether. The solid so obtained was isolated and dried under vacuum at 50° C. There was thus obtained the title compound (0.135 g); ¹H NMR: (DMSOd₆) 2.25 (s, 3H), 2.61 (s, 2H), 3.98 (s, 3H), 3.99 (s, 3H), 5.03 (d, 2H), 7.03 (d, 1H), 7.26 (m, 1H), 7.39 (s, 1H), 7.48 (d, 1H), 7.54 (s, 1H), 7.56 (s, 1H), 7.67 (s, 1H), 8.12 (s, 1H), 8.66 (s, 1H), 10.3 (s, 1H); Mass Spectrum: M+H⁺ 449.

The N-[3-(N-tert-butoxycarbonyl-N-methylaminomethyl)phenyl]-2-[4-(6,7-dimethoxyquinazolin-4-yloxy)pyrazol-1-yl]acetamide used as a starting material was prepared as follows: —

Under an atmosphere of argon, triethylamine (5.05 g) was added to a stirred mixture of 3-nitrobenzaldehyde (3.78 g), methylamine hydrochloride (3.33 g), titanium (IV) isopropoxide (14.2 g) and ethanol (38 ml) and the resultant mixture was stirred at ambient temperature for 16 hours. Sodium borohydride (37.5 g) was added portionwise and the resultant mixture was stirred at ambient temperature for 8 hours. The reaction mixture was poured into a stirred 2M aqueous ammonium hydroxide solution and the resultant solid was recovered by filtration. The solid was dissolved in 2N aqueous hydrochloric acid and the solution was washed with methylene chloride. The aqueous phase was basified to pH11 by the addition of 4N aqueous sodium hydroxide solution and extracted with a mixture of ethyl acetate and diethyl ether. The organic phase was dried over magnesium sulphate and evaporated. There was thus obtained N-methyl-N-(3-nitrobenzyl)amine as an oil (3.6 g); ¹H NMR: (DMSOd₆) 2.26 (s, 3H), 3.76 (s, 2H), 7.61 (t, 1H), 7.77 (d, 1H), 8.09 (d, 1H), 8.19 (s, 1H); Mass Spectrum: M+H⁺ 167.

A mixture of N-methyl-N-(3-nitrobenzyl)amine (1.66 g), di-tert-butyl dicarbonate (2.62 g) and methylene chloride (17 ml) was stirred at ambient temperature for 2 hours. The solvent was evaporated and the residue was partitioned between water and a 1:1 mixture of diethyl ether and ethyl acetate. The organic phase was dried over magnesium sulphate and evaporated. There was thus obtained N-tert-butoxycarbonyl-N-methyl-N-(3-nitrobenzyl)amine as an oil (2.66 g); ¹H NMR: (DMSOd₆) 1.41 (s, 9H), 2.82 (s, 3H), 4.5 (s, 2H), 7.66 (m, 2H), 8.07 (s, 1H), 8.13 (d, 1H).

A mixture of the material so obtained, platinum oxide (0.2 g), ethyl acetate (20 ml) and ethanol (20 ml) was stirred under 6 atmospheres pressure of hydrogen for 2 hours. The catalyst was removed by filtration and the filtrate was evaporated. There was thus obtained 3-(N-tert-butoxycarbonyl-N-methylaminomethyl)aniline as an oil (2.36 g); ¹H NMR: (DMSOd₆) 1.4 (s, 9H), 2.71 (s, 3H), 4.2 (s, 2H), 5.05 (br s, 2H), 6.33 (m, 1H), 6.4 (s, 1H), 6.44 (m, 1H), 6.96 (t, 1H); Mass Spectrum: M+H⁺ 237.

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-methylaminomethyl)aniline to give the required starting material in 78% yield; ¹H NMR: (DMSOd₆) 1.38 and 1.43 (2 br s, 9H), 2.76 (s, 3H), 3.98 (s, 3H), 3.99 (s, 3H), 4.53 (s, 2H), 5.04 (s, 2H), 6.93 (m, 1H), 7.31 (t, 1H), 7.39 (s, 1H), 7.43-7.5 (m, 2H), 7.54 (s, 1H), 7.68 (s, 1H), 8.12 (s, 1H), 8.66 (s, 1H); Mass Spectrum: M+H⁺ 549.

### EXAMPLE 11

### N-(4-methylaminomethylphenyl)-2-[4-(6,7-dimethoxyquinazolin-4-yloxy)pyrazol-1-yl]acetamide

Using an analogous procedure to that described in Example 10, N-[4-(N-tert-butoxycarbonyl-N-methylaminomethyl)phenyl]-2-[4-(6,7-dimethoxyquinazolin-4-yloxy)pyrazol-1-yl]acetamide was reacted with trifluoroacetic acid to give the title compound in 81% yield; ¹H NMR: (DMSOd₆) 2.25 (s, 3H), 3.6 (s, 2H), 3.98 (s, 3H), 3.99 (s, 3H), 5.03 (d, 2H), 7.27 (d, 2H), 7.39 (s, 1H), 7.53 (d, 2H), 7.54 (s, 1H), 7.67 (s, 1H), 8.12 (s, 1H), 8.66 (s, 1H), 10.3 (s, 1H); Mass Spectrum: M+H⁺ 449.

The N-[4-(N-tert-butoxycarbonyl-N-methylaminomethyl)phenyl]-2-[4-(6,7-dimethoxyquinazolin-4-yloxy)pyrazol-1-yl]acetamide used as a starting material was prepared as follows: —

Using analogous procedures to those described in the portion of Example 10 that is concerned with the preparation of starting materials, 4-nitrobenzaldehyde was converted into N-methyl-N-(4-nitrobenzyl)amine; ¹H NMR: (DMSOd₆) 2.26 (s, 3H), 3.76 (s, 2H), 7.6 (d, 2H), 8.18 (d, 2H); Mass Spectrum: M+H⁺ 167; which in turn was converted into N-tert-butoxycarbonyl-N-methyl-N-(4-nitrobenzyl)amine; ¹H NMR: (DMSOd₆) 1.35-1.44 (2 s, 9H), 2.82 (s, 3H), 4.51 (s, 2H), 7.47 (d, 2H), 8.23 (d, 2H); and which in turn was converted into 4-(N-tert-butoxycarbonyl-N-methylaminomethyl)aniline; ¹H NMR: (DMSOd₆) 1.41 (s, 9H), 2.67 (s, 3H), 4.15 (s, 2H), 4.99 (br s, 2H), 6.51 (d, 2H), 6.88 (d, 2H); Mass Spectrum: M+H⁺ 237.

Using an analogous procedure to that described in Example 8, 2-[4-(6,7-dimethoxyquinazolin-4-yloxy)pyrazol-1-yl]acetic acid was reacted with 4-(N-tert-butoxycarbonyl-N-methylaminomethyl)aniline to give the required starting material in 75% yield; ¹H NMR: (DMSOd₆) 1.39 and 1.42 (2 br s, 9H), 2.74 (s, 3H), 3.98 (s, 3H), 3.99 (s, 3H), 4.32 (s, 2H), 5.03 (s, 2H), 7.18 (d, 2H), 7.39 (s, 1H), 7.54 (s, 1H), 7.57 (m, 2H), 7.67 (s, 1H), 8.12 (s, 1H), 8.66 (s, 1H); Mass Spectrum: M+H⁺ 549.

### EXAMPLE 12

### N-(4-ethylaminomethylphenyl)-2-[4-(6,7-dimethoxyquinazolin-4-yloxy)pyrazol-1-yl]acetamide

Using an analogous procedure to that described in Example 10, N-[4-(N-tert-butoxycarbonyl-N-ethylaminomethyl)phenyl]-2-[4-(6,7-dimethoxyquinazolin-4-yloxy)pyrazol-1′-yl]acetamide was reacted with trifluoroacetic acid to give the title compound in 96% yield; ¹H NMR: (DMSOd₆) 1.17 (t, 3H), 2.9 (q, 2H), 3.98 (s, 3H), 3.99 (s, 3H), 4.03 (s, 2H), 5.06 (d, 2H), 7.39 (s, 1H), 7.42 (d, 2H), 7.54 (s, 1H), 7.64 (d, 2H), 7.68 (s, 1H), 7.96-8.48 (m, 1H), 8.13 (s, 1H), 8.66 (s, 1H), 10.48 (s, 1H); Mass Spectrum: M+H⁺ 463.

The N-[4-(N-tert-butoxycarbonyl-N-ethylaminomethyl)phenyl]-2-[4-(6,7-dimethoxyquinazolin-4-yloxy)pyrazol-1-yl]acetamide used as a starting material was prepared as follows: —

Using analogous procedures to those described in the portion of Example 10 that is concerned with the preparation of starting materials, 4-nitrobenzaldehyde was reacted with ethylamine and converted into N-ethyl-N-(4-nitrobenzyl)amine; ¹H NMR: (DMSOd₆) 1.03 (t, 3H), 2.5 (q, 2H), 3.8 (s, 2H), 7.61 (d, 2H), 8.17 (d, 2H); Mass Spectrum: M+H⁺ 181; which in turn was converted into N-tert-butoxycarbonyl-N-ethyl-N-(4-nitrobenzyl)amine; ¹H NMR: (DMSOd₆) 1.03 (t, 3H), 1.32 and 1.44 (2 s, 9H), 3.22 (br m, 2H), 4.5 (s, 2H), 7.49 (d, 2H), 8.22 (d, 2H); and which in turn was converted into 4-(N-tert-butoxycarbonyl-N-ethylaminomethyl)aniline; ¹H NMR: DMSOd₆) 0.95 (t, 3H), 1.41 (s, 9H), 3.07 (br s, 2H), 4.16 (s, 2H), 4.97 (br s, 2H), 6.5 (d, 2H), 6.9 (d, 2H); Mass Spectrum: M+H⁺ 251.

Using an analogous procedure to that described in Example 8, 2-[4-(6,7-dimethoxyquinazolin-4-yloxy)pyrazol-1-yl]acetic acid was reacted with 4-(N-tert-butoxycarbonyl-N-ethylaminomethyl)aniline to give the required starting material in 87% yield; ¹H NMR: (DMSOd₆) 0.98 (t, 3H), 1.39 (br s, 9H), 3.12 (br s, 2H), 3.98 (s, 3H), 3.99 (s, 3H), 4.31 (s, 2H), 5.03 (s, 2H), 7.2 (d, 2H), 7.39 (s, 1H), 7.54 (s, 1H), 7.56 (m, 2H), 7.67 (s, 1H), 8.12 (s, 1H), 8.66 (s, 1H); Mass Spectrum: M+H⁺ 563.

### EXAMPLE 13

### N-(3-aminomethylphenyl)-2-[4-(6,7-dimethoxyquinazolin-4-yloxy)pyrazol-1-yl]acetamide

Using a similar procedure to that described in Example 10, a mixture of N-[3-(N-tert-butoxycarbonylaminomethyl)phenyl]-2-[4-(6,7-dimethoxyquinazolin-4-yloxy)pyrazol-1-yl]acetamide (0.205 g), trifluoroacetic acid (0.5 ml) and methylene chloride (3 ml) was stirred at ambient temperature for 1 hour. The resultant mixture was evaporated and the residue was dissolved in ethyl acetate. A saturated aqueous sodium carbonate solution was added and a solid was slowly precipitated in the aqueous phase. The solid was recovered by filtration, washed with water and with ethyl acetate and dried. There was thus obtained the title compound (0.092 g); ¹H NMR: (DMSOd₆+CF₃CO₂D) 4.02 (s, 2H), 4.03 (s, 3H), 4.05 (s, 3H), 5.11 (s, 2H), 7.21 (d, 1H), 7.41 (m, 1H), 7.51 (s, 1H), 7.57 (d, 1H), 7.66 (s, 1H), 7.73 (s, 1H), 7.8 (s, 1H), 8.19 (s, 1H), 8.2-8.36 (m, 1H), 8.99 (s, 1H), 10.57 (s, 1H); Mass Spectrum: M+H⁺ 435.

The N-[4-(N-tert-butoxycarbonylaminomethyl)phenyl]-2-[4-(6,7-dimethoxyquinazolin-4-yloxy)pyrazol-1-yl]acetamide used as a starting material was prepared as follows: —

3-(N-tert-Butoxycarbonylaminomethyl)aniline (J. Med. Chem., 2003, 46, 1661-1669) was prepared by the hydrogenation under 1.2 atmospheres pressure of hydrogen for 1 hour of a mixture of N-tert-butoxycarbonyl-N-(3-nitrobenzyl)amine (2.5 g), 10% palladium-on-carbon catalyst (0.25 g) and ethanol (90 ml). The catalyst was removed by filtration and the filtrate was evaporated. There was thus obtained the required compound (2.16 g); ¹H NMR: (CDCl₃) 1.46 (s, 9H), 4.22 (m, 2H), 4.78 (m, 1H), 6.61 (m, 2H), 6.67 (m, 1H), 7.11 (t, 1H).

Using an analogous procedure to that described in Example 1, 2-[4-(6,7-dimethoxyquinazolin-4-yloxy)pyrazol-1-yl]acetic acid was reacted with 3-(N-tert-butoxycarbonylaminomethyl)aniline to give the required starting material in 85% yield; ¹H NMR: (CDCl₃) 1.45 (s, 9H), 2.98 (br s, 1H), 4.07 (s, 3H), 4.08 (s, 3H), 4.28 (m, 2H), 4.88 (br s, 1H), 4.95 (s, 2H), 7.06 (m, 1H), 7.37 (s, 1H), 7.41 (m, 2H), 7.5 (s, 1H), 7.87 (s, 1H), 8.0 (s, 1H), 8.38 (br s, 1H), 8.71 (s, 1H); Mass Spectrum: M+H⁺ 535.

### EXAMPLE 14

### N-[4-methoxy-3-(4-methylpiperazin-1-yl)methylphenyl]-2-[4-(6,7-dimethoxyquinazolin-4-yloxy)pyrazol-1-yl]acetamide

1-Methylpiperazine (0.029 ml) was added to a stirred mixture of N-(3-bromomethyl-4-methoxyphenyl)-2-[4-(6,7-dimethoxyquinazolin-4-yloxy)pyrazol-1-yl]acetamide (0.115 g), potassium carbonate (0.03 g) and DMF (3 ml) and the resultant mixture was stirred under argon at ambient temperature for 2 hours. The mixture was evaporated and the residue was purified by preparative HPLC using a Waters ‘Xterra’ C18 reversed-phase column (5 microns silica, 19 mm diameter, 100 mm length) and decreasingly polar mixtures of water (containing 0.2% ammonium carbonate) and acetonitrile as eluent. There was thus obtained the title compound as a solid (0.046 g); ¹H NMR: (DMSOd₆) 2.15 (s, 3H), 2.18-2.52 (br s, 8H), 3.42 (br s, 2H), 3.74 (s, 3H), 3.98 (s, 3H), 3.99 (s, 3H), 4.99 (s, 2H), 6.93 (d, 1H), 7.39 (d, 1H), 7.52 (s, 1H), 7.53 (d, 1H), 7.52 (s, 1H), 7.67 (s, 1H), 8.11 (s, 1H), 8.66 (s, 1H), 10.2 (s, 1H); Mass Spectrum: M+H⁺ 548.

The N-(3-bromomethyl-4-methoxyphenyl)-2-[4-(6,7-dimethoxyquinazolin-4-yloxy)pyrazol-1-yl]acetamide used as a starting material was prepared as follows: —

Carbon tetrabromide (0.043 g) and triphenylphosphine (0.034 g) were added in turn to a stirred solution of N-(3-hydroxymethyl-4-methoxyphenyl)-2-[4-(6,7-dimethoxyquinazolin-4-yloxy)pyrazol-1-yl]acetamide (0.05 g) in a mixture of THF (2 ml) and acetonitrile (2 ml) and the resultant mixture was heated to 50° C. for 2 hours. Second portions of each of carbon tetrabromide (0.043 g) and triphenylphosphine (0.034 g) were added in turn and the reaction mixture was heated to 50° C. for 1 hour. The mixture was evaporated and the residue was triturated under methylene chloride. There was thus obtained N-(3-bromomethyl-4-methoxyphenyl)-2-[4-(6,7-dimethoxyquinazolin-4-yloxy)pyrazol-1-yl]acetamide (0.04 g); ¹H NMR: (DMSOd₆) 3.83 (s, 3H), 3.99 (s, 3H), 4.0 (s, 3H), 4.63 (s, 2H), 5.01 (s, 2H), 7.02 (d, 1H), 7.4 (s, 1H), 7.56 (m, 2H), 7.65 (s, 1H), 7.68 (s, 1H), 8.13 (s, 1H), 8.7 (s, 1H), 10.27 (s, 1H); Mass Spectrum: M+H⁺ 528 and 530.

### EXAMPLE 15

Using an analogous procedure to that described in Example 14, the appropriate N-(3-bromomethylphenyl)-2-pyrazol-1-ylacetamide was reacted with the appropriate amine or heterocycle to give the compounds described in Table III. Unless otherwise stated, each required amine or heterocycle starting material was commercially available.

TABLE III

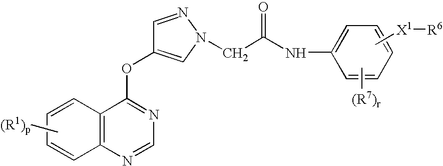

| No. & | | | |
|---|---|---|---|
| Note | (R¹)ₚ | (R⁷)ᵣ | X¹—R⁶ |
| [1] | 6,7-dimethoxy | 4-methoxy | 3-cyclopropylaminomethyl |
| [2] | 6,7-dimethoxy | 4-methoxy | 3-azetidin-1-ylmethyl |
| [3] | 6,7-dimethoxy | 4-methoxy | 3-(3-hydroxypyrrolidin-1-ylmethyl) |

Notes The products gave the characterising data shown below.

[1] ¹H NMR: (DMSOd₆) 0.22-0.28 (m, 2H), 0.31-0.39 (m, 2H), 2.02-2.09 (m, 1H), 3.34-3.51 (br s, 1H), 3.68 (s, 2H), 3.77 (s, 3H), 3.98 (s, 3H), 3.99 (s, 3H), 4.99 (s, 2H), 6.92 (s, 1H), 7.47 (d, 1H), 7.5 (m, 1H), 7.54 (s, 1H), 7.67 (s, 1H), 8.11 (s, 1H), 8.66 (s, 1H), 10.17 (s, 1H); Mass Spectrum: M+H⁺ 505.

[2] ¹H NMR: (DMSOd₆) 1.95-2.03 (m, 2H), 3.13-3.18 (m, 4H), 3.46 (s, 2H), 3.74 (s, 3H), 3.98 (s, 3H), 3.99 (s, 3H), 5.0 (s, 2H), 6.9 (d, 1H), 7.4 (s, 1H), 7.48 (s, 1H), 7.49 (m, 1H), 7.55 (s, 1H), 7.67 (s, 1H), 8.12 (s, 1H), 8.66 (s, 1H), 10.19 (s, 1H); Mass Spectrum: M+H⁺ 505.

[3] ¹H NMR: DMSOd₆) 1.49-1.58 (m, 1H), 1.94-2.03 (m, 1H), 2.31 (m, 1H), 2.42-2.5 (m, 1H), 2.54-2.62 (m, 1H), 2.74 (m, 1H), 3.31 (s, 2H), 3.74 (s, 3H), 3.98 (s, 3H), 3.99 (s, 3H), 4.16-4.23 (m, 1H), 4.68 (br s, 1H), 4.99 (s, 2H), 6.92 (d, 1H), 7.39 (s, 1H), 7.5 (s, 1H), 7.51 (d, 1H), 7.54 (s, 1H), 7.67 (s, 1H), 8.11 (s, 1H), 8.66 (s, 1H), 10.2 (s, 1H); Mass Spectrum: M+H⁺ 535.

### EXAMPLE 16

### N-[4-methoxy-3-(2-methylprop-2-en-1-ylaminomethyl)phenyl]-2-[4-(6,7-dimethoxyquinazolin-4-yloxy)pyrazol-1-yl]acetamide

Sodium triacetoxyborohydride (0.066 g) was added portionwise to a stirred mixture of N-(3-formyl-4-methoxyphenyl)-2-[4-(6,7-dimethoxyquinazolin-4-yloxy)pyrazol-1-yl]acetamide (0.11 g), 2-methylprop-2-en-1-ylamine (0.026 ml), methanol (0.5 ml) and methylene chloride (3 ml) and the resultant mixture was stirred at ambient temperature for 2 hours. The mixture was evaporated and the residue was purified by column chromatography on silica using a 19:1 mixture of methylene chloride and a 3M methanolic ammonia solution as eluent. There was thus obtained the title compound as a solid (0.1 g); ¹H NMR: (DMSOd₆) 1.7 (s, 3H), 1.76-2.06 (br s, 1H), 3.08 (s, 2H), 3.6 (s, 2H), 3.76 (s, 3H), 3.98 (s, 3H), 3.99 (s, 3H), 4.78 (br s, 1H), 4.88 (s, 1H), 4.98 (s, 2H); 6.91 (d, 1H), 7.38 (s, 1H), 7.49 (m, 1H), 7.5 (s, 1H), 7.54 (s, 1H), 7.66 (s, 1H), 8.09 (s, 1H), 8.64 (s, 1H), 10.02 (br s, 1H); Mass Spectrum: M+H⁺ 519.

The N-(3-formyl-4-methoxyphenyl)-2-[4-(6,7-dimethoxyquinazolin-4-yloxy)pyrazol-1-yl]acetamide used as a starting material was prepared as follows: —

Tetra-n-propylammonium ruthenium-tetroxide (0.03 g) and N-methylmorpholine N-oxide (0.227 g) were added in turn to a stirred mixture of N-(3-hydroxymethyl-4-methoxyphenyl)-2-[4-(6,7-dimethoxyquinazolin-4-yloxy)pyrazol-1-yl]acetamide (0.6 g), 4 Å molecular sieves (0.7 g) and acetonitrile (15 ml) that had been cooled to 0° C. The resultant mixture was stirred at ambient temperature under argon for 16 hours. A second portion of each of tetra-n-propylammonium ruthenium tetroxide (0.02 g) and N-methylmorpholine N-oxide (0.076 g) was added and the reaction mixture was stirred at ambient temperature for 2 hours. The mixture was evaporated and the residue was purified by column chromatography on silica using a 98:5 mixture of methylene chloride and methanol as eluent. There was thus obtained the required starting material as a solid (0.45 g); ¹H NMR: (DMSOd₆) 3.91 (s, 3H), 3.98 (s, 3H), 3.99 (s, 3H), 5.03 (s, 2H), 7.25 (d, 1H), 7.39 (s, 1H), 7.54 (s, 1H), 7.68 (s, 1H), 7.86 (m, 1H), 7.96 (d, 1H), 8.13 (s, 1H), 8.66 (s, 1H), 10.35 (s, 1H), 10.43 (s, 1H); Mass Spectrum: M+H⁺ 464.

### EXAMPLE 17

Using an analogous procedure to that described in Example 16, the appropriate N-(3-formylphenyl)-2-pyrazol-1-ylacetamide was reacted with the appropriate amine or heterocycle to give the compounds described in Table IV. Unless otherwise stated, each required amine or heterocycle starting material was commercially available.

TABLE IV

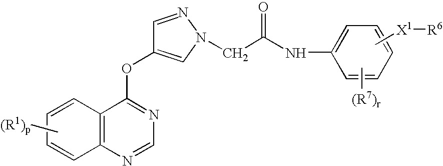

| No. & | | | |
|---|---|---|---|
| Note | (R¹)ₚ | (R⁷)ᵣ | X¹—R⁶ |
| [1] | 6,7-dimethoxy | 4-methoxy | 3-methylaminomethyl |
| [2] | 6,7-dimethoxy | 4-methoxy | 3-ethylaminomethyl |
| [3] | 6,7-dimethoxy | 4-methoxy | 3-propylaminomethyl |
| [4] | 6,7-dimethoxy | 4-methoxy | 3-isopropylaminomethyl |
| [5] | 6,7-dimethoxy | 4-methoxy | 3-(2,2,2- |
| | | | trifluoroethylaminomethyl) |
| [6] | 6,7-dimethoxy | 4-methoxy | 3-cycloprop-1- |
| | | | ylmethylaminomethyl |
| [7] | 6,7-dimethoxy | 4-methoxy | 3-(2-hydroxyethylaminomethyl) |
| [8] | 6,7-dimethoxy | 4-methoxy | 3-(2-methoxyethylaminomethyl) |
| [9] | 6,7-dimethoxy | 4-methoxy | 3-(2-cyanoethylaminomethyl) |
| [10] | 6,7-dimethoxy | 4-methoxy | 3-morpholinomethyl |
| [11] | 6,7-dimethoxy | 4-methoxy | 3-piperazin-1-ylmethyl |
| [12] | 6,7-dimethoxy | 4-methoxy | 3-(3-pyridylmethylaminomethyl) |
| [13] | 6,7-dimethoxy | 6-fluoro | 3-dimethylaminomethyl |
| [14] | 6,7-dimethoxy | 4-fluoro | 3-ethylaminomethyl |
| [15] | 6,7-dimethoxy | 4-fluoro | 3-isopropylaminomethyl |
| [16] | 6,7-dimethoxy | 4-fluoro | 3-(N-ethyl-N-methylaminomethyl) |
| [17] | 6,7-dimethoxy | 4-fluoro | 3-cyclopropylaminomethyl |
| [18] | 6,7-dimethoxy | 4-fluoro | 3-pyrrolidin-1-ylmethyl |
| [19] | 6,7-dimethoxy | 5-chloro | 3-ethylaminomethyl |
| [20] | 6,7-dimethoxy | 5-chloro | 3-(2-methylprop-2-en- |
| | | | 1-ylaminomethyl) |
| [21] | 6,7-dimethoxy | 5-chloro | 3-cyclopropylaminomethyl |

Notes The products gave the charactetising data shown below.

[1] ¹H NMR: (DMSOd₆) 2.27 (s, 3H), 3.58 (s, 2H), 3.76 (s, 3H), 3.98 (s, 3H), 3.99 (s, 3H), 4.99 (s, 2H), 6.92 (d, 1H), 7.39 (s, 1H), 7.47-7.52 (m, 2H), 7.54 (s, 1H), 7.67 (s, 1H), 8.11 (s, 1H), 8.66 (s, 1H), 10.17 (s, 1H); Mass Spectrum: M+H⁺ 479.

[2] ¹H NMR: (DMSOd₆) 1.07 (t, 3H), 2.59-2.67 (m, 2H), 3.72 (s, 2H), 3.77 (s, 3H), 3.98 (s, 3H), 3.99 (s, 3H), 5.0 (s, 2H), 6.95 (d, 1H), 7.39 (s, 1H), 7.52 (m, 1H), 7.54 (s, 1H), 7.55 (d, 1H), 7.67 (s, 1H), 8.12 (s, 1H), 8.66 (s, 1H), 10.22 (s, 1H); Mass Spectrum: M+H⁺ 493.

[3] ¹H NMR: (DMSOd₆) 0.86 (t, 3H), 1.39-1.51 (m, 2H), 2.47 (t, 2H), 3.63 (s, 2H), 3.75 (s, 3H), 3.98 (s, 3H), 3.99 (s, 3H), 5.0 (s, 2H), 6.91 (d, 1H), 7.39 (s, 1H), 7.49 (s, 1H), 7.52 (m, 1H), 7.54 (s, 1H), 7.67 (s, 1H), 8.11 (s, 1H), 8.66 (s, 1H), 10.17 (s, 1H); Mass Spectrum: M+H⁺ 507.

[4] ¹H NMR: (DMSOd₆) 1.0 (d, 6H), 2.67-2.78 (m, 1H), 3.65 (s, 2H), 3.76 (s, 3H), 3.98 (s, 3H), 3.99 (s, 3H), 5.0 (s, 3H), 6.92 (d, 1H), 7.39 (s, 4H), 7.48-7.55 (m, 2H), 7.54 (s, 1H), 7.67 (s, 1H), 8.11 (s, 1H), 8.66 (s, 1H), 10.18 (s, 1H); Mass Spectrum: M+H⁺ 507.

[5] H NMR: (DMSOd₆) 2.59-2.68 (m, 1H), 3.15-3.29 (m, 2H), 3.74 (d, 2H), 3.76 (s, 3H), 3.98 (s, 3H), 3.99 (s, 3H), 5.0 (s, 2H), 6.94 (d, 1H), 7.39 (s, 1H), 7.49 (d, 1H), 7.53 (m, 1H), 7.54 (s, 1H), 7.67 (d, 1H), 8.11 (s, 1H), 8.66 (s, 1H), 10.21 (s, 1H); Mass Spectrum: M+H⁺ 547.

[6] ¹H NMR: (DMSOd₆) 0.07-0.12 (m, 2H), 0.37-0.44 (m, 2H), 0.86-0.95 (m, 1H), 2.39 (d, 2H), 3.66 (s, 2H), 3.75 (s, 3H), 3.98 (s, 3H), 3.99 (s, 3H), 5.0 (s, 2H), 6.92 (d, 1H), 7.39 (s, 1H), 7.48-7.53 (m, 2H), 7.54 (s, 1H), 7.67 (s, 1H), 8.11 (s, 1H), 8.66 (s, 1H), 10.18 (s, 1H); Mass Spectrum: M+H⁺ 519.

[7] ¹H NMR: (DMSOd₆) 2.7 (t, 2H), 3.42 (t, 2H), 3.7 (s, 2H), 3.77 (s, 3H), 3.98 (s, 3H), 3.99 (s, 3H), 5.0 (s, 2H), 6.94 (d, 1H), 7.39 (s, 1H), 7.5 (s, 1H), 7.52 (d, 1H), 7.54 (s, 1H), 6.67 (s, 1H), 8.11 (s, 1H), 8.66 (s, 1H), 10.2 (s, 1H); Mass Spectrum: M+H⁺ 509.

[8] ¹H NMR: (DMSOd₆) 2.7 (t, 2H), 3.24 (s, 3H), 3.42 (t, 2H), 3.7 (s, 2H), 3.77 (s, 3H), 3.98 (s, 3H), 3.99 (s, 3H), 5.0 (s, 1H), 6.94 (d, 1H), 7.39 (s, 1H), 7.5 (s, 1H), 7.52 (d, 1H), 7.54 (s, 1H), 7.67 (s, 1H), 8.12 (s, 1H), 8.66 (s, 1H), 10.19 (s, 1H); Mass Spectrum: M+H⁺ 523.

[9] ¹H NMR: (DMSOd₆) 2.06-2.36 (m, 1H), 2.62 (t, 2H), 2.74 (t, 2H), 3.66 (s, 2H), 3.77 (s, 3H), 3.98 (s, 3H), 3.99 (s, 3H), 5.0 (s, 2H), 6.94 (d, 1H), 7.39 (s, 1H), 7.48 (d, 1H), 7.51 (m, 1H), 7.54 (s, 1H), 7.67 (s, 1H), 8.11 (s, 1H), 8.66 (s, 1H), 8.19 (s, 1H); Mass Spectrum: M+H⁺ 518.

[10] ¹H NMR: (DMSOd₆) 2.34-2.43 (m, 4H), 3.43 (s, 2H), 3.55-3.62 (m, 4H), 3.75 (s, 3H), 3.98 (s, 3H), 3.99 (s, 3H), 5.0 (s, 2H), 6.94 (d, 1H), 7.39 (s, 1H), 7.52 (m, 1H), 7.54 (s, 2H), 7.67 (s, 1H), 8.12 (s, 1H), 8.66 (s, 1H), 10.19 (s, 1H); Mass Spectrum: M+H⁺ 535.

[11] ¹H NMR: (DMSOd₆) 2.28-2.41 (m, 4H), 2.7-2.8 (m, 4H), 3.41 (s, 2H), 3.75 (s, 3H), 3.98 (s, 3H), 3.99 (s, 3H), 5.0 (s, 2H), 6.93 (d, 1H), 7.39 (s, 1H), 7.49-7.55 (m, 2H), 7.54 (s, 1H), 7.67 (s, 1H), 8.11 (s, 1H), 8.66 (s, 1H), 10.2 (s, 1H); Mass Spectrum: M+H⁺ 534.

[12] ¹H NMR: (DMSOd₆) 3.63 (s, 2H), 3.73 (s, 2H), 3.74 (s, 3H), 3.98 (s, 3H), 3.99 (s, 3H), 5.0 (s, 2H), 6.93 (d, 1H), 7.35 (m, 1H), 7.39 (s, 1H), 7.52-7.57 (m, 3H), 7.67 (s, 1H), 7.77 (m, 1H), 8.12 (s, 1H), 8.44 (m, 1H), 8.55 (d, 1H), 8.66 (s, 1H), 10.19 (s, 1H); Mass Spectrum: M+H⁺ 556.

[13] ¹H NMR: (DMSOd₆) 2.12 (s, 6H), 3.33 (s, 2H), 3.98 (s, 3H), 3.99 (s, 3H), 5.13 (s, 2H), 7.06 (m, 1H), 7.22 (m, 1H), 7.39 (s, 1H), 7.54 (s, 1H), 7.68 (s, 1H), 7.9 (m, 1H), 8.12 (s, 1H), 8.66 (s, 1H), 10.11 (s, 1H); Mass Spectrum: M+H⁺ 481.

The N-(6-fluoro-3-formylphenyl)-2-[4-(6,7-dimethoxyquinazolin-4-yloxy)pyrazol-1-yl]acetamide used as a starting material was prepared as follows: —

Tetra-n-propylammonium ruthenium tetroxide (0.156 g) and N-methylmorpholine N-oxide (0.042 g) were added in turn to a stirred mixture of N-(6-fluoro-3-hydroxymethylphenyl)-2-[4-(6,7-dimethoxyquinazolin-4-yloxy)pyrazol-1-yl]acetamide (0.136 g), 4 Å molecular sieves (0.136 g), methylene chloride (2 ml) and acetonitrile (3 ml). The resultant mixture was stirred and heated to 40° C. under argon for 4 hours. The mixture was filtered and the filtrate was evaporated. The residue was purified by column chromatography on silica using a 19:1 mixture of methylene chloride and methanol as eluent. There was thus obtained N-(6-fluoro-3-formylphenyl)-2-[4-(6,7-dimethoxyquinazolin-4-yloxy)pyrazol-1-yl]acetamide (0.076 g); ¹H NMR: (DMSOd₆) 3.98 (s, 6H), 5.19 (s, 2H), 7.39 (s, 1H), 7.55 (s, 1H), 7.58 (m, 1H), 7.69 (s, 1H), 7.77 (m, 1H), 8.14 (s, 1H), 8.56 (m, 1H), 8.66 (s, 1H), 9.95 (s, 1H), 10.41 (s, 1H); Mass Spectrum: M+H⁺ 452.

[14] ¹H NMR: (DMSOd₆) 1.02 (t, 3H), 1.98 (br s, 1H), 2.53 (q, 2H), 3.69 (s, 2H), 3.98 (s, 3H), 3.99 (s, 3H), 5.02 (s, 2H), 7.11 (m, 1H), 7.39 (s, 1H), 7.52 (m, 1H), 7.54 (s, 1H), 7.66 (m, 1H), 7.68 (s, 1H), 8.12 (s, 1H), 8.66 (s, 1H), 10.38 (s, 1H); Mass Spectrum: M+H⁺ 481.

The N-(4-fluoro-3-formylphenyl)-2-[4-(6,7-dimethoxyquinazolin-4-yloxy)pyrazol-1-yl]acetamide used as a starting material was prepared as follows: —

Using an analogous procedure to that described in the portion of Note [13] above that is concerned with the preparation of starting materials, N-(4-fluoro-3-hydroxymethylphenyl)-2-[4-(6,7-dimethoxyquinazolin-4-yloxy)pyrazol-1-yl]acetamide was oxidised to give the required product in 51% yield; ¹H NMR: (DMSOd₆) 3.98 (s, 3H), 3.99 (s, 3H), 5.07 (s, 2H), 7.39 (s, 1H), 7.42 (t, 1H), 7.54 (s, 1H), 7.69 (s, 1H), 7.89 (m, 1H), 8.11 (m, 1H), 8.14 (s, 1H), 8.66 (s, 1H), 10.22 (s, 1H); Mass Spectrum: M+H⁺ 452.

[15] ¹H NMR: (DMSOd₆) 1.01 (d, 6H), 2.67-2.76 (m, 1H), 3.31 (br s, 1H), 3.7 (s, 2H), 3.98 (s, 3H), 3.99 (s, 3H), 5.02 (s, 2H), 7.1 (m, 1H), 7.39 (s, 1H), 7.53 (m, 1H), 7.54 (s, 1H), 7.67 (m, 1H), 7.68 (s, 1H), 8.12 (s, 1H), 8.66 (s, 1H), 10.37 (s, 1H); Mass Spectrum: M+H⁺ 495.

[16] ¹H NMR: (DMSOd₆) 1.02 (t, 3H), 2.12 (s, 3H), 2.4 (q, 2H), 3.47 (s, 2H), 3.98 (s, 3H), 3.99 (s, 3H), 5.02 (s, 2H), 7.13 (m, 1H), 7.39 (s, 1H), 7.53 (m, 1H), 7.54 (s, 1H), 7.66 (m, 1H), 7.68 (s, 1H), 8.12 (s, 1H), 8.66 (s, 1H), 10.39 (br s, 1H); Mass Spectrum: M+H⁺ 495.

[17] ¹H NMR: (DMSOd₆) 0.22-0.29 (m, 2H), 0.32-0.38 (m, 2H), 2.03-2.1 (m, 1H), 2.61 (br s, 1H), 3.74 (s, 2H), 3.98 (s, 3H), 3.99 (s, 3H), 5.02 (s, 2H), 7.11 (m, 1H), 7.39 (s 1H), 7.52 (m, 1H), 7.54 (s, 1H), 7.64 (m, 1H), 7.67 (s, 1H), 8.12 (s, 1H), 8.66 (s, 1H), 10.37 (s, 1H); Mass Spectrum: M+H⁺ 493.

[18] ¹H NMR: (DMSOd₆) 1.66-1.74 (m, 4H), 2.43-2.49 (m, 4H), 3.59 (s, 2H), 3.98 (s, 3H), 3.99 (s, 3H), 5.02 (s, 1H), 7.13 (m, 1H), 7.39 (s, 1H), 7.52 (m, 1H), 7.54 (s, 1H), 7.66 (m, 1H), 7.68 (s, 1H), 8.12 (s, 1H), 8.66 (s, 1H), 10.39 (s, 1H); Mass Spectrum: M+H⁺ 507.

[19] ¹H NMR: (DMSOd₆) 1.02 (t, 3H), 2.14 (br s, 1H), 2.5 (q, 2H), 3.66 (s, 2H), 3.98 s, 3H), 3.99 (s, 3H), 5.05 (s, 2H), 7.11 (s, 1H), 7.39 (s, 1H), 7.43 (s, 1H), 7.54 (s, 1H), 7.67 (br s, 1H), 7.68 (s, 1H), 8.13 (s, 1H), 8.66 (s, 1H), 10.52 (br s, 1H); Mass Spectrum: M+H⁺ 497 and 499.

The N-(5-chloro-3-formylphenyl)-2-[4-(6,7-dimethoxyquinazolin-4-yloxy)pyrazol-1-yl]acetamide used as a starting material was prepared as follows: —

Using an analogous procedure to that described in the portion of Note [13] above that is concerned with the preparation of starting materials, N-(5-chloro-3-hydroxymethylphenyl)-2-[4-(6,7-dimethoxyquinazolin-4-yloxy)pyrazol-1-yl]acetamide was oxidised to give the required product in 68% yield; ¹H NMR: (DMSOd₆) 3.98 (s, 3H), 3.99 (s, 3H), 5.1 (s, 2H), 7.39 (s, 1H), 7.54 (s, 1H), 7.69 (s, 1H), 8.04 (s, 1H), 8.15 (s, 1H), 8.66 (s, 1H), 9.96 (s, 1H), 10.84 (br s, 1H); Mass Spectrum: M+H⁺ 468 and 470.

[20] ¹H NMR: (DMSOd₆) 1.7 (s, 3H), 2.4 (br s, 1H), 3.02 (s, 2H), 3.62 (s, 2H), 3.98 (s, 3H), 3.99 (s, 3H), 4.8 (s, 1H), 4.87 (s, 1H), 5.05 (s, 2H), 7.12 (s, 1H), 7.4 (s, 1H), 7.42 (s, 1H), 7.54 (s, 1H), 7.66-7.71 (m, 2H), 8.12 (s, 1H), 8.66 (s, 1H), 10.15 (s, 1H); Mass Spectrum: M−H⁻ 521 and 523.

[21] ¹H NMR: (DMSOd₆) 0.22-0.27 (m, 2H), 0.32-0.39 (m, 2H), 2.0-2.03 (m, 1H), 2.81 (br s, 1H), 3.69 (s, 2H), 3.98 (s; 3H), 3.99 (s, 3H), 5.04 (s, 2H), 7.11 (s, 1H), 7.39 (s, 1H), 7.41 (s, 1H), 7.54 (s, 1H), 7.67 (s, 1H), 7.68 (s, 1H), 8.12 (s, 1H), 8.66 (s, 1H), 10.5 (s, 1H); Mass Spectrum: M+H⁺ 509 and 511.

### EXAMPLE 18

### N-[3-(2-aminoethyl)phenyl]-2-[4-(6,7-dimethoxyquinazolin-4-yloxy)pyrazol-1-yl]acetamide

A mixture of N-{3-[2-(N-tert-butoxycarbonylamino)ethyl]phenyl}-2-[4-(6,7-dimethoxyquinazolin-4-yloxy)pyrazol-1-yl]acetamide (0.197 g), trifluoroacetic acid (0.5 ml) and methylene chloride (3 ml) was stirred at ambient temperature for 1 hour. The resultant mixture was evaporated and the residue was treated with a mixture of ethyl acetate, methanol and a saturated aqueous sodium carbonate solution. The solid which precipitated in the aqueous phase was recovered, washed with water and with ethyl acetate and dried under vacuum. There was thus obtained the title compound (0.097 g); ¹H NMR: (DMSOd₆) 2.57-2.67 (m, 2H), 2.72-2.79 (m, 1H), 3.07-3.14 (m, 1H), 3.98 (s, 3H), 3.99 (s, 3H), 5.04 (s, 2H), 6.91 (d, 1H), 7.22 (d, 1H), 7.33-7.52 (3H), 7.54 (s, 1H), 7.67 (s, 1H), 8.12 (s, 1H), 8.66 (s, 1H), 10.39-10.53 (m, 1H); Mass Spectrum: M+H⁺ 449.

The N-{3-[2-(N-tert-butoxycarbonylamino)ethyl]phenyl}-2-[4-(6,7-dimethoxyquinazolin-4-yloxy)pyrazol-1-yl]acetamide used as a starting material was prepared as follows: —

Using an analogous procedure to that described in the first paragraph of the portion of Example 13 that is concerned with the preparation of starting materials, tert-butyl N-[2-(3-nitrophenyl)ethyl]carbamate (Bioorganic & Medicinal Chemistry, 2003, 11, 4189-4206) was hydrogenated to give 3-[2-(N-tert-butoxycarbonylamino)ethyl]aniline in 67% yield; ¹H NMR: (CDCl₃) 1.43 (s, 9H), 2.7 (t, 2H), 3.36 (m, 2H), 4.53 (br s, 1H), 6.59 (m, 3H), 7.1 (t, 1H).

Using an analogous procedure to that described in Example 1, 2-[4-(6,7-dimethoxyquinazolin-4-yloxy)pyrazol-1-yl]acetic acid was reacted with 3-[2-(N-tert-butoxycarbonylamino)ethyl]aniline to give the required starting material in 79% yield; ¹H NMR: (CDCl₃) 1.42 (s, 9H), 2.77 (t, 2H), 2.99 (br s, 1H), 3.36 (m, 2H), 4.07 (s, 3H), 4.08 (s, 3H), 4.54 (br s, 1H), 4.96 (s, 2H), 6.97 (d, 1H), 7.26 (m, 1H), 7.36 (s, 1H), 7.4 (m, 1H), 7.51 (s, 1H), 7.87 (s, 1H), 8.01 (s, 1H), 8.34 (s, 1H), 8.71 (s, 1H); Mass Spectrum: M+H⁺ 549.

### EXAMPLE 19

### N-(3-cyclopropylaminomethylphenyl)-2-[4-(6,7-dimethoxyquinazolin-4-yloxy)pyrazol-1-yl]acetamide

A mixture of N-[3-(N-tert-butoxycarbonyl-N-cyclopropylaminomethyl)phenyl]-2-[4-(6,7-dimethoxyquinazolin-4-yloxy)pyrazol-1-yl]acetamide (0.2 g), trifluoroacetic acid (4 ml) and methylene chloride (2 ml) was stirred at ambient temperature for 1 hour. The resultant mixture was evaporated and the residue was purified by column chromatography on silica using a 19:1 mixture of methylene chloride and a 3M methanolic ammonia solution as eluent. The solid so obtained was dried under vacuum at 50° C. for 12 hours. There was thus obtained the title compound (0.103 g); ¹H NMR: (DMSOd₆) 0.22-0.37 (m, 4H), 2.01-2.07 (m, 1H), 2.68 (br s, 1H), 2.24-2.46 (br s, 1H), 3.69 (s, 2H), 3.98 (s, 3H), 3.99 (s, 3H), 5.03 (s, 2H), 7.03 (d, 1H), 7.25 (m, 1H), 7.39 (s, 1H), 7.48 (m, 1H), 7.54 (s, 1H), 7.55 (br s, 1H), 7.67 (d, 1H), 8.12 (s, 1H), 8.66 (s, 1H), 10.3 (s, 1H); Mass Spectrum: M+H⁺ 475.

The N-[3-(N-tert-butoxycarbonyl-N-cyclopropylaminomethyl)phenyl]-2-[4-(6,7-dimethoxyquinazolin-4-yloxy)pyrazol-1-yl]acetamide used as a starting material was prepared as follows: —

Cyclopropylamine (0.274 g) was added slowly to a mixture of titanium (IV) isopropoxide (1.42 g) and THF (8 ml) and the mixture was stirred at ambient temperature for 10 minutes. 3-Nitrobenzaldehyde (0.604 g) and sodium borohydride (0.151 g) were added in turn and the resultant mixture was stirred at ambient temperature for 1 hour. A 2N aqueous potassium carbonate solution was added and the mixture was extracted with diethyl ether. The organic phase was extracted with 2N aqueous hydrochloric acid. The resultant aqueous phase was washed with diethyl ether, basified by the addition of 2N aqueous potassium carbonate solution and extracted with diethyl ether. The resultant organic phase was dried over magnesium sulphate and evaporated. There was thus obtained N-cyclopropyl-N-(3-nitrobenzyl)amine as an oil (0.22 g); ¹H NMR: (DMSOd₆) 0.24 (m, 2H), 0.35 (m, 2H), 2.03 (m, 1H), 2.92 (br s, 1H), 3.84 (s, 2H), 7.6 (t, 1H), 7.78 (d, 1H), 8.08 (m, 1H), 8.19 (s, 1H); Mass Spectrum: M+H⁺ 193.

A mixture of N-cyclopropyl-N-(3-nitrobenzyl)amine (1 g), di-tert-butyl dicarbonate (1.25 g) and methylene chloride (20 ml) was stirred at ambient temperature for 4 hours. The solvent was evaporated and the residue was purified by column chromatography on silica using methylene chloride as eluent. There was thus obtained N-tert-butoxycarbonyl-N-cyclopropyl-N-(3-nitrobenzyl)amine as an oil (1.35 g); ¹H NMR: DMSOd₆) 0.61 (m, 2H), 0.67 (m, 2H), 1.4 (s, 9H), 2.48 (m, 1H), 4.49 (s, 2H), 7.66 (m, 2H), 8.05 (s, 1H), 8.13 (m, 1H).

A mixture of the material so obtained, platinum oxide (0.2 g) and ethyl acetate (25 ml) was stirred under 1.8 atmospheres pressure of hydrogen for 30 minutes. The catalyst was removed by filtration and the filtrate was evaporated. There was thus obtained 3-(N-tert-butoxycarbonyl-N-cyclopropylaminomethyl)aniline as an oil (1.25 g); ¹H NMR: (DMSOd₆) 0.57 (m, 2H), 0.63 (m, 2H), 1.4 (s, 9H), 2.38 (m, 1H), 4.2 (s, 2H), 5.03 (s, 2H), 6.33 (d, 1H), 6.42 (m, 2H), 6.95 (t, 1H).

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)aniline. The material so obtained was purified by column chromatography on silica using increasingly polar mixtures of methanol and a 1:1 mixture of methylene chloride and ethyl acetate as eluent. There was thus obtained the required starting material in 87% yield; ¹H NMR: (DMSOd₆+CF₃CO₂D) 0.6 (m, 2H), 0.67 (m, 2H), 1.41 (s, 9H), 2.45 (m, 1H), 4.06 (s, 3H), 4.08 (s, 3H), 4.36 (s, 2H), 5.01 (s, 2H), 6.95 (d, 1H), 7.3 (t, 1H), 7.54 (m, 3H), 7.74 (d, 2H), 8.2 (s, 1H), 9.17 (m, 1H); Mass Spectrum: M+H⁺ 575.

### EXAMPLE 20

Using an analogous procedure to that described in Example 19, the appropriate N-tert-butoxycarbonyl-protected amine was reacted with trifluoroacetic acid to give the compounds described in Table V.

TABLE V

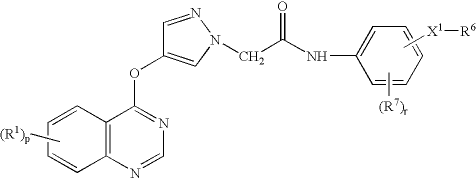

| No. & | | | |
|---|---|---|---|
| Note | (R¹)ₚ | (R⁷)ᵣ | X¹—R⁶ |
| [1] | 6,7-dimethoxy | 5-methyl | 3-ethylaminomethyl |
| [2] | 6,7-dimethoxy | 5-methyl | 3-isopropylaminomethyl |
| [3] | 6,7-dimethoxy | 5-methyl | 3-cyclopropylaminomethyl |
| [4] | 6-methoxy- | 5-methyl | 3-cyclopropylaminomethyl |
| | 7-(2-methoxyethoxy) | | |
| [5] | 6,7-di-(2-methoxyethoxy) | 5-methyl | 3-cyclopropylaminomethyl |
| [6] | 6,7-dimethoxy | 5-methoxy | 3-cyclopropylaminomethyl |
| [7] | 6,7-dimethoxy | 5-ethoxy | 3-cyclopropylaminomethyl |
| [8] | 6,7-dimethoxy | 3-methoxy | 4-cyclopropylaminomethyl |
| [9] | 6,7-dimethoxy | 5-fluoro | 3-cyclopropylaminomethyl |
| [10] | 6,7-dimethoxy | 4-chloro | 3-cyclopropylaminomethyl |
| [11] | 6,7-dimethoxy | 3-chloro | 4-cyclopropylaminomethyl |
| [12] | 6,7-dimethoxy | 5-hydroxymethyl | 3-cyclopropylaminomethyl |
| [13] | 6,7-dimethoxy | 5-methoxymethyl | 3-cyclopropylaminomethyl |
| [14] | 6,7-dimethoxy | H | 3-(2-cyclopropylaminoethyl) |
| [15] | 6,7-dimethoxy | H | 3-(2-cyclopropylaminoethoxy) |
| [16] | 6,7-dimethoxy | 4-methoxy | 3-azetidin-3-yloxy |
| [17] | 6,7-dimethoxy | 4-methoxy | 3-[(2S)-pyrrolidin-2-ylmethoxy] |
| [18] | 6,7-dimethoxy | 4-chloro | 3-[(2S)-pyrrolidin-2-ylmethoxy] |
| [19] | 6,7-dimethoxy | 3-chloro | 4-[(2S)-pyrrolidin-2-ylmethoxy] |
| [20] | 6,7-dimethoxy | H | 3-(2-cyclopropylaminoacetamido) |

Notes The products gave the characterising data shown below.

[1] ¹H NMR: (DMSOd₆) 1.02 (t, 3H), 2.07 (br s, 1H), 2.26 (s, 3H), 2.46-2.54 (m, 2H), 3.6 (s, 2H), 3.98 (s, 3H), 3.99 (s, 3H), 5.01 (s, 2H), 6.86 (s, 1H), 7.32 (s, 1H), 7.35 (s, 1H), 7.39 (s, 1H), 7.54 (s, 1H), 7.67 (s, 1H), 8.11 (s, 1H), 8.66 (s, 1H), 10.21 (s, 1H); Mass Spectrum: M+H⁺ 477.

The N-[3-(N-tert-butoxycarbonyl-N-ethylaminomethyl)-5-methylphenyl]-2-[4-(6,7-dimethoxyquinazolin-4-yloxy)pyrazol-1-yl]acetamide used as a starting material was prepared as follows: —

Using an analogous procedure to that described in the portion of Note [20] below Table II in Example 9 that is concerned with the preparation of starting materials, 3-methyl-5-nitrobenzyl bromide was reacted with ethylamine to give N-ethyl-N-(3-methyl-5-nitrobenzyl)amine in 49% yield; ¹H NMR: (DMSOd₆) 1.03 (t, 3H), 2.29 (br s, 1H), 2.42 (s, 3H), 2.51 (q, 2H), 3.76 (s, 2H), 7.6 (s, 1H), 7.92 (s, 1H), 8.0 (s, 1H).

Using analogous procedures to those described in the portion of Example 19 that is concerned with the preparation of starting materials, N-ethyl-N-(3-methyl-5-nitrobenzyl)amine was converted into N-tert-butoxycarbonyl-N-ethyl-N-(3-methyl-5-nitrobenzyl)amine in 100% yield; ¹H NMR: (DMSOd₆) 1.02 (t, 3H), 1.4 (m, 9H), 2.43 (s, 3H), 3.2 (m, 2H), 4.45 (s, 2H), 7.52 (s, 1H), 7.89 (s, 1H), 7.97 (s, 1H); and which in turn was converted into 3-(N-tert-butoxycarbonyl-N-ethylaminomethyl)-5-methylaniline in 66% yield; ¹H NMR: (DMSOd₆) 0.98 (t, 3H), 1.42 (br s, 9H), 2.12 (s, 3H), 3.09 (m, 2H), 4.16 (s, 2H), 4.95 (s, 2H), 6.18 (s, 1H), 6.22 (s, 1H), 6.25 (s, 1H); Mass Spectrum: M+H⁺ 265.

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-ethylaminomethyl)-5-methylaniline to give the required starting material in 96% yield; ¹H NMR: (DMSOd₆) 1.01 (t, 3H), 1.4 (br s, 9H), 2.27 (s, 3H), 3.13 (m, 2H), 3.98 (s, 3H), 3.99 (s, 3H), 4.31 (s, 2H), 5.02 (s, 2H), 6.77 (s, 1H), 7.27 (br s, 1H), 7.37 (brs, 1H), 7.39 (s, 1H), 7.54 (s, 1H), 7.67 (s, 1H), 8.12 (s, 1H), 8.66 (s, 1H); Mass Spectrum: M+H⁺ 577.

[2] ¹H NMR: (DMSOd₆) 0.99 (d, 6H), 1.8 (br s, 1H), 2.26 (s, 3H), 2.65-2.74 (m, 1H), 3.62 (s, 2H), 3.98 (s, 3H), 3.99 (s, 3H), 5.01 (s, 2H), 6.87 (s, 1H), 7.32 (s, 1H), 7.36 (s, 1H), 7.39 (s, 1H), 7.54 (s, 1H), 7.66 (s, 1H), 8.11 (s, 1H), 8.66 (s, 1H), 10.22 (s, 1H); Mass Spectrum: M+H⁺ 491.

The N-[3-(N-tert-butoxycarbonyl-N-isopropylaminomethyl)-5-methylphenyl]-2-[4-(6,7-dimethoxyquinazolin-4-yloxy)pyrazol-1-yl]acetamide used as a starting material was prepared as follows: —

Using an analogous procedure to that described in the portion of Note [20] below Table II in Example 9 that is concerned with the preparation of starting materials, 3-methyl-5-nitrobenzyl bromide was reacted with isopropylamine to give N-isopropyl-N-(3-methyl-5-nitrobenzyl)amine in 53% yield; ¹H NMR: (DMSOd₆) 1.0 (d, 6H), 2.15 (br s, 1H), 2.42 (s, 3H), 2.69 (m, 1H), 3.76 (s, 2H), 7.61 (s, 1H), 7.91 (s, 1H), 8.02 (s, 1H).

Using analogous procedures to those described in the portion of Example 19 that is concerned with the preparation of starting materials, N-isopropyl-N-(3-methyl-5-nitrobenzyl)amine was converted into N-tert-butoxycarbonyl-N-isopropyl-N-(3-methyl-5-nitrobenzyl)amine in 100% yield; ¹H NMR: (DMSOd₆) 1.04 (d, 6H), 1.39 (m, 9H), 2.42 (s, 3H), 3.3 (m, 1H), 4.39 (s, 2H), 7.51 (s, 1H), 7.88 (s, 1H), 7.94 (s, 1H); and which in turn was converted into 3-(N-tert-butoxycarbonyl-N-isopropylaminomethyl)-5-methylaniline in 57% yield; ¹H NMR: (DMSOd₆) 1.04 (d, 6H), 1.38 (br s, 9H), 2.11 (s, 3H), 3.83 (m, 1H), 4.12 (br s, 2H), 4.9 (s, 2H), 6.18 (s, 1H), 6.21 (s, 1H), 6.23 (s, 1H); Mass Spectrum: M+H⁺ 279.

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-isopropylaminomethyl)-5-methylaniline to give the required starting material in 96% yield; ¹H NMR: (DMSOd₆) 1.05 (d, 6H), 1.28-1.45 (br s, 9H), 2.26 (s, 3H), 3.31 (m, 1H), 3.98 (s, 3H), 3.99 (s, 3H), 4.24 (br s, 2H), 5.02 (s, 2H), 6.77 (s, 1H), 7.27 (s, 1H), 7.33 (s, 1H), 7.39 (s, 1H), 7.54 (s, 1H), 7.67 (s, 1H), 8.11 (s, 1H), 8.66 (s, 1H); Mass Spectrum: M+H⁺ 591.

[3] ¹H NMR: (DMSOd₆) 0.21-0.27 (m, 2H), 0.31-0.37 (m, 2H), 2.0-2.08 (m, 1H), 2.27 (s, 3H), 2.54 (br s, 1H), 3.65 (s, 2H), 3.98 (s, 3H), 3.99 (s, 3H), 5.01 (s, 2H), 6.86 (s, 1H), 7.32 (s, 1H), 7.34 (s, 1H), 7.39 (s, 1H), 7.54 (s, 1H), 7.67 (s, 1H), 8.11 (s, 1H), 8.66 (s, 1H), 10.22 (s, 1H); Mass Spectrum: M+H⁺ 489.

The N-[3-(N-tert-butoxycarbonyl-N-cyclopropylaminomethyl)-5-methylphenyl]-2-[4-(6,7-dimethoxyquinazolin-4-yloxy)pyrazol-1-yl]acetamide used as a starting material was prepared as follows: —

Using an analogous procedure to that described in the portion of Note [20] below Table II in Example 9 that is concerned with the preparation of starting materials, 3-methyl-5-nitrobenzyl bromide was reacted with cyclopropylamine to give N-cyclopropyl-N-(3-methyl-5-nitrobenzyl)amine in 52% yield; ¹H NMR: (DMSOd₆) 0.24 (m, 2H), 0.34 (m, 2H), 2.03 (m, 1H), 2.42 (s, 3H), 2.88 (br s, 1H), 3.8 (s, 2H), 7.6 (s, 1H), 7.92 (s, 1H), 7.99 (s, 1H).

Using analogous procedures to those described in the portion of Example 19 that is concerned with the preparation of starting materials, N-cyclopropyl-N-(3-methyl-5-nitrobenzyl)amine was converted into N-tert-butoxycarbonyl-N-cyclopropyl-N-(3-methyl-5-nitrobenzyl)amine in 100% yield; ¹H NMR: (DMSOd₆) 0.6 (m, 2H), 0.67 (m, 2H), 1.34 (s, 9H), 2.44 (s, 3H), 2.48 (m, 1H), 4.45 (s, 2H), 7.48 (s, 1H), 7.84 (s, 1H), 7.97 (s, 1H); and which in turn was converted into 3-(N-tert-butoxycarbonyl-N-cyclopropylaminomethyl)-5-methylaniline; ¹H NMR: (DMSOd₆) 0.56 (m, 2H), 0.63 (m, 2H), 1.4 (s, 9H), 2.12 (s, 3H), 2.37 (m, 1H), 4.16 (s, 2H), 4.95 (s, 2H), 6.16 (s, 1H), 6.21 (s, 1H), 6.24 (s, 1H); Mass Spectrum: M+H⁺ 277.

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)-5-methylaniline to give the required starting material in 79% yield; ¹H NMR: (DMSOd₆) 0.6 (m, 2H), 0.65 (m, 2H), 1.39 (s, 9H), 2.27 (s, 3H), 2.42 (m, 1H), 3.98 (s, 3H), 3.99 (s, 3H), 4.29 (s, 2H), 5.02 (s, 2H), 6.75 (s, 1H), 7.27 (s, 1H), 7.35 (s, 1H), 7.39 (s, 1H), 7.54 (s, 1H), 7.67 (s, 1H), 8.12 (s, 1H), 8.66 (s, 1H); Mass Spectrum: M+H⁺ 589.

[4] ¹H NMR: (DMSOd₆) 0.21-0.26 (m, 2H), 0.31-0.37 (m, 2H), 2.0-2.06 (m, 1H), 2.23 (s, 3H), 2.55 (br s, 1H), 3.35 (s, 3H), 3.65 (s, 2H), 3.74-3.79 (m, 2H), 3.99 (s, 3H), 4.29-4.36 (m, 2H), 5.01 (s, 2H), 6.86 (s, 1H), 7.32 (s, 1H), 7.34 (s, 1H), 7.41 (s, 1H), 7.55 (s, 1H), 7.67 (s, 1H), 8.11 (s, 1H), 8.66 (s, 1H), 10.22 (s, 1H); Mass Spectrum: M+H⁺ 533.

The N-[3-(N-tert-butoxycarbonyl-N-cyclopropylaminomethyl)-5-methylphenyl]-2-{4-[6-methoxy-7-(2-methoxyethoxy)quinazolin-4-yloxy]pyrazol-1-yl}acetamide used as a starting material was prepared as follows: —

Using an analogous procedure to that described in Example 8, 2-{4-[6-methoxy-7-(2-methoxyethoxy)quinazolin-4-yloxy]pyrazol-1-yl}acetic acid was reacted with 3-(N-tert-butoxycarbonyl-N-cyclopropylaminomethyl)-5-methylaniline to give the required starting material in 80% yield; ¹H NMR: (DMSOd₆) 0.6 (m, 2H), 0.65 (m, 2H), 1.39 (s, 9H), 2.27 (s, 3H), 2.42 (m, 1H), 3.46 (s, 3H), 3.76 (m, 2H), 4.02 (s, 3H), 4.29 (s, 2H), 4.33 (m, 2H), 5.02 (s, 2H), 6.75 (s, 1H), 7.27 (s, 1H), 7.35 (s, 1H), 7.41 (s, 1H), 7.55 (s, 1H), 7.67 (s, 1H), 8.11 (s, 1H), 8.65 (s, 1H); Mass Spectrum: M+H⁺ 633.

[5] ¹H NMR: (DMSOd₆) 0.21-0.27 (m, 2H), 0.31-0.37 (m, 2H), 2.0-2.07 (m, 1H), 2.26 (s, 3H), 2.55 (br s, 1H), 3.36 (s, 3H), 3.37 (s, 3H), 3.65 (s, 2H), 3.74-3.79 (m, 4H), 4.3-4.37 (m, 4H), 5.01 (s, 2H), 6.86 (s, 1H), 7.32 (s, 1H), 7.33 (s, 1H), 7.42 (s, 1H), 7.58 (s, 1H), 7.67 (s, 1H), 8.11 (s, 1H), 8.65 (s, 1H), 10.22 (s, 1H); Mass Spectrum: M+H⁺ 577:

The N-[3-(N-tert-butoxycarbonyl-N-cyclopropylaminomethyl)-5-methylphenyl]-2-{4-[6,7-di-(2-methoxyethoxy)quinazolin-4-yloxy]pyrazol-1-yl}acetamide used as a starting material was prepared as follows: —

Using an analogous procedure to that described in Example 8, 2-{4-[6,7-di-(2-methoxyethoxy)quinazolin-4-yloxy]pyrazol-1-yl}acetic acid was reacted with 3-(N-tert-butoxycarbonyl-N-cyclopropylaminomethyl)-5-methylaniline to give the required starting material in 80% yield; ¹H NMR: (DMSOd₆) 0.6 (m, 2H), 0.65 (m, 2H), 1.39 (s, 9H), 2.27 (s, 3H), 2.42 (m, 1H), 3.36 (s, 6H), 3.77 (m, 4H), 4.29 (s, 2H), 4.33 (m, 4H), 5.02 (s, 2H), 6.75 (s, 1H), 7.27 (s, 1H), 7.35 (s, 1H), 7.42 (s, 1H), 7.58 (s, 1H), 7.63 (s, 1H), 8.11 (s, 1H), 8.65 (s, 1H); Mass Spectrum: M+H⁺ 677.

[6] The reaction mixture was evaporated and the residue was dissolved in a 4:1 mixture of methylene chloride and methanol. A basic polystrene resin (methylpolystyrene carbonate resin) was added and the mixture was stirred at ambient temperature for 5 hours. The mixture was filtered and the filtrate was evaporated. The residue was dissolved in methylene chloride and diethyl ether was added. The resultant precipitate was isolated. The product gave the following characterising data: —¹H NMR: (DMSOd₆) 0.22-0.27 (m, 2H), 0.31-0.37 (m, 2H), 2.0-2.08 (m, 1H), 2.61 (br s, 1H), 3.66 (s, 2H), 3.72 (s, 3H), 3.98 (s, 3H), 3.99 (s, 3H), 5.02 (s, 2H), 6.65 (s, 1H), 7.07 (s, 1H), 7.19 (m, 1H), 7.39 (s, 1H), 7.54 (s, 1H), 7.67 (s, 1H), 8.12 (s, 1H), 8.66 (s, 1H), 10.29 (s, 1H); Mass Spectrum: M+H⁺ 505.

The N-[3-(N-tert-butoxycarbonyl-N-cyclopropylaminomethyl)-5-methoxyphenyl]-2-[4-(6,7-dimethoxyquinazolin-4-yloxy)pyrazol-1-yl]acetamide used as a starting material was prepared as follows: —

Using an analogous procedure to that described in the second paragraph of the portion of Note [24] below Table II in Example 9, 3-methoxy-5-nitrobenzoic acid (J. Med. Chem., 2004, 2897-2905) was reduced with diborane. The reaction product was purified by column chromatography on silica using a 19:1 mixture of methylene chloride and methanol as eluent. There was thus obtained 3-methoxy-5-nitrobenzyl alcohol in 84% yield; ¹H NMR: (DMSOd₆) 3.87 (s, 3H), 4.58 (d, 2H), 5.53 (t, 1H), 7.34 (s, 1H), 7.58 (s, 1H), 7.79 (s, 1H).

A solution of phosphorus tribromide (0.94 ml) in diethyl ether (5 ml) was slowly added to a solution of 3-methoxy-5-nitrobenzyl alcohol (1.83 g) in methylene chloride (30 ml) that had been cooled to 5° C. and the resultant mixture was stirred at ambient temperature for 5 hours. The mixture was poured into cooled water, neutralised by the addition of solid sodium bicarbonate and extracted with diethyl ether. The organic solution was dried over magnesium sulphate and evaporated. There was thus obtained 3-methoxy-5-nitrobenzyl bromide as a solid (1.48 g); ¹H NMR: (DMSOd₆) 3.89 (s, 3H), 4.8 (s, 2H), 7.51 (s, 1H), 7.67 (m, 1H), 7.94 (s, 1H).

Cyclopropylamine (1.67 ml) was slowly added to a solution of 3-methoxy-5-nitrobenzyl bromide (1.48 g) in methylene chloride (4 ml) and the mixture was stirred at ambient temperature for 6 hours. The mixture was washed with a 2N aqueous potassium carbonate solution. The organic phase was dried over magnesium sulphate and evaporated. There was thus obtained N-cyclopropyl-N-(3-methoxy-5-nitrobenzyl)amine (1.35 g; containing about 10% of dialkylated amine); ¹H NMR: (DMSOd₆) 0.26 (m, 2H), 0.37 (m, 2H), 2.04 (s, 1H), 2.96 (br s, 1H), 3.82 (s, 2H), 3.88 (s, 3H), 7.39 (s, 1H), 7.59 (s, 1H), 7.82 (s, 1H); Mass Spectrum: M+H⁺ 223.

Using analogous procedures to those described in relevant portions of Example 19 that are concerned with the preparation of starting materials, N-cyclopropyl-N-(3-methoxy-5-nitrobenzyl)amine was converted into N-tert-butoxycarbonyl-N-cyclopropyl-N-(3-methoxy-5-nitrobenzyl)amine in 82% yield; ¹H NMR: (DMSOd₆) 0.59 (m, 2H), 0.67 (m, 2H), 1.41 (s, 9H), 2.48 (m, 1H), 3.87 (s, 3H), 4.45 (s, 2H), 7.21 (s, 1H), 7.63 (m, 2H); which in turn was converted into 3-(N-tert-butoxycarbonyl-N-cyclopropylaminomethyl)-5-methoxyaniline in 94% yield; ¹H NMR: (DMSOd₆) 0.57 (m, 2H), 0.63 (m, 2H), 1.41 (s, 9H), 2.39 (m, 1H), 3.62 (s, 3H), 4.16 (s, 2H), 5.07 (s, 2H), 5.91 (m, 1H), 6.01 (m, 2H); Mass Spectrum: M+H⁺ 293.

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)-5-methoxyaniline to give the required starting material in 94% yield; ¹H NMR: (DMSOd₆) 0.6 (m, 2H), 0.65 (m, 2H), 1.4 (s, 9H), 2.49 (m, 1H), 3.72 (s, 3H), 3.98 (s, 3H), 3.99 (s, 3H), 4.3 (s, 2H), 5.03 (s, 2H), 6.49 (s, 1H), 7.0 (s, 1H), 7.24 (s, 1H), 7.39 (s, 1H), 7.54 (s, 1H), 7.67 (s, 1H), 8.12 (s, 1H), 8.66 (s, 1H); Mass Spectrum: M+H⁺ 605.

[7] ¹H NMR: (DMSOd₆) 0.25 (m, 2H), 0.34 (m, 2H), 1.32 (t, 3H), 2.04 (m, 1H), 3.66 (s, 2H), 3.98 (m, 8H), 5.03 (s, 2H), 6.43 (s, 1H), 7.06 (s, 1H), 7.19 (s, 1H), 7.4 (s, 1H), 7.55 (s, 1H), 7.68 (s, 1H), 8.13 (s, 1H), 8.67 (s, 1H), 10.28 (s, 1H); Mass Spectrum: M+H⁺ 519.

The N-[3-(N-tert-butoxycarbonyl-N-cyclopropylaminomethyl)-5-ethoxyphenyl]-2-[4-(6,7-dimethoxyquinazolin-4-yloxy)pyrazol-1-yl]acetamide used as a starting material was prepared as follows: —

Diethyl sulphate (7.14 ml) was added slowly to a mixture of 3-hydroxy-5-nitrobenzoic acid (1.15 g) and 1N aqueous sodium hydroxide solution (22 ml) and the resultant mixture was stirred at ambient temperature for 48 hours. The mixture was concentrated by evaporation and extracted with methylene chloride. The organic solution was dried over magnesium sulphate and evaporated. There was thus obtained 3-ethoxy-5-nitrobenzoic acid (0.62 g); ¹H NMR: (DMSOd₆) 1.37 (t, 3H), 4.22 (q, 2H), 7.79 (s, 1H), 7.91 (s, 1H), 8.2 (s, 1H).

1-(3-Dimethylaminopropyl)-3-ethylcarbodiimide hydrochloride (0.544 g) was added to a mixture of 3-ethoxy-5-nitrobenzoic acid (0.2 g), cyclopropylamine (0.131 ml), 2-hydroxypyridine N-oxide (0.316 g), diisopropylethylamine (0.495 ml) and DMF (3 ml) and the resultant mixture was stirred at ambient temperature for 12 hours. Water was added and the precipitate was isolated and dried under vacuum. There was thus obtained N-cyclopropyl-3-ethoxy-5-nitrobenzamide (0.135 g); ¹H NMR: (DMSOd₆) 0.6 (m, 2H), 0.73 (m, 2H), 1.37 (t, 3H), 2.87 (m, 1H), 4.2 (q, 2H), 7.81 (m, 1H), 8.24 (s, 1H), 8.75 (m, 1H).

Borane dimethyl sulphide complex-(2M in toluene; 3.43 ml) was added to a solution of N-cyclopropyl-3-ethoxy-5-nitrobenzamide (0.286 g) in THF (10 ml) and the resultant mixture was stirred at 65° C. for 1 hour. The mixture was cooled to ambient temperature. An excess of a mixture of hydrochloric acid, 1,4-dioxane and methanol was added. The resultant mixture was evaporated and the residue was partitioned between ethyl acetate and a 2N aqueous sodium bicarbonate solution. The organic solution was evaporated and the residue was purified by column chromatography on silica using a gradient of 100:0 to 50:50 of methylene chloride and ethyl acetate as eluent. There was thus obtained N-cyclopropyl-N-(3-ethoxy-5-nitrobenzyl)amine (0.182 g); ¹H NMR: (DMSOd₆) 0.25 (m, 2H), 0.34 (m, 2H), 1.36 (t, 3H), 2.02 (m, 1H), 2.92 (br s, 1H), 3.79 (s, 2H), 4.14 (q, 2H), 7.36 (s, 1H), 7.54 (s, 1H), 7.78 (s, 1H); Mass Spectrum: M+H⁺: 237.

Using analogous procedures to those described in relevant portions of Example 19 that are concerned with the preparation of starting materials, N-cyclopropyl-N-(3-ethoxy-5-nitrobenzyl)amine was converted into N-tert-butoxycarbonyl-N-cyclopropyl-N-(3-ethoxy-5-nitrobenzyl)amine in 81% yield; ¹H NMR: (DMSOd₆) 0.59 (m, 2H), 0.66 (m, 2H), 1.34 (t, 3H), 1.41 (s, 9H), 2.48 (m, 1H), 4.14 (q, 2H), 4.44 (s, 2H), 7.19 (s, 1H), 7.59 (s, 1H), 7.63 (s, 1H); which in turn was converted into 3-(N-tert-butoxycarbonyl-N-cyclopropylaminomethyl)-5-ethoxyaniline in 100% yield; ¹H NMR: (DMSOd₆) 0.56 (m, 2H), 0.63 (m, 2H), 1.27 (t, 3H), 1.4 (s, 9H), 2.38 (m, 1H), 3.87 (q, 2H), 4.15 (s, 2H), 5.04 (s, 2H), 5.9 (s, 1H), 6.0 (m, 2H); Mass Spectrum: M+H⁺ 307.

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)-5-ethoxyaniline to give the required starting material in 52% yield; Mass Spectrum: M+H⁺ 619.

[8] ¹H NMR: (DMSOd₆) 0.2-0.25 (m, 2H), 0.31-0.37 (m, 2H), 1.99-2.05 (m, 1H), 2.38 (br s, 1H), 3.65 (s, 2H), 3.76 (s, 3H), 3.98 (s, 3H), 3.99 (s, 3H), 5.03 (s, 2H), 7.07 (m, 1H), 7.2 (d, 1H), 7.35 (d, 1H), 7.39 (s, 1H), 7.54 (s, 1H), 7.68 (s, 1H), 8.12 (s, 1H), 8.66 (s, 1H), 10.32 (s, 1H); Mass Spectrum: M+H⁺ 505.

The N-[4-(N-tert-butoxycarbonyl-N-cyclopropylaminomethyl)-3-methoxyphenyl]-2-[4-(6,7-dimethoxyquinazolin-4-yloxy)pyrazol-1-yl]acetamide used as a starting material was prepared as follows: —

Diborane (1M in THF; 40 ml) was added dropwise to a mixture of 2-methoxy-4-nitrobenzoic acid (1.97 g) and THF (20 ml) and the resultant mixture was stirred at ambient temperature for 16 hours. Water was added slowly to the reaction mixture to destroy the excess of reducing agent. The reaction mixture was extracted with diethyl ether and the organic phase was dried over magnesium sulphate and evaporated. The residue was purified by column chromatography on silica using a gradient of 100:0 to 95:5 of methylene chloride and methanol as eluent. There was thus obtained 2-methoxy-4-nitrobenzyl alcohol (1.24 g); ¹H NMR: (DMSOd₆) 3.91 (s, 3H), 4.56 (d, 2H), 5.41 (t, 1H), 7.64 (d, 1H), 7.71 (m, 1H), 7.88 (m, 1H).

Using an analogous procedure to that described in the second paragraph of the portion of Note [9] immediately below that is concerned with the preparation of starting materials, 2-methoxy-4-nitrobenzyl alcohol was reacted with carbon tetrabromide and triphenylphosphine to give 2-methoxy-4-nitrobenzyl bromide in 90% yield; ¹H NMR: (DMSOd₆) 4.0 (s, 3H), 4.7 (s, 2H), 7.7 (d, 1H), 7.83 (m, 2H).

Using an analogous procedure to that described in the third paragraph of the portion of Note [6] above that is concerned with the preparation of starting materials, 2-methoxy-4-nitrobenzyl bromide was reacted with cyclopropylamine to give N-cyclopropyl-N-(2-methoxy-4-nitrobenzyl)amine in 92% yield; ¹H NMR: (DMSOd₆) 0.25 (m, 2H), 0.36 (m, 2H), 2.06 (m, 1H), 2.7 (br s, 1H), 3.79 (s, 2H), 3.92 (s, 3H), 7.58 (d, 1H), 7.19 (m, 1H), 7.82 (m, 1H); Mass Spectrum: M+H⁺: 223.

Using analogous procedures to those described in relevant portions of Example 19 that are concerned with the preparation of starting materials, N-cyclopropyl-N-(2-methoxy-4-nitrobenzyl)amine was converted into N-tert-butoxycarbonyl-N-cyclopropyl-N-(2-methoxy-4-nitrobenzyl)amine in 90% yield; ¹H NMR: (DMSOd₆) 0.59 (m, 2H), 0.64 (m, 2H), 1.38 (br s, 1H), 2.55 (m, 1H), 3.94 (s, 3M), 4.39 (s, 2H), 7.24 (d, 1H), 7.76 (m, 1H), 7.86 (m, 1H); which in turn was converted into 4-(N-tert-butoxycarbonyl-N-cyclopropylaminomethyl)-3-methoxyaniline in 100% yield; ¹H NMR: (DMSOd₆) 0.52 (m, 2H), 0.58 (m, 2H), 1.38 (s, 9H), 2.3 (m, 1H), 3.68 (s, 3H), 4.17 (s, 2H), 4.99 (s, 2H), 6.08 (m, 1H), 6.2 (s, 1H), 6.69 (m, 1H); Mass Spectrum: M+H⁺ 293.

Using an analogous procedure to that described in Example 8, 2-[4-(6,7-dimethoxyquinazolin-4-yloxy)pyrazol-1-yl]acetic acid was reacted with 4-(N-tert-butoxycarbonyl-N-cyclopropylaminomethyl)-3-methoxyaniline to give the required starting material in 86% yield; ¹H NMR: (DMSOd₆) 0.56 (m, 2H), 0.6 (m, 2H), 1.38 (br s, 9H), 2.42 (m, 1H), 3.76 (s, 3H), 3.98 (s, 3H), 3.99 (s, 3H), 4.28 (s, 2H), 5.03 (s, 2H), 6.96 (d, 1H), 7.09 (m, 1H), 7.39 (s, 1H), 7.54 (s, 1H), 7.68 (s, 1H), 8.12 (s, 1H), 8.66 (s, 1H); Mass Spectrum: M+H⁺ 605.

[9] ¹H NMR: (DMSOd₆) 0.21-0.27 (m, 2H), 0.31-0.38 (m, 2H), 2.0-2.07 (m, 1H), 2.73 (br s, 1H), 3.7 (s, 2H), 3.98 (s, 3H), 3.99 (s, 3H), 5.04 (s, 2H), 6.88 (d, 1H), 7.27 (s, 1H), 7.4 (s, 1H), 7.45 (d, 1H), 7.55 (s, 1H), 7.68 (s, 1H), 8.12 (s, 1H), 8.66 (s, 1H), 10.52 (s, 1H); Mass Spectrum: M+H⁺ 493.

The N-[3-(N-tert-butoxycarbonyl-N-cyclopropylaminomethyl)-5-fluorophenyl]-2-[4-(6,7-dimethoxyquinazolin-4-yloxy)pyrazol-1-yl]acetamide used as a starting material was prepared as follows: —

A solution of lithium borohydride (2M in THF; 7.3 ml) was added slowly to a mixture of methyl-3-fluoro-5-nitrobenzoate (JCS Chem. Comm., 1993, 921-922; 2.9 g) and diethyl ether (60 ml) and the resultant mixture was stirred at ambient temperature for 2 hours. Water and a 2N aqueous hydrochloric acid solution were added in turn and the mixture was extracted with diethyl ether. The organic solution was evaporated and the residue was purified by column chromatography on silica using methylene chloride as eluent. There was thus obtained an oil which crystallised on standing to give 3-fluoro-5-nitrobenzyl alcohol (2.05 g); ¹H NMR (DMSOd₆) 4.64 (s, 2H), 5.65 (s, 1H), 7.64 (m, 1H), 7.96 (m, 1H), 8.06 (s, 1H).

Carbon tetrabromide (5 g) was added dropwise to a mixture of 3-fluoro-5-nitrobenzyl alcohol (1.72 g), triphenylphosphine (3.43 g) and methylene chloride (25 ml) and the mixture was stirred at ambient temperature for 3 hours. The mixture was evaporated and the residue was purified by column chromatography on silica using a 1:1 mixture of petroleum ether (b.p. 40-60° C.) and methylene chloride as eluent. There was thus obtained 3-fluoro-5-nitrobenzyl bromide as an oil (2.2 g); ¹H NMR: (DMSOd₆) 4.84 (s, 2H), 7.87 (m, 1H), 8.07 (m, 1H), 8.24 (s, 1H).

Using an analogous procedure to that described in the third paragraph of the portion of Note [6] above that is concerned with the preparation of starting materials, 3-fluoro-5-nitrobenzyl bromide was reacted with cyclopropylamine to give N-cyclopropyl-N-(3-fluoro-5-nitrobenzyl)amine in 89% yield; ¹H NMR: (DMSOd₆) 0.25 (m, 2H), 0.35 (m, 2H), 2.03 (m, 1H), 3.02 (br s, 1H), 3.85 (s, 2H), 7.69 (m, 1H), 7.95 (m, 1H), 8.08 (s, 1H); Mass Spectrum: M+H⁺: 211.

Using analogous procedures to those described in relevant portions of Example 19 that are concerned with the preparation of starting materials, N-cyclopropyl-N-(3-fluoro-5-nitrobenzyl)amine was converted into N-tert-butoxycarbonyl-N-cyclopropyl-N-(3-fluoro-5-nitrobenzyl)amine in 95% yield; ¹H NMR: (DMSOd₆) 0.61 (m, 2H), 0.68 (m, 2H), 1.4 (s, 9H), 2.54 (m, 1H), 4.5 (s, 2H), 7.54 (m, 1H), 7.91 (s, 1H), 8.02 (m, 1H); which in turn was converted into 3-(N-tert-butoxycarbonyl-N-cyclopropylaminomethyl)-5-fluoroaniline in 99% yield; ¹H NMR: (DMSOd₆) 0.57 (m, 2H), 0.64 (m, 2H), 1.4 (s, 9H), 2.42 (m, 1H), 4.19 (s, 2H), 5.41 (s, 2H), 6.05 (m, 1H), 6.19 (m, 1H), 6.22 (s, 1H).

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)-5-fluoroaniline to give the required starting material in 49% yield; ¹H NMR: (DMSOd₆) 0.61 (m, 2H), 0.67 (m, 2M), 1.39 (s, 9H), 2.49 (m, 1H), 3.98 (s, 3H), 3.99 (s, 3H), 4.34 (s, 2H), 5.05 (s, 2H), 6.74 (m, 1H), 7.19 (s, 1H), 7.39 (s, 1H), 7.49 (d, 1H), 7.54 (s, 1H), 7.68 (s, 1H), 8.12 (s, 1H), 8.66 (s, 1H); Mass Spectrum: M+H⁺ 593.

[10] H NMR: DMSOd₆) 0.25-0.32 (m, 2H), 0.33-0.42 (m, 2M), 2.07-2.15 (m, 1H), 2.65 (br s, 1H), 3.79 (s, 2H), 3.98 (s, 3H), 3.99 (s, 3H), 5.04 (s, 2H), 7.36 (d, 1H), 7.39 (s, 1H), 7.54 (s, 1H), 7.57 (m, 1H), 7.68 (s, 1H), 7.71 (d, 1H), 8.12 (s, 1H), 8.66 (s, 1H), 10.46 (s, 1H); Mass Spectrum: M+H⁺ 509 and 511.

The N-[3-(N-tert-butoxycarbonyl-N-cyclopropylaminomethyl)-4-chlorophenyl]-2-[4-(6,7-dimethoxyquinazolin-4-yloxy)pyrazol-1-yl]acetamide used as a starting material was prepared as follows: —

Cyclopropylamine (0.9 ml) and acetic acid (0.744 ml) were added in turn to a stirred solution of 2-chloro-5-nitrobenzaldehyde (1.86 g) in methylene chloride (20 ml) and methanol (2 ml). Sodium borohydride (0.744 g) was added portionwise during 1 hour. The resultant mixture was stirred at ambient temperature for 2 hours. The mixture was concentrated by partial evaporation and partitioned between diethyl ether and a saturated aqueous potassium bicarbonate solution. The organic solution was dried over magnesium sulphate and evaporated. There was thus obtained N-cyclopropyl-N-(2-chloro-5-nitrobenzyl)amine as an oil (2.05 g); ¹H NMR: (DMSOd₆) 0.31 (ma, 2H), 0.41 (m, 2H), 2.13 (m, 1H), 3.01 (br s, 1H), 3.91 (s, 2H), 7.74 (d, 1H), 8.13 (m, 1H), 8.37 (m, 1H).

Using analogous procedures to those described in relevant portions of Example 19 that are concerned with the preparation of starting materials, N-cyclopropyl-N-(2-chloro-5-nitrobenzyl)amine was converted into N-tert-butoxycarbonyl-N-cyclopropyl-N-(2-chloro-5-nitrobenzyl)amine in 97% yield; ¹H NMR: (DMSOd₆) 0.63 (m, 2H), 0.68 (m, 2H), 1.4 (s, 9H), 2.56 (m, 1H), 4.53 (s, 2H), 7.79 (d, 1H), 7.96 (m, 1H), 8.16 (m, 1H); which in turn was converted into 3-(N-tert-butoxycarbonyl-N-cyclopropylaminomethyl)-4-chloroaniline in 59% yield; ¹H NMR: (DMSOd₆) 0.6 (m, 2H), 0.66 (m, 2H), 1.39 (s, 9H), 2.52 (m, 1H), 4.29 (s, 2H), 5.24 (s, 2H), 6.39 (m, 1H), 6.44 (m, 1H), 6.99 (m, 1H).

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)-4-chloroaniline to give the required starting material in 81% yield; ¹H NMR: (DMSOd₆) 0.64 (m, 2H), 0.67 (m, 2H), 1.37 (br s, 9H), 2.58 (m, 1H), 3.98 (s, 3H), 3.99 (s, 3H), 4.41 (s, 2H), 5.04 (s, 2H), 7.39 (m, 2H), 7.47 (m, 1H), 7.55 (s, 1H), 7.58 (m, 1H), 7.68 (s, 1H), 8.12 (s, 1H), 8.65 (s, 1H); Mass Spectrum: M+H⁺ 609 and 611.

[11] ¹H NMR: (DMSOd₆) 0.22-0.28 (m, 2H), 0.33-0.4 (m, 2H), 2.01-2.1 (m, 1H), 2.6 (br s, 1H), 3.76 (s, 2H), 3.98 (s, 3H), 3.99 (s, 3H), 5.05 (s, 2H), 7.39 (s, 1H), 7.44 (br s, 2H), 7.54 (s, 1H), 7.68 (s, 1H), 7.79 (s, 1H), 8.13 (s, 1H), 8.66 (s, 1H), 10.44 (s, 1H); Mass Spectrum: M+H⁺ 509 and 511.

The N-[4-(N-tert-butoxycarbonyl-N-cyclopropylaminomethyl)-3-chlorophenyl]-2-[4-(6,7-dimethoxyquinazolin-4-yloxy)pyrazol-1-yl]acetamide used as a starting material was prepared as follows: —

A mixture of 2-chloro-4-nitrotoluene (0.5 g), N-bromosuccinimide (0.55 g), benzoyl peroxide (0.01 g) and carbon tetrachloride (15 ml) was stirred and heated to reflux for 3 hours. The reaction mixture was evaporated and the residue was purified by column chromatography on silica using a gradient of 95:5 to 93:7 of petroleum ether (b.p. 60-80° C.) and ethyl acetate as eluent. There was thus obtained 2-chloro-4-nitrobenzyl bromide (0.367 g); ¹H NMR: (CDCl₃) 4.6 (s, 2H), 7.65 (m, 1H), 8.12 (m, 1H), 8.28 (m, 1H).

Using an analogous procedure to that described in the second paragraph of the portion of Note [20] below Table II in Example 9 that is concerned with the preparation of starting materials, 2-chloro-4-nitrobenzyl bromide was reacted with cyclopropylamine to give N-cyclopropyl-N-(2-chloro-4-nitrobenzyl)amine in 84% yield; ¹H NMR: (DMSOd₆) 0.27 (m, 2H), 0.39 (m, 2H), 2.11 (m, 1H), 2.94 (br s, 1H), 3.9 (s, 3H), 7.79 (m, 1H), 8.19 (m, 1H), 8.25 (s, 1H).

Using analogous procedures to those described in relevant portions of Example 19 that are concerned with the preparation of starting materials, N-cyclopropyl-N-(2-chloro-4-nitrobenzyl)amine was converted into N-tert-butoxycarbonyl-N-cyclopropyl-N-(2-chloro-4-nitrobenzyl)amine in 98% yield; ¹H NMR: (DMSOd₆) 0.63 (m, 2H), 0.68 (m, 2H), 1.38 (s, 9H), 2.61 (m, 1H), 4.52 (s, 2H), 7.43 (d, 1H), 8.22 (m, 1H), 8.29 (s, 1H); which in turn was converted into 4-(N-tert-butoxycarbonyl-N-cyclopropylaminomethyl)-3-chloroaniline in 83% yield; ¹H NMR: (DMSOd₆) 0.55 (m, 2H), 0.61 (m, 2H), 1.39 (s, 9H), 2.34 (m, 1H), 4.28 (s, 2H), 5.29 (s, 2H), 6.48 (m, 1H), 6.59 (s, 1H), 6.86 (m, 1H); Mass Spectrum: M+H⁺ 297 and 299.

Using an analogous procedure to that described in Example 8, 2-[4-(6,7-dimethoxyquinazolin-4-yloxy)pyrazol-1-yl]acetic acid was reacted with 4-(N-tert-butoxycarbonyl-N-cyclopropylaminomethyl)-3-chloroaniline to give the required starting material in 81% yield; ¹H NMR: (DMSOd₆) 0.6 (m, 2H), 0.64 (m, 2H), 1.39 (br s, 9H), 2.47 (m, 1H), 3.98 (s, 3H), 3.99 (s, 3H), 4.4 (s, 2H), 5.05 (s, 2H), 7.16 (d, 1H), 7.39 (s, 1H), 7.45 (m, 1H), 7.54 (s, 1H), 7.68 (s, 1H), 7.83 (m, 1H), 8.13 (s, 1H), 8.66 (s, 1H); Mass Spectrum: M+H⁺ 609 and 611.

[12] The reaction mixture was evaporated and the residue was dissolved in a 5:1 mixture of methylene chloride and ethanol. A basic polystrene resin (methylpolystyrene carbonate resin) was added and the mixture was stirred at ambient temperature for 12 hours. The mixture was filtered and the filtrate was evaporated. The residue was purified by column chromatography on silica using a 19:1 mixture of methylene chloride and methanol as eluent. The material so obtained was dissolved in methylene chloride and maleic acid (1 equivalent) was added. The resultant precipitate was isolated. The maleate salt so obtained gave the following characterising data: —¹H NMR: (DMSOd₆) 0.76 (m, 2H), 0.78 (m, 2H), 2.69 (m, 1H), 4.0 (s, 3H), 4.01 (s, 3H), 4.2 (s, 2H), 4.51 (d, 2H), 5.06 (s, 2M), 5.36 (t, 1H), 6.04 (s, 2H), 7.17 (s, 1H), 7.4 (s, 1H), 7.48 (s, 1H), 7.53 (s, 1H), 7.68 (s, 1H), 7.76 (s, 1H), 8.13 (s, 1H), 8.66 (s, 1H), 10.47 (s, 1H); Mass Spectrum: M+H⁺ 505.

The N-[3-(N-tert-butoxycarbonyl-N-cyclopropylaminomethyl)-5-hydroxymethylphenyl]-2-[4-(6,7-dimethoxyquinazolin-4-yloxy)pyrazol-1-yl]acetamide used as a starting material was prepared as follows: —

Using an analogous procedure to that described in the second paragraph of the portion of Note [7] above that is concerned with the preparation of starting materials, mono-methyl 5-nitroisophthalate was reacted with cyclopropylamine to give N-cyclopropyl-3-methoxycarbonyl-5-nitrobenzamide in 47% yield; ¹H NMR: (DMSOd₆) 0.63 (m, 2H), 0.74 (m, 2H), 2.92 (m, 1H), 3.96 (s, 3M), 8.72 (s, 1H), 8.79 (s, 1H), 8.89 (s, 1H), 9.03 (m, 1H); Mass Spectrum: M−H⁻ 263.

A solution of sodium hydroxide (1.52 g) in water (2 ml) was added to a solution of N-cyclopropyl-3-methoxycarbonyl-5-nitrobenzamide (2 g) in TIE (40 ml) and water (10 ml) and the mixture was stirred at ambient temperature for 1 hour. The mixture was: acidified to pH2 by the addition of 2N aqueous hydrochloric acid solution and extracted with methylene chloride. The organic solution was dried over magnesium sulphate and evaporated. There was thus obtained N-cyclopropyl-3-carboxy-5-nitrobenzamide (1.74 g); ¹H NMR: (DMSOd₆) 0.63 (m, 2H), 0.73 (m, 2H), 2.92 (m, 1H), 8.71 (s, 1H), 8.79 (s, 1H), 8.86 (s, 1H), 9.01 (m, 1H); Mass Spectrum: M+H⁺ 251.

Using an analogous procedure to that described in the third paragraph of the portion of Note [7] above that is concerned with the preparation of starting materials, N-cyclopropyl-3-carboxy-5-nitrobenzamide was reduced with borane dimethyl sulphide complex to give N-cyclopropyl-N-(3-hydroxymethyl-5-nitrobenzyl)amine in 52% yield; ¹H NMR: (DMSOd₆) 0.26 (m; 2H), 0.35 (m, 2H), 2.04 (m, 1H), 2.89 (br s, 1H), 3.83 (s, 2H), 4.61 (d, 2H), 5.11 (t, 1H), 7.71 (s, 1H), 8.04 (s, 1H), 8.06 (s, 1H); Mass Spectrum: M+H⁺ 223.

Using analogous procedures to those described in relevant portions of Example 19 that are concerned with the preparation of starting materials, N-cyclopropyl-N-(3-hydroxymethyl-5-nitrobenzyl)amine was converted into N-tert-butoxycarbonyl-N-cyclopropyl-N-(3-hydroxymethyl-5-nitrobenzyl)amine in 89% yield; ¹H NMR: (DMSOd₆) 0.61 (m, 2H), 0.68 (m, 2H), 1.41 (s, 9H), 2.48 (m, 1H), 4.48 (s, 2H), 4.62 (d, 2H); 5.54 (t, 1H), 7.62 (s, 1H), 7.91 (s, 1H), 8.07 (s, 1H); which in turn was converted into 3-(N-tert-butoxycarbonyl-N-cyclopropylaminomethyl)-5-hydroxymethylaniline in 100% yield; ¹H NMR: (DMSOd₆) 0.57 (m, 2H), 0.63 (m, 2H), 1.41 (s, 9H), 2.38 (m, 1H), 4.19 (s, 2H), 4.32 (s, 2H), 5.0 (br s, 3H), 6.27 (s, 1H), 6.32 (s, 1H), 6.41 (s, 1H).

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)-5-hydroxymethylaniline to give the required starting material in 94% yield.

[13] The reaction mixture was evaporated and the residue was dissolved in a 5:1 mixture of methylene chloride and ethanol. A basic polystrene resin (methylpolystyrene carbonate resin) was added and the mixture was stirred at ambient temperature for 12 hours. The mixture was filtered and the filtrate was evaporated. The residue was purified by column chromatography on silica using a 19:1 mixture of methylene-chloride and methanol as eluent. The material so obtained was dissolved in methylene chloride and maleic acid (1 equivalent) was added. The resultant precipitate was isolated. The maleate salt so obtained gave the following characterising data: —¹H NMR: (DMSOd₆) 0.73 (m, 2H), 0.77 (m; 2H), 2.68 (m, 1H), 3.33 (s, 3H), 3.98 (s, 3H), 3.99 (s, 3H), 4.2 (s, 2H), 4.43 (s, 2H), 5.06 (s, 2H), 6.02 (s, 2H), 7.18 (s, 1H), 7.4 (s, 1H), 7.49 (s, 1H), 7.53 (s, 1H), 7.68 (s, 1H), 7.77 (s, 1H), 8.14 (s, 1H), 8.66 (s, 1H), 10.49 (s, 1H); Mass Spectrum: M+H⁺ 519.

The N-[3-(N-tert-butoxycarbonyl-N-cyclopropylaminomethyl)-5-methoxymethylphenyl]-2-[4-(6,7-dimethoxyquinazolin-4-yloxy)pyrazol-1-yl]acetamide used as a starting material was prepared as follows: —

An excess of methyl iodide was added to a stirred mixture of N-tert-butoxycarbonyl-N-cyclopropyl-N-(3-hydroxymethyl-5-nitrobenzyl)amine (0.63 g), sodium hydride (60% dispersion in mineral oil, 0.234 g) and THF (8 ml) and the resultant mixture was stirred at ambient temperature for 1 hour. The solvent was evaporated and the residue was partitioned between diethyl ether and a saturated aqueous sodium bicarbonate solution. The organic phase was dried and evaporated. The residue was purified by column chromatography on silica using increasingly polar mixtures of methylene chloride and ethyl acetate as eluent. There was thus obtained N-tert-butoxycarbonyl-N-cyclopropyl-N-(3-methoxymethyl-5-nitrobenzyl)amine (0.657 g); ¹H NMR: (DMSOd₆) 0.61 (m, 2H), 0.68 (m, 2H), 1.4 (s, 9H), 2.48 (m, 1H), 3.33 (s, 3H), 4.9 (s, 2H), 4.55 (s, 2H), 7.61 (s, 1H), 7.96 (s, 1H), 8.06 (s, 1H).

Using an analogous procedure to that described in the relevant portion of Example 19 that is concerned with the preparation of starting materials, N-tert-butoxycarbonyl-N-cyclopropyl-N-(3-methoxymethyl-5-nitrobenzyl)amine was converted into 3-(N-tert-butoxycarbonyl-N-cyclopropylaminomethyl)-5-methoxymethylaniline in 100% yield; ¹H NMR: (DMSOd₆) 0.57 (m, 2H), 0.63 (m, 2H), 1.4 (s, 9H), 2.38 (m, 1H), 3.24 (s, 3H), 4.19 (s, 2H), 4.23 (s, 2H), 5.07 (s, 2H), 6.3 (s, 1H), 6.32 (s, 1H), 6.39 (s, 1H).

Using an analogous procedure to that described in Example 8, 2-[4-(6,7-dimethoxyquinazolin-4-yloxy)pyrazol-1-yl]acetic acid was reacted with 3-(N-tert-butoxycarbonyl-N-cyclopropylaminomethyl)-5-methoxymethylaniline to give the required starting material in 84% yield.

[14] ¹H NMR: (DMSOd₆) 0.16-0.22 (m, 2H), 3.31-0.38 (m, 2H), 2.04-2.11 (m, 1H), 2.1 (br s, 1H), 2.67 (t, 2H), 2.78 (t, 2H), 3.98 (s, 3H), 3.99 (s, 3H), 5.03 (s, 2H), 6.93 (s, 1H), 7.23 (m, 1H), 7.39 (s, 1H), 7.42 (d, 1H), 7.47 (m, 1H), 7.54 (s, 1H), 7.68 (s, 1H), 8.12 (s, 1H), 8.66 (s, 1H), 10.28 (br s, 1H); Mass Spectrum: M+H 489.

The N-{3-[2-(N-tert-butoxycarbonyl-N-cyclopropylamino)ethyl]phenyl}-2-[4-(6,7-dimethoxyquinazolin-4-yloxy)pyrazol-1-yl]acetamide used as a starting material was prepared as follows: —

2-(3-Nitrophenyl)ethyl bromide (J. Med. Chem., 1977, 20, 1020; 0.69 g) was added slowly at ambient temperature to a solution of cyclopropylamine (0.684 g) in methylene chloride (3 ml). The resultant mixture was stirred and heated to reflux for 12 hours. The mixture was evaporated and the residue was partitioned between diethyl ether and a dilute aqueous sodium bicarbonate solution. The organic phase was dried over magnesium sulphate and evaporated. There was thus obtained N-cyclopropyl-N-[2-(3-nitrophenyl)ethyl]amine as an oil (0.550 g); ¹H NMR: (DMSOd₆) 0.17 (m, 2H), 0.34 (m, 2H), 2.08 (m, 1H), 2.19 (br s, 1H), 2.85 (s, 2H), 3.33 (s, 2H), 7.58 (m, 1H), 7.69 (m, 1H), 8.06 (m, 2H); Mass Spectrum: M+H⁺ 207.

Using analogous procedures to those described in relevant portions of Example 19 that are concerned with the preparation of starting materials, N-cyclopropyl N-[2-(3-nitrophenyl)ethyl]amine was converted into N-tert-butoxycarbonyl-N-cyclopropyl-N-[2-(3-nitrophenyl)ethyl]amine in 81% yield; ¹H NMR: (DMSOd₆) 0.49 (m, 2H), 0.67 (m, 2H), 1.31 (s, 9H), 2.37 (m, 1H), 2.93 (t, 2H), 3.43 (t, 2H), 7.59 (m, 1H), 7.7 (d, lI), 8.08 (m, 2H); which in turn was converted into 3-[2-(N-tert-butoxycarbonyl-N-cyclopropylamino)ethyl]aniline in 100% yield; ¹H NMR: (DMSOd₆) 0.49 (m, 2H), 0.69 (m, 2H), 1.38 (s, 9H), 2.44 (m, 1H), 2.59 (t, 2H), 3.27 (t, 2H), 4.93 (s, 2H), 6.31 (d, 1H), 6.37 (m, 2H), 6.9 (m, 1H).

Using an analogous procedure to that described in Example 8, 2-[4-(6,7-dimethoxyquinazolin-4-yloxy)pyrazol-1-yl]acetic acid was reacted with 3-[2-(N-tert-butoxycarbonyl-N-cyclopropylamino)ethyl]aniline to give the required starting material in 84% yield; ¹H NMR: (DMSOd₆+CF₃CO₂D) 0.51 (m, 2H), 0.67 (m, 2H), 1.37 (s, 9H), 2.45 (m, 1H), 2.76 (t, 2H), 3.36 (t, 2H), 4.06 (t, 3H), 4.08 (s, 3H), 5.09 (s, 2H), 6.94 (d, 1H), 7.26 (t, 1H), 7.49 (m, 3H), 7.73 (s, 1H), 7.75 (s, 1H), 8.2 (s, 1H), 9.16 (s, 1H); Mass Spectrum: M+H⁺ 589.

[15] ¹H NMR: (DMSOd₆) 0.18-0.25 (m, 2H), 0.33-0.4 (m, 2H), 2.1-2.16 (m, 1H), 2.4-2.46 (br s, 1H), 2.92 (t, 2H), 3.97 (t, 2H), 3.98 (s, 3H), 3.99 (s, 3H), 5.03 (s, 2H), 6.67 (m, 1H), 7.1 (d, 1H), 7.22 (m, 1H), 7.33 (m, 1H), 7.39 (s, 1H), 7.54 (s, 1H), 7.68 (s, 1H), 8.12 (s, 1H), 8.66 (s, 1H), 10.32 (s, 1H); Mass Spectrum: M+H⁺ 505.

The N-{3-[2-(N-tert-butoxycarbonyl-N-cyclopropylamino)ethoxy]phenyl}-2-[4-(6,7-dimethoxyquinazolin-4-yloxy)pyrazol-1-yl]acetamide used as a starting material was prepared as follows: —

Sodium hydride (60% dispersion in mineral oil; 0.12 g) was added portionwise to a stirred solution of 2-cyclopropylaminoethanol (J. Med. Chem., 1973, 16, 736; 0.303 g) in DMF (7 ml) that had been cooled to 5° C. The reaction mixture was stirred at 5° C. for 30 minutes. A solution of 3-fluoronitrobenzene (0.282 g) in DMF (1 ml) was added dropwise and the resultant mixture was stirred at ambient temperature for 4 hours. The solvent was evaporated and the residue was acidified to pH 2 by the addition of 2N aqueous hydrochloric acid solution. The aqueous mixture was washed with diethyl ether. The aqueous phase was basified with 4M aqueous sodium carbonate solution and extracted with diethyl ether. The resultant organic phase was dried over magnesium sulphate and evaporated. The residue was purified by column chromatography on silica using a 97:3 mixture of methylene chloride and methanol as eluent. There was thus obtained N-cyclopropyl-N-[2-(3-nitrophenoxy)ethyl]amine (0.27 g).

Using analogous procedures to those described in relevant portions of Example 19 that are concerned with the preparation of starting materials, N-cyclopropyl-N-[2-(3-nitrophenoxy)ethyl]amine was converted into N-tert-butoxycarbonyl-N-cyclopropyl-N-[2-(3-nitrophenoxy)ethyl]amine in 90% yield; ¹H NMR: (DMSOd₆) 0.59 (m, 2H), 0.69 (m, 2H), 1.38 (s, 9H), 2.55 (m, 1H), 3.55 (t, 2H), 4.23 (t, 2H), 7.43 (m, 1H), 7.59 (m, 1H), 7.72 (m, 1H), 7.83 (m, 1H); which in turn was converted into 3-[2-(N-tert-butoxycarbonyl-N-cyclopropylamino)ethoxy]aniline in 100% yield; ¹H NMR: (DMSOd₆) 0.59 (m, 2H), 0.69 (m, 2H), 2.54 (m, 1H), 3.46 (t, 2H), 3.95 (t, 2H), 5.03 (s, 2H), 6.06 (s, 1H), 6.12 (m, 2H), 6.88 (t, 1H).

Using an analogous procedure to that described in Example 8, 2-[4-(6,7-dimethoxyquinazolin-4-yloxy)pyrazol-1-yl]acetic acid was reacted with 3-[2-(N-tert-butoxycarbonyl-N-cyclopropylamino)ethoxy]aniline to give the required starting material in 91% yield; ¹H NMR: (DMSOd₆+CF₃CO₂D) 0.6 (m, 2H), 0.69 (m, 2H), 1.4 (s, 9H), 2.5 (m, 1H), 3.53 (t, 2H), 4.05 (m, 8H), 5.08 (s, 2H), 6.68 (m, 1H), 7.14 (d, 1H), 7.23 (t, 2H), 7.33 (s, 1H), 7.5 (s, 1H), 7.72 (s, 1H), 7.74 (s, 1H), 8.19 (s, 1H), 9.12 (s, 1H); Mass Spectrum: M+H⁺ 605.

[16] ¹H NMR: (DMSOd₆) 3.28-3.33 (m, 1H), 3.48-3.55 (m, 2H), 3.68-3.72 (m, 2H), 3.73 (s, 3H), 3.98 (s, 3H), 3.99 (s, 3H), 4.8-4.87 (m, 1H), 5.02 (s, 2H), 6.93 (d, 1H), 7.09 (s, 1H), 7.1 (m, 1H), 7.39 (s, 1H), 7.54 (s, 1H), 7.67 (s, 1H), 8.11 (s, 1H), 8.66 (s, 1H), 10.2 (br s, 1H); Mass Spectrum: M+H⁺ 507.

The N-[3-(N-tert-butoxycarbonylazetidin-3-yloxy)-4-methoxyphenyl]-2-[4-(6,7-dimethoxyquinazolin-4-yloxy)pyrazol-1-yl]acetamide used as a starting material was prepared as follows: —

Sodium hydride (60% dispersion in mineral oil; 0.048 g) was added to a stirred solution of tert-butyl 3-hydroxyazetidine-1-carboxylate (Organic Letters 2002, 4, 1859; 0.21 g) in DMF (5 ml) that had been cooled to 5° C. and the mixture was stirred at 5° C. for 5 minutes. 2-Fluoro-4-nitroanisole (0.171 g) was added and the reaction mixture was heated to 60° C. for 3 hours. The resultant mixture was cooled to ambient temperature acidified to pH6 by the addition of 1N aqueous hydrochloric acid solution and extracted with diethyl ether. The organic extract was dried over magnesium sulphate and evaporated. The residue was purified by column chromatography om silica using a 19:1 mixture of methylene chloride and diethyl ether as eluent. There was thus obtained tert-butyl 3-(2-methoxy-5-nitrophenoxy)azetidine-1-carboxylate as an oil (0.198 g); ¹H NMR: (DMSOd₆) 1.39 (s, 9H), 3.84 (m, 2H), 3.93 (s, 3H), 4.34 (m, 2H), 5.14 (m, 1H), 7.24 (d, 1H), 7.48 (m, 1H), 7.95 (m, 1H).

A mixture of tert-butyl 3-(2-methoxy-5-nitrophenoxy)azetidine-1-carboxylate (0.19 g), 10% platinum-on-carbon (0.03 g) and ethyl acetate (10 ml) was stirred under 1.8 atmospheres pressure of hydrogen for 1.5 hours. The resultant mixture was filtered and the filtrate was evaporated. There was thus obtained tert-butyl 3-(5-amino-2-methoxyphenoxy)azetidine-1-carboxylate (0.168 g); ¹H NMR: (DMSOd₆) 1.39 (s, 9H), 3.62 (s, 3H), 3.77 (m, 2H), 4.22 (m, 2H), 4.65 (s, 2H), 4.8 (m, 1H), 6.0 (m, 1H), 6.11 (m, 1H), 6.68 (d, 1H).

Using an analogous procedure to that described in Example 8, 2-[4-(6,7-dimethoxyquinazolin-4-yloxy)pyrazol-1-yl]acetic acid was reacted with tert-butyl 3-(5-amino-2-methoxyphenoxy)azetidine-1-carboxylate to give the required starting material in 84% yield; ¹H NMR: (DMSOd₆) 1.39 (s, 9H), 3.75 (s, 3H), 3.81 (m, 2H), 3.98 (s, 3H), 3.89 (s, 3H), 4.23 (m, 2H), 4.87 (m, 1H), 4.99 (s, 2H), 6.97 (d, 1H), 7.08 (m, 1H), 7.14 (m, 1H), 7.39 (s, 1H), 7.54 (s, 1H), 7.68 (s, 1H), 8.11 (s, 1H), 8.66 (s, 1H); Mass Spectrum: M−H⁻ 605.

[17] ¹H NMR: (DMSOd₆) 1.39-1.48 (m, 1H), 1.57-1.74 (m, 2H), 1.78-1.88 (m, 1H), 2.74-2.85 (m, 2H), 3.34-3.42 (m, 1H), 3.67-3.77 (m, 2H), 3.73 (s, 3H), 3.98 (s, 3H), 3.99 (s, 3H), 5.0 (s, 2H), 6.91 (d, 1H), 7.07 (m, 1H), 7.35 (d, 1H), 7.39 (s, 1H), 7.54 (s, 1H), 7.67 (s, 1H), 8.11 (s, 1H), 8.66 (s, 1H), 10.19 (s, 1H); Mass Spectrum: M+H⁺ 535.

The N-{3-[(2S)—N-tert-butoxycarbonylpyrrolidin-2-ylmethoxy]-4-methoxyphenyl}-2-[4-(6,7-dimethoxyquinazolin-4-yloxy)pyrazol-1-yl]acetamide used as a starting material was prepared as follows: —

A mixture of N-tert-butoxycarbonyl-(2S)-2-pyrrolidinemethanol (0.552 g), 2-methoxy-5-nitrophenol (0.338 g), di-tert-butyl azodicarboxylate (0.552 g), triphenylphosphine (0.629 g) and THF (5 ml) was stirred and heated to 60° C. for 4 hours. The mixture was evaporated and the residue was purified by column chromatography on silica using a 19:1 mixture of methylene chloride and diethyl ether as eluent. There was thus obtained tert-butyl (2S)-2-(2-methoxy-5-nitrophenoxymethyl)pyrrolidine-1-carboxylate (0.605 g); ¹H NMR: (DMSOd₆) 1.39 (s, 9H), 1.79 (m, 1H), 1.92-1.99 (m, 3H), 3.27 (m, 2H), 3.92 (s, 3H), 4.05 (m, 2H), 4.13 (m, 1H), 7.19 (d, 1H), 7.78 (d, 1H), 7.92 (d, 1H).

A mixture of tert-butyl (2S)-2-(2-methoxy-5-nitrophenoxymethyl)pyrrolidine-1-carboxylate (0.91 g), 10% palladium-on-carbon catalyst (0.15 g) and ethyl acetate (25 ml) was stirred under 1.8 atmospheres pressure of hydrogen for 1 hour. The resultant mixture was filtered and the filtrate was evaporated. There was thus obtained tert-butyl (2S)-2-(5-amino-2-methoxyphenoxymethyl)pyrrolidine-1-carboxylate (0.83 g); ¹H NMR: (DMSOd₆) 1.39 (s, 9H), 1.78 (m, 1H), 1.97 (m, 3H), 3.27 (m, 2H), 3.61 (s, 3H), 3.75 (m, 1H), 3.91 (m, 2H), 4.01 (m, 1H), 4.63 (s, 2H), 6.07 (m, 1H), 6.26 (m, 1H), 6.64 (d, 1H).

Using an analogous procedure to that described in Example 8, 2-[4-(6,7-dimethoxyquinazolin-4-yloxy)pyrazol-1-yl]acetic acid was reacted with tert-butyl (2S)-2-(5-amino-2-methoxyphenoxymethyl)pyrrolidine-1-carboxylate to give the required starting material in 93% yield; ¹H NMR: (DMSOd₆+CF₃CO₂D) 1.38-1.41 (m, 9H), 1.81 (m, 1H), 1.99 (m, 3H), 3.3 (m, 2H), 3.76 (s, 3H), 3.86 (m, 1H), 4.03 (m, 2H), 4.07 (s, 3H), 4.1 (s, 3H), 5.07 (s, 2H), 6.93 (m, 1H), 7.09 (m, 1H), 7.32 (m, 1H), 7.39 (s, 1H), 7.54 (s, 1H), 7.67 (s, 1H), 8.11 (s, 1H), 8.65 (s, 1H); Mass Spectrum: M+H⁺ 635.

[18] ¹H NMR: (DMSOd₆) 1.44-1.54 (m, 1H), 1.58-1.77 (m, 2H); 1.8-1.89 (m, 1H), 2.76-2.87 (m, 2H), 3.39-3.46 (m, 1H), 3.77-3.88 (m, 2H), 3.98 (s, 3H), 3.99 (s, 3H), 5.04 (s, 2H), 7.12 (m, 1H), 7.36 (d, 1H), 7.39 (s, 1H), 7.54 (s, 1H), 7.55 (d, 1H), 7.68 (s, 1H), 8.12 (s, 1H), 8.66 (s, 1H), 10.48 (s, 1H); Mass Spectrum: M+H⁺ 539 and 541.

The N-{3-[(2S)—N-tert-butoxycarbonylpyrrolidin-2-ylmethoxy]-4-chlorophenyl}-2-[4-(6,7-dimethoxyquinazolin-4-yloxy)pyrazol-1-yl]acetamide used as a starting material was prepared as follows: —

Using analogous procedures to those described in the first two paragraphs of the portion of Note [17] immediately above that is concerned with the preparation of starting materials, N-tert-butoxycarbonyl-(2S)-2-pyrrolidinemethanol was reacted with 2-chloro-5-nitrophenol to give tert-butyl (2S)-2-(2-chloro-5-nitrophenoxymethyl)pyrrolidine-1-carboxylate in 90% yield; ¹H NMR: (DMSOd₆) 1.39 (s, 9H), 1.81 (m, 1H), 1.94 (m, 1H), 2.04 (m, 2H), 3.3 (m, 2H), 4.09 (m, 1H), 4.26 (m, 2H), 7.76 (m, 1H), 7.84 (m, 1H), 7.96 (m, 1H); which in turn was converted into tert-butyl (2S)-2-(5-amino-2-chlorophenoxymethyl)pyrrolidine-1-carboxylate in 82% yield; ¹H NMR: (DMSOd₆) 1.4 (s, 9H), 1.78 (m, 1H), 2.0 (m, 3H), 3.3 (m, 2H), 3.95 (m, 1H), 4.01 (m, 2H), 6.53 (s, 1H), 6.7 (s, 1H), 7.25 (s, 1H).

Using an analogous procedure to that described in Example 8, 2-[4-(6,7-dimethoxyquinazolin-4-yloxy)pyrazol-1-yl]acetic acid was reacted with tert-butyl (2S)-2-(5-amino-2-chlorophenoxymethyl)pyrrolidine-1-carboxylate to give the required starting material in 94% yield; ¹H NMR: (DMSOd₆) 1.35-1.38 (m, 9H), 1.8 (m, 1H), 1.95-2.0 (m, 3H), 3.31 (m, 2H), 3.98 (s, 3H), 3.99 (s, 3H), 4.04 (m, 3H), 5.04 (s, 2H), 7.15 (m, 1H), 7.37 (m, 1H), 7.39 (s, 1H), 7.5 (m, 1H), 7.54 (s, 1H), 7.68 (s, 1H), 8.12 (s, 1H), 8.65 (s, 1H); Mass Spectrum: M+H⁺ 639 and 641.

[19] H NMR: (DMSOd₆) 1.48-1.59 (m, 1H), 1.62-1.82 (m, 2H), 1.84-1.94 (m, 1H), 2.83-2.95 (m, 2H), 3.45-3.55 (m, 1H), 3.92 (d, 2H), 3.98 (s, 3H), 3.99 (s, 3H), 5.02 (s, 2H), 7.14 (d, 1H), 7.39 (s, 1H), 7.44 (m, 1H), 7.54 (s, 1H), 7.68 (s, 1H), 7.77 (d, 1H), 8.12 (s, 1H), 8.66 (s, 1H), 10.37 (s, 1H); Mass Spectrum: M+H^{+ 539} and 541.

The N-{4-[(2S)—N-tert-butoxycarbonylpyrrolidin-2-ylmethoxy]-3-chlorophenyl}-2-[4-(6,7-dimethoxyquinazolin-4-yloxy)pyrazol-1-yl]acetamide used as a starting material was prepared as follows: —

Using analogous procedures to those described in the first two paragraphs of the portion of Note [17] immediately above that is concerned with the preparation of starting materials, N-tert-butoxycarbonyl-(2S)-2-pyrrolidinemethanol was reacted with 2-chloro-4-nitrophenol to give tert-butyl (2S)-2-(2-chloro-4-nitrophenoxymethyl)pyrrolidine-1-carboxylate in 81% yield; ¹H NMR: (DMSOd₆) 1.39 (s, 9H), 1.81 (m, 1H), 1.83 (m, 1H), 2.04 (m, 2H), 3.3 (m, 2H), 4.09 (m, 1H), 4.26 (m, 2H), 7.42 (d, 1H), 8.22 (m, 1H), 8.32 (m, 1H); which in turn was converted into tert-butyl (2S)-2-(4-amino-2-chlorophenoxymethyl)pyrrolidine-1-carboxylate in 92% yield; ¹H NMR: (DMSOd₆) 1.38 (s, 9H), 1.77 (m, 1H), 1.96 (m, 3H), 3.26 (m, 2H), 3.82 (m, 1H), 3.93 (br s, 2H), 4.91 (m, 2H), 6.45 (m, 1H), 6.62 (m, 1H), 6.85 (m, 1H).

Using an analogous procedure to that described in Example 8, 2-[4-(6,7-dimethoxyquinazolin-4-yloxy)pyrazol-1-yl]acetic acid was reacted with tert-butyl (2S)-2-(4-amino-2-chlorophenoxymethyl)pyrrolidine-1-carboxylate to give the required starting material in 94% yield; ¹H NMR: (DMSOd₆+CF₃CO₂D) 1.41 (s, 9H), 1.81 (m, 1H), 2.0 (m, 3H), 3.31 (m, 2H), 4.05 (m, 3H), 4.09 (s, 3H), 4.12 (s, 3H), 5.09 (s, 2H), 7.17 (m, 1H), 7.46 (m, 1H), 7.53 (s, 1H), 7.75 (m, 2H), 7.82 (m, 1H), 8.21 (s, 1H), 9.23 (s, 1H); Mass Spectrum: M+H⁺ 639 and 641.

[20] ¹H NMR: (DMSOd₆) 0.25-0.31 (m, 2H), 0.34-0.4 (m, 2H), 2.13-2.19 (m, 1H), 2.74-2.84 (m, 1H), 3.31 (s, 2H), 3.98 (s, 3H), 3.99 (s, 3H), 5.04 (s, 2H), 7.24 (d, 1H), 7.27 (d, 1H), 7.28 (d, 1H), 7.35 (d, 1H), 7.39 (s, 1H), 7.55 (s, 1H), 7.67 (s, 1H), 7.99 (br s, 1H), 8.12 (s, 1H), 8.66 (s, 1H), 8.79 (s, 1H); Mass Spectrum: M+H⁺ 518.

The N-{3-[2-(N-tert-butoxycarbonyl-N-cyclopropylamino)acetamido]phenyl}-2-[4-(6,7-dimethoxyquinazolin-4-yloxy)pyrazol-1-yl]acetamide used as a starting material was prepared as follows: —

A mixture of x-chloro-3-nitroacetanilide (0.86 g), cyclopropylamine (0.912 g) and methylene chloride (5 ml) was stirred and heated to reflux for 16 hours. The mixture was concentrated by evaporation and the residue was partitioned between diethyl ether and a 2N aqueous sodium bicarbonate solution. The organic phase was dried over magnesium sulphate and evaporated. There was thus obtained α-cyclopropylamino-3-nitroacetanilide as an oil (0.9 g); ¹H NMR: (DMSOd₆) 0.3 (m, 2H), 0.38 (m, 2H), 2.18 (m, 1H), 3.33 (s, 2H), 7.62 (m, 1H), 7.9 (m, 1H), 7.97 (m, 1H), 8.69 (s, 1H); Mass Spectrum: M+H⁺ 236.

Using analogous procedures to those described in relevant portions of Example 19 that are concerned with the preparation of starting materials, x-cyclopropylamino-3-nitroacetanilide was converted into α-(N-tert-butoxycarbonyl-N-cyclopropylamino)-3-nitroacetanilide in 70% yield; ¹H NMR: (DMSOd₆) 0.61 (m, 2H), 0.67 (m, 2H), 1.1-1.47 (br s, 9H), 2.68 (m, 1H); 3.98 (br s, 2H), 7.62 (t, 1H), 7.91 (m, 6H), 8.63 (s, 1H); Mass Spectrum: M+H⁺ 336; which in turn was converted into x-(N-tert-butoxycarbonyl-N-cyclopropylamino)-3-aminoacetanilide in 95% yield; ¹H NMR: (DMSOd₆) 0.57 (m, 2H), 0.64 (m, 2H), 1.33-1.47 (br s, 9H), 2.63 (m, 1H), 3.87 (br s, 2H), 5.05 (s, 2H), 6.23 (m, 1H), 6.64 (m, 1H), 6.9 (m, 2H).

Using an analogous procedure to that described in Example 8, 2-[4-(6,7-dimethoxyquinazolin-4-yloxy)pyrazol-1-yl]acetic acid was reacted with α-(N-tert-butoxycarbonyl-N-cyclopropylamino)-3-aminoacetanilide to give the required starting material in 81% yield; ¹H NMR: (DMSOd₆) 0.59 (m, 2H), 0.66 (m, 2H), 1.32-1.41 (br s, 9H), 2.65 (m, 1H), 3.93 (br s, 2H), 3.98 (s, 3H), 3.99 (s, 3H), 5.03 (s, 2H), 7.25 (m, 2H), 7.34 (m, 1H), 7.39 (s, 1H), 7.55 (s, 1H), 7.67 (s, 1H), 8.0 (s, 1H), 8.12 (s, 1H), 8.66 (s, 1H); Mass Spectrum: M+H⁺ 618.

### EXAMPLE 21

### N-(4-chloro-3-ethylaminomethylphenyl)-2-[4-(6,7-dimethoxyquinazolin-4-yloxy)pyrazol-1-yl]acetamide

Ethylamine (0.024 g) was added to a stirred mixture of N-(4-chloro-3-formylphenyl)-2-[4-(6,7-dimethoxyquinazolin-4-yloxy)pyrazol-1-yl]acetamide (0.08 g), sodium triacetoxyborohydride (0.072 g), trimethyl orthoformate (0.2 ml), methanol (0.2 ml) and methylene chloride (0.6 ml) and the reaction mixture was stirred at ambient temperature for 12 hours. The solvent was evaporated and the residue was purified by preparative HPLC using a Waters ‘β Basic Hypersil’ reversed-phase column (5 microns silica, 30 mm diameter, 250 mm length) and decreasingly polar mixtures of water (containing 0.2% ammonium carbonate) and acetonitrile as eluent. There was thus obtained the title compound (0.021 g); ¹H NMR: (DMSOd₆) 1.05 (t, 3H), 2.04 (br s 1H), 2.57 (q, 2H), 3.73 (s, 2H), 3.98 s, 3H), 3.99 (s, 3H), 5.04 (s, 2H), 7.35 (d, 1H), 7.39 (d, 1H), 7.54 (s, 1H), 7.58 (m, 1H), 7.68 (s, 1H), 7.74 (d, 1H), 8.12 (s, 1H), 8.66 (s, 1H), 10.47 (s, 1H); Mass Spectrum: M+H⁺ 497.

The N-(4-chloro-3-formylphenyl)-2-[4-(6,7-dimethoxyquinazolin-4-yloxy)pyrazol-1-yl]acetamide used as a starting material was prepared as follows: —

Using an analogous procedure to that described in the portion of Note [13] below Table TV in Example 17 that is concerned with the preparation of starting materials, N-(4-chloro-3-hydroxymethylphenyl)-2-[4-(6,7-dimethoxyquinazolin-4-yloxy)pyrazol-1-yl]acetamide was oxidised to give the required product in 39% yield; ¹H NMR: (DMSOd₆) 3.98 (s, 3H), 3.99 (s, 3H), 5.07 (s, 2H), 7.39 (s, 1H), 7.54 (s, 1H), 7.61 (d, 1H), 7.69 (s, 1H), 7.88 (m, 1H), 8.16 (m, 2H), 8.66 (s, 1H); Mass Spectrum: M+H⁺ 468.

### EXAMPLE 22

Using an analogous procedure to that described in Example 21, the appropriate N-(3-formylphenyl)-2-pyrazol-1-ylacetamide was reacted with the appropriate amine or heterocycle to give the compounds described in Table VI. Unless otherwise stated, each required amine or heterocycle starting material was commercially available.

TABLE VI

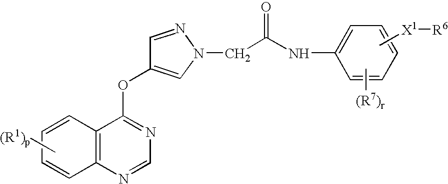

| No. & | | | |
|---|---|---|---|
| Note | (R¹)ₚ | (R⁷)ᵣ | X¹—R⁶ |
| [1] | 6,7-dimethoxy | 4-chloro | 3-(N-ethyl-N-methylaminomethyl) |
| [2] | 6,7-dimethoxy | 4-chloro | 3-(2-methylprop-2-en-1- |
| | | | ylaminomethyl) |
| [3] | 6,7-dimethoxy | 4-chloro | 3-(2-hydroxypropylaminomethyl) |
| [4] | 6,7-dimethoxy | 4-chloro | 3-cycloprop-1-ylmethylaminomethyl |
| [5] | 6,7-dimethoxy | 4-chloro | 3-(3-hydroxypyrrolidin-1-ylmethyl) |
| [6] | 6,7-dimethoxy | 4-chloro | 3-[(2R)-2-hydroxymethylpyrrolidin- |
| | | | 1-ylmethyl] |

Notes The products gave the characterising data shown below.

[1] Mass Spectrum: M+H⁺ 511 and 513.

[2] ¹H NMR: (DMSOd₆) 1.72 (s, 3H), 2.28 (br s, 1H), 3.10 (s, 2H), 3.68 (s, 2H), 3.98 (s, 3H), 3.99 (s, 3H), 4.82 (br s, 1H), 4.9 (br s, 1H), 5.04 (s, 1H), 7.36 (d, 1H), 7.4 (s, 1H), 7.54 (s, 1H), 7.59 (m, 1H), 7.68 (s, 1H), 7.76 (d, 1H), 8.12 (s, 1H), 8.66 (s, 1H), 10.49 (s, 1H); Mass Spectrum: M+H⁺ 523 and 525.

[3] ¹H NMR: (DMSOd₆) 1.03 (s, 3H), 2.42-2.48 (m, 2H), 3.69-3.74 (m, 1H), 3.75 (s, 2H), 3.98 (s, 3H), 3.99 (s, 3H), 5.05 (s, 2H), 7.36 (d, 1H), 7.39 (s, 1H), 7.54 (s, 1H), 7.59 (m, 1H), 7.68 (s, 1H), 7.74 (d, 1H), 8.12 (s, 1H), 8.66 (s, 1H), 10.63 (br s, 1H); Mass Spectrum: M+H⁺ 527 and 529.

[4] ¹H NMR: (DMSOd₆) 0.08-0.14 (m, 2H), 0.38-0.44 (m, 2H), 0.88-0.96 (m, 1H), 2.1 (br s, 1H), 2.41 (d, 2H), 3.77 (s, 2H), 3.98 (s, 3H), 3.99 (s, 3H), 5.04 (s, 2H), 7.35 (d, 1H), 7.39 (s, 1H), 7.54 (s, 1H), 7.58 (m, 1H), 7.68 (d, 1H), 7.75 (d, 1H), 8.13 (s, 1H), 8.66 (s, 1H), 10.47 (s, 1H); Mass Spectrum: M+H⁺ 523 and 525.

[5] ¹H NMR: (DMSOd₆) 1.52-1.61 (m, 1H), 1.95-2.05 (m, 1H), 2.35 (m, 1H), 2.49-2.55 (m, 1H), 2.59-2.66 (m, 1H), 2.8 (m, 1H), 3.64 (s, 2H), 3.98 (s, 3H), 3.99 (s, 3H), 4.18-4.27 (m, 1H), 4.72 (d, 1H), 5.04 (s, 2H), 7.37 (d, 1H), 7.39 (s, 1H), 7.54 (s, 1H), 7.58 (m, 1H), 7.68 (s, 1H), 7.72 (d, 1H), 8.12 (s, 1H), 8.66 (s, 1H), 10.48 (br s, 1H); Mass Spectrum: M+H⁺ 539 and 541.

[6]¹H NMR: (DMSOd₆) 1.51-1.72 (m, 3H), 1.83-1.93 (s, 1H), 2.14-2.22 (m, 1H), 2.61-2.69 (m, 1H), 2.87-2.94 (m, 1H), 3.26-3.33 (m, 1H), 3.44-3.53 (m, 2H), 3.5 (d, 1H), 3.98 (s, 3H), 3.99 (s, 3H), 4.07 (d, 1H), 4.44 (t, 1H), 5.04 (s, 2H), 7.35 (d, 1H), 7.39 (s, 1H), 7.54 (s, 1H), 7.60 (m, 1H), 7.68 (s, 1H), 7.72 (d, 1H), 8.12 (s, 1H), 8.66 (s, 1H), 10.53 (br s, 1H); Mass Spectrum: M+H⁺ 553 and 555.

### EXAMPLE 23

### N-[4-methoxy-3-(N-methylazetidin-3-yloxy)phenyl]-2-[4-(6,7-dimethoxyquinazolin-4-yloxy)pyrazol-1-yl]acetamide

Sodium triacetoxyborohydride (0.045 g) and acetic acid (0.013 g) were added in turn to a stirred mixture of N-(3-azetidin-3-yloxy-4-methoxyphenyl)-2-[4-(6,7-dimethoxyquinazolin-4-yloxy)pyrazol-1-yl]acetamide (0.1 g), formaldehyde (37% aqueous solution; 0.02 ml), methanol (1 ml) and methylene chloride (2 ml) and the reaction mixture was stirred at ambient temperature for 2 hours. The solvent was evaporated and the residue was purified by column chromatography on silica using a 19:1 mixture of methylene chloride and a 3M methanolic ammonia solution as eluent. There was thus obtained the title compound (0.075 g); ¹H NMR: (DMSOd₆) 2.28 (s, 3H), 2.93-2.99 (m, 2H), 3.67-3.74 (m, 2H), 3.73 (s, 3H), 3.98 (s, 3H), 3.99 (s, 3H), 4.57-4.64 (m, 1H), 4.99 (s, 2M), 6.93 (d, 1H), 7.09 (m, 1H), 7.15 (d, 1H), 7.4 (s, 1H), 7.54 (s, 1H), 7.67 (s, 1H), 8.11 (s, 1H), 8.66 (s, 1H), 10.2 (br s, 1H); Mass Spectrum: M+H⁺ 521.

### EXAMPLE 24

### N-(5-hydroxymethylthiazol-2-yl)-2-[4-(6,7-dimethoxyquinazolin-4-yloxy)pyrazol-1-yl]acetamide

Using an analogous procedure to that described in Example 8, 2-[4-(6,7-dimethoxyquinazolin-4-yloxy)pyrazol-1-yl]acetic acid was reacted with 2-amino-5-hydroxymethylthiazole (Farmaco, 1989, 44, 1011-30) to give the title compound in 55% yield; ¹H NMR: (DMSOd₆) 3.98 (s, 3H), 3.99 (s, 3H), 4.59 (d, 2H), 5.17 (s, 2H), 5.41 (t, 1H), 7.32 (s, 1H), 7.39 (s, 1H), 7.55 (s, 1H), 7.7 (s, 1H), 8.15 (s, 1H), 8.66 (s, 1H); Mass Spectrum: M+H⁺ 443;

### EXAMPLE 25

### N-(5-dimethylaminomethylthiazol-2-yl)-2-[4-(6,7-dimethoxyquinazolin-4-yloxy)pyrazol-1-yl]acetamide

Thionyl chloride (0.066 ml) was added to a stirred suspension of N-(5-hydroxymethylthiazol-2-yl)-2-[4-(6,7-dimethoxyquinazolin-4-yloxy)pyrazol-1-yl]acetamide (0.1 g) in methylene chloride (3 ml) that had been cooled to 0° C. and the mixture was stirred at ambient temperature for 2 hours. The solvent was evaporated to give N-(5 chloromethylthiazol-2-yl)-2-[4-(6,7-dimethoxyquinazolin-4-yloxy)pyrazol-1-yl]acetamide which was used without further purification. DMA (1.2 ml) and dimethylamine solution (2M in THF, 0.91 ml) were added in turn and the mixture was stirred at ambient temperature for 5 minutes. The mixture was evaporated and the residue was purified by preparative HPLC using a Waters ‘Symmetry’ C18 reversed-phase column (5 microns silica, 19 mm diameter, 100 mm length) and decreasingly polar mixtures of water (containing 2% acetic acid) and acetonitrile as eluent. There was thus obtained the title compound as a solid (0.055 g); ¹H NMR: (DMSOd₆) 2.14 (s, 6H), 3.55 (s, 2H), 3.98 (s, 3H), 3.99 (s, 3H), 5.16 (s, 2H), 7.31 (s, 1H), 7.39 (s, 1H), 7.55 (s, 1H), 7.69 (s, 1H), 8.14 (s, 1H), 8.66 (s, 1H), 12.37 (br s, 1H); Mass Spectrum: M+W 470.

### EXAMPLE 26

### N-[5-(N-cyclopropylaminomethyl)thiazol-2-yl]-2-[4-(6,7-dimethoxyquinazolin-4-yloxy)pyrazol-1-yl]acetamide

Using an analogous procedure to that described in Example 25, N-(5-chloromethylthiazol-2-yl)-2-[4-(6,7-dimethoxyquinazolin-4-yloxy)pyrazol-1-yl]acetamide was reacted with cyclopropylamine to give the title compound in 62% yield; ¹H NMR: (DMSOd₆) 0.19-0.26 (m, 2H), 0.3-0.37 (m, 2H), 2.02-2.09 (m, 1H), 0.84 (s, 2H), 3.98 (s, 3H), 3.99 (s, 3H), 5.15 (s, 2H), 7.29 (s, 1H), 7.39 (s, 1H), 7.55 (s; 1H), 7.68 (s, 1H), 8.1.3 (s, 1H), 8.66 (s, 1H), 12.32 (br s, 1H); Mass Spectrum: M+H⁺ 482.

### EXAMPLE 27

### N-{5-[N-(2-methylprop-2-en-1-ylamino)methyl]thiazol-2-yl}-2-[4-(6,7-dimethoxyquinazolin-4-yloxy)pyrazol-1-yl]acetamide

Using an analogous procedure to that described in Example 25, N-(5-chloromethylthiazol-2-yl)-2-[4-(6,7-dimethoxyquinazolin-4-yloxy)pyrazol-1-yl]acetamide was reacted with 2-methylprop-2-en-lylamine to give the title compound in 57% yield; ¹H NMR: (DMSOd₆) 1.68 (s, 3H), 3.04 (s, 2H), 3.78 (s, 2H), 3.98 (s, 3H), 3.99 (s, 3H), 4.79 (br s, 2H), 4.85 (br s, 1H), 5.15 (s, 2H), 7.27 (s, 1H), 7.39 (s, 1H), 7.54 (s, 1H), 7.69 (s, 1H), 8.14 (s, 1H), 8.66 (s, 1H), 12.33 (br s, 1H); Mass Spectrum: M+H⁺ 496.

### EXAMPLE 28

### N-(4-hydroxymethylpyridin-2-yl)-2-[4-(6,7-dimethoxyquinazolin-4-yloxy)pyrazol-1-yl]acetamide

Using an analogous procedure to that described in Example 8, 2-[4-(6,7-dimethoxyquinazolin-4-yloxy)pyrazol-1-yl]acetic acid was reacted with 2-amino-4-(tert-butyldimethylsilyloxymethyl)pyridine to give N-[4-(tert-butyldimethylsilyloxymethyl)pyridin-2-yl]-2-[4-(6,7-dimethoxyquinazolin-4-yloxy)pyrazol-1-yl]acetamide in 75% yield; ¹H NMR: (DMSOd₆) 0.1 (s, 6H), 0.82 (s, 9H), 3.9 (2s, 6H), 4.66 (s, 2H), 5.04 (s, 2H), 6.97 (m, 1H), 7.31 (s, 1H), 7.46 (s, 1H), 7.6 (s, 1H), 7.98 (m, 1H), 8.05 (s, 1H), 8.19 (m, 1H), 8.57 (s, 1H), 10.68 (s, 1H).

A mixture of the material so obtained (1.32 g), tetra-n-butyl ammonium fluoride (1M solution in THF, 2.64 ml) and THF (20 ml) was stirred at ambient temperature for 2 hours. Water was added to the reaction mixture. The resultant precipitate was collected, washed in turn with water and with ethyl acetate and dried under vacuum over phosphorous pentoxide. There was thus obtained the title compound (0.883 g); ¹H NMR: (DMSOd₆) 3.98 (s, 3H), 3.99 (s, 3H), 4.52 (d, 2H), 5.13 (s, 2H), 5.43 (t, 1H), 7.07 (m, 1H), 7.39 (s, 1H), 7.54 (s, 1H), 7.69 (s, 1H), 8.05 (m, 1H), 8.13 (s, 1H), 8.26 (m, 1H), 8.66 (m, 1H), 10.76 (s, 1H).

The 2-amino-4-(tert-butyldimethylsilyloxymethyl)pyridine used as a starting material was prepared as follows: —

tert-Butyldimethylsilyl chloride (1.44 g) and imidazole (0.65 g) were added in turn to a stirred solution of 2-amino-4-hydroxymethylpyridine (J. Med. Chem., 2001, 44, 78-93; 1.08 g) in DMF (9 ml). The mixture was stirred at ambient temperature for 2.5 hours. Water was added and the precipitate was isolated, washed with water and dried under vacuum. There was thus obtained 2-amino-4-(tert-butyldimethylsilyloxymethyl)pyridine (1.61 g); ¹H NMR: (DMSOd₆) 0.1 (s, 6H), 0.93 (s, 9H), 4.58 (s, 2H), 5.86 (s, 2H), 6.4 (m, 2H), 7.82 (m, 1H).

### EXAMPLE 29

### N-(6-hydroxymethylpyridin-2-yl)-2-[4-(6,7-dimethoxyquinazolin-4-yloxy)pyrazol-1-yl]acetamide

Using an analogous procedure to that described in the first paragraph of Example 28, 2-[4-(6,7-dimethoxyquinazolin-4-yloxy)pyrazol-1-yl]acetic acid was reacted with 2-amino-6-(tert-butyldimethylsilyloxymethyl)pyridine to give N-[6-(tert-butyldimethylsilyloxymethyl)pyridin-2-yl]-2-[4-(6,7-dimethoxyquinazolin-4-yloxy)pyrazol-1-yl]acetamide in 70% yield; ¹H NMR: (DMSOd₆) 0.1 (s, 6H), 0.82 (s, 9H), 3.88 (s, 3H), 3.89 (s, 3H), 4.59 (s, 2H), 5.01 (s, 2H), 7.09 (m, 1H), 7.29 (s, 1H), 7.44 (s, 1H), 7.58 (s, 1H), 7.73 (t, 1H), 7.83 (m, 1H), 8.02 (s, 1H), 8.55 (s, 1H), 10.69 (s, 1H); Mass Spectrum: M+H⁺ 551.

The material so obtained was reacted with tetra-n-butyl ammonium fluoride using an analogous procedure to that described in the second paragraph of Example 28. There was thus obtained the title compound in 77% yield; ¹H NMR: (DMSOd₆) 3.98 (s, 3H), 3.99 (s, 3H), 4.52 (d, 2H), 5.11 (s, 2H), 5.43 (t, 1H), 7.23 (d, 1H), 7.39 (s, 1H), 7.54 (s, 1H), 7.68 (s, 1H), 7.81 (t, 1H), 7.9 (m, 1H), 8.12 (s, 1H), 8.66 (s, 1H), 10.8 (s, 1H); Mass Spectrum: M+H⁺ 437.

The 2-amino-6-(tert-butyldimethylsilyloxymethyl)pyridine used as a starting material was prepared as follows: —

Using an analogous procedure to that described in the portion of Example 28 that is concerned with the preparation of starting materials, 2-amino-6-hydroxymethylpyridine (International Patent Application WO 01/17995) was reacted with tert-butyldimethylsilyl chloride to give 2-amino-6-(tert-butyldimethylsilyloxymethyl)pyridine in 79% yield; ¹H NMR: (DMSOd₆) 0.1 (s, 6H), 0.93 (s, 9H), 4.51 (s, 2H), 5.84 (s, 2H), 6.31 (d, 1H), 6.56 (d, 1H), 7.38 (t, 1H).

### EXAMPLE 30

### N-(4-dimethylaminomethylpyridin-2-yl)-2-[4-(6,7-dimethoxyquinazolin-4-yloxy)pyrazol-1-yl]acetamide

Methanesulphonyl chloride (0.275 ml) was added to a stirred mixture of N-(4-hydroxymethylpyridin-2-yl)-2-[4-(6,7-dimethoxyquinazolin-4-yloxy)pyrazol-1-yl]acetamide (0.85 g), triethylamine (0.562 ml) and DMA (20 ml) that had been cooled to 0° C. The resultant mixture was stirred at 0° C. for 10 minutes. Water was added to the mixture and the precipitate was recovered and purified by column chromatography on silica using a 10:1 mixture of methylene chloride and methanol as eluent. There was thus obtained N-(4-mesyloxymethylpyridin-2-yl)-2-[4-(6,7-dimethoxyquinazolin-4-yloxy)pyrazol-1-yl]acetamide (0.984 g); ¹H NMR: (DMSOd₆) 3.28 (s, 3H), 3.98 (s, 3H), 3.99 (s, 3H), 5.14 (s, 2H), 5.32 (s, 2H), 7.17 (m, 1H), 7.39 (s, 1H), 7.55 (s, 1H), 7.69 (s, 1H), 8.12 (m, 2H), 8.38 (m, 1H), 8.66 (s, 1H), 10.95 (s, 1H); Mass Spectrum: M+H⁺ 515.

A portion (0.13 g) of the material so obtained was dissolved in DMA (1 ml) and a solution of dimethylamine (0.165 ml) in THF (1 ml), potassium iodide (0.013 g) and potassium carbonate (0.052 g) were added in turn. The resultant mixture was heated to 90° C. for 1.5 minutes in a microwave oven. The solvent was evaporated and the residue was purified by column chromatography on silica using a gradient of 95:5 to 88:12 of methylene chloride and methanol as eluent. There was thus obtained the title compound (0.047 g); ¹H NMR: (DMSOd₆) 2.15 (s, 6H), 3.4 (s, 2H), 3.98 (s, 3H), 3.99 (s, 3H), 5.12 (s, 2H), 7.06 (d, 1H), 7.4 (s, 1H), 7.54 (s, 1H), 7.68 (s, 1H), 8.04 (br s, 1H), 8.13 (s, 1H), 8.27 (d, 1H), 8.66 (s, 1H), 10.79 (s, 1H); Mass Spectrum: M+H⁺: 464.

### EXAMPLE 31

Using analogous procedures to those described in Example 30, the appropriate N-(mesyloxymethylpyridin-2-yl)-2-(4-quinazolin-4-yloxypyrazol-1-yl)acetamide was reacted with the appropriate amine or heterocycle to give the compounds described in Table VII. Unless otherwise stated, each required amine or heterocycle starting material was commercially available.

TABLE VII

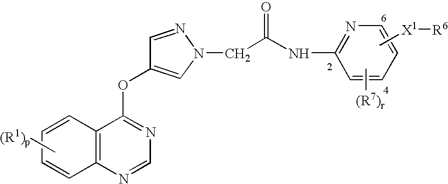

| No.& | | | |
|---|---|---|---|
| Note | (R¹)ₚ | (R⁷)ᵣ | X¹—R⁶ |
| [1] | 6,7-dimethoxy | H | 4-ethylaminomethyl |
| [2] | 6,7-dimethoxy | H | 4-(2-methylprop-2-en-1-ylaminomethyl) |
| [3] | 6,7-dimethoxy | H | 4-cyclopropylaminomethyl |
| [4] | 6,7-dimethoxy | H | 4-cyclopropylmethylaminomethyl |
| [5] | 6,7-dimethoxy | H | 4-pyrrolidin-1-ylmethyl |
| [6] | 6,7-dimethoxy | H | 6-dimethylaminomethyl |
| [7] | 6,7-dimethoxy | H | 6-(2-methylprop-2-en-1-ylaminomethyl) |
| [8] | 6,7-dimethoxy | H | 6-cyclopropylaminomethyl |
| [9] | 6,7-dimethoxy | H | 6-pyrrolidin-1-ylmethyl |

Notes The products gave the characterising data shown below.

[1] ¹H NMR: (DMSOd₆) 1.01 (t, 3H), 2.37 (br s, 1H), 2.51 (q, 2H), 3.69 (s, 2H), 3.98 (s, 3H), 3.99 (s, 3H), 5.12 (s, 2H), 7.1 (d, 1H), 7.39 (s, 1H), 7.54 (s, 1H), 7.68 (s, 1H), 8.05 (br s, 1H), 8.13 (s, 1H), 8.25 (d, 1H), 8.66 (s, 1H), 10.75 (br s, 1H); Mass Spectrum: M+H⁺ 464.

[2] ¹H NMR: (DMSOd₆) 1.68 (s, 3H), 2.44 (br s, 1H), 3.03 (s, 2H), 3.65 (s, 2H), 3.98 (s, 3H), 3.99 (s, 3H), 3.78 (br s, 1H), 3.85 (br s, 1H), 5.12 (s, 2H), 7.11 (d, 1H), 7.39 (s, 1H), 7.55 (s, 1H), 7.68 (s, 1H), 8.06 (br s, 1H), 8.13 (s, 1H), 8.25 (d, 1H), 8.66 (s, 1H), 10.75 (s, 1H); Mass Spectrum: M+H⁺ 490.

[3] ¹H NMR: (DMSOd₆) 0.2-0.26 (m, 2H), 0.3-0.36 (m, 2H), 2.0-2.07 (m, 1H), 2.91 (br s, 1H), 3.72 (s, 2H), 3.98 (s, 3H), 3.99 (s, 3H), 5.12 (s, 2H), 7.1 (d, 1H), 7.39 (s, 1H), 7.54 (s, 1H), 7.69 (s, 1H), 8.04 (br s, 1H), 8.13 (s, 1H), 8.24 (d, 1H), 8.66 (s, 1H), 10375 (s, 1H); Mass Spectrum: M+H⁺ 476.

[4] ¹H NMR: (DMSOd₆) 0.05-0.11 (m, 2H), 0.34-0.42 (m, 2H), 0.82-0.93 (m, 1H), 2.35 (d, 2H), 3.73 (s, 2H), 3.98 (s, 3H), 3.99 (s, 3H), 5.12 (s, 2H), 7.11 (d, 1H), 7.39 (s, 1H), 7.54 (s, 1H), 7.68 (s, 1H), 8.05 (br s, 1H), 8.13 (s, 1H), 8.25 (d, 1H), 8.66 (s, 1H), 10.75 (s, 1H); Mass Spectrum: M+H⁺ 490.

[5] ¹H NMR: (DMSOd₆) 1.63-1.76 (m, 4H), 2.39-2.47 (m, 4H), 3.58 (s, 2H), 3.98 (s, 3H), 3.99 (s, 3H), 5.12 (s, 2H), 7.07 (d, 1H), 7.39 (s, 1H), 7.54 (s, 1H), 7.68 (s, 1H), 8.05 (br s, 1H), 8.13 (s, 1H), 8.26 (d, 1H), 8.66 (s, 1H), 10.78 (br s, 1H); Mass Spectrum: M+H⁺ 490.

[6] ¹H NMR: (DMSOd₆) 2.2 (s, 6H), 3.46 (s, 2H), 3.98 (s, 3H), 3.99 (s, 3H), 5.11 (s, 2H), 7.16 (d, 1H), 7.39 (s, 1H), 7.54 (s, 1H), 7.68 (s, 1H), 7.78 (m, 1H), 7.93 (d, 1H), 8.12 (s, 1H), 8.66 (s, 1H), 10.85 (s, 1H); Mass Spectrum: M+H⁺ 464.

N-(6-Mesyloxymethylpyridin-2-yl)-2-[4-(6,7-dimethoxyquinazolin-4-yloxy)pyrazol-1-yl]acetamide was obtained as an intermediate in 86% yield and gave the following characterising data; ¹H NMR: (DMSOd₆) 3.3 (s, 3H), 3.98 (s, 3H), 3.99 (s, 3H), 5.14 (s, 2H), 5.26 (s, 2H), 7.29 (d, 1H), 7.39 (s, 1H), 7.55 (s, 1H), 7.68 (s, 1H), 7.88 (t, 1H), 8.05 (m, 1H), 8.13 (s, 1H), 8.66 (s, 1H), 10.98 (s, 1H); Mass Spectrum: M−H⁻ 513.

[7] ¹H NMR: (DMSOd₆) 1.72 (s, 3H), 3.14 (s, 2H), 3.75 (s, 2H), 3.98 (s, 3H), 3.99 (s, 3H), 4.83 (br s, 1H), 3.91 (br s, 1H), 5.12 (s, 2H), 7.21 (d, 1H), 7.39 (s, 1H), 7.54 (s, 1H), 7.68 (s, 1H), 7.72 (m, 1H), 7.91 (d, 1H), 8.12 (s, 1H), 8.66 (s, 1H), 10.79 (s, 1H); Mass Spectrum: M+H⁺ 490.

[8] ¹H NMR: (DMSOd₆) 0.22-0.29 (m, 2H), 0.32-0.41 (m, 2H), 2.05-2.13 (m, 1H), 3.78 (s, 2H), 3.98 (s, 3H), 3.99 (s, 3H), 5.11 (s, 2H), 7.17 (d, 1H), 7.39 (s, 1H), 7.54 (s, 1H), 7.67 (s, 1H), 7.75 (m, 1H), 7.89 (d, 1H), 8.12 (s, 1H), 8.66 (s, 1H), 10.8 (s, 1H); Mass Spectrum: M+H⁺ 476.

[9] ¹H NMR: (DMSOd₆) 1.66-1.75 (m, 4H), 2.47-2.53 (m, 4H), 3.64 (s, 2H), 3.98 (s, 3H), 3.99 (s, 3H), 5.11 (s, 2H), 7.16 (d, 1H), 7.39 (s, 1H), 7.55 (s, 1H), 7.68 (s, 1H), 7.76 (m, 1H), 7.91 (d, 1H), 8.12 (s, 1H), 8.66 (s, 1H), 10.85 (br s, 1H); Mass Spectrum: M+H⁺ 490.

### EXAMPLE 32

### N-{5-[N-(2-hydroxyethyl)-N-methylamino]pyridin-2-yl}-2-[4-(6,7-dimethoxyquinazolin-4-yloxy)pyrazol-1-yl]acetamide

1-(3-Dimethylaminopropyl)-3-ethylcarbodiimide hydrochloride (0.105 g) was added to a mixture of 2-[4-(6,7-dimethoxyquinazolin-4-yloxy)pyrazol-1-yl]acetic acid (0.15 g), 2-amino-5-[N-(2-hydroxyethyl)-N-methylamino]pyridine (0.092 g), 2-hydroxypyridine N-oxide (0.061 g) and DMF (2 ml) and the resultant mixture was stirred at ambient temperature for 16 hours. Water was added and the precipitate was isolated, washed in turn with water and ethyl acetate, and purified by preparative HPLC using a Waters ‘β Basic Hypersil’ reversed-phase column (5 microns silica, 30 mm diameter, 250 mm length) and decreasingly polar mixtures of water (containing 0.2% ammonium carbonate) and acetonitrile as eluent. There was thus obtained the title compound (0.112 g); Mass Spectrum: M+H⁺ 480.

The 2-amino-5-[N-(2-hydroxyethyl)-N-methylamino]pyridine used as a starting material was prepared as follows: —

2-Methylaminoethanol (0.481 ml) was added to a stirred suspension of 5-bromo-2-nitropyridine (0.3 g) and ethanol (4 ml) and the mixture was heated to reflux for 3 days. The mixture was cooled to ambient temperature and concentrated by evaporation. The residue was partitioned between methylene chloride and water. The organic phase was dried over magnesium sulphate and evaporated. The resultant residue was purified by column chromatography on silica using increasingly polar mixtures of methylene chloride and ethyl acetate as eluent. There was thus obtained 5-[N-(2-hydroxyethyl)-N-methylamino]-2-nitropyridine (0.25 g); ¹H NMR: (CDCl₃) 1.84 (br s, 1H), 3.18 (s, 3H), 3.67 (m, 2H), 3.93 (m, 2H), 7.06 (m, 1H), 8.01 (m, 1H), 8.14 (m, 1H).

A mixture of the material so obtained, an excess of platinum oxide catalyst, ethanol (5 ml) and ethyl acetate (5 ml) was stirred under 5 atmospheres pressure of hydrogen for 3 hours. The catalyst was filtered off and the filtrate was evaporated. There was thus obtained 2-amino-5-[N-(2-hydroxyethyl)-N-methylamino]pyridine (0.205 g); ¹H NMR: (CDCl₃) 2.83 (s, 1H), 3.25 (t, 2H), 3.74 (t, 2H), 4.12 (br s, 2H), 6.48 (d, 1H), 7.14 (m, 1H), 7.74 (m, 1H); Mass Spectrum: M+H⁺ 168.

### EXAMPLE 33

Using an analogous procedure to that described in Example 32, the appropriate 2-(pyrazol-1-yl)acetic acid was reacted with the appropriate pyridylamine to give the compounds described in Table VIII. Unless otherwise stated, each pyridylamine starting material was commercially available.

TABLE VIII

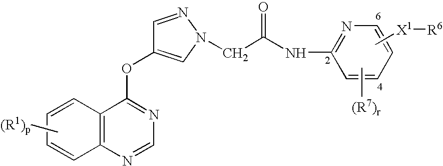

| No. & | | | |
|---|---|---|---|
| Note | (R¹)ₚ | (R⁷)ᵣ | X¹—R⁶ |
| [1] | 6,7-dimethoxy | H | 5-(3-hydroxypyrrolidin-1-yl) |
| [2] | 6,7-dimethoxy | H | 5-cyclopropylamino |
| [3] | 6,7-dimethoxy | H | 5-(2-methoxyethylamino) |
| [4] | 6-methoxy-7- | H | 5-cyclopropylamino |
| | (2-methoxyethoxy) | | |
| [5] | 6,7-di-(2- | H | 5-cyclopropylamino |
| | methoxyethoxy | | |
| [6] | 6,7-dimethoxy | H | 4-cyclopropylamino |
| [7] | 6,7-di- | H | 4-cyclopropylamino |
| | (2-methoxyethoxy | | |
| [8] | 6,7-dimethoxy | H | 4-pyrrolidin-1-yl |
| [9] | 6-methoxy-7- | H | 4-pyrrolidin-1-yl |
| | (2-methoxyethoxy) | | |
| [10] | 6,7-di- | H | 4-pyrrolidin-1-yl |
| | (2-methoxyethoxy | | |
| [11] | 6,7-dimethoxy | H | 4-morpholino |
| [12] | 6-methoxy-7- | H | 4-morpholino |
| | (2-methoxyethoxy) | | |
| [13] | 6,7-di- | H | 4-morpholino |
| | (2-methoxyethoxy) | | |
| [14] | 6,7-dimethoxy | H | 6-(2-methoxyethylamino) |
| [15] | 6-methoxy-7- | H | 6-(2-methoxyethylamino) |
| | (2-methoxyethoxy) | | |
| [16] | 6,7-di-(2- | H | 6-(2-methoxyethylamino) |
| | (2-methoxyethoxy) | | |
| [17] | 6,7-dimethoxy | H | 6-pyrrolidin-1-yl |
| [18] | 6-methoxy-7- | H | 6-pyrrolidin-1-yl |
| | (2-methoxyethoxy) | | |
| [19] | 6,7-di- | H | 6-pyrrolidin-1-yl |
| | (2-methoxyethoxy | | |
| [20] | 6,7-dimethoxy | H | 6-morpholino |
| [21] | 6-methoxy-7- | H | 6-morpholino |
| | (2-methoxyethoxy) | | |
| [22] | 6,7-di- | H | 6-morpholino |
| | (2-methoxyethoxy | | |

Notes The products gave the characterising data shown below.

[1] ¹H NMR: (DMSOd₆) 1.85-1.93 (m, 2H), 1.99-2.08 (m, 1H), 3.08 (m, 1H), 3.25-3.32 (m, 1H), 3.39-3.44 (m, 1H), 3.98 (s, 3H), 3.99 (s, 3H), 4.4 (br s, 1H), 4.97 (d, 1H), 5.05 (s, 2H), 6.98 (m, 1H), 6.39 (s, 1H), 6.54 (s, 1H), 7.66 (d, 1H), 7.67 (s, 1H), 7.86 (d, 1H), 8.11 (s, 1H), 8.66 (s, 1H), 10.42 (s, 1H); Mass Spectrum: M+H⁺ 492.

The 2-amino-5-(3-hydroxypyrrolidin-1-yl)pyridine used as a starting material was prepared as follows: —

Using analogous procedures to those described in the portion of Example 32 that is concerned with the preparation of starting materials, 5-bromo-2-nitropyridine was reacted with 3-hydroxypyrrolidine to give 5-(3-hydroxypyrrolidin-1-yl)-2-nitropyridine in 64% yield; ¹H NMR: (CDCl₃) 1.78 (s, 1H), 2.21 (m, 2H), 3.45 (d, 1H), 3.56 (m, 1H), 3.66 (m, 2H), 4.74 (br s, 1H), 6.67 (d, 1H), 7.84 (s, 1H), 8.18 (m, 1H); which in turn was converted into 2-amino-5-(3-hydroxypyrrolidin-1-yl)pyridine in 98% yield; ¹H NMR: (CDCl₃): 2.05 (m, 1H), 2.19 (m, 1H), 3.23 (m, 2H), 3.45 (m, 2H), 3.99 (br s, 2H), 4.59 (m, 1H), 6.49 (d, 1H), 6.85 (m, 1H), 7.49 (m, 1H).

[2] 2-Amino-5-(N-tert-butoxycarbonyl-N-cyclopropylamino)pyridine was used as the heteroarylamine. There was thus obtained N-[5-(N-tert-butoxycarbonyl-N-cyclopropylamino)pyridin-2-yl]-2-[4-(6,7-dimethoxyquinazolin-4-yloxy)pyrazol-1-yl]acetamide in 70% yield; ¹H NMR: (CDCl₃) 0.49 (m, 2H), 0.84 (m, 2H), 1.45 (s, 9H), 2.95 (m, 1H), 4.07 (s, 3H), 4.08 (s, 3H), 5.0 (s; 2H), 7.34 (s, 1-H), 7.5 (s, 1H), 7.58 (m, 1H), 7.88 (s, 1H), 8.03 (s, 1H), 8.15 (m, 1H), 8.68 (m, 1H), 8.71 (s, 1H); Mass Spectrum: M+H⁺ 562. The material so obtained was dissolved in methylene chloride and treated with a solution of 4N aqueous hydrochloric acid in 1,4-dioxane at ambient temperature for 1 hour. The mixture was evaporated and the residue was triturated under a 1:19 mixture of a 7M methanolic ammonia solution and methylene chloride. The resultant mixture was filtered and the filtrate was evaporated to give the required product in 61% yield which gave the following characterising data: —Mass Spectrum: M+H⁺ 462.

The 2-amino-5-(N-tert-butoxycarbonyl-N-cyclopropylamino)pyridine used as a starting material was prepared as follows: —

Cyclopropylamine (0.21 ml) was added to a stirred mixture of 5-bromo-2-nitropyridine (0.41 g), caesium carbonate (1.44 g), 1,1′-bis(diphenylphosphino)ferrocene (0.338 g), palladium(II) acetate (0.045 g) and toluene (10 ml) and the resultant mixture was heated to 90° C. for 30 minutes. The mixture was cooled to ambient temperature and filtered. The filtrate was evaporated and the residue was purified by column chromatography on silica using increasingly polar mixtures of methylene chloride and ethyl acetate as eluent. There was thus obtained 5-cyclopropylamino-2-nitropyridine (0.322 g); ¹H NMR: (CDCl₃) 0.62 (m, 2H), 0.91 (m, 2H), 2.57 (m, 1H), 4.96 (br s, 1H), 7.19 (m, 1H), 7.99 (d, 1H), 8.16 (m, 1H).

A mixture of 5-cyclopropylamino-2-nitropyridine (0.29 g), di-tert-butyl dicarbonate (0.39 g), 4-(N,N-dimethylamino)pyridine (0.02 g) and THF (10 ml) was stirred and heated to 75° C. for 2.5 hours. The solvent was evaporated and the residue was purified by column chromatography on silica using a gradient of 90:10 to 60:40 of petroleum ether (b.p. 40-60° C.) and ethyl acetate as eluent. There was thus obtained 5-(N-tert-butoxycarbonyl-N-cyclopropylamino)-2-nitropyridine (0.48 g); ¹H NMR: (CDCl₃) 0.59 (m, 2H), 1.05 (m, 2H), 1.53 (s, 9H), 3.02 (m, 1H), 8.05 (m, 1H), 8.22 (d, 1H), 8.64 (m, 1H).

A mixture of the material so obtained, platinium oxide (0.027 g), ethanol (10 ml) and ethyl acetate (10 ml) was stirred under 1.8 atmospheres pressure of hydrogen for 30 minutes. The mixture was filtered and the filtrate was evaporated. There was thus obtained 2-amino-5-(N-tert-butoxycarbonyl-N-cyclopropylamino)pyridine (0.349 g); ¹H NMR: (CDCl₃) 0.5 (m, 2H), 0.79 (m, 2H), 1.44 (s, 9H), 2.95 (m, 1H), 4.39 (br s, 2H), 6.46 (d, 1H), 7.24 (m, 1H), 7.87 (m, 1H); Mass Spectrum: M+W 250.

[3] 2-Amino-5-[N-tert-butoxycarbonyl-N-(2-methoxyethyl)amino]pyridine was used as the heteroarylamine. There was thus obtained N-[5-(2-methoxyethylamino)pyridin-2-yl]-2-[4-(6,7-dimethoxyquinazolin-4-yloxy)pyrazol-1-yl]acetamide in 54% yield; ¹H NMR: (CDCl₃) 1.41 (br s, 9H), 3.3 (s, 3H), 3.52 (t, 2H), 3.74 (t, 2H), 4.07 (s, 3H), 4.08 (s, 3H), 5.0 (s, 2H), 7.34 (s, 1H), 7.5 (s, 1H), 7.62 (m, 1H), 7.88 (s, 1H), 8.03 (s, 1H), 8.19 (m, 2H), 8.7 (m, 2H). The material so obtained was treated with hydrochloric acid using an analogous procedure to that described in Note [2] immediately above. There was thus obtained the required product in 56% yield which gave the following characterising data: —¹H NMR: (DMSOd₆) 3.2 (m, 2H), 3.28 (s, 3H), 3.49 (t, 2H), 3.98 (s, 3H), 3.99 (s, 3H), 5.04 (s, 2H), 5.69 (t, 1H), 7.03 (m, 1H), 7.39 (s, 1H), 7.54 (s, 1H), 7.67 (s, 1H), 7.75 (m, 2H), 8.11 (s, 1H), 8.66 (s, 1H); Mass Spectrum: M+H⁺ 462.

The 2-amino-5-[N-tert-butoxycarbonyl-N-(2-methoxyethyl)amino]pyridine used as a starting material was prepared as follows: —

Using analogous procedures to those described in the portion of Note [2] above that is concerned with the preparation of starting materials, 5-bromo-2-nitropyridine was reacted with 2-methoxyethylamine and converted into 5-(2-methoxyethylamino)-2-nitropyridine in 79% yield; ¹H NMR: (CDCl₃) 3.41 (t, 2H), 3.43 (s, 3H), 3.65 (t, 2H), 4.97 (br s, 1H), 6.95 (m, 1H), 7.91 (m, 1H), 8.15 (m, 1H); which in turn was converted into 5-[N-tert-butoxycarbonyl-N-(2-methoxyethyl)amino]-2-nitropyridine in 100% yield; ¹H NMR: (CDCl₃) 1.49 (s, 9H), 3.34 (s, 3H), 3.63 (t, 2H), 3.86 (t, 2H), 8.07 (m, 1H), 8.22 (d, 1H), 8.67 (m, 1H); and which in turn was converted into 2-amino-5-[N-tert-butoxycarbonyl-N-(2-methoxyethyl)amino]pyridine in 77% yield; ¹H NMR: (CDCl₃) 1.26 (br s, 9H), 3.32 (s, 3H), 3.5 (t, 2H), 3.71 (t, 2H), 4.44 (s, 2H), 6.47 (d, 1H), 7.3 (m, 1H), 7.93 (m, 1H); Mass Spectrum: M+H⁺ 268.

[4] ¹H NMR: (DMSOd₆) 0.35-0.43 (m, 2H), 0.67-0.75 (m, 2H), 2.31-2.39 (m, 1H), 3.35 (s, 3H), 3.75-3.79 (m, 2H), 3.99 (s, 3H), 4.3-4.37 (m, 2H), 5.06 (s, 2H), 7.16 (m, 1H), 7.42 (s, 1H), 7.55 (s, 1H), 7.68 (s, 1H), 7.79 (d, 1H), 7.81 (d, 1H), 8.12 (s, 1H), 8.66 (s, 1H), 10.51 (s, 1H); Mass Spectrum: M+H⁺ 506.

[5] ¹H NMR: (DMSOd₆) 0.36-0.42 (m, 2H), 0.68-0.74 (m, 2H), 2.32-2:39 (m, 1H), 3.36 (s, 3H), 3.37 (s, 3H), 3.75-3.79 (m, 4H) 4.29-4.4 (m, 4H), 5.07 (s, 2H), 7.22 (m, 1H), 7.42 (s, 1H), 7.59 (s, 1H), 7.68 (s, 1H), 7.76 (d, 1H), 7.8 (d, 1H), 8.13 (s, 1H), 8.68 (s, 1H), 10.64 (s, 1H); Mass Spectrum: M+H⁺ 550.

[6] ¹H NMR: (DMSOd₆) 0.36-0.43 (m, 2H), 0.67-0.74 (m, 2H), 2.31-2.39 (m, 1H), 3.98 (s, 3H), 3.99 (s, 3H), 5.07 (s, 2H), 6.38 (d, 1H), 6.98 (s, 1H), 7.39 (s, 1H), 7.47 (br s, 1H), 7.54 (s, 1H), 7.68 (s, 1H), 7.82 (d, 1H), 8.11 (s, 1H), 8.66 (s, 1H), 10.32 (s, 1H); Mass Spectrum: M+H⁺ 462.

The 2-amino-4-cyclopropylaminopyridine used as a starting material was prepared as follows: —

A mixture of 4-chloropyridine-2-carboxylic acid (1 g), cyclopropylamine (0.66 ml), diisopropylethylamine (2.2 ml) and water (1 ml) was heated to 170° C. in a microwave oven for 5 minutes. The mixture was cooled to ambient temperature and delivered to a preparative HPLC C18 reversed-phase column (a Waters ‘Oasis MCX 6’ column; 5 microns silica, 19 mm diameter, 100 mm length). The column was eluted with a gradient of 100:0 to 17:3 of water and methanol. There was thus obtained 4-cyclopropylaminopyridine-2-carboxylic acid (0.51 g); ¹H NMR: (DMSOd₆) 0.51 (m, 2H), 0.83 (m, 2H), 2.6 (m, 1H), 6.81 (m, 1H), 7.31 (m, 1H), 8.09 (m, 1H), 8.3 (m, 1H).

Diphenylphosphoryl azide (1.45 ml) and triethylamine (0.39 ml) were added in turn to a stirred mixture of 4-cyclopropylaminopyridine-2-carboxylic acid (0.5 g), tert-butanol (5 ml) and 1,4-dioxane (20 ml) and the resultant mixture was heated to reflux for 5 hours. The solvent was evaporated and the residue was purified by column chromatography on silica using a gradient of 50:50:0 to 9:9:2 of methylene chloride, ethyl acetate and methanol as eluent. There was thus obtained 2-(tert-butoxycarbonylamino)-4-cyclopropylaminopyridine (0.56 g); ¹H NMR: (CDCl₃) 0.54 (m, 2H), 0.82 (m, 2H), 1.51 (s, 9H), 2.51 (m, 1H), 4.65 (br s, 1H), 6.37 (m, 1H), 7.3 (m, 1H), 7.84 (m, 1H); Mass Spectrum: M+H 250.

The material so obtained was dissolved in methylene chloride and treated with a solution of 4N aqueous hydrochloric acid in 1,4-dioxane at ambient temperature for 1 hour. The mixture was evaporated and the residue was triturated under a 1:19 mixture of a 7M methanolic ammonia solution and methylene chloride. The resultant mixture was filtered and the filtrate was evaporated. The residue was purified by column chromatography on silica using a gradient of 19:1 of methylene chloride and methanol to 19:1 of methylene chloride and a 3M methanolic ammonia solution as eluent. There was thus obtained 2-amino-4-cyclopropylaminopyridine in 32% yield; ¹H NMR: (CDCl₃) 0.54 (m, 2H), 0.76 (m, 2H), 2.43 (m, 1H), 4.52 (br s, 1H), 4.57 (br s, 1H), 5.89 (m, 1H), 6.04 (m, 1H), 7.65 (m, 1H); Mass Spectrum: M+H⁺ 150.

[7] ¹H NMR: (DMSOd₆) 0.37-0.42 (m, 2H), 0.68-0.73 (m, 2H), 2.32-2.39 (m, 1H), 3.36 (s, 3H), 3.37 (s, 3H), 3.74-3.8 (m, 4H), 4.3-4.38 (m, 4H), 5.07 (s, 2H), 6.38 (m, 1H), 6.98 (s, 1H), 7.42 (s, 1H), 7.47 (br s, 1H), 7.59 (s, 1H), 7.68 (s, 1H), 7.83 (d, 1H), 8.12 (s, 1H), 8.66 (s, 1H), 10.33 (br s, 1H); Mass Spectrum: M+H⁺ 550.

[8] ¹H NMR: (DMSOd₆) 1.91-1.98 (m, 4H), 3.2-3.27 (m, 4H), 3.98 (s, 3H), 3.99 (s, 3H), 5.08 (s, 2H), 6.26 (m, 1H), 7.26 (br s, 1H), 7.4 (s, 1H), 7.54 (s, 1H), 7.68 (s, 1H), 7.87 (d, 1H), 8.11 (s, 1H), 8.66 (s, 1H), 10.38 (s, 1H); Mass Spectrum: M+H⁺ 476.

The 2-amino-4-pyrrolidin-1-ylpyridine used as a starting material was prepared as follows: —

A mixture of 2-amino-4-chloropyridine (Org. Prep. and Proced., 1997, 29, 117; 1 g) and pyrrolidine (2.59 ml) was heated to 205° C. in a microwave oven for 30 minutes. The reaction mixture was purified by column chromatography on silica using a 10:1 mixture of methylene chloride and a 3M methanolic ammonia solution as eluent. There was thus obtained the required starting material (1.05 g); ¹H NMR: (CDCl₃) 2.0 (m, 4H), 3.28 (m, 4H), 4.49 (br s, 2H), 5.56 (m, 1H), 5.95 (m, 1H), 7.69 (d, 1H); Mass Spectrum: M+H⁺ 164.

[9] ¹H NMR: (DMSOd₆) 198-2.01 (m, 4H), 3.19-3.29 (m, 4H), 3.35 (s, 3H), 3.73-3.81 (m, 2H), 3.99 (s, 3H), 4.29-4.37 (m, 2H), 5.08 (s, 2H), 6.27 (m, 1H), 7.27 (br s, 1H), 7.41 (s, 1H), 7.55 (s, 1H), 7.68 (s, 1H), 7.88 (d, 1H), 8.12 (s, 1H), 8.66 (s, 1H), 10.39 (s, 1H); Mass Spectrum: M+W 520.

[10] Mass Spectrum: M+H⁺ 564.

[11] ¹H NMR: (DMSOd₆) 3.19-3.27 (m, 4H), 3.67-3.75 (m, 4H), 3.98 (s, 3H), 3.99 (s, 3H), 5.09 (s, 2H), 6.65 (m, 1H), 7.39 (s, 1H), 7.54 (s, 1H), 7.61 (br s, 1H), 7.68 (s, 1H), 7.98 (d, 1H), 8.11 (s, 1H), 8.66 (s, 1H), 10.54 (s, 1H); Mass Spectrum: M+H 492.

The 2-amino-4-morpholinopyridine used as a starting material was prepared by the reaction of 2-amino-4-chloropyridine and morpholine using an analogous procedure to that described in Note [8] immediately above. The required starting material gave the following characterising data; ¹H NMR: (CDCl₃) 3.26 (m, 4H), 3.81 (m, 4H), 4.71 (br s, 2H), 5.87 (m, 1H), 6.19 (m, 1H), 7.76 (d, 1H); Mass Spectrum: M+H⁺ 180.

[12] ¹H NMR: (DMSOd₆) 3.21-3.25 (m, 4H), 3.35 (s, 3H), 3.68-3.72 (m, 4H), 3.74-3.79 (m, 2H), 3.98 (s, 3H), 4.3-4.35 (m, 2H), 5.09 (s, 2H), 6.65 (m, 1H), 7.41 (s, 1H), 7.54 (s, 1H), 7.61 (br s, 1H), 7.67 (s, 1H), 7.98 (s, 1H), 8.11 (s, 1H), 8.65 (s, 1H), 10.53 (br s, 1H); Mass Spectrum: M+H⁺ 536.

[13] ¹H NMR: (DMSOd₆) 3.2-3.27 (m, 4H), 3.36 (s, 3H), 3.37 (s, 3H), 3.67-3.73 (m, 4H), 3.74-3.8 (m, 4H), 4.29-4.37 (m, 4H), 5.1 (s, 2H), 6.66 (m, 1H), 7.42 (s, 1H), 7.58 (s, 1H), 7.62 (br s, 1H), 7.66 (s, 1H), 7.99 (d, 1H), 8.11 (s, 1H), 8.66 (s, 1H), 10.44 (br s, 1H); Mass Spectrum: M+H⁺ 580.

[14] ¹H NMR: (DMSOd₆) 3.28 (s, 3H), 3.41-3.5 (m, 4H), 3.98 (s, 3H), 3.99 (s, 3H), 5.08 (br s, 2H), 6.25 (d, 1H), 6.54 (t, 1H), 7.16 (br s, 1H), 7.34 (m, 1H), 7.39 (s, 1H), 7.54 (s, 1H), 7.67 (s, 1H), 8.1 (s, 1H), 8.66 (s, 1H), 10.14 (br s, 1H); Mass Spectrum: M−H⁻ 478.

The 2-amino-6-(2-methoxyethylamino)pyridine used as a starting material was prepared as follows: —

A mixture of 2-amino-6-chloropyridine (1.5 g), 2-methoxyethylamine (3.04 ml) and water (0.5 ml) was heated to 210° C. in a microwave oven for 30 minutes. The reaction mixture was purified by column chromatography on silica using increasingly polar mixtures of methylene chloride and ethyl acetate as eluent. There was thus obtained the required starting material (0.38 g); ¹H NMR: (CDCl₃) 3.76 (s, 3H), 3.42 (t, 2H), 3.56 (t, 2H), 4.14 (br s, 2H), 4.58 (br s, 1H), 5.79 (d, 1H), 5.82 (d, 1H), 7.21 (t, 1H).

[15] NH N: (DMSOd₆) 3.28 (s, 3H), 3.34 (s, 3H), 3.41-3.35 (m, 4H), 3.74-3.79 (m, 2H), 3.99 (s, 3H), 4.3-4.35 (m, 2H), 5.09 (s, 2H), 6.25 (d, 1H), 6.54 (t, 1H), 7.16 (br s, 1H), 7.34 (m, 1H), 7.41 (s, 1H), 7.55 (s, 1H), 7.67 (s, 1H), 8.1 (s, 1H), 8.65 (s, 1H), 10.14 (s, 1H); Mass Spectrum: M−H⁻ 522.

[16] ¹H NMR: (DMSOd₆) 3.28 (s, 3H), 3.36 (s, 6H), 3.4-3.49 (m, 4H), 3.74-3.8 (m, 4H), 4.3-4.37 (m, 4H), 5.09 (br s, 2H), 6.25 (d, 1H), 6.54 (t, 1H), 7.16 (br s, 1H), 7.34 (m, 1H), 7.42 (s, 1H), 7.58 (s, 1H), 7.67 (s, 1H), 8.1 (s, 1H), 8.65 (s, 1H), 10.14 (s, 1H); Mass Spectrum: M−H⁻ 566.

[17] ¹H NMR: (DMSOd₆) 1.89-1.99 (m, 4H), 3.35-3.43 (m, 4H), 3.98 (s, 3H), 3.99 (s, 3H), 5.10 (br s, 2H), 6.18 (d, 1H), 7.23 (br s, 1H), 7.39 (s, 1H), 7.46 (m, 1H), 7.54 (s, 1H), 7.67 (s, 1H), 8.11 (s, 1H), 8.66 (s, 1H), 10.21 (br s, 1H); Mass Spectrum: M+H⁺ 476.

The 2-amino-6-pyrrolidin-1-ylpyridine used as a starting material was prepared as follows: —

A mixture of 2-amino-6-chloropyridine (0.5 g), pyrrolidine (1.3 ml) and water (0.5 ml) was heated to 205° C. in a microwave oven for 30 minutes. The reaction mixture was purified by column chromatography on silica using increasingly polar mixtures of methylene chloride and ethyl acetate as eluent. There was thus obtained the required starting material (0.36 g); ¹H NMR: (CDCl₃) 1.94 (m, 4H), 3.95 (m, 4H), 4.15 (br s, 2H), 5.75 (m, 2H), 7.23 (m, 1H); Mass Spectrum: M+H⁺ 164.

[18] ¹H NMR: DMSOd₆) 1.88-2.01 (m, 4H), 3.33-3.39 (m, 4H), 3.38 (s, 3H), 3.73-3.8 (m, 2H), 3.98 (s, 3H), 4.28-4.39 (m, 2H), 5.1 (br s, 2H), 6.18 (d, 1H), 7.23 (br s, 1H), 7.41 (s, 1H), 7.46 (m, 1H), 7.55 (s, 1H), 7.67 (s, 1H), 8.11 (s, 1H), 8.65 (s, 1H), 10.21 (s, 1H); Mass Spectrum: M+H⁺ 520.

[19]. ¹H NMR: (DMSOd₆) 1.89-1.99 (m, 4H), 3.6 (s, 6H), 3.37-3.41 (m, 4H), 3.74-3.79 (m, 4H), 4.3-4.36 (m, 4H), 5.1 (br s, 2H), 6.18 (d, 1H) 7.23 (br s, 1H), 7.42 (s, 1H), 7.46 (m, 1H), 7.58 (s, 1H), 7.67 (s, 1H), 8.1 (s, 1H), 8.65 (s, 1H), 10.21 (s, 1H); Mass Spectrum: M+H⁺ 564.

[20] ¹H NMR: (DMSOd₆) 3.43-3.49 (m, 4H), 3.67-3.75 (m, 4H), 3.98 (s, 3H), 3.99 (s, 3H), 5.10 (s, 2H), 6.56 (d, 1H), 7.34 (br s, 1H), 7.39 (s, 1H), 7.54 (s, 1H), 7.56 (d, 1H), 7.67 (s, 1H), 8.11 (s, 1H), 8.6 (s, 1H), 10.33 (br s, 1H); Mass Spectrum: M−H⁻ 490.

The 2-amino-6-morpholinopyridine used as a starting material was prepared as follows: —

Using an analogous procedure to that described in the portion of Note [17] above that is concerned with the preparation of starting materials, 2-amino-6-chloropyridine was reacted with morpholine to give the required starting material in 69% yield; ¹H NMR: (CDCl₃) 3.43 (m, 4H), 3.79 (m, 4H), 4.2 (br s, 2H), 5.91 (d, 1H), 5.98 (d, 1H), 7.31 (t, 1H); Mass Spectrum: M+H⁺ 180.

[21] ¹H NMR: (DMSOd₆) 3.34 (s, 3H), 3.41-3.49 (m, 4H), 3.67-3.73 (m, 4H), 3.73-3.79 (m, 2H), 3.98 (s, 3H), 4.29-4.36 (m, 2H), 5.1 (s, 2H), 6.56 (d, 1H), 7.34 (br s, 1H), 7.41 (s, 1H), 7.52-7.59 (m, 2H), 7.67 (s, 1H), 8.11 (s, 1H), 8.65 (s, 1H), 10.32 (s, 1H); Mass Spectrum: M−H⁻ 534.

[22] ¹H NMR: (DMSOd₆) 3.36 (s, 6H), 3.43-3.48 (m, 4H), 3.68-3.73 (m, 4H), 3.74-3.79 (m, 4H), 4.29-4.38 (m, 4H), 5.1 (br s, 2H), 6.56 (d, 1H), 7.34 (br s, 1H), 7.42 (s, 1H), 7.56 (m, 1H), 7.58 (s, 1H), 7.67 (s, 1H), 8.11 (s, 1H), 8.65 (s, 1H), 10.32 (s, 1H); Mass Spectrum: M−H⁻ 578.

### EXAMPLE 34

### N-[5-cyclopropylpyrazol-3-yl)-2-[4-(6,7-dimethoxyquinazolin-4-yloxy)pyrazol-1-yl]acetamide

1-(3-Dimethylaminopropyl)-3-ethylcarbodiimide hydrochloride (0.13 g) and 2-hydroxypyridine N-oxide (0.076 g) were added in turn to a stirred mixture of 2-[4-(6,7-dimethoxyquinazolin-4-yloxy)pyrazol-1-yl]acetic acid (0.15 g) 3-amino-5-cyclopropylpyrazole (0.084 g), diisopropylethylamine (0.16 ml) and DMF (3 ml) and the resultant mixture was stirred at ambient temperature for 12 hours. Water was added and the precipitate was isolated and purified by preparative HPLC using a Waters ‘β Basic Hypersil’ reversed-phase column (5 microns silica, 30 mm diameter, 250 mm length) and decreasingly polar mixtures of water (containing 0.2% ammonium carbonate) and acetonitrile as eluent. There was thus obtained the title compound (0.09 g); ¹H NMR: (DMSOd₆) 0.61-0.67 (m, 2H), 0.87-0.94 (m, 2H), 1.82-1.9 (m, 1H), 3.98 (s, 3H), 3.99 (s, 3H), 4.98 (s, 2H), 6.14 (s, 1H), 7.39 (s, 1H), 7.54 (s; 1H), 7.65 (s, 1H), 8.09 (s, 1H), 8.65 (s, 1H), 10.63 (s, 1H); Mass Spectrum: M+H⁺ 436.

### EXAMPLE 35

Using an analogous procedure to that described in Example 34, the appropriate 2-(pyrazol-1-yl)acetic acid was reacted with the appropriate aniline or heteroarylamine to give the compounds described in Table IX. Unless otherwise stated, each aniline or heteroarylamine that was required as a starting material was commercially available.

TABLE IX

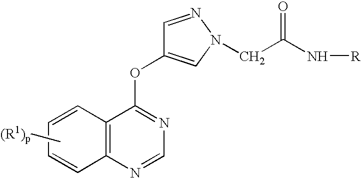

| No. & | | |
|---|---|---|
| Note | (R¹)ₚ | R |
| [1] | 6-methoxy-7-ethoxy | 3-hydroxymethylphenyl |
| [2] | 6-methoxy- | 3-hydroxymethylphenyl |
| | 7-(2-methoxyethoxy) | |
| [3] | 6-methoxy- | 3-oxo-1,3-dihydroisobenzofuran-5-yl |
| | 7-(2-methoxyethoxy) | |
| [4] | 6-methoxy-7-ethoxy | 3-oxo-1,3-dihydroisobenzofuran-5-yl |
| [5] | 6,7-dimethoxy | 4-chloro-3-(2-hydroxyethyl)phenyl |
| [6] | 6,7-dimethoxy | 4-[(2S)-2-hydroxypropylamino]- |
| | | 2-methoxyphenyl |
| [7] | 6,7-dimethoxy | 4-{N-[(2R)-2-hydroxypropyl]- |
| | | N-methylamino}-2-methoxyphenyl |
| [8] | 6,7-dimethoxy | 4-(2-hydroxyethylamino)-2- |
| | | methoxyphenyl |
| [9] | 6,7-dimethoxy | 3-cyclopropylaminophenyl |
| [10] | 6,7-dimethoxy | 4-oxazol-5-ylphenyl |
| [11] | 6-methoxy-7-ethoxy | 2,3-ethylenedioxyphenyl |
| [12] | 6-methoxy-7-ethoxy | 3,4-ethylenedioxyphenyl |
| [13] | 6-methoxy- | 2,3-methylenedioxyphenyl |
| | 7-(2-methoxyethoxy) | |
| [14] | 6-methoxy- | 3,4-methylenedioxyphenyl |
| | 7-(2-methoxyethoxy) | |
| [15] | 6,7-dimethoxy | 2-oxoindolin-5-yl |
| [16] | 6,7-dimethoxy | 2-oxoindolin-6-yl |
| [17] | 6,7-dimethoxy | 3-chloro-4-(2-methoxyethoxy)phenyl |
| [18] | 6,7-dimethoxy | 3-(3-methoxypropyl)-1H-pyrazol-5-yl |
| [19] | 6,7-dimethoxy | 6-(2-isobutoxyethoxy)pyridin-3-yl |
| [20] | 6,7-dimethoxy | 2-(2-isobutoxyethoxy)pyrimidin-5-yl |
| [21] | 6,7-dimethoxy | 2,3-methylenedioxypyridin-4-yl |
| [22] | 6,7-dimethoxy | 2,3-dihydrofuro[3,2-b]pyridin-5-yl |
| [23] | 6,7-dimethoxy | 1-(2-methoxyethyl)-1H-pyrrolo[3,2-b] |
| | | pyridin-5-yl |
| [24] | 6,7-dimethoxy | 1-(2-pyrrolidin-1-ylethyl)indol-6-yl |
| [25] | 6,7-dimethoxy | 1-(2-pyrrolidin-1-ylethyl)indol-5-yl |

Notes The products gave the characterising data shown below.

[1] ¹H NMR: (DMSOd₆) 1.44 (t, 3H), 3.99 (s, 3H), 4.26 (q, 2H), 4.48 (d, 2H), 5.04 (s, 2H), 5.22 (t, 1H), 7.02 (d, 1H), 7.28 (m, 1H), 7.37 (s, 1H), 7.5 (d, 1H), 7.54 (s, 1H), 7.57 (br s, 1H), 7.68 (s, 1H), 8.13 (s, 1H), 8.65 (s, 1H), 10.32 (br s, 1H); Mass Spectrum: M+H⁺ 450.

[2] ¹H NMR: (DMSOd₆) 3.35 (s, 3H), 3.74-3.8 (m, 2H), 3.99 (s, 3H), 4.31-4.36 (m, 2H), 4.48 (d, 2H), 5.04 (s, 2H), 5.22 (t, 1H), 7.03 (d, 1H), 7.28 (m, 1H), 7.41 (s, 1H), 7.5 (d, 1H), 7.55 (s, 1H), 7.57 (br s, 1H), 7.68 (s, 1H), 8.12 (s, 1H), 8.66 (s, 1H), 10.32 (s, 1H); Mass Spectrum: M+H⁺ 480.

[3] ¹H NMR: (DMSOd₆) 3.35 (s, 3H), 3.74-3.79 (m, 2H), 3.99 (s, 3H), 4.31-4.36 (m, 2H), 5.10 (s, 2H), 5.39 (s, 2H), 7.41 (s, 1H), 7.56 (s, 1H), 7.66 (d, 1H), 7.7 (s, 1H), 7.86 (m, 1H), 8.15 (s, 1H), 8.2 (d, 1H), 8.66 (s, 1H), 10.73 (s, 1H); Mass Spectrum: M+H⁺ 506.

[4] ¹H NMR: (DMSOd₆) 1.44 (t, 3H), 3.99 (s, 3H), 4.26 (q, 2H), 5.1 (s, 2H), 5.39 (s, 2H), 7.37 (s, 1H), 7.55 (s, 1H), 7.66 (d, 1H), 7.7 (s, 1H), 7.85 (m, 1H), 8.15 (s, 1H), 8.2 (d, 1H), 8.65 (s, 1H), 10.73 (s, 1H); Mass Spectrum: M+H⁺ 476.

[5] ¹H NMR: (DMSOd₆) 2.82 (t, 2H), 3.56-3.64 (m, 2H), 3.98 (s, 3H), 3.99 (s, 3H), 4.77 (t, 1H), 5.04 (s, 2H), 7.36 (d, 1H), 7.39 (s, 1H), 7.51 (m, 1H), 7.54 (s, 1H), 7.59 (d, 1H), 7.68 (s, 1H), 8.12 (s, 1H), 8.66 (s, 1H), 10.44 (br s, 1H); Mass Spectrum: M+H⁺ 484 and 486.

The 4-chloro-3-(2-hydroxyethyl)aniline used as starting material was prepared as follows: —

Diborane (1M in THF, 80 ml) was added dropwise to a stirred mixture of 2-(2-chloro-5-nitrophenyl)acetic acid (International Application WO 02/28825; 8.64 g) and THF (50 ml) and the reaction mixture was stirred at ambient temperature for 12 hours. Water was added slowly to the reaction mixture followed by 2N aqueous hydrochloric acid solution. The mixture was extracted with diethyl ether and the organic phase was dried over magnesium sulphate and evaporated. The residue was purified by column chromatography on silica using a 20:1 mixture of methylene chloride and methanol as eluent. There was thus obtained 2-(2-chloro-5-nitrophenyl)ethanol as a solid (6.4 g); ¹H NMR: (DMSOd₆) 2.97 (t, 2H), 3.69 (q, 2H), 4.82 (t, 1H), 7.73 (d, 1H), 8.09 (m, 1H), 8.24 (m, 1H).

A mixture of a portion (1 g) of the material so obtained, platinium on carbon catalyst (0.15 g) and ethyl acetate (20 ml) was stirred under 1.8 atmospheres pressure of hydrogen for 30 minutes. The reaction mixture was filtered and the filtrate was evaporated. There was thus obtained 4-chloro-3-(2-hydroxyethyl)aniline (0.86 g); ¹H NMR: (DMSOd₆) 2.68 (t, 2H), 3.53 (q, 2H), 4.69 (t, 1H), 5.3 (s, 2H), 6.4 (m, 1H), 6.5 (s, 1H), 6.97 (m, 1H); Mass Spectrum: M+H⁺ 172.

[6] ¹H NMR: (DMSOd₆) 1.11 (d, 3H), 2.88-3.0 (m, 2H), 3.76 (s, 3H), 3.78 (d, 1H), 3.98 (s, 3H), 3.99 (s, 3H), 4.68 (d, 1H), 5.03 (s, 2H), 5.39-5.44 (m, 1H), 6.12 (m, 1H), 6.32 (d, 1H), 7.39 (s, 1H), 7.5 (d, 1H), 7.54 (s, 1H), 7.7 (d, 1H), 8.11 (s, 1H), 8.6 (s, 1H), 9.1 (s, 1H); Mass Spectrum: M+H⁺ 509.

The 4-[(2S)-2-hydroxypropylamino]-2-methoxyaniline used as starting material was prepared as follows: —

(2S)-2-Hydroxypropylamine (0.164 g) was added to a stirred mixture of 4-fluoro-2-methoxy-1-nitrobenzene (J. Med. Chem., 1997, 2674; 0.25 g), potassium carbonate (0.3 g) and DMF (2 ml) and the reaction mixture was stirred at ambient temperature for 2 days. Water (10 ml) was added and the mixture was extracted with ethyl acetate. The organic phase was dried over magnesium sulphate and evaporated. The residue was purified by column chromatography on silica using a gradient of 100:0 to 95:5 of methylene chloride and methanol as eluent. There was thus obtained 4-[(2S)-2-hydroxypropylamino]-2-methoxynitrobenzene (0.283 g); ¹H NMR: (CDCl₃) 1.32 (d, 3H), 1.59 (br s, 1H), 3.1 (m, 1H), 3.32 (m, 1H), 3.93 (s, 3H), 4.1 (m, 1H), 6.11 (s, 1H), 6.16 (m, 1H), 7.97 (d, 1H).

A mixture of the material so obtained, platinium oxide (0.027 g) and ethyl acetate was stirred under 1.5 atmospheres pressure of hydrogen at ambient temperature for 1 hour. The mixture was filtered and the filtrate was evaporated. There was thus obtained 4-[(2S)-2-hydroxypropylamino]-2-methoxyaniline (0.26 g); ¹H NMR: (DMSOd₆) 1.09 (d, 3H), 2.83 (m, 2H), 3.69 (s, 1H), 3.75 (m, 1H), 3.88 (s, 2H), 4.58 (t, 1H), 4.6 (d, 1H), 5.98 (m, 1H), 6.22 (m, 1H), 6.43 (d, 1H); Mass Spectrum: M+H⁺ 197.

[7] ¹H NMR: (DMSOd₆) 1.07 (d, 3H), 2.94 (s, 3H), 3.21 (d, 2H), 3.91 (s, 3H), 3.84-3.92 (m, 1H), 3.98 (s, 3H), 3.99 (s, 3H), 4.66 (d, 1H), 5.04 (s, 2H), 6.12 (m, 1H), 6.32 (d, 1H), 7.39 (s, 1H), 7.54 (s, 1H), 7.6 (d, 1H), 7.7 (s, 1H), 8.12 (s, 1H), 8.6 (s, 1H), 9.17 (s, 1H); Mass Spectrum: M+H⁺ 523.

The 4-{N-[(2R)-2-hydroxypropyl]-N-methylamino}-2-methoxyaniline used as starting material was prepared as follows: —

Using analogous procedures to those described in the portion of Note [6] above that is concerned with the preparation of starting materials, 4-fluoro-2-methoxy-1-nitrobenzene was reacted with N-[(2R)-2-hydroxypropyl]-N-methylamine and converted into 4-[N-[(2R)-2-hydroxypropyl]-N-methylamino}-2-methoxynitrobenzene in 92% yield; ¹H NMR: (CDCl₃) 1.27 (d, 3H), 1.75 (br s, 1H), 3.14 (s, 3H), 3.41 (s, 2H), 3.94 (s, 3H), 4.18 (q, 1H), 6.17 (m, 1H), 6.27 (m, 1H), 7.98 (d, 1 μl); which in turn was converted by reduction to the required starting material in 100% yield; ¹H NMR: (DMSOd₆) 1.05 (d, 3H), 2.8 (s, 3H), 3.03 (m, 2H), 3.73 (s, 3H), 3.83 (m, 1H), 3.98 (s, 2H), 4.53 (s, 1H), 6.1 (m, 1H), 6.29 (m, 1H), 6.5 (d, 1H); Mass Spectrum: M+H⁺ 211.

[8] ¹H NMR: (DMSOd₆) 3.04-3.12 (m, 2H), 3.51-3.59 (m, 2H), 3.76 (s, 3H), 3.98 (s, 3H), 3.99 (s, 3H), 4.65-4.72 (m, 1H), 5.02 (s, 2H), 5.42-5.48 (m, 1H), 6.11 (m, 1H), 6.31 (d, 1H), 7.39 (s, 1H), 7.51 (d, 1H), 7.54 (s, 1H), 7.69 (s, 1H), 8.11 (s, 1H), 8.65 (s, 1H), 9.11 (s, 1H); Mass Spectrum: M+H⁺ 495.

The 4-(2-hydroxyethylamino)-2-methoxyaniline used as starting material was prepared as follows: —

Using analogous procedures to those described in the portion of Note [6] above that is concerned with the preparation of starting materials, 4-fluoro-2-methoxy-1-nitrobenzene was reacted with 2-hydroxyethylamine and converted into 4-(2-hydroxyethylamino)-2-methoxynitrobenzene in 79% yield; ¹H NMR: (CDCl₃) δ: 39 (t, 2H), 3.91 (t, 2H), 3.93 (s, 3H), 6.12 (m, 1H), 6.19 (m, 1H), 7.99 (d, 1H); which in turn was converted by reduction to the required starting material in 78% yield; ¹H NMR: (DMSOd₆) 2.98 (m, 2H), 3.52 (m, 2H), 3.88 (s, 2H), 4.6 (m, 2H), 5.98 (m, 1H), 6.21 (m, 1H), 6.42 (m, 1H); Mass Spectrum: M+H⁺ 183.

[9] ¹H NMR: (DMSOd₆) 0.33-0.4 (m, 2H), 0.62-0.7 (m, 2H), 2.24-2.32 (m, 1H), 3.98 (s, 3H), 3.99 (s, 3H), 5.0 (s, 2H), 6.12 (s, 1H), 6.42 (m, 1H), 6.82 (m, 1H), 7.01 (m, 1H), 7.06 (br s, 1H), 7.39 (s, 1H), 7.54 (s, 1H), 7.67 (s, 1H), 8.11 (s, 1H), 8.66 (s, 1H), 10.08 (s, 1H); Mass Spectrum: M+H⁺ 461.

The 3-cyclopropylaminoaniline used as starting material was prepared as follows: —

(1-Ethoxycyclopropyloxy)trimethylsilane (0.736 ml) was added to a mixture of 3′-aminoacetanilide (0.5 g), acetic acid (1.9 ml), molecular sieves 3 (0.5 g) and methanol (15 ml) and the reaction mixture was stirred at ambient temperature for 30 minutes. Sodium cyanoborohydride (0.943 g) was added and the resultant mixture was heated to reflux for 16 hours. The mixture was cooled to ambient temperature and filtered. The filtrate was evaporated and the residue was purified by column chromatography on silica using a 3:1 mixture of methylene chloride and ethyl acetate as eluent. There was thus obtained 3′-cyclopropylaminoacetanilide (0.18 g); ¹H NMR: (CDCl₃) 0.51 (m, 2H), 0.73 (m, 2H), 2.16 (s, 3H), 2.42 (m, 1H), 4.3 (br s, 1H), 6.53 (m, 1H), 6.7 (m, 1H), 7.1 (m, 2H), 7.16 (s, 1H).

A mixture of the material so obtained, 6N aqueous sodium hydroxide solution (2 ml) and ethanol (6 ml) was heated to 80° C. for 7 hours. The mixture was neutralised to pH7 with 6N aqueous hydrochloric acid and extracted with ethyl acetate. The organic extract was dried over magnesium sulphate and evaporated. The residue was purified by column chromatography on silica using a solvent gradient of 19:1 to 9:1 of methylene chloride and ethyl acetate as eluent. There was thus obtained 3-cyclopropylaminoaniline (0.094 g); ¹H NMR: (CDCl₃) 0.5 (m, 2H), 0.69 (m, 2H), 2.4 (m, 1H), 6.1 (m, 1H), 6.2 (m, 2H), 6.96 (t, 1H); Mass Spectrum: M+H⁺ 149.

[10] ¹H NMR: (DMSOd₆+CF₃CO₂D) 4.03 (s, 3H), 4.05 (s, 3H), 5.1 (s, 2H), 7.5 (s, 1H), 7.54 (s, 1H), 6.67 (d, 2H), 7.7-7.74 (m, 4H), 8.19 (s, 1H), 8.34 (s, 1H), 9.23 (s, 1H); Mass Spectrum: M+H⁺ 473.

[11] 2,3-Ethylenedioxyaniline (J. Med. Chem., 1995, 38, 4044) was used as a starting material. The product gave the following characterising data: —¹H NMR: (DMSOd₆) 1.43 (t, 3H), 3.98 (s, 3H), 4.21-4.31 (m, 4H), 4.31-4.37 (m, 2H), 5.13 (s, 2H), 6.64 (d, 1H), 6.77 (m, 1H), 7.36 (s, 1H), 7.52-7.56 (m, 2H), 7.7 (s, 1H), 8.12 (s, 1H), 8.64 (s, 1H), 9.52 (br s, 1H); Mass Spectrum: M+H⁺ 478.

[12] ¹H NMR: (DMSOd₆) 1.43 (t, 3H), 3.98 (s, 3H), 4.17-4.29 (m, 6H), 4.98 (s, 2H), 6.81 (d, 1H), 6.98 (m, 1H), 7.23 (d, 1H), 7.37 (s, 1H), 7.54 (s, 1H), 7.67 (s, 1H), 8.11 (s, 1H), 8.64 (s, 1H), 10.18 (s, 1H); Mass Spectrum: M+H⁺ 478.

[13] ¹H NMR: (DMSOd₆) 3.36 (s, 3H), 3.75-3.8 (m, 2H), 4.0 (s, 3H), 4.32-4.37 (m, 2H), 5.11 (s, 2H), 6.08 (s, 2H), 6.76 (d, 1H), 6.83 (m, 1H), 7.32 (d, 1H), 7.42 (s, 1H), 7.56 (s, 1H), 7.69 (s, 1H), 8.13 (s, 1H), 8.66 (s, 1H), 10.1 (br s, 1H); Mass Spectrum: M+H⁺ 494.

[14] ¹H NMR: (DMSOd₆) 3.35 (s, 3H), 3.73-3.79 (m, 2H), 3.99 (s, 3H), 4.3-4.35 (m, 2H), 5.0 (s, 2H), 6.0 (s, 2H), 6.88 (d, 1H), 6.98 (m, 1H), 7.3 (d, 1H), 7.41 (s, 1H), 7.54 (s, 1H), 7.67 (s, 1H), 8.11 (s, 1H), 8.65 (s, 1H), 10.27 (br s, 1H); Mass Spectrum: M+H⁺ 494.

[15] ¹H NMR: (DMSOd₆) 3.48 (s, 2H), 3.98 (s, 3H), 3.99 (s, 3H), 5.01 (s, 2H), 6.77 (d, 1H), 7.38 (m, 1H), 7.39 (s, 1H), 7.5 (s, 1H), 7.54 (s, 1H), 7.68 (s, 1H), 8.12 (s, 1H), 8.66 (s, 1H), 10.2 (s, 1H), 10.32 (s, 1H); Mass Spectrum: M+H⁺ 461.

[16] ¹H NMR: (DMSOd₆) 3.42 (s, 2H), 3.98 (s, 3H), 3.99 (s, 3H), 5.03 (s; 2H), 7.05 (m, 1H), 7.14 (d, 1H), 7.32 (d, 1H), 7.4 (s, 1H), 7.55 (s, 1H), 7.68 (s, 1H), 8.12 (s, 1H), 8.66 (s, 1H), 10.31 (s, 1H), 10.41 (s, 1H); Mass Spectrum: M+H⁺ 461.

The 6-amino-2-oxoindoline used as a starting material is described in German Patent Application DE 10228090.

[17] ¹H NMR: (DMSOd₆) 3.3 (s, 3H), 3.65 (m, 2H), 4.0 (m, 6H), 4.15 (m, 2H), 5.0 (s, 2H), 7.15 (d, 1H), 7.4 (s, 1H), 7.45 (m, 1H), 7.55 (s, 1H), 7.7 (s, 1H), 7.75 (m, 1H), 8.1 (s, 1H), 8.65 (s, 1H); Mass Spectrum: M+H⁺ 514.

The 3-chloro-4-(2-methoxyethoxy)aniline used as a starting material was prepared from 3-chloro-4-fluoronitrobenzene using analogous procedures to those described in International Patent Application WO 95/15952 (Example 3 thereof). 3-Chloro-4-fluoronitrobenzene was reacted with 2-methoxyethanol to give 3-chloro-4-(2-methoxyethoxy)nitrobenzene which in turn was reduced to give 3-chloro-4-(2-methoxyethoxy)aniline; Mass Spectrum: M+H⁺ 201.

[18] The reaction mixture was heated to 45° C. for 2.5 hours. Water (5 ml) was added and the solid was collected by filtration, washed with diethyl ether and dried overnight at 40° C. under vacuum. The product so obtained gave the following characterising data: —¹H NMR: (DMSOd₆) 1.8 (m, 2H), 2.6 (m, 2H), 3.2 (s, 3H), 3.3 (m, 2H), 4.0 (m, 6H), 5.0 (s, 2H), 6.25 (s, 1H), 7.4 (s, 1H), 7.55 (s, 1H), 7.65 (s, 1H), 8.1 (s, 1H), 8.65 (s, 1H); Mass Spectrum: M+H⁺ 468.

The 5-amino-3-(3-methoxypropyl)-1H-pyrazole used as a starting material was prepared as follows: —

Acetonitrile (11.48 ml) was added to a stirred suspension of sodium hydride (60% dispersion in mineral oil; 8.79 g) in anhydrous 1,4-dioxane (200 ml) and the mixture was stirred at ambient temperature for 30 minutes. Methyl 4-methoxybutyrate (25 ml) was added was added and the resultant mixture was heated to 100° C. for 12 hours. The reaction mixture was evaporated and the residue was partitioned between methylene chloride and water. The aqueous layer was acidified to pH2 with concentrated hydrochloric acid and extracted with methylene chloride. The organic solution was evaporated. A mixture of the residual oil so obtained (25.8 g), hydrazine monohydrate (10.6 ml) and ethanol (100 ml) was heated to 70° C. for 12 hours. The resultant mixture was evaporated and the residue was purified by column chromatography on silica using increasingly polar mixtures of methylene chloride and methanol as eluent. There was thus obtained 5-amino-3-(3-methoxypropyl)-1H-pyrazole as an oil (4.88 g); ¹H NMR: (CDCl₃) 1.85 (q, 2H), 2.6 (t, 2H), 3.35 (s, 3H), 3.4 (t, 2H), 5.4 (s, 1H).

[19] ¹H NMR: (DMSOd₆) 0.85 (d, 6H), 1.8 (m, 1H), 3.2 (d, 2H), 3.7 (m, 2H), 4.0 (m, 6H), 4.35 (m, 2H), 5.05 (s, 2H), 6.85 (d, 1H), 7.4 (s, 1H), 7.55 (s, 1H), 7.65 (s, 1H), 7.9 (m, 1H), 8.1 (s, 1H), 8.35 (m, 1H), 8.65 (s, 1H); Mass Spectrum: M+H⁺ 523.

The 5-amino-2-(2-isobutoxyethoxy)pyridine used as a starting material was prepared as follows: —

2-Isobutoxyethanol (6.98 g) was added dropwise to a stirred suspension of sodium hydride [60% dispersion in mineral oil, 2.36 g (subsequently-washed with pentane)] in DMA (50 ml) and the mixture was stirred at ambient temperature until the evolution of hydrogen gas had ceased. The solution so obtained was added dropwise over a 15 minute period to a stirred solution of 2-bromo-5-nitropyridine (10 g) in DMA (40 ml) that had been cooled to 0° C. The resultant mixture was stirred 0° C. for 15 minutes and allowed to warm to ambient temperature during a further 15 minutes. The mixture was poured into an ice water mixture and the resultant mixture was extracted with ethyl acetate. The organic phase was washed with brine, dried over magnesium sulphate, and evaporated. There was thus obtained 2-(2-isobutoxyethoxy)-5-nitropyridine (containing traces of DMA and 2-isobutoxyethanol) as an oil (12.9 g); ¹H NMR: (CDCl₃) 0.91 (d, 6H), 1.89 (m, 1H), 3.27 (d, 2H), 3.79 (m, 2H), 4.59 (m, 2H), 6.87 (d, 1H), 8.34 (m, 1H), 9.06 (d, 1H); Mass Spectrum: M+H⁺ 241.

A mixture of material so obtained, 10% palladium-on-carbon catalyst (1 g) and ethanol (320 ml) was stirred under 1.3 atmospheres pressure of hydrogen for 1 hour. The catalyst was removed by filtration and the solvent was evaporated. The residue was purified by column chromatography on silica using increasingly polar mixtures of methylene chloride and ethyl acetate (from a 1:1 mixture to a 2:3 mixture) as eluent. There was thus obtained 5-amino-2-(2-isobutoxyethoxy)pyridine as an oil (8.74 g); ¹H NMR: (CDCl₃) 0.91 (d, 6H), 1.89 (m, 1H), 3.27 (d, 2H), 3.75 (m, 2H), 4.37 (m, 2H), 6.65 (d, 1H), 7.03 (m, 1H), 7.63 (m, 1H); Mass Spectrum: M+H⁺ 211.

[20] ¹H NMR: (DMSOd₆) 0.85 (d, 6H), 1.8 (m, 1H), 3.2 (d, 2H), 3.7 (m, 2H), 4.0 (m, 6H), 4.4 (m, 2H), 5.1 (s, 2H), 7.4 (s, 1H), 7.55 (s, 1H), 7.7 (s, 1H), 8.15 (s, 1M), 8.65 (s, 1H), 8.75 (s, 2H); Mass Spectrum: M+H⁺ 524.

The 5-amino-2-(2-isobutoxyethoxy)pyrimidine used as a starting material was prepared as follows: —

2-Isobutoxyethanol (1.53 g) was added to a stirred suspension of sodium hydride (50% dispersion in mineral oil, 0.62 g) in THF (30 ml) and the mixture was stirred at ambient temperature for 10 minutes. 5-Bromo-2-chloropyrimidine (3.9 g) was added and the resultant mixture was heated to reflux for 1 hour. The mixture was cooled to ambient temperature and a saturated aqueous ammonium chloride solution (80 ml) was added. The mixture was extracted with diethyl ether and the organic extract was dried over magnesium sulphate and evaporated. The residual oil was purified by column chromatography on silica using a 3:2 mixture of isohexane and diethyl ether as eluent. There was thus obtained 5-bromo-2-(2-isobutoxyethoxy)pyrimidine as an oil (2.35 g).

A mixture of the material so obtained, benzophenone imine (1.63 g), palladium acetate (0.058 g), 9,9-dimethyl-4,5-bis(diphenylphosphino)xanthene (0.22 g), caesium carbonate (4.2 g) and 1,4-dioxane (30 ml) was stirred and heated to reflux for 5 hours. The mixture was cooled to ambient temperature and partitioned between methylene chloride and a saturated aqueous ammonium chloride solution. The organic solution was dried over magnesium sulphate and evaporated. The residual oil so obtained was dissolved in ethyl acetate (20 ml) and a 2N aqueous hydrochloric acid solution (60 ml) was added and the resultant mixture was stirred vigorously for 1 hour. The aqueous layer was separated, washed with ethyl acetate, basified by the addition of a saturated aqueous potassium carbonate solution and extracted with diethyl ether. The organic extract was dried over magnesium sulphate and evaporated. The residual oil was purified by column chromatography on silica using increasingly polar mixtures of diethyl ether and methanol as eluent. The oil so obtained was triturated under a mixture of isohexane and diethyl ether. The resultant solid was collected and dried under vacuum. There was thus obtained 5-amino-2-(2-isobutoxyethoxy)pyridine (1.22 g); NMR Spectrum: (CDCl₃) 0.89 (d, 6H), 1.87 (m, 1H), 3.28 (d, 2H), 3.39 (s, 2H), 3.78 (t, 2H), 4.42 (t, 2H), 8.03 (s, 2H); Mass Spectrum: M+H⁺ 212.

[21] The reaction mixture was heated to 40° C. for 16 hours. The reaction mixture was cooled to ambient temperature. The precipitated solid was collected by filtration and washed with diethyl ether. The material so obtained was suspended in a 10% aqueous sodium bicarbonate solution and stirred for 1 hour. The resultant solid was collected by filtration, washed with water and dried overnight under vacuum. The product so obtained gave the following characterising data: —¹H NMR: (DMSOd₆) 4.0 (m, 6H), 5.15 (s, 2H), 6.2 (s, 2H), 7.4 (s, 1H), 7.5 (m, 3H), 7.7 (s, 1H), 8.15 (s, 1H), 8.7 (s, 1H); Mass Spectrum: M+H⁺ 451.

4-Amino-2,3-methylenedioxypyridine that was used as a starting material is described in International Patent Application WO 2004/041829 (Example 14 thereof).

[22] ¹H NMR: (DMSOd₆) 3.2 (t, 2H), 3.98 (s, 3H), 3.99 (s, 3H), 4.65 (t, 2H), 5.05 (s, 2H), 7.2 (d, 1H), 7.4 (s, 1H), 7.55 (s, 1H), 7.7 (s, 1H), 7.8 (d, 1H), 8.1 (s, 1H), 8.65 (s, 1H), 10.65 (s, 1H); Mass Spectrum: M−H⁻ 447.

5-Amino-2,3-dihydrofuro[3,2-b]pyridine that was used as a starting material is described in Chem. Pharm. Bull., 1984, 32, 4914.

[23] The reaction product was purified by column chromatography on silica using a 47:3 mixture of methylene chloride and methanol as eluent. The product so obtained gave the following characterising data: —¹H NMR: (DMSOd₆) 3.2 (s, 3H), 3.65 (t, 2H), 3.98 (s, 3H), 3.99 (s, 3H), 4.35 (t, 2H); 5.1 (s, 2H), 6.45 (d, 1H), 7.4 (s, 1H), 7.55 (s, 1H), 7.6 (d, 1H), 7.65 (s, 1H), 7.9 (d, 1H), 7.95 (d, 1H), 8.15 (s, 1H), 8.65 (s, 1H), 10.65 (s, 1H); Mass Spectrum: M+H⁺ 504.

The 5-amino-1-(2-methoxyethyl)-1H-pyrrolo[3,2-b]pyridine used as a starting material was prepared as follows: —

Sodium hydride (60% dispersion in oil; 0.126 g) was added to a stirred mixture of N¹,N¹-dimethyl-N²-(1H-pyrrolo[3,2-b]pyridin-5-yl)formamidine (J. Med. Chem., 2003, 46, 3060; 0.564 g) and DMF (10 ml) and the reaction mixture was stirred at ambient temperature for 10 minutes. 2-Methoxyethyl bromide (0.282 ml) was added and the reaction mixture was stirred at ambient temperature for 1 hour. Water was added and the mixture was extracted with methylene chloride. The organic phase was washed with water and with brine, dried over magnesium sulphate and evaporated. There was thus obtained N¹,N¹-dimethyl-N²-[1-(2-methoxyethyl)-1H-pyrrolo[3,2-b]pyridin-5-yl]formamidine (0.537 g); ¹H NMR: (DMSOd₆) 3.1 (s, 6H), 3.3 (s, 3H), 3.7 (t, 2H), 4.2 (t, 2H), 6.5 (d, 1H), 6.9 (d, 1H), 7.25 (d, 1H), 7.55 (d, 1H), 7.7 (d, 1H), 8.4 (s, 1H).

A mixture of the material so obtained, potassium hydroxide (0.302 g), water (1 ml) and methanol (5 ml) was stirred and heated to 75° C. for 16 hours. The resultant mixture was diluted with water and extracted with a mixture of methylene chloride and methanol. The organic phase was washed with water and with brine, dried over magnesium sulphate and evaporated. There was thus obtained 5-amino-1-(2-methoxyethyl)-1H-pyrrolo[3,2-b]pyridine (0.29 g); ¹H NMR: (DMSOd₆) 3.2 (s, 3M), 3.6 (t, 2H), 4.2 (t, 2H), 5.35 (s, 2H), 6.1 (d, 1H), 6.3 (d, 1H), 7.3 (d, 1H), 7.55 (d, 1H).

[24] The reaction mixture was heated to 100° C. in a microwave oven for 4 minutes. The reaction mixture was cooled to ambient temperature and the precipitated solid was collected by filtration and purified by preparative HPLC as described in Example 34. The product so obtained gave the following characterising data: —¹H NMR: (DMSOd₆) 1.65 (br s, 4H), 2.45 (br s, 4H), 2.75 (t, 2H), 3.95 (s, 6H), 4.2 (t, 2H), 5.05 (s, 2H), 6.35 (d, 1H), 7.1 (d, 1H), 7.35 (d, 1H), 7.4 (s, 1H), 7.45 (d, 1H), 7.55 (s, 1H), 7.7 (s, 1H), 8.0 (s, 1H), 8.15 (s, 1H), 8.7 (s, 1H), 10.3 (s, 1H); Mass Spectrum: M+H⁺ 542.

The 6-amino-1-(2-pyrrolidin-1-ylethyl)indole used as a starting material was prepared as follows: —

A solution of 6-nitroindole (3.25 g) in DMF (12 ml) was added dropwise to a stirred suspension of sodium hydride (60% dispersion in mineral oil; 1.69 g) in DMF (75 ml) and the mixture was stirred at ambient temperature for 30 minutes. The resultant mixture was cooled to 0° C. and 2-pyrrolidin-1-ylethyl chloride hydrochloride (7.05 g) was added portionwise. The resultant mixture was stirred and heated to 80° C. for 3 hour. The mixture was partitioned between methylene chloride and water. The organic phase was washed with water and with brine, dried over magnesium sulphate and evaporated. The residue was diluted with ethanol and cooled to 0° C. and a saturated solution of hydrogen chloride in diethyl ether was added. The precipitate was isolated and the filtrate was evaporated to afford a second batch of product. There was thus obtained 6-nitro-1-(2-pyrrolidin-1-ylethyl)indole hydrochloride (2.89 g); ¹H NMR: (DMSOd₆) 1.85 (m, 2H), 1.95 (m, 2H), 3.0 (m, 2H), 3.5 (m, 2H), 3.7 (m, 2H), 4.8 (m, 2H), 6.75 (d, 1H), 7.8 (d, 1H), 7.9 (s, 1H), 7.95 (d, 1H), 8.7 (s, 1H).

A mixture of a portion (0.95 g) of the materials obtained, 10% palladium-on-carbon (0.2 g) and ethanol (15 ml) was stirred under 3 atmospheres pressure of hydrogen for 3 hours. The resultant mixture was filtered and the filtrate was evaporated. There was thus obtained 6-amino-1-(2-pyrrolidin-1-ylethyl)indole as an oil (0.863 g); ¹H NMR: (DMSOd₆) 1.65 (m, 4H), 2.5 (m, 4H), 2.7 (t, 2H), 4.05 (t, 2H), 4.75 (br s, 2H), 6.15 (d, 1H), 6.4 (d, 1H), 6.55 (s, 1H), 7.0 (s, 1H), 7.2 (d, 1H).

[25] DMA was used in place of DMF as the reaction solvent and the reaction mixture was heated to 100° C. in a microwave oven for 4 minutes. The reaction mixture was cooled to ambient temperature and the precipitated solid was collected by filtration and purified by preparative HPLC as described in Example 34. The product so obtained gave the following characterising data: —¹H NMR: (DMSOd₆) 1.65 (br s, 4H), 2.45 (br s, 4H), 2.8 (t, 2H), 4.0 (s, 6H), 4.25 (t, 2H), 5.05 (s, 2H), 6.35 (d, 1H), 7.3 (d, 1H), 7.4 (m, 3H), 7.55 (s, 1H), 7.7 (s, 1H), 7.9 (s, 1H), 8.15 (s, 1H), 8.7 (s, 1H), 10.2 (s, 1H); Mass Spectrum: M+H⁺ 542.

The 5-amino-1-(2-pyrrolidin-1-ylethyl)indole used as a starting material was prepared as follows: —

Using analogous procedures to those described in the portion of Note [24] above that is concerned with the preparation of starting materials, 5-nitroindole was reacted with 2-pyrrolidin-1-ylethyl chloride. On completion of the reaction, the reaction mixture was partitioned between methylene chloride and water. The organic phase was washed with water and with brine, dried over magnesium sulphate and evaporated. The residue was purified by column chromatography on silica using increasingly polar mixtures of methylene chloride and methanol as eluent. There was thus obtained 5-nitro-1-(2-pyrrolidin-1-ylethyl)indole; ¹H NMR: (DMSOd₆) 1.6 (m, 4H), 2.5 (m, 4H), 2.8 (t, 2H), 4.4 (t, 2H), 6.75 (d, 1H), 7.65 (d, 1H), 7.7 (d, 1H), 8.05 (d, 1H), 8.55 (s, 1H); which in turn was hydrogenated to give 5-amino-1-(2-pyrrolidin-1-ylethyl)indole; ¹H NMR: (DMSOd₆) 1.65 (m, 4H), 2.5 (m, 4H), 2.7 (t, 2H), 4.15 (t, 2H), 4.45 (br s, 2H), 6.1 (d, 1H), 6.4 (d, 1H), 6.55 (s, 1H), 7.1 (d, 1H), 7.15 (s, 1H).

### EXAMPLE 36

### N-[3-dimethylaminomethyl-2-methylindol-5-yl]-2-[4-(6,7-dimethoxyquinazolin-4-yloxy)pyrazol-1-yl]acetamide

Sodium triacetoxyborohydride (0.07 g) was added portionwise to a stirred mixture of N-(3-formyl-2-methylindol-5-yl)-2-[4-(6,7-dimethoxyquinazolin-4-yloxy)pyrazol-1-yl]acetamide (0.125 g) and a dimethylamine solution (2M in THF, 0.3 ml) and the resultant mixture was stirred at ambient temperature for 12 hours. The mixture was evaporated and the residue was purified by column chromatography on silica using a 19:1 mixture of methylene chloride and methanol as eluent. There was thus obtained the title compound as a solid (0.075 g); ¹H NMR: (DMSOd₆) 2.11 (s, 6H), 2.32 (s, 3H), 3.39 (s, 2H), 3.98 (s, 3H), 3.99 (s, 3H), 5.01 (s, 2H), 7.15-7.22 (m, 2H), 7.39 (s, 1H), 7.55 (s, 1H), 7.68 (s, 1H), 7.77 (s, 1H), 8.13 (s, 1H), 8.66 (s, 1H), 10.09 (s, 1H), 10.77 (s, 1H); Mass Spectrum: M−H⁻ 514.

The N-(3-formyl-2-methylindol-5-yl)-2-[4-(6,7-dimethoxyquinazolin-4-yloxy)pyrazol-1-yl]acetamide used as a starting material was prepared as follows: —

A mixture of 3-formyl-2-methyl-5-nitroindole (0.3 g), platinium oxide (0.03 g), ethyl acetate (15 ml) and ethanol (15 ml) was stirred under 1.8 atmospheres pressure of hydrogen for 2 hours. The catalyst was filtered off and the filtrate was evaporated. The residue was purified by column chromatography on silica using a 19:1 mixture of methylene chloride and methanol as eluent. There was thus obtained 5-amino-2-methylindole-3-carbaldehyde (0.133 g); ¹H NMR: (CDCl₃) 2.68 (s, 3H), 3.64 (m, 2H), 6.65 (d, 1H), 7.1 (d, 1H), 7.59 (s, 1H), 10.1 (s, 1H).

The material so obtained was added to a stirred mixture of 2-[4-(6,7-dimethoxyquinazolin-4-yloxy)pyrazol-1-yl]acetic acid (0.2 g), 1-(3-dimethylamino-propyl)-3-ethylcarbodiimide hydrochloride (0.14 g), 2-hydroxypyridine N-oxide (0.081 g) and DMF (3 ml) and the resultant mixture was stirred at ambient temperature for 3 hours. Water was added and the precipitate was isolated, washed in turn with water and with ethyl acetate and dried under vacuum. There was thus obtained N-(3-formyl-2-methylindol-5-yl)-2-[4-(6,7-dimethoxyquinazolin-4-yloxy)pyrazol-1-yl]acetamide (0.25 g); ¹H NMR: (DMSOd₆) 2.67 (s, 3H), 3.98 (s, 3H), 3.99 (s, 3H), 5.03 (s, 2H), 7.34 (d, 1H), 7.39 (s, 1H), 7.48 (m, 1H), 7.55 (s, 1H), 7.68 (s, 1H), 8.14 (s, 1H), 8.31 (m, 1H), 8.67 (s, 1H), 10.02 (s, 1H), 10.29 (s, 1H), 11.95 (s, 1H); Mass Spectrum: M+H⁺ 487.

### EXAMPLE 37

Using a similar procedure to that described in Example 8, the appropriate 2-(pyrazol-1-yl)acetic acid was reacted with the appropriate aniline to give the compounds described in Table X. Unless otherwise stated, each aniline or heterocyclyl amine starting material was commercially available.

For each of the Compounds [1] to [14] below, 2-(benzotriazol-1-yl)-1,1,3,3-tetramethyluronium tetrafluoroborate was used in place of 2-(7-azabenzotriazol-1-yl)-1,1,3,3-tetramethyluronium hexafluorophosphate(V), the reaction mixture was stirred at ambient temperature for 16 hours, water was added and the precipitate was recovered by filtration and purified by preparative HPLC using a Waters ‘Symmetry’ C18 reversed-phase column (5 microns silica, 19 mm diameter, 100 mm length) and decreasingly polar mixtures of water (containing 2% acetic acid) and acetonitrile as eluent.

Unless otherwise stated, for the Compounds [15] to [27] below, the reaction mixture was stirred at ambient temperature for 1 hour and the resultant reaction mixture was injected into a Waters ‘β Basic Hypersil’ reversed-phase column (5 microns silica, 30 mm diameter, 250 mm length) which was eluted with decreasingly polar mixtures of water (containing 0.2% ammonium carbonate) and acetonitrile.

TABLE X

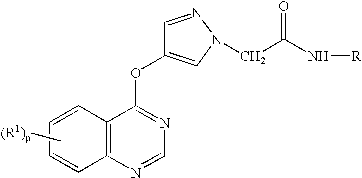

| No. & | | |
|---|---|---|
| Note | (R¹)ₚ | R |
| [1] | 6-methoxy-7-(2-pyrrolidin-1-ylethoxy) | 2,3-methylenedioxyphenyl |
| [2] | 6-methoxy- | 6-fluoro-2,3-methylenedioxyphenyl |
| | 7-[2-(4-acetylpiperazin-1-yl)ethoxy] | |
| [3] | 6-methoxy- | 6-fluoro-2,3-methylenedioxyphenyl |
| | 7-[2-(4-methylpiperazin-1-yl)ethoxy] | |
| [4] | 6-methoxy- | 6-fluoro-2,3-methylenedioxyphenyl |
| | 7-[2-(4-hydroxypiperidin-1-yl)ethoxy] | |
| [5] | 6-methoxy-7-(2-pyrrolidin-1-ylethoxy) | 3 ,4-methylenedioxyphenyl |
| [6] | 6-methoxy- | 3,4-methylenedioxyphenyl |
| | 7-[2-(4-acetylpiperazin-1-yl)ethoxy]. | |
| [7] | 6-methoxy- | 3,4-methylenedioxyphenyl |
| | 7-[2-(4-methylpiperazin-1-yl)ethoxy] | |
| [8] | 6-methoxy- | 3,4-methylenedioxyphenyl |
| | 7-[2-(4-hydroxypiperidin-1-yl)ethoxy] | |
| [9] | 6-methoxy-7-(2-pyrrolidin-1-ylethoxy) | 2,2-dimethyl-1,3-benzodioxol-5-yl |
| [10] | 6-methoxy-7-(2-pyrrolidin-1-ylethoxy) | 3,4-ethylenedioxyphenyl |
| [11] | 6-methoxy- | 3,4-ethylenedioxyphenyl |
| | 7-[2-(4-methylpiperazin-1-yl)ethoxy] | |
| [12] | 6-methoxy- | 3,4-ethylenedioxyphenyl |
| | 7-[2-(4-hydroxypiperidin-1-yl)ethoxy] | |
| [13] | 6-methoxy-7-(2-hydroxyethoxy) | 2,3-ethylenedioxyphenyl |
| [14] | 6-methoxy-7-(2,3-dihydroxypropoxy) | 2,3-ethylenedioxyphenyl |
| [15] | 6,7-dimethoxy | 2,2-dimethyl-1,3-benzodioxol-5-yl |
| [16] | 6-methoxy-7-(3-pyrrolidin-1-ylpropoxy) | 2,3-methylenedioxyphenyl |
| [17] | 6,7-dimethoxy | 2,3-ethylenedioxyphenyl |
| [18] | 6-methoxy- | 3,4-ethylenedioxyphenyl |
| | 7-[2-(4-acetylpiperazin-1-yl)ethoxy] | |
| [19] | 6,7-dimethoxy | 2-methylisoindolin-5-yl |
| [20] | 6,7-dimethoxy | N-methylindolin-5-yl |
| [21] | 6-methoxy-7-(2-pyrrolidin-1-ylethoxy) | N-methylindolin-5-yl |
| [22] | 6,7-dimethoxy | N-acetylindolin-6-yl |
| [23] | 6,7-dimethoxy | N-methylindolin-6-yl |
| [24] | 6-methoxy-7-(2-pyrrolidin-1-ylethoxy) | N-methylindolin-6-yl |
| [25] | 6,7-dimethoxy | N-methyl-1,2,3,4-tetrahydroquinolin-7-yl |
| [26] | 6,7-dimethoxy | 4-methyl-2H-1,4-benzoxazin-7-yl |
| [27] | 7-methoxy-6-(3-pyrrolidin-1-ylpropoxy) | 2,3-methylenedioxyphenyl |

Notes The products gave the characterising data shown below.

[1] ¹H NMR: (DMSOd₆) 1.7 (m, 4H), 2.55 (m, 4H), 2.9 (t, 2H), 4.0 (s, 3H), 4.3 (t, 2H), 5.1 (s, 2H), 6.1 (s, 2H), 6.75 (d, 1H), 6.85 (m, 1H), 7.3 (d, 1H), 7.4 (s, 1H), 7.55 (s, 1H), 7.7 (s, 1H), 8.1 (s, 1H), 8.65 (s, 1H); Mass Spectrum: M+H⁺ 533.

The 2-{4-[6-methoxy-7-(2-pyrrolidin-1-ylethoxy)quinazolin-4-yloxy]pyrazol-1-yl}acetic acid used as a starting material was prepared as follows: —

7-Benzyloxy-4-chloro-6-methoxyquinazoline (International; Patent Application WO 02/16352, Example 1 thereof; 2.5 g) was added to a stirred mixture of tert-butyl 2-(4-hydroxypyrazol-1-yl)acetate (1.81 g), potassium carbonate (1.73 g) and DMA and the resultant mixture was heated at 100° C. for 1 hour under argon. The mixture was cooled to ambient temperature, water was added and the precipitate was collected by filtration, washed with water and with diethyl ether and dried over phosphorus pentoxide under vacuum. There were thus obtained tert-butyl 2-[4-(7-benzyloxy-6-methoxyquinazolin-4-yloxy)pyrazol-1-yl]acetate (3.5 g) which was used without further purification.

A mixture of the material so obtained, 10% palladium-on-carbon (0.5 g), ammonium formate (3.81 g) and DMF (50 ml) was stirred at ambient temperature for 2 hours. The mixture was filtered and the filtrate was evaporated to give a solid that was washed successively with diethyl ether, water and more diethyl ether. There was thus obtained tert-butyl 2-[4-(7-hydroxy-6-methoxyquinazolin-4-yloxy)pyrazol-1-yl]acetate (2.66 g); ¹H NMR: (DMSOd₆) 1.45 (s, 9H), 4.0 (s, 3H), 4.95 (s, 2H), 7.2 (s, 1H), 7.5 (s, 1H), 7.65 (s, 1H), 8.05 (s, 1H), 8.55 (s, 1H).

Diethyl azodicarboxylate (0.19 ml) was added to a stirred mixture of tert-butyl 2-[4-(7-hydroxy-6-methoxyquinazolin-4-yloxy)pyrazol-1-yl]acetate (0.3 g), N-(2-hydroxyethyl)pyrrolidine (0.104 ml), triphenylphosphine (0.32 g) and methylene chloride (15 ml) and the resultant mixture was stirred at ambient temperature for 10 minutes. The mixture was evaporated and the residue was purified by column chromatography on silica using a 9:1 mixture of methylene chloride and methanol as eluent. There was thus obtained tert-butyl 2-{4-[6-methoxy-7-(2-pyrrolidin-1-ylethoxy)quinazolin-4-yloxy]pyrazol-1-yl}acetate as a gum (0.31 g); ¹H NMR: (CDCl₃) 1.5 (s, 9H), 1.85 (m, 4H), 2.7 (m, 4H), 3.05 (m, 2H), 4.05 (s, 3H), 4.35 (t, 2H), 4.85 (t, 2H), 7.3 (s, 1H), 7.5 (s, 1H), 7.7 (s, 1H), 7.9 (s, 1H), 8.7 (s, 1H); Mass Spectrum: M+H⁺ 470.

A mixture of the material so obtained, trifluoroacetic acid (4 ml) and methylene chloride (1 ml) was stirred at ambient temperature for 2 hours. The reaction mixture was evaporated, toluene was added and the solvent was evaporated off under vacuum. This process was repeated twice. The residual oil was triturated under a mixture of diethyl ether and ethyl acetate. The resultant solid was collected by filtration and washed with diethyl ether. There was thus obtained 2-{4-[6-methoxy-7-(2-pyrrolidin-1-ylethoxy)quinazolin-4-yloxy]pyrazol-1-yl}acetic acid (0.22 g); Mass Spectrum: M+H⁺ 414.

[2] ¹H NMR: (DMSOd₆) 2.0 (s, 3H), 2.5 (m, 4H), 2.85 (t, 2H), 3.45 (m, 4H), 3.95 (s, 3H), 4.35 (t, 2H), 5.1 (s, 2H), 6.1 (s, 2H), 6.75 (m, 1H), 6.85 (m, 1H), 7.4 (s, 1H), 7.55 (s, 1H), 7.7 (s, 1H), 8.1 (s, 1H), 8.65 (s, 1H); Mass Spectrum: M+H⁺ 608.

The 2-(4-{7-[2-(4-acetylpiperazin-1-yl)ethoxy]-6-methoxyquinazolin-4-yloxy}pyrazol-1-yl)acetic acid used as a starting material was prepared as follows: —

Diethyl azodicarboxylate (0.709 ml) was added to a stirred mixture of tert-butyl 2-[4-(7-hydroxy-6-methoxyquinazolin-4-yloxy)pyrazol-1-yl]acetate (1.12 g), 2-bromoethanol (0.234 ml), triphenylphosphine (1.18 g) and methylene chloride (25 ml) and the resultant mixture was stirred at ambient temperature for 30 minutes. The mixture was evaporated and the residue was purified by column chromatography on silica using increasingly polar mixtures of methylene chloride and methanol as eluent. There was thus obtained tert-butyl 2-{4-[7-(2-bromoethoxy)-6-methoxyquinazolin-4-yloxy]pyrazol-1-yl}acetate (1.285 g); Mass Spectrum: M+H⁺ 479.

A mixture of a portion (0.383 g) of the material so obtained, 1-acetylpiperazine (0.174 g), potassium carbonate (0.221 g), potassium iodide (0.133 g) and DMA (10 ml) was heated to 100° C. for 2 hours. The mixture was cooled, poured in water and extracted with ethyl acetate. The organic phase was washed with water and with brine, dried over magnesium sulphate and evaporated. There was thus obtained tert-butyl 2-(4-{7-[2-(4-acetylpiperazin-1-yl)ethoxy]-6-methoxyquinazolin-4-yloxy}pyrazol-1-yl)acetate; ¹H NMR: (CDCl₃) 1.55 (s, 9H), 2.1 (s, 3H), 2.65 (m, 4H), 2.95 (m, 2H), 3.5 (t, 2H), 3.7 (t, 2H), 3.85 (s, 3H), 4.35 (t, 2H), 4.85 (s, 2H), 7.3 (s, 1H), 7.5 (s, 1H), 7.7 (s, 1H), 7.9 (s, 1H), 8.7 (s, 1H); Mass Spectrum: M+H⁺ 527.

The tert-butyl group was cleaved from the product so obtained using an analogous procedure to that described in the last paragraph of the portion of Note [1] immediately above. There was thus obtained 2-(4-{7-[2-(4-acetylpiperazin-1-yl)ethoxy]-6-methoxyquinazolin-4-yloxy}pyrazol-1-yl)acetic acid (0.363 g); Mass Spectrum: M-IT 469.

[3] Mass Spectrum: M+H⁺ 580.

The 2-(4-{7-[2-(4-methylpiperazin-1-yl)ethoxy]-6-methoxyquinazolin-4-yloxy}pyrazol-1-yl)acetic acid used as a starting material was prepared as follows: —

Using analogous procedures to those described in the last two paragraphs of the portion of Note [2] immediately above that is concerned with the preparation of starting materials, tert-butyl 2-{4-[7-(2-bromoethoxy)-6-methoxyquinazolin-4-yloxy]pyrazol-1-yl}acetate was reacted with 1-methylpiperazine to give tert-butyl 2-(4-{7-[2-(4-methylpiperazin-1-yl)ethoxy]-6-methoxyquinazolin-4-yloxy}pyrazol-1-yl)acetate; ¹H NMR: (CDCl₃) 1.55 (s, 9H), 2.4 (s, 3H), 2.6-2.9 (m, 8H), 3.0 (t, 2H), 4.05 (s, 3H), 4.35 (t, 2H), 4.85 (s, 2H); 7.3 (s, 1H), 7.5 (s, 1H), 7.7 (s, 1H), 7.95 (s, 1H), 8.7 (s, 1H); Mass Spectrum: M+H⁺ 498; which in turn was treated with trifluoroacetic acid to provide the required starting material; Mass Spectrum: M+H⁺ 443.

[4] ¹H NMR: (DMSOd₆+CF₃CO₂D) 1.4 (m, 2H), 1.7 (m, 2H), 2.2 (m, 2H), 2.8 (m, 2H), 3.5 (m, 2H), 3.95 (s, 3H), 4.3 (t, 2H), 4.55 (d, 1H), 5.1 (s, 2H), 6.1 (s, 2H), 6.75 (m, 1H), 6.85 (m, 1H), 7.4 (s, 1H), 7.55 (s, 1H), 7.7 (s, 1H), 8.1 (s, 1H), 8.65 (s, 1H); Mass Spectrum: M+H⁺ 581.

The 2-(4-{7-[2-(4-hydroxypiperidin-1-yl)ethoxy]-6-methoxyquinazolin-4-yloxy}pyrazol-1-yl)acetic acid used as a starting material was prepared as follows: —

Using analogous procedures to those described in the last two paragraphs of the portion of Note [2] immediately above that is concerned with the preparation of starting materials, tert-butyl 2-{4-[7-(2-bromoethoxy)-6-methoxyquinazolin-4-yloxy]pyrazol-1-yl}acetate was reacted with 4-hydroxypiperidine to give tert-butyl 2-(4-{7-[2-(4-hydroxypiperidin-1-yl)ethoxy]-6-methoxyquinazolin-4-yloxy}pyrazol-1-yl)acetate; Mass Spectrum: M+H⁺ 500; which in turn was treated with trifluoroacetic acid to provide the required starting material; Mass Spectrum: M+H⁺ 444.

[5] ¹H NMR: (DMSOd₆) 1.7 (m, 4H), 2.55 (m, 4H), 2.9 (t, 2H), 4.0 (s, 3H), 4.3 (t, 2H), 5.0 (s, 2H), 6.0 (s, 2H), 6.8 (d, 1H), 7.0 (d, 1H), 7.3 (s, 1H), 7.4 (s, 1H), 7.55 (s, 1H), 7.65 (s, 1H), 8.1 (s, 1H), 8.65 (s, 1H); Mass Spectrum: M+H⁺ 533.

[6] ¹H NMR: (DMSOd₆) 2.0 (s, 3H), 2.45 (m, 4H), 2.85 (t, 2H), 3.45 (m, 4H), 3.95 (s, 3H), 4.35 (t, 2H), 5.0 (s, 2H), 6.0 (s, 2H), 6.85 (d, 1H), 6.95 (d, 1H), 7.3 (s, 1H), 7.4 (s, 1H), 7.55 (s, 1H), 7.65 (s, 1H), 8.1 (s, 1H), 8.65 (s, 1H); Mass Spectrum: M+H⁺ 590.

[7] ¹H NMR: (DMSOd₆ and CF₃CO₂D) 2.15 (s, 3H), 2.3 (br s, 4H), 2.5 (br s, 4H), 2.8 (t, 2H), 4.0 (s, 3H), 4.30 (t, 2H), 5.0 (s, 2H), 6.0 (s, 2H), 6.85 (d, 1H), 7.0 (d, 1H), 7.3 (s, 1H), 7.4 (s, 1H), 7.55 (s, 1H), 7.7 (s, 1H), 8.1 (s, 1H), 8.65 (s, 1H); Mass Spectrum: M+H⁺ 562.

[8] ¹H NMR: (DMSOd₆) 1.4 (m, 2H), 1.75 (m, 2H), 2.2 (m, 2H), 2.75 (t, 2H), 2.8 (m, 2H), 3.45 (m, 1H), 4.0 (s, 3H), 4.3 (t, 2H), 4.55 (d, 1H), 5.0 (s, 2H), 6.0 (s, 2H), 6.9 (d, 1H), 7.0 (d, 1H), 7.3 (s, 1H), 7.4 (s, 1H), 7.55 (s, 1H), 7.7 (s, 1H), 8.1 (s, 1H), 8.65 (s, 1H); Mass Spectrum: M+H⁺ 563.

[9] ¹H NMR: (DMSOd₆ and CF₃CO₂D) 1.6 (s, 6H), 1.7 (m, 4H), 2.55 (m, 4H), 2.9 (t, 2H), 4.0 (s, 3H), 4.3 (t, 2H), 5.0 (s, 2H), 6.75 (d, 1H), 6.95 (d, 1H), 7.2 (s, 1H), 7.4 (s, 1H), 7.55 (s, 1H), 7.65 (s, 1H), 8.1 (s, 1H), 8.75 (s, 1H); Mass Spectrum: M+H⁺ 561.

The 2,2-dimethyl-1,3-benzodioxol-5-ylamine used as a starting material was prepared by the procedures described in the Australian Journal of Chemistry, 1980, 33, 675-680.

[10] ¹H NMR: (DMSOd₆) 1.7 (m, 4H), 2.55 (m, 4H), 2.9 (t, 2H), 4.0 (s, 3H), 4.2 (m, 4H), 4.3 (t, 2H), 5.0 (s, 2H), 6.8 (d, 1H), 7.0 (d, 1H), 7.25 (s, 1H), 7.4 (s, 1H), 7.5 (s, 1H), 7.65 (s, 1H), 8.1 (s, 1H), 8.75 (s, 1H); Mass Spectrum: M+H⁺ 547.

[11] ¹H NMR: (DMSOd₆) 2.1 (s, 3H), 2.3 (br m, 4H), 2.8 (t, 2H), 3.95 (s, 3H), 4.2 (m, 4H), 4.3 (t, 2H), 4.95 (s, 2H), 6.8 (d, 1H), 6.95 (d, 1H), 7.25 (s, 1H), 7.45 (s, 1H), 7.55 (s, 1H), 7.7 (s, 1H), 8.1 (s, 1H), 8.65 (s, 1H); Mass Spectrum: M+H⁺ 576.

[12]. ¹H NMR: (DMSOd₆) 1.4 (m, 2H), 1.75 (m, 2H), 2.2 (m, 2H), 2.75 (t, 2H), 2.8 (m, 2H), 3.45 (m, 1H), 4.0 (s, 3H), 4.2 (m, 4H), 4.3 (t, 2H), 4.55 (d, 1H), 5.0 (s, 2H), 6.8 (d, 1H), 7.0 (d, 1H), 7.25 (s, 1H), 7.4 (s, 1H), 7.55 (s, 1H), 7.65 (s, 1H), 8.1 (s, 1H), 8.65 (s, 1H); Mass Spectrum: M+H⁺ 577.

[13] The acetic acid starting material was a mixture of 2-{4-[7-(2-hydroxyethoxy)-6-methoxyquinazolin-4-yloxy]pyrazol-1-yl}acetic acid and the corresponding 7-(2-trifluoroacetoxyethoxy) compound. The product gave the following characterising data ¹H NMR: (DMSOd₆) 3.8 (m, 2H), 3.95 (s, 3H), 4.2 (m, 2H), 4.25 (m, 2H), 4.35 (m, 2H), 4.95 (m, 1H), 5.15 (s, 2H), 6.65 (d, 1H), 6.8 (t, 1H), 7.4 (s, 1H), 7.53 (s, 1H), 7.54 (s, 1H), 7.7 (s, 1H), 8.15 (s, 1H), 8.65 (s, 1H), 9.5 (s, 1H); Mass Spectrum: M+H⁺ 494.

The starting material mixture was prepared as follows: —

Diethyl azodicarboxylate (0.93 g) was added to a stirred mixture of tert-butyl 2-[4-(7-hydroxy-6-methoxyquinazolin-4-yloxy)pyrazol-1-yl]acetate (1 g), 2-(2-hydroxyethoxy)tetrahydropyran (0.437 ml), triphenylphosphine (1.06 g) and methylene chloride (25 ml) and the resultant mixture was stirred at ambient temperature for 4 hours. The mixture was evaporated and the residue was purified by column chromatography on silica using increasingly polar mixtures of methylene chloride and diethyl ether as eluent. There was thus obtained tert-butyl 2-{4-[6-methoxy-7-(2-tetrahydropyran-2-yloxyethoxy)quinazolin-4-yloxy]pyrazol-1-yl}acetate as a foam (0.9 g); Mass Spectrum: M+H⁺ 376.

Trifluoroacetic acid (12 ml) was added dropwise to a stirred mixture of the material so obtained and methylene chloride (10 ml) that had been cooled to 0° C. The resultant mixture was stirred at ambient temperature for 16 hours. The reaction mixture was evaporated, toluene was added and the solvent was evaporated off under vacuum. This process was repeated. The residual oil was triturated under ethyl acetate. The resultant solid was collected by filtration and dried under vacuum. There was thus obtained a mixture (0.86 g) of 2-{4-[7-(2-hydroxyethoxy)-6-methoxyquinazolin-4-yloxy]pyrazol-1-yl}acetic acid and the corresponding 7-(2-trifluoroacetoxyethoxy) compound; Mass Spectrum: M+H⁺ 361 and 457 respectively.

[14] The acetic acid starting material was a mixture of 2-{4-[7-(2,3-dihydroxypropoxy)-6-methoxyquinazolin-4-yloxy]pyrazol-1-yl}acetic acid and the corresponding 7-(2-hydroxy-3-trifluoroacetoxypropoxy) compound. The product gave the following characterising data: —¹H NMR: (DMSOd₆) 3.5 (m, 2H), 3.9 (m, 1H), 4.0 (s, 3H), 4.1 (m, 1H), 4.2 (m, 1H), 4.25 (m, 2H), 4.35 (m, 2H), 4.75 (m, 1H), 5.1 (d, 1H), 5.15 (s, 2H), 6.65 (d, 1H), 6.8 (t, 1H), 7.4 (s, 1H), 7.5 (d, 1H), 7.55 (s, 1H), 7.7 (s, 1H), 8.15 (s, 1H), 8.65 (s, 1H), 9.5 (s, 1H); Mass Spectrum: M+H⁺ 524.

The starting material mixture was prepared as follows: —

Diethyl azodicarboxylate (2.78 g) was added to a stirred mixture of tert-butyl 2-[4-(7-hydroxy-6-methoxyquinazolin-4-yloxy)pyrazol-1-yl]acetate (3 g), 2,2-dimethyl-1,3-dioxolan-4-ylmethanol (1.2 ml), triphenylphosphine (3.17 g) and methylene chloride (75 ml) and the resultant mixture was stirred at ambient temperature for 16 hours. The mixture was evaporated and the residue was purified by column chromatography on silica using increasingly polar mixtures of methylene chloride and ethyl acetate as eluent. There was thus obtained tert-butyl 2-{4-[7-(2,2-dimethyl-1,3-dioxolan-4-ylmethoxy)-6-methoxyquinazolin-4-yloxy]pyrazol-1-yl}acetate as a foam (3.55 g); Mass Spectrum: M+H⁺ 487.

Trifluoroacetic acid (45 ml) was added dropwise to a stirred mixture of the material so obtained and methylene chloride (40 ml) that had been cooled to 0° C. The resultant mixture was stirred at ambient temperature for 16 hours. The reaction mixture was evaporated, toluene was added and the solvent was evaporated off under vacuum. This process was repeated. The residual oil was triturated under diethyl ether. The resultant solid was collected by filtration and dried under vacuum. There was thus obtained a 1:1 mixture (3.5 g) of 2-{4-[7-(2,3-dihydroxypropoxy)-6-methoxyquinazolin-4-yloxy]pyrazol-1-yl}acetic acid and the corresponding 7-(2-hydroxy-3-trifluoroacetoxypropoxy) compound; Mass Spectrum: M+H⁺ 391 and 487 respectively.

[15] ¹H NMR: (DMSOd₆) 1.6 (s, 6H), 4.0 (s, 6H), 5.0 (s, 2H), 6.8 (d, 1H), 6.95 (d, 1H), 7.2 (s, 1H), 7.4 (s, 1H), 7.55 (s, 1H), 7.7 (s, 1H), 8.1 (s, 1H), 8.7 (s, 1H); Mass Spectrum: M+H⁺ 478.

[16] ¹H NMR: (DMSOd₆) 1.8 (br m, 4H), 2.2 (br m, 2H), 3.2 (br m, 6H), 4.0 (s, 3H), 4.3 (t, 2H), 5.0 (s, 2H), 6.0 (s, 2H), 6.9 (d, 1H), 7.0 (d, 1H), 7.3 (s, 1H), 7.45 (s, 1H), 7.55 (s, 1H), 7.65 (s, 1H), 8.1 (s, 1H), 8.65 (s, 1H); Mass Spectrum: M+H⁺ 547.

The 2-{4-[6-methoxy-7-(3-pyrrolidin-1-ylpropoxy)quinazolin-4-yloxy]pyrazol-1-yl}acetic acid used as a starting material was prepared as follows: —

Diethyl azodicarboxylate (0.45 ml) was added to a stirred mixture of tert-butyl 2-[4-(7-hydroxy-6-methoxyquinazolin-4-yloxy)pyrazol-1-yl]acetate (0.71 g), N-(3-hydroxypropyl)pyrrolidine (0.3 ml), triphenylphosphine (0.75 g) and methylene chloride (15 ml) and the resultant mixture was stirred at ambient temperature for 10 minutes. The mixture was evaporated and the residue was purified by column chromatography on silica using increasingly polar mixtures of methylene chloride and methanol as eluent. There was thus obtained tert-butyl 2-{4-[6-methoxy-7-(3-pyrrolidin-1-ylpropoxy)quinazolin-4-yloxy]pyrazol-1-yl}acetate as a foam (0.61 g); Mass Spectrum: M+H⁺ 484.

A mixture of a portion (0.435 g) of the material so obtained, trifluoroacetic acid (4 ml) and methylene chloride (1 ml) was stirred at ambient temperature for 2 hours. The reaction mixture was evaporated, toluene was added and the solvent was evaporated off under vacuum. This process was repeated twice. The residual gum was suspended in methylene chloride and diisopropylethylamine was added dropwise until complete dissolution occurred. The resultant solution was evaporated and the residue so obtained was triturated under ethyl acetate. The resultant solid was collected by filtration and washed in turn with ethyl acetate and with diethyl ether. There was thus obtained 2-{4-[6-methoxy-7-(3-pyrrolidin-1-ylpropoxy)quinazolin-4-yloxy]pyrazol-1-yl}acetic acid (0.4 g); ¹H NMR: (DMSOd₆+CF₃CO₂D) 1.9 (m, 2H), 2.1 (m, 2H), 2.25 (m, 2H), 3.1 (m, 2H), 3.35 (m, 2H), 3.7 (m, 2H), 4.05 (s, 3H), 4.35 (t, 2H), 5.05 (s, 2H), 7.5 (s, 1H), 7.65 (s, 1H), 7.7 (s, 1H), 8.15 (s, 1H), 8.9 (s, 1H); Mass Spectrum: M+H⁺ 428.

[17] ¹H NMR: (DMSOd₆) 3.95 (s, 6H), 4.25 (m, 2H), 4.35 (m, 2H), 5.15 (s, 2H), 6.65 (m, 1H), 6.8 (m, 1H), 7.4 (s, 1H), 7.55 (br s, 2H), 7.7 (s, 1H), 8.1 (s, 1H), 8.65 (s, 1H), 9.5 (s, 1H); Mass Spectrum: M+H⁺ 464.

[18] ¹H NMR: (DMSOd₆) 2.0 (s, 3H), 2.5 (m, 4H), 2.85 (t, 2H), 3.45 (m, 4H), 4.0 (s, 3H), 4.2 (m, 4H), 4.35 (t, 2H), 5.0 (s, 2H), 6.8 (d, 1H), 6.95 (d, 1H), 7.25 (d, 1H), 7.45 (s, 1H), 7.55 (s, 1H), 7.65 (s, 1H), 8.1 (s, 1H), 8.65 (s, 1H); Mass Spectrum: M+H⁺ 604.

[19] ¹H NMR: (CDCl₃) 2.6 (s, 3H), 3.88 (s, 2H), 3.9 (s, 2M), 4.07 (s, 3H), 4.08 (s, 3H), 4.95 (s, 2H), 7.1 (d, 1H), 7.2 (d, 1H), 7.35 (s, 1H), 7.47 (s, 1H), 7.5 (s, 1H), 7.85 (s, 1H), 8.0 (s, 1H), 8.3 (s, 1H), 8.7 (s, 1H); Mass Spectrum: M+H⁺ 461.

The 5-amino-2-methylisoindoline used as a starting material was prepared as follows: —

Lithium aluminium hydride (0.76 g) was added portionwise to a stirred mixture of 4-amino-1-methylphthalimide (0.88 g) and THF (8 ml) and the reaction mixture was heated to 60° C. for 1 hour. Ethyl acetate and water (1 ml) were added in turn and the resultant mixture was filtered. The filtrate was evaporated. The residue was triturated under diethyl ether and filtered. The diethyl ether solution was evaporated to give 5-amino-2-methylisoindoline as an oil (0.31 g); Mass Spectrum: M+H⁺ 149.

[20] ¹H NMR: (CDCl₃) 2.7 (s, 3H), 2.95 (t, 2H), 3.3 (t, 2H), 4.07 (s, 3H), 4.08 (s, 3H), 4.95 (s, 2H), 6.4 (d, 1H), 7.05 (d, 1H), 7.35 (s, 1H), 7.5 (s, 1H), 7.85 (s, 1H), 7.95 (s, 1H), 8.0 (s, 1H), 8.7 (s, 1H); Mass Spectrum: M+H⁺ 461.

The 5-amino-N-methylindoline used as a starting material was prepared as follows: —

Methyl iodide (1.59 ml) was added to a stirred mixture of 5-nitroindoline (2.46 g), potassium carbonate (6.22 g) and DMF (25 ml) and the reaction mixture was stirred at ambient temperature for 2 days. The mixture was evaporated and the residue was diluted with water and extracted with diethyl ether. The organic phase was washed with water and brine, dried over magnesium sulphate and evaporated. The residue was purified by column chromatography on silica using increasingly polar mixtures of petroleum ether (b.p. 40-60° C.) and ethyl-acetate as eluent. There was thus obtained N-methyl-5-nitroindoline (1.5 g); ¹H NMR: (CDCl₃) 2.9 (s, 3H), 3.05 (t, 2H), 3.6 (t, 2H), 6.3 (d, 1H), 7.9 (s, 1H), 8.1 (d, 1H).

A mixture of a portion (1.07 g) of the material so obtained, ammonium formate (1.89 g), 10% palladium-on-carbon (0.3 g) and methanol (11 ml) was stirred at ambient temperature for 2 hours. The mixture was filtered and the filtrate was evaporated. The residue was purified by column chromatography on silica using increasingly polar mixtures of petroleum ether (b.p. 40-60° C.) and ethyl acetate as eluent. There was thus obtained 5-amino-N-methylindoline (0.122 g); ¹H NMR: (CDCl₃ and CF₃CO₂D) 2.8 (s, 3H), 2.95 (t, 2H), 3.4 (t, 2H), 6.45 (d, 1H), 7.05 (d, 1H), 7.45 (s, 1H).

[21] ¹H NMR: (DMSOd₆+CF₃CO₂D) 1.9 (m, 2H), 2.1 (m, 2H), 3.15 (s, 3H), 3.25 (m, 4H), 3.7 (m, 2H), 3.8 (t, 2H), 3.85 (t, 2H), 4.1 (s, 3H), 4.6 (t, 2H), 5.1 (s, 2H), 7.55 (d, 1H), 7.6 (m, 2H), 7.75 (m, 3H), 8.2 (s, 1H), 9.0 (s, 1H); Mass Spectrum: M+H⁺ 544.

[22] The reaction product was purified by chromatography on silica using increasingly polar mixtures of methylene chloride and methanol as eluent. The product gave the following characterising data: —¹H NMR: (DMSOd₆) 2.15 (s, 3H), 3.1 (t, 2H), 3.98 (s, 3H), 3.99 (s, 3H), 4.1 (t, 2M), 5.05 (s, 2H), 7.15 (d, 1H), 7.4 (s, 1H), 7.45 (d, 1H), 7.55 (s, 1H), 7.65 (s, 1H), 8.1 (s, 1H), 8.2 (s, 1H), 8.65 (s, 1H); Mass Spectrum: M+H⁺ 489.

[23] Mass Spectrum: M+H⁺ 461.

The 6-amino-N-methylindoline used as a starting material was prepared as follows: —

Using analogous procedures to those described in the portion of Note [20] above that is concerned with the preparation of starting materials, 6-nitroindoline was converted into N-methyl-6-nitroindoline; ¹H NMR: (CDCl₃) 2.8 (s, 3H), 3.05 (m, 2H), 3.45 (m, 2H), 7.1 (d, 1H), 7.15 (s, 1H), 7.55 (d, 1H); which in turn was converted into 6-amino-N-methylindoline; Mass Spectrum: M+H⁺ 149.

[24] On completion of the reaction, the reaction mixture was diluted with a saturated aqueous sodium bicarbonate solution and extracted with methylene chloride. The organic extract was dried over magnesium sulphate and evaporated. The resultant product was purified by chromatography on silica using increasingly polar mixtures of methylene chloride and a 3M methanolic ammonia solution as eluent. The product gave the following characterising data: —¹H NMR: (DMSOd₆) 1.7 (br s, 4H), 2.6 (m, 4H), 2.65 (s, 3H), 2.85 (t, 2H), 2.9 (br s, 2H), 3.25 (t, 2H), 4.0 (s, 3H), 4.3 (br s, 2H), 4.95 (s, 2H), 6.75 (d, 1H), 6.85 (s, 1H), 6.95 (d, 1H), 7.4 (s, 1H), 7.55 (s, 1H), 7.65 (s, 1H), 8.1 (s, 1H), 8.65 (s, 1H); Mass Spectrum: M+H⁺ 544.

[25] ¹H NMR: (CDCl₃) 1.95 (m, 2H), 2.7 (m, 2H), 2.9 (s, 3H), 3.2 (m, 2H), 4.07 (s, 3H), 4.08 (s, 3H), 4.95 (s, 2H), 6.6 (d, 1H), 6.8 (s, 1H), 6.85 (d, 1H), 7.35 (s, 1H), 7.5 (s, 1H), 7.85 (s, 1H), 8.0 (s, 1H), 8.05 (s, 1H), 8.7 (s, 1H); Mass Spectrum: M+H⁺ 475.

The 7-amino-N-methyl-1,2,3,4-tetrahydroquinoline used as a starting material was prepared as follows: —

A mixture of concentrated sulphuric acid (12.03 ml) and concentrated nitric acid (4.9 ml) was added dropwise during 15 minutes to a stirred mixture of 1,2,3,4 tetrahydroquinoline (6.66 g) and concentrated sulphuric acid (118 ml) that had been cooled to 0° C. The rate of addition was such that the temperature of the reaction mixture was maintained at below 5° C. The resultant mixture was stirred at 5° C. for 15 minutes. The mixture was poured onto ice (300 ml) and neutralised by the addition of solid sodium carbonate. The aqueous mixture was extracted with ethyl acetate and the organic phase was washed with water and brine, dried over magnesium sulphate and evaporated. The residue was purified by column chromatography on silica using increasingly polar mixtures of petroleum ether (b.p. 40-60° C.) and ethyl acetate as eluent. There was thus obtained 7-nitro-1,2,3,4 tetrahydroquinoline (6.14 g) as an oil; ¹H NMR: (CDCl₃) 1.95 (m, 2H), 2.8 (t, 2H), 3.35 (t, 2H), 7.05 (d, 1H), 7.3 (s, 1H), 7.4 (d, 1H).

Using analogous procedures to those described in the portion of Note [20] above that is concerned with the preparation of starting materials, 7-nitro-1,2,3,4-tetrahydroquinoline was converted into N-methyl-7-nitro-1,2,3,4-tetrahydroquinoline; ¹H NMR: (CDCl₃) 2.0 (m, 2H), 2.8 (t, 2H), 3.0 (s, 3H), 3.3 (t, 2H), 7.0 (d, 1H), 7.35 (s, 1H), 7.4 (d, 1H); which in turn was converted into 7-amino-N-methyl-1,2,3,4-tetrahydroquinoline.

[26] ¹H NMR: (DMSOd₆) 2.8 (s, 3H), 3.2 (m, 2H), 3.98 (s, 3H), 3.99 (s, 3H), 4.25 (m, 2H), 4.95 (s, 2H), 6.65 (d, 1H), 6.95 (d, 1H), 7.0 (s, 1H), 7.4 (s, 1H), 7.55 (s, 1H), 7.65 (s, 1H), 8.1 (s, 1H), 8.65 (s, 1H), 10.05 (s, 1H); Mass Spectrum: M+H⁺ 477.

The 7-amino-4-methyl-2H-1,4-benzoxazine used as a starting material was prepared as follows: —

3-Chloropropanoyl chloride (11.73 ml) was added portionwise to a stirred mixture of 2-amino-5-nitrophenol (23.15 g), benzyltrimethylammonium chloride (27.86 g), sodium bicarbonate (63.1 g) and chloroform (750 ml) that had been cooled to 0° C. The resultant mixture was stirred at 0° C. for 1 hour and at ambient temperature for 16 hours. The solvent was evaporated and the residue was diluted with water. The precipitate was collected, washed with water and recrystallized from ethanol. There was thus obtained 7-nitro-2H-1,4-benzoxazin-3(4H)-one (11.47 g); ¹H NMR: (DMSOd₆) 4.75 (s, 2H), 7.1 (d, 1H), 7.8 (s, 1H), 7.9 (d, 1H).

Methyl iodide (2.8 ml) was added to a stirred mixture of 7-nitro-2H-1,4-benzoxazin-3(4H)-one (5.82 g), benzyltrimethylammonium chloride (0.56 g), caesium hydroxide (25.19 g) and methylene chloride (60 ml) and the resultant mixture was stirred at ambient temperature for 1.5 hours. The mixture was diluted with water (15 ml) and extracted with methylene chloride. The organic phase was washed with water and brine, dried over magnesium sulphate and evaporated. There was thus obtained 4-methyl-7-nitro-2H-1,4-benzoxazin-3(4H)-one (0.675 g). The aqueous phase was acidified with concentrated sulphuric acid and extracted with ethyl acetate. The organic phase was washed with water and brine, dried over magnesium sulphate and evaporated to give a second batch (3.053 g) of the material; ¹H NMR: (DMSOd₆ and CD₃CO₂D) 3.35 (s, 3H), 4.8 (s, 2H), 7.35 (d, 1H), 7.8 (s, 1H), 8.0 (d, 1H).

Sodium borohydride (1.01 g) was added portionwise to a stirred suspension of 4-methyl-7-nitro-2H-1,4-benzoxazin-3(4H)-one (3.7 g), nickel(II) chloride hexahydrate (2.11 g), and methanol (70 ml) that had been cooled to 0° C. The resultant mixture was stirred at 0° C. for 30 minutes and at ambient temperature for 2 hours. The solvent was evaporated and the residue was purified by column chromatography on silica using increasingly polar mixtures of methylene chloride and ethyl acetate as eluent. There was thus obtained 7-amino-4-methyl-2H-1,4-benzoxazin-3(4H)-one (1.45 g); ¹H NMR: (CDCl₃) 3.3 (s, 3H), 3.6 (br s, 2H), 4.45 (s, 2H), 6.3 (s, 1H), 6.35 (m, 1H), 6.75 (d, 1H).

A portion (1.08 g) of the material so obtained was added portionwise to a stirred solution of lithium aluminium hydride (1M in THF; 12 ml) and THF (8 ml) that had been cooled to 0° C. The mixture was stirred at 0° C. for 30 minutes. Water (1 ml) was added and the reaction mixture was diluted with ethyl acetate. The organic phase was dried over magnesium sulphate and evaporated. The residue was purified by column chromatography on silica using increasingly polar mixtures of petroleum ether (b.p. 40-60° C.) and ethyl acetate as eluent. There was thus obtained 7-amino-4-methyl-2H-1,4-benzoxazine (0.54 g); ¹H NMR: (CDCl₃) 2.8 (br s, 3H), 3.15 (br s, 2H), 4.3 (m, 2H), 6.2 (s, 1H), 6.25 (d, 1H), 6.55 (d, 1H).

[27] ¹H NMR: (DMSOd₆) 1.7 (m, 4H), 2.0 (t, 2H), 2.55 (m, 4H), 2.65 (m, 2H), 4.0 (s, 3H), 4.25 (t, 2H), 5.1 (s, 2H), 6.05 (s, 2H), 6.75 (d, 1H), 6.8 (t, 1H), 7.3 (d, 1H), 7.4 (s, 1H), 7.55 (s, 1H), 7.65 (s, 1H), 8.1 (s, 1H), 8.65 (s, 1H); Mass Spectrum: M+H⁺ 547.

The 2-{4-[7-methoxy-6-(3-pyrrolidin-1-ylpropoxy)quinazolin-4-yloxy]pyrazol-1-yl}acetic acid used as a starting material was prepared as follows: —

Di-tert-butyl azodicarboxylate (1.64 g) was added to a stirred mixture of 4-chloro-6-hydroxy-7-methoxyquinazoline (1 g), 3-pyrrolidin-1-ylpropanol (0.74 g), triphenylphosphine (1.87 g) and methylene chloride (50 ml). The resultant mixture was stirred at ambient temperature for 16 hours. The mixture was evaporated and the residue was purified by column chromatography on silica using increasingly polar mixtures of methylene chloride and methanol as eluent. There was thus obtained 4-chloro-7-methoxy-6-(3-pyrrolidin-1-ylpropoxy)quinazoline as a solid (1.38 g); Mass Spectrum: M+H^{+ 322} and 324.

A mixture of the material so obtained, tert-butyl 2-(4-hydroxypyrazol-1-yl)acetate (0.89 g), potassium carbonate (0.89 g) and DMF (15 ml) was stirred and heated to 90° C. for 2 hours. The resultant mixture was filtered and the filtrate was evaporated. The residue was purified by column chromatography on silica using increasingly polar mixtures of methylene chloride and methanol as eluent. There was thus obtained tert-butyl 2-{4-[7-methoxy-6-(3-pyrrolidin-1-ylpropoxy)quinazolin-4-yloxy]pyrazol-1-yl}acetate (1.4 g); ¹H NMR: (DMSOd₆) 1.45 (s, 9H), 1.7 (m, 4H), 2.0 (m, 2H), 2.5 (m, 4H), 2.65 (m, 2H), 4.0 (s, 3H), 4.2 (t, 2H), 4.95 (s, 2H), 7.4 (s, 1H), 7.55 (s, 1H), 7.65 (s, 1H), 8.1 (s, 1H), 8.65 (s, 1.11); Mass Spectrum: M+H⁺ 484.

A mixture of a portion (1.02 g) of the material so obtained, trifluoroacetic acid (10 ml) and methylene chloride (10 ml) was stirred at ambient temperature for 2 hours. The resultant solution was evaporated. Toluene was added and the mixture was re-evaporated. The residue was triturated under diethyl ether. The solid so obtained was suspended in methylene chloride and sufficient diisopropylethylamine was added to obtain a solution. The resultant solution was evaporated and the solid so obtained was triturated under diethyl ether. The material so obtained was purified by column chromatography on silica using increasingly polar mixtures of methylene chloride and methanol as eluent. There was thus obtained 2-{4-[7-methoxy-6-(3-pyrrolidin-1-ylpropoxy)quinazolin-4-yloxy]pyrazol-1-yl}acetic acid in 77% yield; Mass Spectrum: M+H⁺ 428.

### EXAMPLE 38

### N-[1-(2-hydroxyethyl)indol-6-yl]-2-[4-(6,7-dimethoxyquinazolin-4-yloxy)pyrazol-1-yl]acetamide

Using an analogous procedure to that described in Example 34, 2-[4-(6,7-dimethoxyquinazolin-4-yloxy)pyrazol-1-yl]acetic acid (0.31 g) was reacted with 6-amino-1-[2-(tert-butyldimethylsilyloxy)ethyl]indole (0.28 g). The reaction mixture was diluted with water and extracted with ethyl acetate. The organic phase was washed with water and with brine, dried over magnesium sulphate and evaporated. There was thus obtained N-{1-[2-(tert-butyldimethylsilyloxy)ethyl]indol-6-yl}-2-[4-(6,7-dimethoxyquinazolin-4-yloxy)pyrazol-1-yl]acetamide as a foam (0.49 g) which was used without further purification; ¹H NMR: (DMSOd₆) 0.05 (s, 6H), 0.85 (s, 9H), 3.95 (t, 2H), 4.0 (s, 6H), 4.2 (t, 2H), 5.1 (s, 2H), 6.4 (d, 1H), 7.1 (d, 1H), 7.3 (d, 1H), 7.4 (s, 1H), 7.5 (d, 1H), 7.55 (s, 1H), 7.8 (s, 1H), 7.95 (s, 1H), 8.2 (s, 1H), 8.7 (s, 1H), 10.3 (s, 1H).

A mixture of the material so obtained, tetra-n-butyl ammonium fluoride (1M solution in THF, 0.32 ml) and THF (50 ml) was stirred at ambient temperature for 2 hours. Water was added and the reaction mixture was extracted with ethyl acetate. The organic phase was washed with water and with brine, dried over magnesium sulphate and evaporated. The residue was purified by column chromatography on silica using increasingly polar mixtures of methylene chloride and methanol as eluent. There was thus obtained the title compound (0.04 g); ¹H NMR: (DMSOd₆) 3.7 (m, 2H), 4.0 (s, 6H), 4.15 (m, 2H), 4.9 (t, 1H), 5.1 (s, 2H), 6.35 (d, 1H), 7.05 (d, 1H), 7.3 (d, 1H), 7.4 (s, 1H), 7.5 (d, 1H), 7.55 (s, 1H), 7.7 (s, 1H), 7.95 (s, 1H), 8.15 (s, 1H), 8.65 (s, 1H), 10.3 (s, 1H); Mass Spectrum: M+H⁺ 489.

The 6-amino-1-[2-(tert-butyldimethylsilyloxy)ethyl]indole used as a starting material was prepared as follows: —

Sodium hydride (60% dispersion in mineral oil; 0.404 g) was added portionwise to a stirred solution of 6-nitroindole (1.63 g) in DMF (20 ml) and the mixture was stirred at ambient temperature for 5 minutes. 2-(tert-Butyldimethylsilyloxy)ethyl bromide (2.17 ml) was added and the resultant mixture was stirred and heated to 80° C. for 20 minutes. Water was added and the mixture was extracted with ethyl acetate. The organic phase was washed with water and with brine, dried over magnesium sulphate and evaporated. The residue was purified by column chromatography on silica using increasingly polar mixtures of petroleum ether (b.p. 40-60° C.) and ethyl acetate as eluent. There was thus obtained 1-[2-(tert-butyldimethylsilyloxy)ethyl]-6-nitroindole as an oil (3 g); ¹H NMR: (CDCl₃) 0.05 (s, 6H), 0.95 (s, 9H), 4.1 (t, 2H), 4.5 (t, 2H), 6.75 (d, 1H), 7.6 (d, 1H), 7.8 (d, 1H), 8.2 (d, 1H), 8.55 (s, 1H).

Using an analogous procedure to that described in the portion of Note [24] below Example 35 that is concerned with the preparation of starting materials, 1-[2-(tert-butyldimethylsilyloxy)ethyl]-6-nitroindole was hydrogenated to give 6-amino-1-[2-(tert-butyldimethylsilyloxy)ethyl]indole; ¹H NMR: (CDCl₃) 0.05 (s, 6H), 0.95 (s, 9H), 4.0 (t, 2H), 4.25 (t, 2H), 6.45 (d, 1H), 6.65 (d, 1H), 6.75 (s, 1H), 7.05 (d, 1H), 7.5 (d, 1H).

### EXAMPLE 39

### N-(6-indolinyl)-2-{4-[6-methoxy-7-(2-pyrrolidin-1-ylethoxy)quinazolin-4-yloxy]pyrazol-1-yl}acetamide

A mixture of N-[1-(N-tert-butoxycarbonyl)indolin-6-yl]-2-{4-[6-methoxy-7-(2-pyrrolidin-1-ylethoxy)quinazolin-4-yloxy]pyrazol-1-yl}acetamide (0.282 g), trifluoroacetic acid (4 ml) and methylene chloride (1 ml) was stirred at ambient temperature for 1 hour. The resultant mixture was evaporated and the residue was purified by preparative HPLC using a Waters ‘β Basic Hypersil’ reversed-phase column (5 microns silica, 30 mm diameter, 250 mm length) and decreasingly polar mixtures of water (containing 0.2% ammonium carbonate) and acetonitrile as eluent. The solid so obtained was triturated under diethyl ether. The resultant solid was dried under vacuum. There was thus obtained the title compound (0.12 g); ¹H NMR: (DMSOd₆+CF₃CO₂D) 1.9 (m, 2H), 2.1 (m, 2H), 3.2 (m, 4H), 3.7 (m, 2H), 3.75 (m, 4H), 4.05 (s, 3H), 4.6 (t, 2H), 5.15 (s, 2H), 7.45 (d, 1H), 7.5 (d, 1H), 7.6 (s, 1H), 7.7 (s, 1H), 7.75 (s, 1H), 7.95 (s, 1H), 8.2 (s, 1H), 8.9 (s, 1H); Mass Spectrum: M+H⁺ 530.

The N-[1-(N-tert-butoxycarbonyl)indolin-6-yl]-2-{4-[6-methoxy-7-(2-pyrrolidin-1-ylethoxy)quinazolin-4-yloxy]pyrazol-1-yl}acetamide used as a starting material was prepared as follows: —

Di-tert-butyl dicarbonate (7.2 g) was added to a mixture of 6-nitroindoline (4.92 g) and methylene chloride (50 ml) and the mixture was stirred at ambient temperature for 1 hour. 4-Dimethylaminopyridine (0.37 g) was added and the reaction mixture was stirred at ambient temperature for 16 hours. The resultant mixture was evaporated and the residue was purified by column chromatography on silica using increasingly polar mixtures of petroleum ether (b.p. 40-60° C.) and ethyl acetate as eluent. There was thus obtained tert-butyl 6-nitroindoline-1-carboxylate as a solid (5.45 g); ¹H NMR: (CDCl₃) 1.6 (s, 9H), 3.2 (t, 2H), 4.1 (t, 2H), 7.2 (d, 1H), 7.85 (d, 1H), 8.3 (br s, 0.5H), 8.7 (br s, 0.5H).

A mixture of a portion (2.64 g) of the material so obtained, 10% palladium-on-carbon catalyst (0.5 g) and ethyl acetate (200 ml) was stirred under 2.7 atmospheres pressure of hydrogen for 5 hours. The resultant mixture was filtered and the filtrate was evaporated. The residue was purified by column chromatography on silica using increasingly polar mixtures of petroleum ether (b.p. 40-60° C.) and ethyl acetate as eluent. There was thus obtained tert-butyl 6-aminoindoline-1-carboxylate (1.5 g). ¹H NMR: (CDCl₃) 2.95 (m, 2H), 3.9 (m, 2H), 6.3 (d, 1H), 6.9 (d, 1H), 7.25 (s, 1H).

Using a similar procedure to that described in Example 8, 2-{4-[6-methoxy-7-(2-pyrrolidin-1-ylethoxy)quinazolin-4-yloxy]pyrazol-1-yl}acetic acid (0.322 g) was reacted with tert-butyl 6-aminoindoline-1-carboxylate (0.22 g). The reaction mixture was stirred at ambient temperature for 1 hour and the resultant reaction mixture was injected into a Waters ‘β Basic Hypersil’ reversed-phase column (5 microns silica, 30 mm diameter, 250 mm length) which was eluted with decreasingly polar mixtures of water (containing 0.2% ammonium carbonate) and acetonitrile. There was thus obtained N-[1-(N-tert-butoxycarbonyl)indolin-6-yl]-2-{4-[6-methoxy-7-(2-pyrrolidin-1-ylethoxy)quinazolin-4-yloxy]pyrazol-1-yl}acetamide as a solid (0.256 g); Mass Spectrum: M+H⁺ 630.

### EXAMPLE 40

### N-(6-indolinyl)-2-[4-(6,7-dimethoxyquinazolin-4-yloxy)pyrazol-1-yl]acetamide

Using an analogous procedure to that described in Example 39, N-[1-(N-tert-butoxycarbonyl)indolin-6-yl]-2-[4-(6,7-dimethoxyquinazolin-4-yloxy)pyrazol-1-yl]acetamide (0.7 g) was reacted with trifluoroacetic acid to give the title compound as a solid (0.348 g); ¹H NMR: (DMSOd₆) 2.85 (t, 2H), 3.4 (t, 2H), 3.98 (s, 3H), 3.99 (s, 3H), 5.0 (s, 2H), 5.6 (s, 1H), 6.7 (d, 1H), 6.9 (s, 1H), 6.95 (d, 1H), 7.4 (s, 1H), 7.55 (s, 1H), 7.7 (s, 1H), 8.1 (s, 1H), 8.65 (s, 1H), 10.0 (s, 1H); Mass Spectrum: M+H⁺ 447.

The N-[1-(N-tert-butoxycarbonyl)indolin-6-yl]-2-[4-(6,7-dimethoxyquinazolin-4-yloxy)pyrazol-1-yl]acetamide used as a starting material was prepared as follows: —

Using a similar procedure to that described in Example 8 and a similar purification procedure to that described in the last paragraph of the portion of Example 39 that is concerned with the preparation of starting materials, 2-[4-(6,7-dimethoxyquinazolin-4-yloxy)pyrazol-1-yl]acetic acid was reacted with tert-butyl 6-aminoindoline-1-carboxylate. There was thus obtained the required starting material in 55% yield; Mass Spectrum: M+H⁺ 547.

### EXAMPLE 41

### N-(6-indolinyl)-2-[4-(7-ethoxy-6-methoxyquinazolin-4-yloxy)pyrazol-1-yl]acetamide

Using an analogous procedure to that described in Example 39, N-[1-(N-tert-butoxycarbonyl)indolin-6-yl]-2-[4-(7-ethoxy-6-methoxyquinazolin-4-yloxy)pyrazol-1-yl]acetamide (059 g) was reacted with trifluoroacetic acid. On completion of the reaction, the reaction mixture was evaporated and the residue was purified by column chromatography on silica using methylene chloride as eluent. The material so obtained was triturated under diethyl ether. The solid so obtained was dried under vacuum. There was thus obtained the title compound as a solid (0.216 g); ¹H NMR: (DMSOd₆) 1.4 (t, 3H), 2.85 (t, 2H), 3.4 (t, 2H), 3.95 (s, 3H), 4.25 (q, 2H), 4.95 (s, 2H), 5.6 (s, 1H), 6.7 (d, 1H), 6.9 (s, 1H), 6.95 (d, 1H), 7.4 (s, 1H), 7.55 (s, 1H), 7.65 (s, 1H), 8.1 (s, 1H), 8.65 (s, 1H), 10.0 (s, 1H); Mass Spectrum: M+Hi 461.

The N-[1-(N-tert-butoxycarbonyl)indolin-6-yl]-2-[4-(7-ethoxy-6-methoxyquinazolin-4-yloxy)pyrazol-1-yl]acetamide used as a starting material was prepared as follows: —

A mixture of 4-(tert-butyldimethylsilyloxy)-1H-pyrazole (17.1 g), benzyl bromoacetate (15.73 ml), potassium carbonate (24 g) and DMF (150 ml) was stirred and heated to 50° C. for 1.5 hours. The mixture was filtered. The filtrate was concentrated by evaporation and partitioned between diethyl ether and water. The organic phase was washed with water and brine, dried over magnesium sulphate and evaporated. The residue was purified by column chromatography on silica using a 1:1 mixture of petroleum ether (b.p. 40-60° C.) and ethyl acetate as eluent. There was thus obtained benzyl 2-[4-(tert-butyldimethylsilyloxy)pyrazol-1-yl]acetate as an oil (29.4 g); ¹H NMR: (CDCl₃) 0.0 (s, 6H), 0.8 (s, 9H), 4.65 (s, 2H), 5.05 (s, 2H), 6.9 (s, 1H), 7.05 (s, 1H), 7.2 (m, 5H).

A mixture of the material so obtained, 10% palladium-on-carbon catalyst (1.5 g), ethyl acetate (50 ml) and ethanol (400 ml) was stirred under an atmospheres pressure of hydrogen for 40 minutes. The catalyst was removed by filtration and the solvent was evaporated to leave a solid that was triturated under petroleum ether. The resultant solid was collected by filtration and dried under vacuum. There was thus obtained 2-[4-(tert-butyldimethylsilyloxy)pyrazol-1-yl]acetic acid (15 g); ¹H NMR: (DMSOd₆) 0.0 (s, 6H), 0.8 (s, 9H), 4.65 (s, 2H), 6.95 (s, 1H), 7.25 (s, 1H); Mass Spectrum: M+H⁺ 257.

Diisopropylethylamine (0.9 ml) and 2-(7-azabenzotriazol-1-yl)-1,1,3,3-tetramethyluronium hexafluorophosphate(V) (1.98 g) were added in turn to a stirred mixture of 2-[4-(tert-butyldimethylsilyloxy)pyrazol-1-yl]acetic acid (1.15 g), tert-butyl 6-aminoindoline-1-carboxylate (1.03 g) and DMF (10 ml) and the resultant mixture was stirred at ambient temperature for 16 hours. The reaction mixture was diluted with water and extracted with ethyl acetate. The organic phase was washed with water and with brine, dried over magnesium sulphate and evaporated. The residue was purified by column chromatography on silica using methylene chloride as eluent. The material so obtained was triturated under diethyl ether. There was thus obtained N-[1-(N-tert-butoxycarbonyl)indolin-6-yl]-2-[4-(tert-butyldimethylsilyloxy)pyrazol-1-yl]acetamide as a solid (1.42 g); Mass Spectrum: M+H⁺ 473.

A mixture of a portion (1.19 g) of the material so obtained and THF (10 ml) was cooled to 0° C. Acetic acid (0.358 ml) and tetrabutylammonium fluoride (1M in THF; 2.76 ml) were added in turn and the reaction mixture was stirred for 1.5 hours whilst being allowed to warm to ambient temperature. A saturated aqueous sodium bicarbonate solution was added and the mixture was extracted with ethyl acetate. The organic phase was washed with water and brine, dried over magnesium sulphate and evaporated. The residue was purified by column chromatography on silica using methylene chloride as eluent. There was thus obtained N-[1-(N-tert-butoxycarbonyl)indolin-6-yl]-2-(4-hydroxypyrazol-1-yl)acetamide (0.9 g).

A mixture of a portion (0.415 g) of the material so obtained, 4-chloro-7-ethoxy-6-methoxyquinazoline (0.268 g), caesium carbonate (0.413 g) and DMA (3 ml) was stirred and heated to 90° C. for 2 hours. The resultant mixture was cooled to ambient temperature, poured into water and extracted with ethyl acetate. The organic phase was washed with water, dried over magnesium sulphate and evaporated. The residue was triturated under diethyl ether. The resultant solid was dried under vacuum. There was thus obtained N-[1-(N-tert-butoxycarbonyl)indolin-6-yl]-2-[4-(7-ethoxy-6-methoxyquinazolin-4-yloxy)pyrazol-1-yl]acetamide (0.593 g); Mass Spectrum: M+H⁺ 561.

### EXAMPLE 42

### N-(5-indolinyl)-2-[4-(6,7-dimethoxyquinazolin-4-yloxy)pyrazol-1-yl]acetamide

Using an analogous procedure to that described in Example 39, N-[1-(N-tert-butoxycarbonyl)indolin-5-yl]-2-[4-(6,7-dimethoxyquinazolin-4-yloxy)pyrazol-1-yl]acetamide (045 g) was reacted with trifluoroacetic acid to give the title compound as a solid (0.33 g); ¹H NMR: (DMSOd₆) 2.85 (t, 2H), 3.4 (t, 2H), 3.98 (s, 3H), 3.99 (s, 3H), 4.95 (s, 2H), 5.35 (s, 1H), 6.45 (d, 1H), 7.1 (d, 1H), 7.3 (d, 1H), 7.4 (s, 1H), 7.55 (s, 1H), 7.65 (s, 1H), 8.1 (s, 1H), 8.65 (s, 1H); Mass Spectrum: M+H⁺ 447.

The N-[1-(N-tert-butoxycarbonyl)indolin-5-yl]-2-[4-(6,7-dimethoxyquinazolin-4-yloxy)pyrazol-1-yl]acetamide used as a starting material was prepared as follows: —

Di-tert-butyl dicarbonate (4.8 g) was added to a stirred mixture of 5-nitroindoline (3.28 g), 4-dimethylaminopyridine (0.24 g) and methylene chloride (50 ml) and the mixture was stirred at ambient temperature for 16 hours. The resultant mixture was evaporated and the residue was purified by column chromatography on silica using increasingly polar mixtures of petroleum ether (b.p. 40-60° C.) and ethyl acetate as eluent. There was thus obtained tert-butyl 5-nitroindoline-1-carboxylate as a solid (4.7 g); ¹H NMR: (CDCl₃) 1.55 (s, 9H), 3.2 (t, 2H), 4.1 (t, 2H), 7.9 (br s, 1H), 8.0 (s, 1H), 8.1 (d, 1H).

A mixture of a portion (1.3 g) of the material so obtained, 10% palladium-on-carbon catalyst (0.2 g), ammonium formate (1.86 g) and methanol (10 ml) was stirred at ambient temperature for 1.5 hours. The resultant mixture was filtered and the filtrate was evaporated. The residue was purified by column chromatography on silica using increasingly polar mixtures of petroleum ether (b.p. 40-60° C.) and ethyl acetate as eluent. There was thus obtained tert-butyl 5-aminoindoline-1-carboxylate as an oil (1.05 g).

Using a similar procedure to that described in Example 8 and a similar purification procedure to that described in the last paragraph of the portion of Example 39 that is concerned with the preparation of starting materials, 2-[4-(6,7-dimethoxyquinazolin-4-yloxy)pyrazol-1-yl]acetic acid was reacted with tert-butyl 5-aminoindoline-1-carboxylate. There was thus obtained the required starting material in 67% yield; Mass Spectrum: M+H⁺ 547.

### EXAMPLE 43

### N-(6-indolinyl)-2-{4-[7-(2-hydroxyethoxy)-6-methoxyquinazolin-4-yloxy]pyrazol-1-yl}acetamide

A mixture of N-[1-(N-tert-butoxycarbonyl)indolin-6-yl]-2-{4-[7-(2-hydroxyethoxy)-6-methoxyquinazolin-4-yloxy]pyrazol-1-yl}acetamide (0.07 g), a 4M solution of hydrogen chloride in 1,4-dioxane (0.5 ml), acetonitrile (1 ml) and 1,4-dioxane (1 ml) was stirred at ambient temperature for 1 hour. The resultant solid was collected by filtration and washed with water. The solid was suspended in water and the mixture was neutralised by the addition of a saturated aqueous solution of sodium bicarbonate. The resultant solid was collected by filtration and dried under vacuum. There was thus obtained the title compound (0.058 g); ¹H NMR: (DMSOd₆+CF₃CO₂D) 2.85 (t, 2H), 3.4 (t, 2H), 3.85 (m, 2H), 4.0 (s, 3H), 4.25 (m, 2H), 5.0 (s, 2H), 6.7 (d, 1H), 6.9 (s, 1H), 6.95 (d, 1H), 7.4 (s, 1H), 7.55 (s, 1H), 7.65 (s, 1H), 8.10 (s, 1H), 8.65 (s, 1H); Mass Spectrum: M+H⁺ 477.

The N-[1-(N-tert-butoxycarbonyl)indolin-6-yl]-2-{4-[7-(2-hydroxyethoxy)-6-methoxyquinazolin-4-yloxy]pyrazol-1-yl}acetamide used as a starting material was prepared as follows: —

Using a similar procedure to that described in Example 8 and a similar purification procedure to that described in the last paragraph of the portion of Example 39 that is concerned with the preparation of starting materials, a mixture of 2-{4-[7-(2-hydroxyethoxy)-6-methoxyquinazolin-4-yloxy]pyrazol-1-yl}acetic acid and the corresponding 7-(2-trifluoroacetoxyethoxy) compound was reacted with tert-butyl 6-aminoindoline-1-carboxylate. There was thus obtained the required starting material in 25% yield; Mass Spectrum: M+H⁺ 577.

### EXAMPLE 44

### N-(6-indolinyl)-2-{4-[7-(2-methoxyethoxy)-6-methoxyquinazolin-4-yloxy]pyrazol-1-yl}acetamide

Using an analogous procedure to that described in Example 42, N-[1-(N-tert-butoxycarbonyl)indolin-6-yl]-2-{4-[7-(2-methoxyethoxy)-6-methoxyquinazolin-4-yloxy]pyrazol-1-yl}acetamide was reacted with 4M hydrogen chloride in 1,4-dioxane. The resultant mixture was evaporated and the residue was purified by preparative HPLC using a Waters ‘β Basic Hypersil’ reversed-phase column (5 microns silica, 30 mm diameter, 250 mm length) and decreasingly polar mixtures of water (containing 0.2% ammonium carbonate) and acetonitrile as eluent. The resultant solid was dried under vacuum. There was thus obtained the title compound (0.075 g); ¹H NMR: (DMSOd₆) 2.85 (t, 2H), 3.3 (s, 3H), 3.4 (t, 2H), 3.75 (m, 2H), 4.0 (s, 3H), 4.35 (m, 2H), 4.95 (s, 2H), 5.6 (s, 1H), 6.7 (d, 1H), 6.9 (s, 1H), 6.95 (d, 1H), 7.4 (s, 1H), 7.55 (s, 1H), 7.65 (s, 1H), 8.1 (s, 1H), 8.65 (s, 1H); Mass Spectrum: M+H⁺ 491.

The N-[1-(N-tert-butoxycarbonyl)indolin-6-yl]-2-{4-[7-(2-methoxyethoxy)-6-methoxyquinazolin-4-yloxy]pyrazol-1-yl}acetamide used as a starting material was prepared as follows: —

Using a similar procedure to that described in Example 32, 2-{4-[7-(2-methoxyethoxy)-6-methoxyquinazolin-4-yloxy]pyrazol-1-yl}acetic acid was reacted with tert-butyl 6-aminoindoline-1-carboxylate. There was thus obtained the required starting material in 69% yield; Mass Spectrum: M−H⁻ 89.

### EXAMPLE 45

### N-(6-indolinyl)-2-{4-[6,7-di-(2-methoxyethoxy)quinazolin-4-yloxy]pyrazol-1-yl}acetamide

Using an analogous procedure to that described in Example 42, N-[1-(N-tert-butoxycarbonyl)indolin-6-yl]-2-{4-[6,7-di-(2-methoxyethoxy)quinazolin-4-yloxy]pyrazol-1-yl}acetamide (0.4 g) was reacted with 4M hydrogen chloride in 1,4-dioxane. The precipitated solid was suspended in methylene chloride and a saturated irethanolic ammonia solution was added to obtain a solution. The resultant solvents were evaporated and the residual solid was washed with water and with diethyl ether and dried under vacuum at 40° C. for 16 hours. There was thus obtained the title compound (0.212 g); ¹H NMR: (DMSOd₆ and CF₃CO₂D) 3.2 (m, 2H), 3.4 (s, 6H), 3.8 (m, 6H), 4.4 (m, 4H), 5.1 (s, 2H), 7.45 (d, 1H), 7.5 (m, 2H), 7.75 (m, 2H), 7.95 (s, 1H), 8.2 (s, 1H), 9.05 (br s, 1H); Mass Spectrum: M+H⁺ 535.

The N-[1-(N-tert-butoxycarbonyl)indolin-6-yl]-2-{4-[6,7-di-(2-methoxyethoxy)quinazolin-4-yloxy]pyrazol-1-yl}acetamide used as a starting material was prepared as follows: —

Using a similar procedure to that described in Example 32 except that the reaction mixture was stirred at 45° C. for 3 hours, 2-{4-[6,7-di-(2-methoxyethoxy)quinazolin-4-yloxy]pyrazol-1-yl}acetic acid was reacted with tert-butyl 6-aminoindoline-1-carboxylate. On completion of the reaction, water was added and the precipitate was collected by filtration, washed with diethyl ether and dried under vacuum. There was thus obtained the required starting material in 62% yield; Mass Spectrum: M+H⁺ 635.

### EXAMPLE 46

### N-(6-indolinyl)-2-{4-[7-(2,3-dihydroxypropoxy)-6-methoxyquinazolin-4-yloxy]pyrazol-1-yl}acetamide

Using an analogous procedure to that described in Example 42, N-[1-(N-tert-butoxycarbonyl)indolin-6-yl]-2-{4-[7-(2,3-dihydroxypropoxy)-6-methoxyquinazolin-4-yloxy]pyrazol-1-yl}acetamide (0.07 g) was reacted with 4M hydrogen chloride in 1,4-dioxane. The resultant solid was collected by filtration and washed with water. The solid was suspended in water and the mixture was neutralised by the addition of a saturated aqueous solution of sodium bicarbonate. The resultant solid was collected by filtration and dried under vacuum. There was thus obtained the title compound (0.042 g); ¹H NMR: (DMSOd₆ and CF₃CO₂D) 2.85 (t, 2H), 3.4 (t, 2H), 3.5 (d, 2H), 3.9 (m, 1H), 4.0 (s, 3H), 4.1 (m, 1H), 4.25 (m, 1H), 5.0 (s, 2H), 6.7 (d, 1H), 6.9 (s, 1H), 6.95 (d, 1H), 7.4 (s, 1H), 7.55 (s, 1H), 7.7 (s, 1H), 8.1 (s, 1H), 8.65 (s, 1H); Mass Spectrum: M+H⁺ 507.

The N-[1-(N-tert-butoxycarbonyl)indolin-6-yl]-2-{4-[7-(2,3-dihydroxypropoxy)-6-methoxyquinazolin-4-yloxy]pyrazol-1-yl}acetamide used as a starting material was prepared as follows: —

Using a similar procedure to that described in Example 8 except that 2-(benzotriazol-1-yl)-1,1,3,3-tetramethyluronium tetrafluoroborate was used in pace of 2-(7-azabenzotriazol-1-yl)-1,1,3,3-tetramethyluronium hexafluorophosphate(V), a 1:1 mixture of 2-{4-[7-(2,3-dihydroxypropoxy)-6-methoxyquinazolin-4-yloxy]pyrazol-1-yl}acetic acid and the corresponding 7-(2-hydroxy-3-trifluoroacetoxypropoxy) compound was reacted with tert-butyl 6-aminoindoline-1-carboxylate. The reaction mixture was stirred at ambient temperature for 12 hours. A 2N aqueous sodium carbonate solution was added and the reaction mixture was stirred at ambient temperature for 30 minutes. The precipitate was collected by filtration, washed with diethyl ether and dried under vacuum. There was thus obtained the required starting material in 28% yield; Mass Spectrum: M+H⁺ 607.

### EXAMPLE 47

### N-[3-cyclopropylaminomethyl-5-methylphenyl]-2-{4-[7-(2-hydroxyethoxy)-6-methoxyquinazolin-4-yloxy]pyrazol-1-yl}acetamide

A mixture of N-[3-(N-tert-butoxycarbonyl-N-cyclopropylaminomethyl)-5-methylphenyl]-2-{4-[6-methoxy-7-(2-tetrahydropyranyloxyethoxy)quinazolin-4-yloxy]pyrazol-1-yl}acetamide (0.285 g), glacial acetic acid (6 ml), water (3 ml) and THF (1 ml) was stirred and heated to 50° C. for 1 hour. The resultant mixture was cooled and the solvents were evaporated. The residue was dissolved in methylene chloride (10 ml) and the solution was cooled to 0° C. Trifluoroacetic acid (5 ml) was added and the mixture was stirred at 0° C. for 1 hour. The resultant mixture was evaporated, toluene was added and the mixture was evaporated. The material so obtained was triturated under ethyl acetate. There was thus obtained the title compound as a white solid (0.17 g); ¹H NMR: (DMSOd₆) 0.3 (m, 2H), 0.4 (m, 2H), 2.1 (m, 1H), 2.25 (s, 3H), 3.7 (s, 2H), 3.85 (s, 2H), 4.0 (s, 3H), 4.2 (t, 2H), 4.95 (m, 1H), 5.0 (s, 2H), 6.9 (s, 1H), 7.3 (s, 1H), 7.35 (s, 1H), 7.4 (s, 1H), 7.55 (s, 1H), 7.7 (s, 1H), 8.15 (s, 1H), 8.65 (s, 1H); Mass Spectrum: M+H⁺ 519.

The N-[3-(N-tert-butoxycarbonyl-N-cyclopropylaminomethyl)-5-methylphenyl]-2-{4-[6-methoxy-7-(2-tetrahydropyranyloxyethoxy)quinazolin-4-yloxy]pyrazol-1-yl}acetamide used as a starting material was prepared as follows: —

Di-tert-butyl azodicarboxylate (16.42 g) was added portionwise to a stirred mixture of 4-chloro-7-hydroxy-6-methoxyquinazoline (10 g), 2-(2-hydroxyethoxy)tetrahydropyran (7.72 ml), triphenylphosphine (18.71 g) and methylene chloride (500 ml) that was cooled to about 5° C. The resultant mixture was stirred at ambient temperature for 2.5 hours. The mixture was evaporated and the residue was triturated under diethyl ether. The precipitated triphenylphosphine oxide was separated by filtration and the filtrate was evaporated. The residue was purified by column chromatography on silica using increasingly polar mixtures of petroleum ether (b.p. 40-60° C.) and ethyl acetate as eluent. There was thus obtained 4-chloro-6-methoxy-7-(2-tetrahydropyran-2-yloxyethoxy)quinazoline (10 g).

Tetra-n-butylammonium fluoride (1M in THF; 21.4 ml) and acetic acid (2.79 ml) were added in turn to a solution of benzyl 2-[4-(tert-butyldimethylsilyloxy)pyrazol-1-yl]acetate (6.75 g) in THF (100 ml) that had been cooled to 0° C. The resultant mixture was stirred at ambient temperature for 2 hours. The reaction mixture was diluted with a saturated aqueous sodium bicarbonate solution and extracted with ethyl acetate. The organic phase was recovered, washed with water, dried over magnesium sulphate and evaporated. There was thus obtained benzyl 2-(4-hydroxypyrazol-1-yl)acetate as an oil (5.4 g); Mass Spectrum: M+H⁺ 233.

Using an analogous procedure to that described in the last paragraph of the portion of Example 41 that is concerned with the preparation of starting materials, 4-chloro-6-methoxy-7-(2-tetrahydropyran-2-yloxyethoxy)quinazoline (2.5 g) was reacted with benzyl 2-(4-hydroxypyrazol-1-yl)acetate (1.89 g). The reaction product was purified by column chromatography on silica using increasingly polar mixtures of petroleum ether (b.p. 40-60° C.) and ethyl acetate as eluent. There was thus obtained benzyl 2-{4-[6-methoxy-7-(2-tetrahydropyran-2-yloxyethoxy)quinazolin-4-yloxy]pyrazol-1-yl}acetamide (2.65 g); Mass Spectrum: M+H⁺ 535.

A mixture of the material so obtained, 10% palladium-on-carbon catalyst (0.5 g), DMF (10 ml), methanol (25 ml) and ethyl acetate (25 ml) was stirred under 1.4 atmospheres pressure of hydrogen for 1 hour. The catalyst was removed by filtration and the solvent was evaporated. There was thus obtained 2-{4-[6-methoxy-7-(2-tetrahydropyran-2-yloxyethoxy)quinazolin-4-yloxy]pyrazol-1-yl}acetic acid (1.9 g); Mass Spectrum: M+H⁺ 44-5.

Using an analogous procedure to that described in Example 8, except that 2-(benzotriazol-1-yl)-1,1,3,3-tetramethyluronium tetrafluoroborate was used in place of 2-(7-azabenzotriazol-1-yl)-1,1,3,3-tetramethyluronium hexafluorophosphate(V), 2-{4-[6-methoxy-7-(2-tetrahydropyran-2-yloxyethoxy)quinazolin-4-yloxy]pyrazol-1-yl}acetic acid (0.2 g) was reacted with 3-(N-tert-butoxycarbonyl-N-cyclopropylaminomethyl)-5-methylaniline (0.15 g) to give the required starting material (0.285 g).

### EXAMPLE 48

### N-[3-(N-cyclopropyl-N-methylaminomethyl)-5-methylphenyl]-2-{4-[7-(2-hydroxyethoxy)-6-methoxyquinazolin-4-yloxy]pyrazol-1-yl}acetamide

A mixture of N-[3-(N-cyclopropyl-N-methylaminomethyl)-5-methylphenyl]-2-{4-[6-methoxy-7-(2-tetrahydropyranyloxyethoxy)quinazolin-4-yloxy]pyrazol-1-yl}acetamide (0.282 g), glacial acetic acid (6 ml), water (3 ml) and THF (1 ml) was stirred and heated to 50° C. for 1.5 hours. The resultant mixture was cooled and the solvents were evaporated. Toluene was added and the mixture was evaporated. The material so obtained was triturated under diethyl ether. There was thus obtained the title compound as a solid (0.108 g); ¹H NMR: (DMSOd₆) 0.3 (m, 2H), 0.45 (m, 2H), 1.7 (m, 1H), 2.15 (s, 3H), 2.25 (s, 3H), 3.55 (s, 2H), 3.8 (s, 2H), 4.0 (s, 3H), 4.2 (t, 2H), 4.95 (m, 1H), 5.0 (s, 2H), 6.8 (s, 1H), 7.3 (s, 1H), 7.35 (s, 1H), 7.4 (s, 1H), 7.55 (s, 1H), 7.7 (s, 1H), 8.15 (s, 1H), 8.65 (s, 1H); Mass Spectrum: M+H⁺ 533.

The N-[3-(N-cyclopropyl-N-methylaminomethyl)-5-methylphenyl]-2-{4-[6-methoxy-7-(2-tetrahydropyranyloxyethoxy)quinazolin-4-yloxy]pyrazol-1-yl}acetamide used as a starting material was prepared as follows: —

Using an analogous procedure to that described in Example 8, except that 2-(benzotriazol-1-yl)-1,1,3,3-tetramethyluronium tetrafluoroborate was used in place of 2-(7-azabenzotriazol-1-yl)-1,1,3,3-tetramethyluronium hexafluorophosphate(V), 2-{4-[6-methoxy-7-(2-tetrahydropyran-2-yloxyethoxy)quinazolin-4-yloxy]pyrazol-1-}acetic acid (0.2 g) was reacted with 3-(N-cyclopropyl-N-methylaminomethyl)-5-methylaniline (0.103 g) to give the required starting material (0.282 g).

### EXAMPLE 49

### N-(4-methyl-2H-1,4-benzoxazin-7-yl)-2-{4-[7-(2-hydroxyethoxy)-6-methoxyquinazolin-4-yloxy]pyrazol-1-yl}acetamide

Using an analogous procedure to that described in Example 48, N-(4-methyl-2H-1,4-benzoxazin-7-yl)-2-{4-[6-methoxy-7-(2-tetrahydropyranyloxyethoxy)quinazolin-4-yloxy]pyrazol-1-yl}acetamide (0.37 g) was reacted with glacial acetic acid to give the title compound (0.215 g); ¹H NMR: (DMSOd₆) 2.8 (s, 3H), 3.15 (t, 2H), 3.8 (m, 2H), 4.0 (s, 3H), 4.2 (m, 4H), 4.95 (s, 2H), 5.0 (m, 1H), 6.65 (d, 1H), 6.95 (d, 1H), 7.0 (s, 1H), 7.4 (s, 1H), 7.55 (s, 1H), 7.65 (s, 1H), 8.1 (s, 1H), 8.65 (s, 1H); Mass Spectrum: M+H⁺ 507.

The N-(4-methyl-2H-1,4-benzoxazin-7-yl)-2-{4-[6-methoxy-7-(2-tetrahydropyranyloxyethoxy)quinazolin-4-yloxy]pyrazol-1-yl}acetamide used as a starting material was prepared as follows: —

Using an analogous procedure to that described in Example 8, 2-[4-(tert-butyldimethylsilyloxy)pyrazol-1-yl]acetic acid (1.15 g) was reacted with 7-amino-4-methyl-2H-1,4-benzoxazine (0.4 g) to give N-(4-methyl-2H-1,4-benzoxazin-7-yl)-2-[4-(tert-butyldimethylsilyloxy)pyrazol-1-yl]acetamide as an oil (0.77 g).

A mixture of the material so obtained and THF (10 ml) was cooled to 0° C. Acetic acid (0.43 ml) and tetrabutylammonium fluoride (1M in THF; 3.31 ml) were added in turn and the reaction mixture was stirred for 1.5 hours whilst being allowed to warm to ambient temperature. A saturated aqueous sodium bicarbonate solution was added and the mixture was extracted with ethyl acetate. The organic phase was washed with water and brine, dried over magnesium sulphate and evaporated. There was thus obtained N-(4-methyl-2H-1,4-benzoxazin-7-yl)-2-(4-hydroxypyrazol-1-yl)acetamide (0.45 g); Mass Spectrum: M+H⁺ 289.

A mixture of the material so obtained, 4-chloro-6-methoxy-7-(2-tetrahydropyran-2-yloxyethoxy)quinazoline (0.53 g), caesium carbonate (0.51 g) and DMA (10 ml) was stirred and heated to 90° C. for 1.5 hours. The resultant mixture was concentrated by evaporation, poured into water and extracted with ethyl acetate. The organic phase was washed with water, dried over magnesium sulphate and evaporated. The residue was triturated under diethyl ether. The resultant solid was dried under vacuum. There was thus obtained N-(4-methyl-2H-1,4-benzoxazin-7-yl)-2-{4-[6-methoxy-7-(2-tetrahydropyranyloxyethoxy)quinazolin-4-yloxy]pyrazol-1-yl}acetamide (0.37 g); ¹H NMR: (DMSOd₆) 1.4-1.8 (br m; 6H), 2.8 (s, 3H), 3.2 (t, 2H), 3.45 (m, 1H), 3.8 (m, 2H), 4.0 (s, 3H), 4.05 (m, 1H), 4.25 (t, 2H), 4.4 (m, 2H), 4.7 (m, 1H), 4.95 (s, 2H), 6.65 (d, 1H), 6.95 (d, 1H), 7.05 (s, 1H), 7.45 (s, 1H), 7.55 (s, 1H), 7.65 (s, 1H), 8.1 (s, 1H), 8.65 (s, 1H).

### EXAMPLE 50

### N-(2,3-methylenedioxyphenyl)-2-{4-[6-(2-pyrrolidin-1-ylethoxy)-7-methoxyquinazolin-4-yloxy]pyrazol-1-yl}acetamide

A mixture of N-(2,3-methylenedioxyphenyl)-2-{4-[6-(2-chloroethoxy)-7-methoxyquinazolin-4-yloxy]pyrazol-1-yl}acetamide (0.18 g), pyrrolidine (0.043 ml), potassium carbonate (0.05 g), potassium iodide (0.085 g) and DMA (1.3 ml) was stirred and heated to 120° C. in a microwave oven for 5 minutes. The reaction mixture was purified by preparative HPLC using a Waters ‘β Basic Hypersil’ reversed-phase column (5 microns silica, 30 mm diameter, 250 mm length) and decreasingly polar mixtures of water (containing 0.2% ammonium carbonate) and acetonitrile as eluent. There was thus obtained the title compound as a white solid (0.108 g); ¹H NMR: (DMSOd₆) 1.7 (m, 4H), 2.55 (m, 4H), 2.9 (t, 2H) 4.0 (s, 3H), 4.25 (t, 2H), 5.1 (s, 2H), 6.05 (s, 2H), 6.75 (d, 1H), 6.8 (m, 1H), 7.3 (d, 1H), 7.4 (s, 1H), 7.55 (s, 1H), 7.7 (s, 1H), 8.1 (s, 1H), 8.65 (s, 1H). Mass Spectrum: M+H⁺ 533.

The N-(2,3-methylenedioxyphenyl)-2-{4-[6-(2-chloroethoxy)-7-methoxyquinazolin-4-yloxy]pyrazol-1-yl}acetamide used as a starting material was prepared as follows: —

A mixture of 4-chloro-6-hydroxy-7-methoxyquinazoline (International Patent Application WO 04/041829, within Example 7 thereof, 6.0 g), 1,2-dichloroethane (80 ml), potassium carbonate (7.87 g) and DMF (85 ml) was stirred and heated to 85° C. for 2 hours. The Resultant mixture was cooled to ambient temperature and filtered. The filtrate was evaporated to give a solid that was triturated under diethyl ether. There was thus obtained 4-chloro-6-(2-chloroethoxy)-7-methoxyquinazoline as a white solid (6.3 g); Mass Spectrum: M+H⁺ 273 and 275.

A mixture of the material so obtained, tert-butyl 2-(4-hydroxypyrazol-1-yl)acetate (4.55 g), potassium carbonate (4.83 g) and DMF (60 ml) was stirred and heated to 90° C. for 2 hours. The resultant mixture was filtered and the filtrate was evaporated. The residue was purified by column chromatography on silica using increasingly polar mixtures of methylene chloride and methanol as eluent. There was thus obtained tert-butyl 2-{4-[6-(2-chloroethoxy)-7-methoxyquinazolin-4-yloxy]pyrazol-1-yl}acetate as an oil (7-58 g); ¹H NMR: (DMSOd₆) 1.45 (s, 9H), 4.0 (s, 3M), 4.05 (m, 2H), 4.5 (m, 2H), 4.95 (s, 2H), 7.4 (s, 1H), 7.6 (s, 1H), 7.65 (s, 1H), 8.1 (s, 1H), 8.65 (s, 1H); Mass Spectrum: M+H⁺ 435 and 437.

A mixture of the material so obtained, trifluoroacetic acid (65 ml) and methylene chloride (50 ml) was stirred at ambient temperature for 2 hours. The resultant solution was evaporated. Toluene was added and the mixture was re-evaporated. The residue was triturated under diethyl ether. The solid so obtained was suspended in methylene chloride and sufficient diisopropylethylamine was added to obtain a solution. The resultant solution was evaporated and the solid so obtained was triturated under diethyl ether. The material so obtained was purified by column chromatography on silica using increasingly polar mixtures of methylene chloride and methanol as eluent. There was thus obtained 2-{4-[6-(2-chloroethoxy)-7-methoxyquinazolin-4-yloxy]pyrazol-1-yl}acetic acid (3.5 g); Mass Spectrum: M+H⁺ 379 and 381.

Using a similar procedure to that described in Example 32 except that the reaction mixture was stirred in a microwave oven at 100° C. for 3 minutes, 2-{4-[6-(2-chloroethoxy)-7-methoxyquinazolin-4-yloxy]pyrazol-1-yl}acetic acid (1 g) was reacted with 2,3-methylenedioxyaniline (0.435 g). On completion of the reaction, water was added and the resultant solid was isolated and washed with diethyl ether. The solid was dissolved in a mixture of methylene chloride and toluene and the solution was evaporated. The solid so obtained was dried-under vacuum. There was thus obtained the required starting material (0.8 g); ¹H NMR: (DMSOd₆) 4.0 (s, 3H), 4.05 (m, 2H), 4.5 (m, 2H), 5.1 (s, 2H), 6.05 (s, 2H), 6.75 (d, 1H), 6.8 (t, 1H), 7.3 (d, 1H), 7.4 (s, 1H), 7.6 (s, 1H), 7.65 (s, 1H), 8.1 (s, 1H), 8.65 (s, 1H); Mass Spectrum: M+H⁺ 498 and 500.

### EXAMPLE 51

### N-(2,3-methylenedioxyphenyl)-2-(4-{6-[2-(4-hydroxypiperidin-1-yl)ethoxy]-7-methoxyquinazolin-4-yloxy}pyrazol-1-yl)acetamide

Using an analogous procedure to that described in Example 50, N-(2,3-methylenedioxyphenyl)-2-{4-[6-(2-chloroethoxy)-7-methoxyquinazolin-4-yloxy]pyrazol-1-yl}acetamide was reacted with 4-hydroxypiperidine to give the title compound in 68% yield; ¹H NMR: (DMSOd₆) 1.4 (m, 2H), 1.7 (m, 2H), 2.15 (m, 2H), 2.75 (m, 2H), 2.8 (m, 2H), 3.45 (m, 1H), 4.0 (s, 3H), 4.25 (t, 2H), 4.55 (m, 1H), 5.1 (s, 2H), 6.05 (s, 2H), 6.75 (d, 1H), 6.8 (t, 1H), 7.3 (d, 1H), 7.4 (s, 1H), 7.6 (s, 1H), 7.65 (s, 1H), 8.1 (s, 1H), 8.65 (s, 1H); Mass Spectrum: M+H⁺ 563.

1. A quinazoline derivative of the Formula I 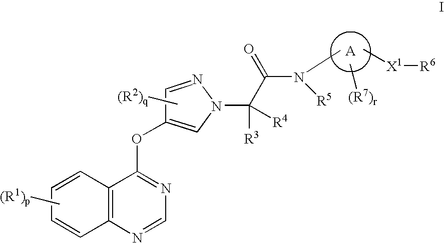
wherein p is 0, 1, 2 or 3;
each R¹ group, which may be the same or different, is selected from halogeno, trifluoromethyl, cyano, hydroxy, mercapto, amino, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkyl sulphinyl, (1-6C)alkylsulphonyl, (1-6C)alkylamino and di-[(1-6C)alkyl]amino,
or from a group of the formula:
Q¹-X²—
wherein X² is a direct bond or is selected from O, S, SO, SO₂, N(R⁸), CO, CON(R⁸), N(R⁸)CO, OC(R⁸)₂ and N(R⁸)C(R⁸)₂, wherein each R⁸ is hydrogen or (1-8C)alkyl, and Q¹ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,
and wherein any aryl, (3-8C)cycloalkyl, (3-8C)cycloalkenyl, heteroaryl or heterocyclyl group within a R¹ substituent optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
—X³—R⁹
wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl or N,N′,N′-tri-[(1-6C)alkyl]ureido-(1-6C)alkyl, or from a group of the formula:
—X⁴-Q²
wherein X⁴ is a direct bond or is selected from O, CO and N(R¹¹), wherein R¹¹ is hydrogen or (1-8C)alkyl, and Q² is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,
and wherein any aryl, heteroaryl or heterocyclyl group within a substituent on R¹ optionally bears a (1-3C)alkylenedioxy group,
and wherein any heterocyclyl group within a R¹ substituent optionally bears 1 or 2 oxo or thioxo substituents,
and wherein any CH, CH₂ or CH₃ group within a R¹ substituent optionally bears on each said CH, CH₂ or CH₃ group one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,
and wherein adjacent carbon atoms in any (2-6C)alkylene chain within a R¹ substituent are optionally separated by the insertion into the chain of a group selected from O, S, SO, SO₂, N(R¹²), CO, CH(OR¹²), CON(R¹²), N(R¹²)CO, N(R¹²)CON(R¹²), SO₂N(R¹²), N(R¹²)SO₂, CH═CH and C≡C wherein R¹² is hydrogen or (1-8C)alkyl, or, when the inserted group is N(R¹²), R¹² may also be (2-6C)alkanoyl;
q is 0, 1 or 2;
each R² group, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino and di-[(1-6C)alkyl]amino;
R³ is hydrogen, (1-8C)alkyl, (2-8C)alkenyl or (2-8C)alkynyl;
R⁴ is hydrogen, (1-8C)alkyl, (2-8C)alkenyl, (2-8C)alkynyl, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl, di-[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkoxycarbonyl-(1-6C)alkyl, (2-6C)alkanoylamino-(1-6C)alkyl or N-(1-6C)alkyl-(2-6C)alkanoylamino-(1-6C)alkyl;
or R³ and R⁴ together with the carbon atom to which they are attached form a (3-8C)cycloalkyl group;
R⁵ is hydrogen, (1-8C)alkyl, (2-8C)alkenyl or (2-8C)alkynyl or a group of the formula:
—X⁵—R¹³
wherein X⁵ is a direct bond or is selected from O and N(R¹⁴), wherein R¹⁴ is hydrogen or (1-8C)alkyl, and R¹³ is halogeno-(1-6C)alkyl, hydroxy-(1-6C)alkyl, (1-6C)alkoxy-(1-6C)alkyl or cyano-(1-6C)alkyl;
Ring A is a 6-membered monocyclic or a 10-membered bicyclic aryl ring or a 5- or 6-membered monocyclic or a 9- or 10-membered bicyclic heteroaryl ring with up to three ring heteroatoms selected from oxygen, nitrogen and sulphur;
X¹ is a direct bond or is selected from O, S, SO, SO₂, N(R¹⁵), CO, CH(OR¹⁵), CON(R¹⁵), N(R¹⁵)CO, N(R¹⁵)CON(R¹⁵), SO₂N(R¹⁵), N(R¹⁵)SO₂, C(R¹⁵)₂O, C(R¹⁵)₂S, C(R¹⁵)₂N(R¹⁵) and C(R¹⁵)₂C(R¹⁵)₂N(R¹⁵), wherein each R¹⁵ is hydrogen or (1-8C)alkyl;
R⁶ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, carboxy-(1-6C)alkyl, (1-6C)alkoxycarbonyl-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulphamoyl-(1-6C)alkyl, N-(1-6C)alkylsulphamoyl-(1-6C)alkyl, N,N-di-[(1-6C)alkyl]sulphamoyl-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl, N,N′,N′-tri-[(1-6C)alkyl]ureido-(1-6C)alkyl, (1-6C)alkanesulphonylamino-(1-6C)alkyl or N-(1-6C)alkyl-(1-6C)alkanesulphonylamino-(1-6C)alkyl,
or R⁶ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,
or, when X¹ is a direct bond, R⁶ may be carboxy, (1-6C)alkoxycarbonyl, sulphamoyl, N-(1-6C)alkylsulphamoyl or N,N-di-[(1-6C)alkyl]sulphamoyl,
or the —X¹—R⁶ group and one R⁷ group together form a bivalent group that spans adjacent ring positions on Ring A selected from OC(R²⁰)₂O, OC(R²⁰)₂C(R²⁰)₂O, OC(R²⁰)₂C(R²⁰)₂, C(R²⁰)₂OC(R²)₂, C(R²⁰)₂C(R²⁰)₂C(R²⁰)₂, C(R²⁰)₂C(R²⁰)₂C(R²⁰)₂C(R²⁰)₂, OC(R²⁰)₂N(R²¹), OC(R²⁰)₂C(R²¹)₂N(R²¹), N(R²¹)C(R²⁰)₂N(R²¹), N(R²¹)C(R²⁰)₂C(R²⁰)₂, N(R²¹)C(R²⁰)₂C(R²⁰)₂C(R²⁰)₂, C(R²⁰)₂N(R²¹)C(R²⁰)₂, CO.N(R²¹)C(R²⁰)₂ N(R²¹)CO.C(R²⁰)₂, N(R²¹)C(R²⁰)₂CO, CO.N(R²¹)CO, N(R²¹)N(R²¹)CO, N(R²¹)CO.N(R²¹), O.CO.N(R²¹), O.CO.C(R²⁰)₂ and CO.OC(R²⁰)₂ wherein each R²⁰ is hydrogen, (1-8C)alkyl, (2-8C)alkenyl or (2-8C)alkynyl, and wherein R²¹ is hydrogen, (1-8C)alkyl, (2-8C)alkenyl, (2-8C)alkynyl or (2-6C)alkanoyl,
and wherein any aryl, (3-8C)cycloalkyl, (3-8C)cycloalkenyl, heteroaryl or heterocyclyl group within the R⁶ group optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
—X⁶—R¹⁶
wherein X⁶ is a direct bond or is selected from O and N(R¹⁷), wherein R¹⁷ is hydrogen or (1-8C)alkyl, and R¹⁶ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:
—X⁷-Q³
wherein X⁷ is a direct bond or is selected from O, CO and N(R¹⁸), wherein R¹⁸ is hydrogen or (1-8C)alkyl, and Q³ is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,
and wherein any heterocyclyl group within the R⁶ group optionally bears 1 or 2 oxo or thioxo substituents,
and wherein any CH, CH₂ or CH₃ group within the R⁶ group optionally bears on each said CH, CH₂ or CH₃ group one or more halogeno or (1-8C)alkyl substituents and/or a substituent selected from hydroxy, mercapto, amino, cyano, carboxy, carbamoyl, ureido, (2-8C)alkenyl, (2-8C)alkynyl, (1-6C)alkoxy, (176C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2′-6C)alkanoyloxy, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,
and wherein adjacent carbon atoms in any (2-6C)alkylene chain within the R⁶ group are optionally separated by the insertion into the chain of a group selected from O, S, SO, SO₂, N(R¹⁹), N(R¹⁹)CO, CON(R¹⁹), N(R¹⁹)CON(R¹⁹), CO, CH(OR¹⁹), N(R¹⁹)SO₂, SO₂N(R¹⁹), CH═CH and C≡C wherein R¹⁹ is hydrogen or (1-8C)alkyl;
r is 0, 1 or 2; and
each R⁷ group, which may be the same or different, is selected from halogeno, trifluoromethyl, cyano, hydroxy, mercapto, amino, carboxy, carbamoyl, sulphamoyl, ureido, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylsulphamoyl, N,N-di-[(1-6C)alkyl]sulphamoyl, (1-6C)alkanesulphonylamino, N-(1-6C)alkyl-(1-6C)alkanesulphonylamino, hydroxy-(1-6C)alkyl and (1-6C)alkoxy-(1-6C)alkyl;
or a pharmaceutically-acceptable salt, solvate or pro-drug thereof.

2. A quinazoline derivative of the Formula I according to claim 1 wherein Ring A is a 6-membered monocyclic aryl ring or a 5- or 6-membered monocyclic heteroaryl ring with up to three ring heteroatoms selected from oxygen, nitrogen and sulphur.

3. A quinazoline derivative of the Formula I according to claim 1 wherein X¹ is a direct bond or is selected from O, S, SO, SO₂, N(R¹⁵) and CO, wherein R¹⁵ is hydrogen, methyl or ethyl, provided that, when a heteroatom in an X¹ group is attached to the R⁶ group, there are at least two carbon atoms between the heteroatom in the X¹ group and any heteroatom in the R⁶ group.

4. A quinazoline derivative of the Formula I according to claim 1 wherein X¹ is a direct bond.

5. A quinazoline derivative of the Formula I according to claim 1 wherein R⁶ is hydroxy-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or R⁶ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,
and wherein any aryl, (3-8C)cycloalkyl, heteroaryl or heterocyclyl group within the R⁶ group optionally bears 1 or 2 substituents, which may be the same or different, selected from halogeno, trifluoromethyl, cyano, hydroxy, amino, (1-8C)alkyl, (1-6C)alkoxy, (1-6C)alkylamino and di-[(1-6C)alkyl]amino, or from a group of the formula:
—X⁶—R¹⁶
wherein X⁶ is a direct bond and R¹⁶ is halogeno-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl or di-[(1-6C)alkyl]amino-(1-6C)alkyl,
and wherein any CH, CH₂ or CH₃ group within the R⁶ group optionally bears on each said CH, CH₂ or CH₃ group 1, 2 or 3 halogeno or (1-8C)alkyl substituents and/or a substituent selected from hydroxy, amino, cyano, (3-8C)alkenyl, (3-8C)alkynyl, (1-6C)alkoxy, (1-6C)alkylsulphonyl, (1-6C)alkylamino, di-[(1-6C)alkyl]amino, (2-6C)alkanoylamino and N-(1-6C)alkyl-(2-6C)alkanoylamino.

6. A quinazoline derivative of the Formula I according to claim 1 wherein the —X¹—R⁶ group and one R⁷ group together form a bivalent group that spans adjacent ring positions on Ring A selected from OC(R²⁰)₂O, OC(R²⁰)₂C(R²⁰)₂, C(R²⁰)₂OC(R²⁰)₂, C(R²⁰)₂C(R²⁰)₂C(R²⁰)₂, C(R²⁰)₂C(R²⁰)₂C(R²⁰)₂C(R²⁰)₂, OC(R²⁰)₂N(R²¹), OC(R²⁰)₂C(R²⁰)₂N(R²¹), N(R²¹)C(R²⁰)₂N(R²¹), N(R²¹)C(R²⁰)₂C(R²⁰)₂, N(R²¹)C(R²⁰)₂C(R²⁰)₂C(R²⁰)₂ and C(R²⁰)₂N(R²¹)C(R²⁰)₂, wherein each of R²⁰ and R²¹ is hydrogen, (1-8C)alkyl, (2-8C)alkenyl or (2-8C)alkynyl.

7. A quinazoline derivative of the Formula I according to claim 1 wherein
p is 2 and the first R¹ group is a 6-methoxy group and the second R¹ group is located at the 7-position and is selected from methoxy, ethoxy, 2-methoxyethoxy, 3-methoxypropoxy, 2-methylsulphonylethoxy, 3-methylsulphonylpropoxy, 2-(2-methoxyethoxy)ethoxy, 2-pyrrolidin-1-ylethoxy, 3-pyrrolidin-1-ylpropoxy, 2-[(3RS,4SR)-3,4-methylenedioxypyrrolidin-1-yl]ethoxy, 3-[(3RS,4SR)-3,4-methylenedioxypyrrolidin-1-yl]propoxy, 2-morpholinoethoxy, 3-morpholinopropoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 2-piperidin-3-ylethoxy, 2-(N-methylpiperidin-3-yl)ethoxy, 3-piperidin-3-ylpropoxy, 3-(N-methylpiperidin-3-yl)propoxy, 2-piperidin-4-ylethoxy, 2-(N-methylpiperidin-4-yl)ethoxy, 3-piperidin-4-ylpropoxy, 3-(N-methylpiperidin-4-yl)propoxy, 2-(1,2,3,6-tetrahydropyridin-1-yl)ethoxy, 3-(1,2,3,6-tetrahydropyridin-1-yl)propoxy, 2-(4-hydroxypiperidin-1-yl)ethoxy, 3-(4-hydroxypiperidin-1-yl)propoxy, 2-piperazin-1-ylethoxy, 3-piperazin-1-ylpropoxy, 4-piperazin-1-ylbutoxy, 2-(4-methylpiperazin-1-yl)ethoxy, 3-(4-methylpiperazin-1-yl)propoxy, 4-(4-methylpiperazin-1-yl)butoxy, 2-(4-allylpiperazin-1-yl)ethoxy, 3-(4-allylpiperazin-1-yl)propoxy, 2-(4-prop-2-ynylpiperazin-1-yl)ethoxy, 3-(4-prop-2-ynylpiperazin-1-yl)propoxy, 2-(4-methylsulphonylpiperazin-1-yl)ethoxy, 3-(4-methylsulphonylpiperazin-1-yl)propoxy, 2-(4-acetylpiperazin-1-yl)ethoxy, 3-(4-acetylpiperazin-1-yl)propoxy, 4-(4-acetylpiperazin-1-yl)butoxy, 2-(4-isobutyrylpiperazin-1-yl)ethoxy, 3-(4-isobutyrylpiperazin-1-yl)propoxy, 4-(4-isobutyrylpiperazin-1-yl)butoxy, 2-[4-(2-fluoroethyl)piperazin-1-yl]ethoxy, 3-[4-(2-fluoroethyl)piperazin-1-yl]propoxy, 2-[4-(2,2,2-trifluoroethyl)piperazin-1-yl]ethoxy, 3-[4-(2,2,2-trifluoroethyl)piperazin-1-yl]propoxy, 2-(4-cyanomethylpiperazin-1-yl)ethoxy, 3-(4-cyanomethylpiperazin-1-yl)propoxy, 2-[2-(4-methylpiperazin-1-yl)ethoxy]ethoxy, 2-(4-pyridyloxy)ethoxy, 3-pyridylmethoxy and 2-cyanopyrid-4-ylmethoxy;
q is 0;
each of R³, R⁴ and R⁵ is hydrogen;
Ring A is phenyl or pyridyl and the —X¹—R⁶ group is located at the 3- or 4-position (relative to the CON(R⁵) group);
X¹ is a direct bond or O, provided that, when X¹ is O, there are at least two carbon atoms between that O heteroatom and any heteroatom in the R⁶ group;
R⁶ is hydroxymethyl, 1-hydroxyethyl, 2-hydroxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, dimethylaminomethyl, 1-dimethylaminoethyl or 2-dimethylaminoethyl, or R⁶ is phenyl, benzyl, cyclopropyl, cyclopentyl, cyclohexyl, pyrrolidinyl, morpholinyl, tetrahydro-1,4-thiazinyl, piperidinyl, piperazinyl, pyrrolidinylmethyl, morpholinylmethyl, piperidinylmethyl or piperazinylmethyl,
and wherein any aryl, (3-8C)cycloalkyl or heterocyclyl group within the R⁶ group optionally bears a substituent selected from fluoro, chloro, trifluoromethyl, hydroxy, amino, methyl, methoxy, methylamino and dimethylamino; and
r is 0 or r is 1 and the R⁷ group is located at an available 3- or 4-position (relative to the CON(R⁵) group) and is selected from fluoro, chloro, trifluoromethyl, hydroxy, amino, methyl, methoxy, methylamino and dimethylamino;
or a pharmaceutically-acceptable salt, solvate or pro-drug thereof.

8. A quinazoline derivative of the Formula I according to claim 1 wherein: —
p is 2 and the first R¹ group is a 6-methoxy group and the second R¹ group is located at the 7-position and is selected from methoxy, ethoxy and 2-methoxyethoxy;
q is 0;
each of R³, R⁴ and R⁵ is hydrogen;
Ring A is phenyl and the —X¹—R⁶ group is located at the 3- or 4-position (relative to the CON(R⁵) group) and is selected from hydroxymethyl, 1-hydroxyethyl, 2-hydroxyethyl, methylaminomethyl, 1-methylaminoethyl, 2-methylaminoethyl, dimethylaminomethyl, 1-dimethylaminoethyl and 2-dimethylaminoethyl, or Ring A is 3-pyridyl and the —X¹—R⁶ group is located at the 4-position (each relative to the CON(R⁵) group) and is a 2-dimethylaminoethoxy group; and
r is 0 or r is 1 and the R⁷ group is located at an available 3- or 4-position (relative to the CON(R⁵) group) and is selected from fluoro, chloro, methoxy, methylamino and dimethylamino;
or a pharmaceutically-acceptable salt, solvate or pro-drug thereof.

9. A quinazoline derivative of the Formula I according to claim 1 wherein: —
p is 2 and the R¹ groups, which may be the same or different, are located at the 6- and 7-positions and are selected from methoxy, ethoxy, propoxy, 2-hydroxyethoxy, 3-hydroxypropoxy, 2-methoxyethoxy, 3-methoxypropoxy, 2-methylsulphonylethoxy, 3-methylsulphonylpropoxy and 2-(2-methoxyethoxy)ethoxy;
q is 0 or q is 1 and the R² group is fluoro, chloro, methyl or methoxy;
each of R³, R⁴ and R⁵ is hydrogen;
Ring A is phenyl and the —X¹—R⁶ group is located at the 3- or 4-position (relative to the CON(R⁵) group) and is selected from hydroxymethyl, aminomethyl, methylaminomethyl, ethylaminomethyl, propylaminomethyl, isopropylaminomethyl, cyclopropylaminomethyl, cyclopropylmethylaminomethyl, dimethylaminomethyl, diethylaminomethyl, N-ethyl-N-methylaminomethyl, N-cyclopropyl-N-methylaminomethyl, N-cyclopropylmethyl-N-methylaminomethyl, azetidin-1-ylmethyl, pyrrolidin-1-ylmethyl, morpholinylmethyl, piperidinylmethyl, homopiperidinylmethyl, piperazinylmethyl, homopiperazinylmethyl and 2-methoxyethoxy; and
r is 0 or r is 1 and any R⁷ group that is present is located at an available 3-, 4- or 5-position (relative to the CON(R⁵) group) and is selected from fluoro, chloro, methyl, ethyl, methoxy, methylamino and dimethylamino;
or a pharmaceutically-acceptable salt, solvate or pro-drug thereof.

10. A quinazoline derivative of the Formula I according to claim 1 wherein: —
p is 2 and the first R¹ group is a 6-methoxy group and the second R¹ group is located at the 7-position and is selected from methoxy, ethoxy, 2-hydroxyethoxy and 2-methoxyethoxy;
q is 0 or q is 1 and the R² group is fluoro;
each of R³, R⁴ and R⁵ is hydrogen;
Ring A is 2-pyridyl and the —X¹—R⁶ group is located at the 4-, 5- or 6-position (relative to the pyridyl nitrogen heteroatom) and is selected from cyclopropylamino, 2-hydroxyethylamino, 2-methoxyethylamino, N-cyclopropyl-N-methylamino, pyrrolidin-1-yl, piperidino, morpholino, piperazin-1-yl, methylaminomethyl, ethylaminomethyl, propylaminomethyl, isopropylaminomethyl, cyclopropylaminomethyl, dimethylaminomethyl, N-ethyl-N-methylaminomethyl and N-cyclopropyl-N-methylaminomethyl;
and r is 0;
or a pharmaceutically-acceptable salt, solvate or pro-drug thereof.

11. A process for the preparation of a quinazoline derivative of the Formula I, or a pharmaceutically-acceptable salt, solvate or pro-drug thereof, according to claim 1 which comprises
(a) the reaction of a quinazoline of the Formula II 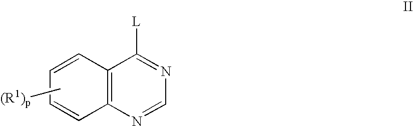
wherein L is a displaceable group and p and R¹ have any of the meanings defined in claim 1 except that any functional group is protected if necessary, with a pyrazole of the Formula III 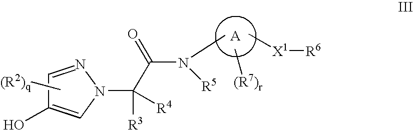
wherein q, R², R³, R⁴, R⁵, Ring A, X¹, R⁶, r and R⁷ have any of the meanings defined in claim 1 except that any functional group is protected if necessary, whereafter any protecting group that is present is removed;
(b) the coupling of a quinazoline of the Formula VII 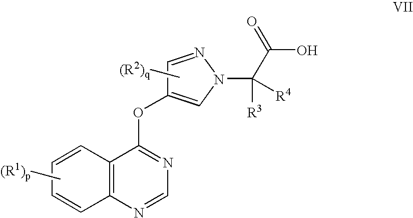
or a reactive derivative thereof, wherein p, R¹, q, R², R³ and R⁴ have any of the meanings defined in claim 1 except that any functional group is protected if necessary, with an amine of the Formula VI 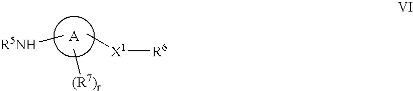
wherein R⁵, Ring A, X¹, R⁶, r and R⁷ have any of the meanings defined in claim 1 except that any functional group is protected if necessary, whereafter any protecting group that is present is removed;
(c) for the production of those compounds of the Formula I wherein at least one R¹ group is a group of the formula
Q¹-X²—
wherein Q¹ is an aryl-(1-6C)alkyl, (3-7C)cycloalkyl-(1-6C)alkyl, (3-7C)cycloalkenyl-(1-6C)alkyl, heteroaryl-(1-6C)alkyl or heterocyclyl-(1-6C)alkyl group or an optionally substituted alkyl group and X² is an oxygen atom, the coupling of a quinazoline of the Formula VI 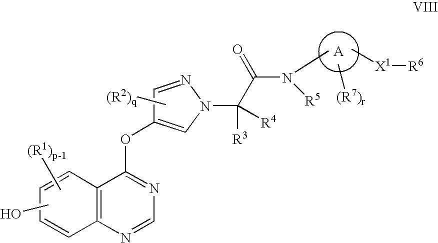
wherein each of p, R¹, q, R², R³, R⁴, R⁵, Ring A, X¹, R⁶, r and R⁷ has any of the meanings defined in claim 1 except that any functional group is protected if necessary, with an appropriate alcohol wherein any functional group is protected if necessary, whereafter any protecting group that is present is removed;
(d) for the production of those compounds of the Formula I wherein the —X¹—R⁶ group is an amino-substituted (1-6C)alkyl group, the reaction of a compound of the Formula I wherein the —X¹—R⁶ group is a halogeno-substituted (1-6C)alkyl group with an appropriate amine or with a nitrogen-containing heterocyclyl compound;
(e) for the production of those compounds of the Formula I wherein the —X¹—R⁶ group is an amino-substituted (1-6C)alkyl group, the reductive amination of a compound of the Formula I wherein the —X¹—R⁶ group is a formyl or (2-6C)alkanoyl group; or
(f) for the production of those compounds of the Formula I wherein an R¹ group is an amino-substituted or heterocyclyl-substituted (1-6C)alkoxy group, the reaction of a compound of the Formula I wherein the R¹ group is a halogeno-substituted (1-6C)alkoxy group with an appropriate amine or with a nitrogen-containing heterocyclyl compound;
and when a pharmaceutically-acceptable salt of a quinazoline derivative of the Formula I is required it may be obtained by reaction of said quinazoline derivative with a suitable acid;
and when a pharmaceutically-acceptable pro-drug of a quinazoline derivative of the Formula I is required, it may be obtained using a conventional procedure.

12. A pharmaceutical composition which comprises a quinazoline derivative of the Formula I, or a pharmaceutically-acceptable salt, solvate or pro-drug thereof, according to claim 1 in association with a pharmaceutically-acceptable diluent or carrier.

13. The use of a quinazoline derivative of the Formula I, or a pharmaceutically-acceptable salt, solvate or pro-drug thereof, according to claim 1 in the manufacture of a medicament for use in the treatment of cell proliferative disorders or in the treatment of disease states associated with angiogenesis and/or vascular permeability.

14. A method for the treatment of cell proliferative disorders in a warm-blooded animal in need of such treatment or for the treatment of disease states associated with angiogenesis and/or vascular permeability in a warm-blooded animal in need of such treatment which comprises administering to said animal an effective amount of a quinazoline derivative of the Formula I, or a pharmaceutically-acceptable salt, solvate or pro-drug thereof, according to claim 1.
